(19) United States
(12) Patent Application Publication
(10) Pub. No.: US 2018/0016319 A1
(43) Pub. Date: Jan. 18, 2018

(54) Acylated Glucagon Analogues

(71) Applicant: Novo Nordisk A/S, Bagsvaerd (DK)

(72) Inventors: Jesper F. Lau, Farum (DK); Thomas Kruse, Herlev (DK); Lars Linderoth, Hilleroed (DK); Henning Thoegersen, Farum (DK)

(21) Appl. No.: 15/660,458

(22) Filed: Jul. 26, 2017

Related U.S. Application Data
(63) Continuation of application No. 15/155,541, filed on May 16, 2016, now abandoned.
Application No. 15/155,541 is a continuation of application No. 14/739,614, filed on Jun. 15, 2015, now abandoned.
Application No. 14/739,614 is a continuation of application No. 13/637,522, filed on Oct. 16, 2012, now abandoned.
(60) Provisional application No. 61/319,994, filed on Apr. 1, 2010.

(30) Foreign Application Priority Data
Mar. 26, 2010 (EP) 10157901.9

(51) Int. Cl. C07K 14/605; A61K 38/26; A61K 38/28; A61K 38/00

(52) U.S. Cl. CPC C07K 14/605; A61K 38/28; A61K 47/545; A61K 38/26; A61K 38/00

(57) ABSTRACT

The present invention relates to novel peptide compounds which have an improved physical stability in solution and improved solubility at neutral pH, to the use of the compounds in therapy, to methods of treatment comprising administration of the compounds to patients in need thereof, and to the use of the compounds in the manufacture of medicaments. The compounds of the invention are of particular interest in relation to the treatment of hyperglycemia, diabetes and obesity, as well as a variety of diseases or conditions associated with hyperglycemia, diabetes and obesity.

### CROSS-REFERENCE TO RELATED APPLICATIONS

This application is a continuation of Ser. No. 15/155,541, filed May 16, 2016, which is a continuation of Ser. No. 14/739,614, filed Jun. 15, 2015 (abandoned) which is a continuation of Ser. No. 13/637,522, filed Oct. 16, 2012 (abandoned) which is a 35 U.S.C. §371 national stage application of International Patent Application PCT/EP2011/054714 (published as WO 2011/117416 A1), filed Mar. 28, 2011, which claims priority to European Patent Application 10157901.9, filed Mar. 26, 2010; this application further claims priority under 35 U.S.C. §119 to U.S. Provisional Application 61/319,994, filed Apr. 1, 2010, the contents of which are incorporated herein by reference.

### FIELD OF THE INVENTION

The present invention relates to novel glucagon peptide analogues with improved physical stability and solubility, and a protracted profile of action, to the use of said peptides in therapy to methods of treatment comprising administration of said peptides to patients, and to the use of said peptides in the manufacture of medicaments.

### INCORPORATION-BY-REFERENCE OF THE SEQUENCE LISTING

The instant application contains a sequence listing which has been submitted in ASCII format via EFS-WEB and is hereby incorporated by reference in its entirety. Said ASCII copy, created on May 5, 2016 and modified Jul. 14, 2017, and is named 8342US03_SeqList_ST25.txt and is 845 bytes in size.

### BACKGROUND OF THE INVENTION

The precise control of blood glucose levels is of vital importance to humans as well as other mammals. It is well established that the two hormones insulin and glucagon are important for maintenance of correct blood glucose levels. While insulin acts in the liver and peripheral tissues by reducing blood glucose levels via increased peripheral uptake of glucose and reduced glucose output from the liver, glucagon acts mainly on the pancreas and liver, by increasing blood glucose levels via up-regulation of gluconeogenesis and glycogenolysis.

Glucagon has also been reported to increase lipolysis, to induce ketosis and to reduce plasma triglyceride levels in plasma [Schade and Eaton, Acta Diabetologica, 1977, 14, 62].

Glucagon is an important part of the defence mechanism against hypoglycaemia and administration of a low dose of glucagon may prevent insulin-induced hypoglycaemia or improve the ability to recover from hypoglycaemia. Studies have also shown that glucagon does reduce food intake and body weight in rats and in humans [Schulman et al. J. Appl. Physiol. 1957, 11, 419]. Hence, glucagon is a plausible signal that may contribute to the termination of food intake. Furthermore, administration of a lower dose of glucagon may induce satiety without affecting the blood glucose. A large number of people suffering from diabetes, in particular Type 2 diabetes, are over-weight or obese. Obesity represents a high risk factor in serious and even fatal common diseases and for most diabetics it is highly desirable that their treatment does not cause weight gain.

Glucagon is however of limited potential use in pharmaceuticals due to fast clearance from circulation with a half live of approximately 5 min. A high clearance of a therapeutic agent is inconvenient in cases where it is desired to maintain a high blood level thereof over a prolonged period of time since repeated administrations will then be necessary. In some cases it is possible to influence the release profile of peptides by applying suitable pharmaceutical compositions, but this approach has various shortcomings and is not generally applicable.

Glucagon is currently available in recombinant form as a freeze-dried formulation, with a short duration of action, restricted to a few hours in spite of a glucagon level that peaks at levels far higher than endogenous glucagon levels. There is therefore a need for chemically modified glucagon compounds in order to be delivered at continuous levels, so that longer biological half-life is achieved, i.e. modified glucagon peptides with a protracted profile of action.

Furthermore, glucagon is not stable for very long when dissolved in aqueous solution since physical stability of glucagon is very poor and solutions of glucagon form gels and fibrils within hours or days (Beaven et al. European J. Biochem. 1969, 11, 37-42), depending on purity of the peptide, salt concentration, pH and temperature. In addition the solubility of human glucagon is very poor at pH 3.5-9.5.

Several patent applications disclosing different glucagon-based analogues and GLP-1/glucagon receptor co-agonists are known in the art, such as e.g. patents WO2008/086086, WO2008/101017, WO2007/056362, WO2008/152403 and WO96/29342. Some of the GLP-1/glucagon receptor co-agonists disclosed in these patents refer to specific mutations relative to native human glucagon. Other glucagon analogs disclosed are PEGylated (e.g. WO2007/056362) or acylated in specific positions of native human glucagon (e.g. WO96/29342). Glucagon for prevention of hypoglycaemia have been disclosed, as e.g. in U.S. Pat. No. 7,314,859.

The peptides of the present invention provide novel modified glucagon peptides with a protracted profile of action in addition to providing such modified glucagon peptides in stable pharmaceutical compositions at physiological pH.

### SUMMARY OF THE INVENTION

The present invention relates to novel glucagon peptides with improved physical stability and solubility at neutral pH, to the use of said peptides in therapy, to methods of treatment comprising administration of said peptides to patients, and to the use of said peptides in the manufacture of medicaments for use in the treatment of diabetes, obesity and related diseases and conditions.

The present inventors have surprisingly found a number of positions in human glucagon where attachment of a substituent comprising three or more negative charged moieties wherein one of the said negatively charged moieties is distal of a lipohilic moiety, leads to glucagon agonists with improved physical stability and solubility.

In a first embodiment (Embodiment 1), the present invention relates to a glucagon peptide comprising SEQ ID 1, up to seven amino acid substitutions in said glucagon peptide and a substituent comprising three or more negatively charged moieties, wherein one of said negatively charged moieties is distal of a lipohilic moiety, and wherein said substituent is attached at the epsilon position of a Lys, at the delta position of an Orn or at the sulphur of a Cys, in one or more of the following amino acid positions of said glucagon peptide: X₁₀, X₁₂, X₁₆, X₁₇, X₁₈, X₂₀, X₂₁, X₂₄, X₂₅, X₂₇, X₂₈, X₂₉, and/or X₃₀

or a pharmaceutically acceptable salt, amide, acid or prodrug thereof.

The present invention further relates to the use of the compounds of the present invention in therapy, to pharmaceutical compositions comprising compounds of the invention and the use of the compounds of the invention in the manufacture of medicaments.

### DESCRIPTION OF THE DRAWINGS

FIG. 1 shows pH dependent solubility (Assay VIII) of glucagon (1, black line) and example 3 (2, grey line).

FIG. 2 shows the accumulated food intake in rats after sc adm. of 100 nmol/kg, 300 nmol/kg or 1000 nmol/kg glucagon analogue of Example 3. Data=Mean+/−SEM, n=5-6.

FIG. 3 shows the accumulated food intake in rats after sc adm. of 300 nmol/kg of glucagon analogue of Example 4. Data=Mean+/−SEM, n=5-6

FIG. 4 shows the accumulated food intake in rats after sc. adm. of 300 nmol/kg of glucagon analogue of Example 5. Data=Mean+/−SEM, n=5-6.

FIG. 5 shows the PK of glucagon analogue of Example 3, after iv. and sc. dosing in rats. Half life (iv.)˜8.6 h±0.5, Half life (sc.)˜9.4 h±0.9, mean±SEM.

FIG. 6 shows the reduction of body weight in diet induced obese (D10) rats dosed with glucagon analogue of Example 3 alone, or with GLP-1 analogue G3. Stippled lines indicate start of dosing and reduction of doses, respectively.

FIG. 7 shows delta body weight at day 14 in diet induced obese rats dosed with glucagon analogue of Example 3 alone, or with GLP-1 analogue G3. Bars show significant difference (1-way ANOVA, Bonferroni's post-test)

FIG. 8 shows blood glucose profiles 11^{th} day of dosing in diet induced obese rats dosed with glucagon analogue of Example 3 alone, or with GLP-1 analogue G3. Stippled lines indicate dosing.

FIG. 9 shows food intake in diet induced obese rats in diet induced obese rats dosed with glucagon analogue of Example 3 alone, or with GLP-1 analogue G3.

FIG. 10 shows insulin levels measured at the end of the study in diet induced obese rats dosed with glucagon analogue of Example 3 alone, or with GLP-1 analogue G3. Groups are compared using 1-way ANOVA and Dunnet's post-test comparing groups to vehicle high fat fed group.

FIG. 11 shows cholesterol levels measured at the end of the study in diet induced obese rats dosed with glucagon analogue of Example 3 alone, or with GLP-1 analogue G3. Groups are compared using 1-way ANOVA and Dunnet's post-test comparing groups to vehicle high fat fed group.

FIG. 12 shows the solubility of glucagon analogues in 10 mM HEPES buffer (pH=7.5). Buffer was added to glucagon analogues to a nominal concentration of 250 μM and the concentration was measured after one hour, upon centrifugation. The concentrations were assessed using a chemiluminescent nitrogen specific HPLC detector.

FIG. 13 shows the stability of glucagon analogues. Glucagon analogues were added buffer to a nominal concentration of 250 μM and a UPLC chromatogram was recorded after one hour. The solutions were kept for 6 days at 30° C. whereupon the samples were filtered and a new UPLC was recorded. The areas under the curves of the peaks (214 nM) were used as a measure of concentration of peptide in solution.

FIG. 14 shows the lag time (left Y-axis) and recovery (right Y-axis) obtained in a ThT (thioflavin T) fibrillation assay. Column 1: Lag time and recovery for Formulation 1. Column 2A: Lag time and recovery of glucagon analogue of Example 3 in Formulation 2. Column 2B: Recovery of insulin analogue G5 in Formulation 2. Column 3A: Lag time and recovery of glucagon analogue of Example 3 in Formulation 3. Column 3B: Recovery of GLP-1 analogue G1 in formulation 3. Column 4: Lag time and recovery of glucagon analogue of Example 3 in Formulation 4 (GLP-1 analogue G3 recovery not determined due to technical reasons). Column 5: Lag time and recovery for insulin analogue G5 in Formulation 5. Column 6: Lag time and recovery for GLP-1 analogue G1 in Formulation 6.

FIG. 15 shows GLP-1, glucagon and glucagon analogue of Example 3, incubated with DPP-IV (2 μg/ml) at 37° C. in a HEPES buffer. The half-lives were determined to 11 min, 32 min and 260 min, respectively.

FIG. 16 shows food intake in rats after single sc. administration of glucagon analogues of example 53 and 54 (Assay V).

FIG. 17 shows pharmacokinetic profile of glucagon analogue of example 51 after single SC or IV administration in rats. Assay (VII).

FIG. 18 shows food intake in rats after single sc. administration of glucagon analogues of example 51 (Assay V).

FIG. 19 shows pH dependent solubility (Assay VIII) of native glucagon (black) and example 51 (grey).

FIG. 20 depicts ThT fluorescence as a function of time t.

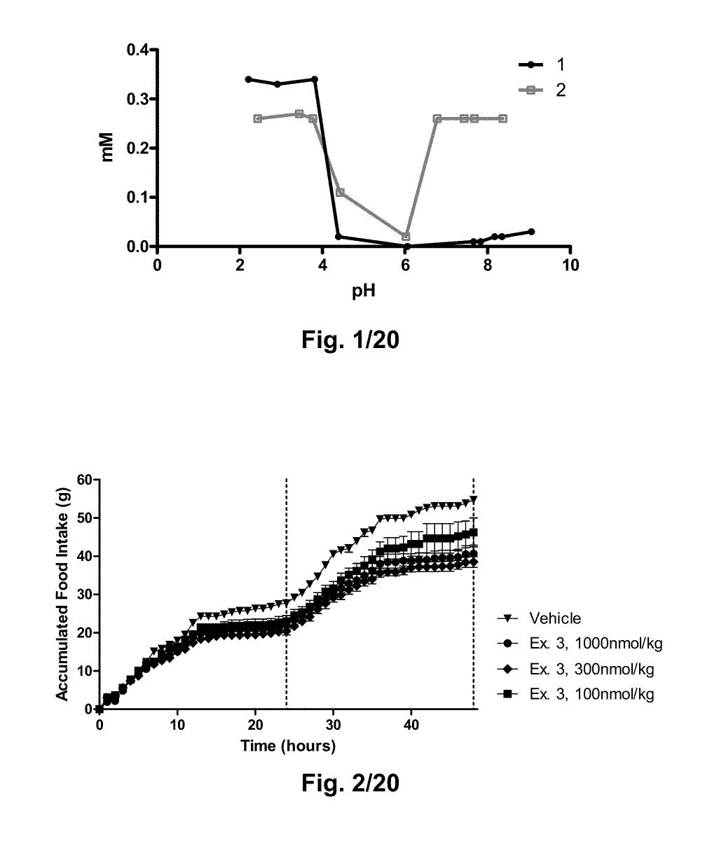

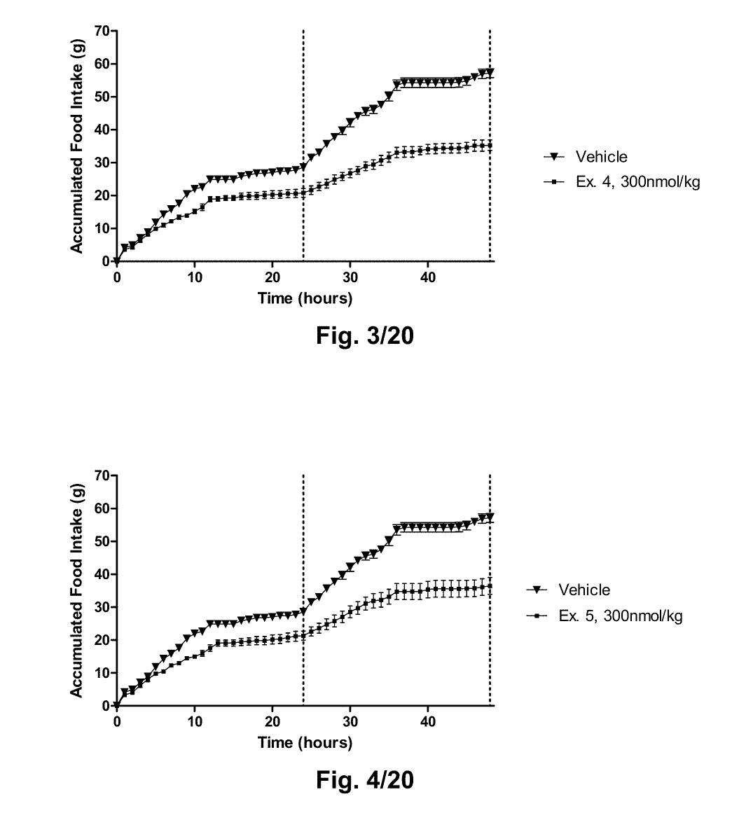

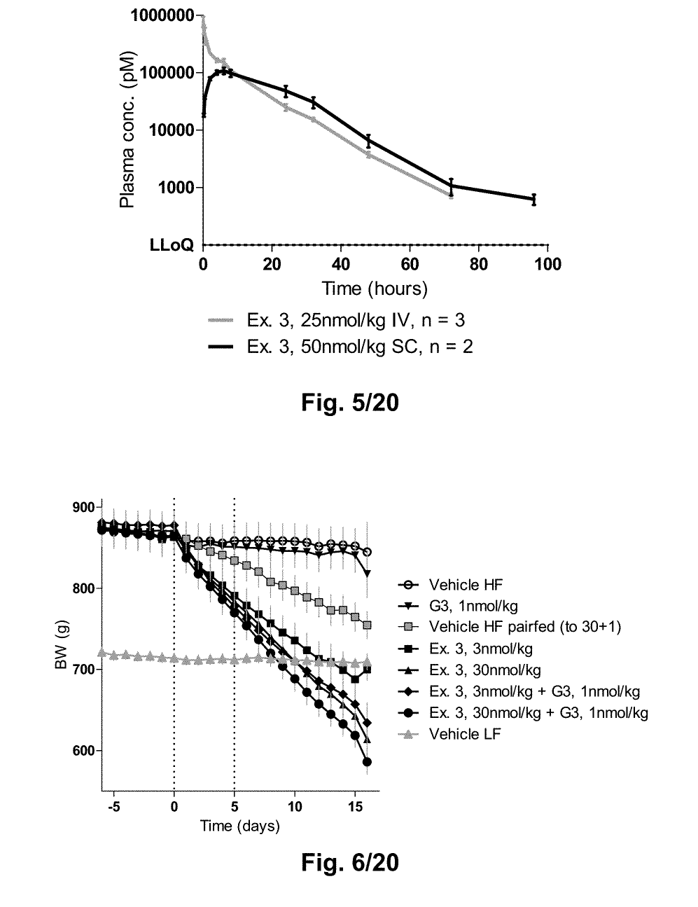

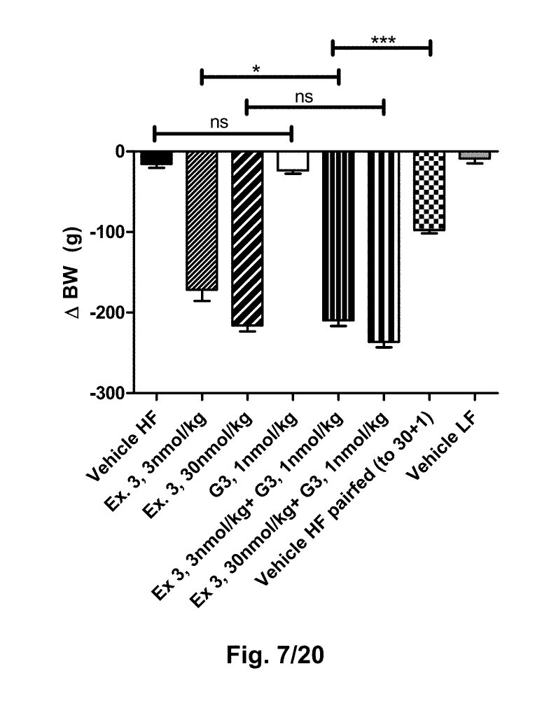

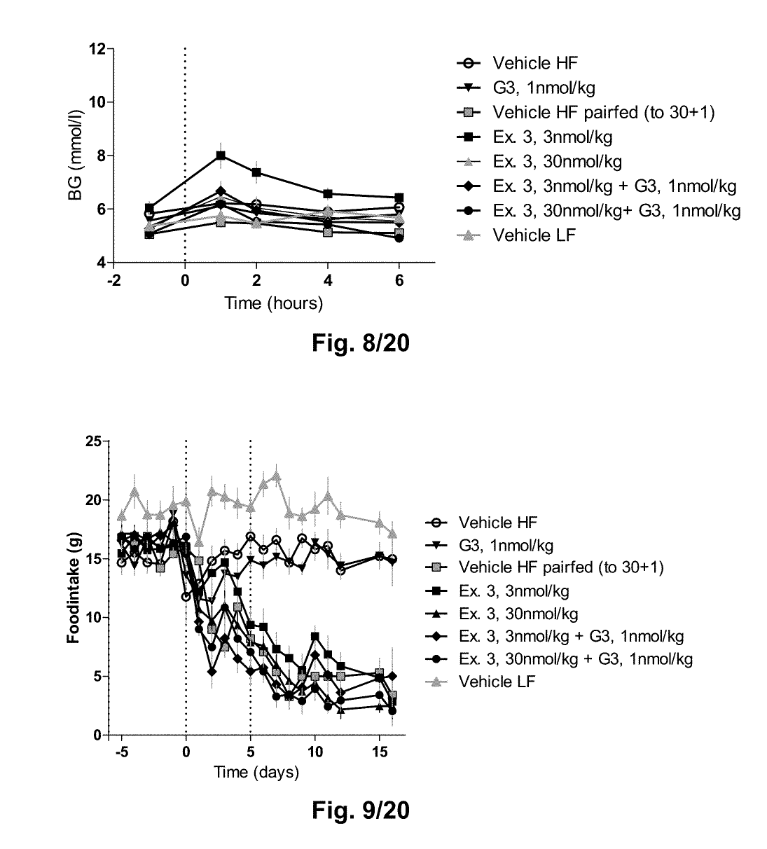

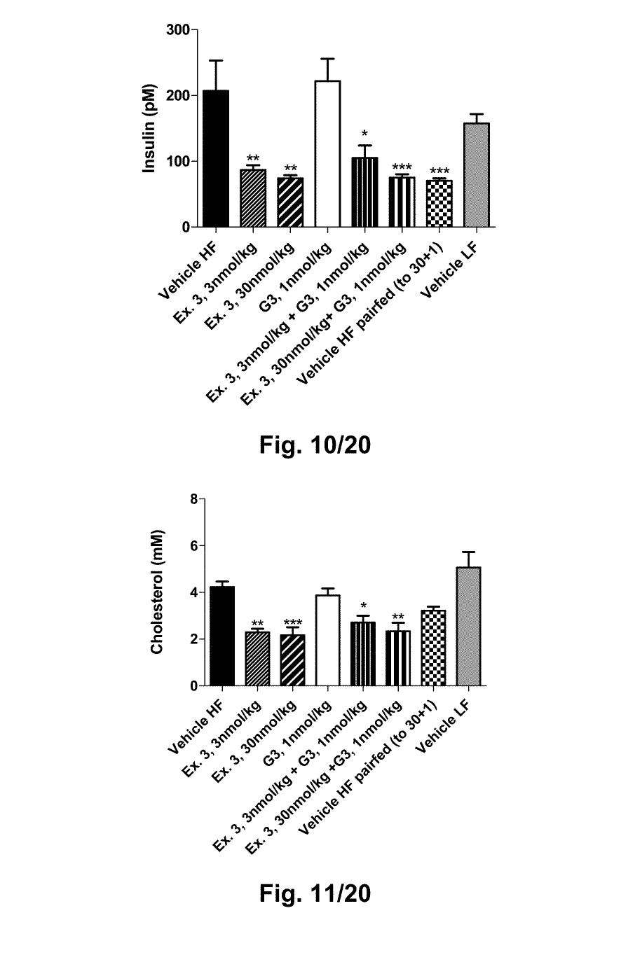

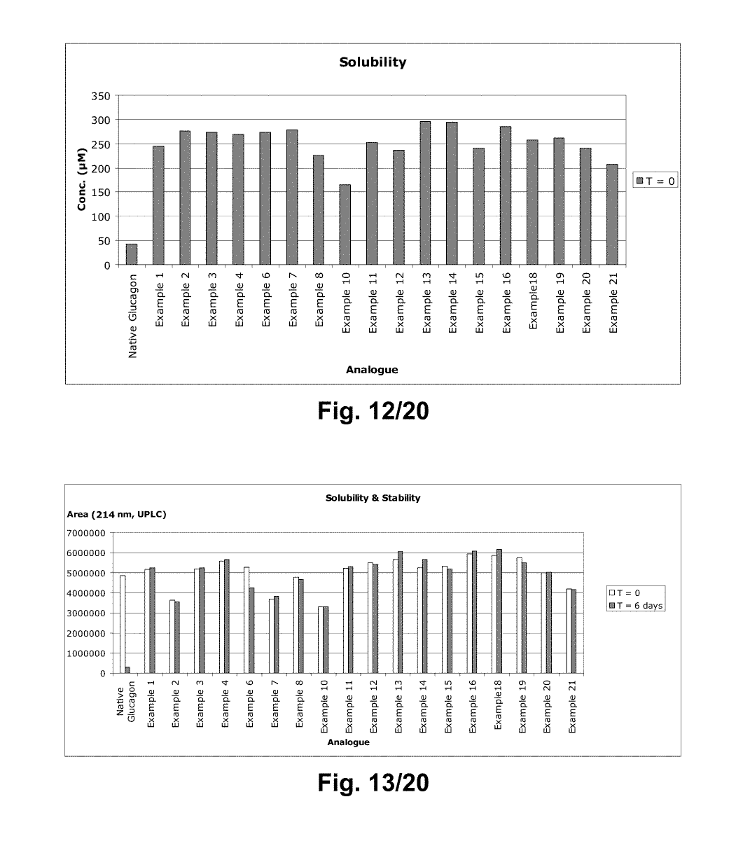

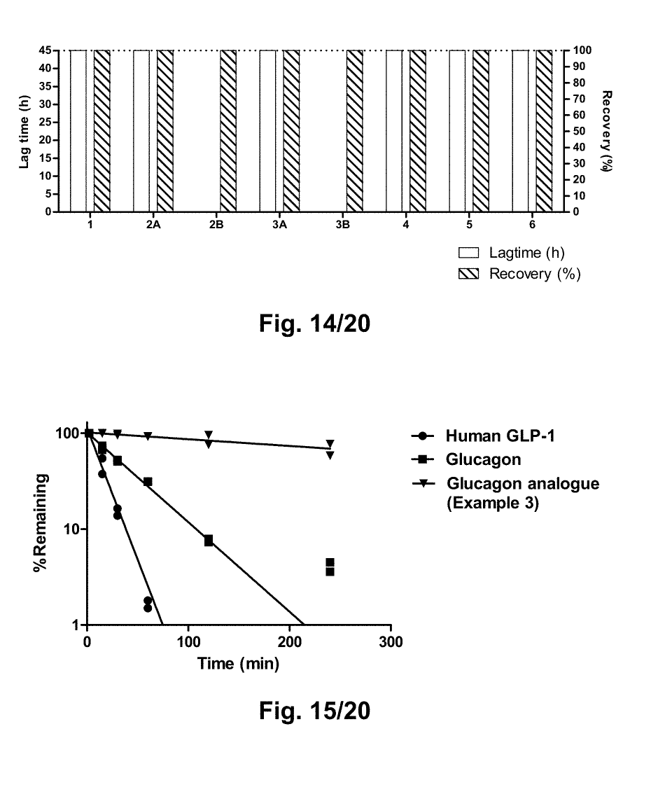

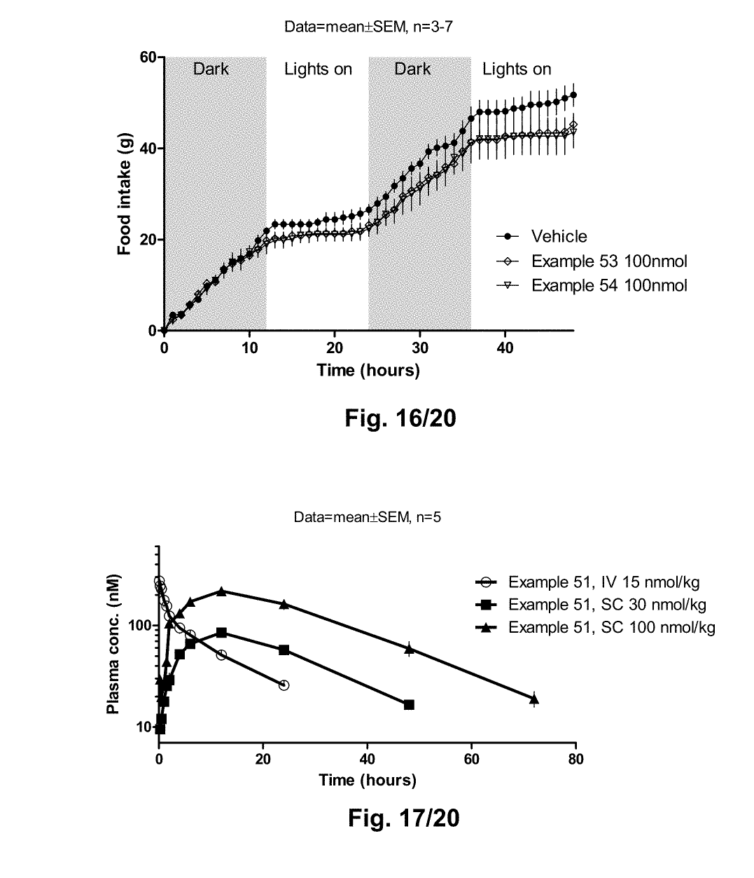

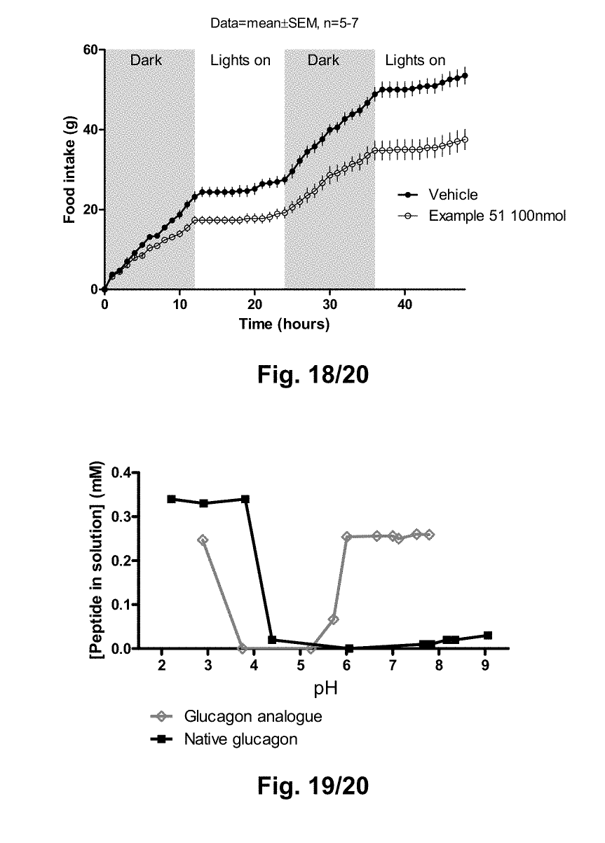

### DESCRIPTION OF THE INVENTION

Among further embodiments of the present invention are the following:

2. The glucagon peptide according to embodiment 1, wherein said glucagon peptide comprises zero, one, two, three, four, five, six or seven amino acid residues substitutions in said glucagon peptide.

3. The glucagon peptide according to any one of the previous embodiments, wherein said glucagon peptide comprises zero amino acid residues substitutions in said glucagon peptide.

4. The glucagon peptide according to any one of embodiments 1-2, wherein said glucagon peptide comprises one amino acid residues substitutions in said glucagon peptide.

5. The glucagon peptide according to any one of embodiments 1-2, wherein said glucagon peptide comprises two amino acid residues substitutions in said glucagon peptide.

6. The glucagon peptide according to any one of embodiments 1-2, wherein said glucagon peptide comprises three amino acid residues substitutions in said glucagon peptide.

7. The glucagon peptide according to any one of embodiments 1-2, wherein said glucagon peptide comprises four amino acid residues substitutions in said glucagon peptide.

8. The glucagon peptide according to any one of embodiments 1-2, wherein said glucagon peptide comprises five amino acid residues substitutions in said glucagon peptide.

9. The glucagon peptide according to any one of embodiments 1-2, wherein said glucagon peptide comprises six amino acid residues substitutions in said glucagon peptide.

10. The glucagon peptide according to any one of embodiments 1-2, wherein said glucagon peptide comprises seven amino acid residues substitutions in said glucagon peptide.

11. A glucagon peptide according to any one of the previous embodiments, wherein said amino acid substitutions are in the following amino acid positions of said glucagon peptide: X₂, X₄, X₉, X₁₀, X₁₂, X₁₆, X₁₇, X₁₈, X₂₀, X₂₁, X₂₄, X₂₅, X₂₇, X₂₈, X₂₉ and/or X₃₀.

12. The glucagon peptide according to any one of the previous embodiments, wherein said amino acid substitutions may be in the following positions of said glucagon peptide

X₂ represents Aib or D-Ser;
X₄ represents D-Phe;
X₉ represents Glu;
X₁₀ represents Cys, Lys, Orn or (p)Tyr;
X₁₂ represents Cys, Lys, Orn, Ile, His, Gln, Tyr, Leu or Arg;
X₁₆ represents Cys, Glu, Lys or Orn;
X₁₇ represents Cys, Gln, Lys, His or Orn;
X₁₈ represents Cys, Gln, Ala, Lys, His or Orn;
X₂₀ represents Cys, Arg, Lys, Glu, His or Orn;
X₂₁ represents Cys, Orn, Glu, Arg, His or Lys;
X₂₄ represents Cys, Lys, Arg, His, Glu, Asp, Gly, Ser or Orn;
X₂₅ represents Cys, Arg, Lys, His, Glu, Asp, Gly, Phe, Ser, Tyr, (p)Tyr or Orn;
X₂₇ represents Met(O), Val, Ile, Leu, Arg, His, Cys, Lys, Glu, Gln or Orn;
X₂₈ represents Cys, Lys, His, Arg, Ser, Thr, Glu, Asp, Ala, Gln or Orn;
X₂₉ represents Cys, Glu, Asp, Lys, His, Arg, Pro or Orn and
X₃₀ is absent or represents Cys, Lys, Arg, Glu, Gly, Pro or Orn.

13. The glucagon peptide according to any of the previous embodiments wherein said amino acid substitutions may be in the following positions of said glucagon peptide: X₄ represents D-Phe, X₉ represents Glu, X₁₂ represents Arg, X₁₆ represents Lys, X₂₀ represents Lys or Glu, X₂₁ represents Glu, X₂₄ represents Lys or His, X₂₅ represents Arg or Lys, X₂₇ represents Leu, Lys, Glu or Gln, X₂₈ represents Lys or Ser, X₂₉ represents Lys or Pro, X₃₀ is absent or represents Lys or Pro.

14. The glucagon peptide according to any one of the previous embodiments, wherein X₁₇ represents Lys, X₁₈ represents Lys, X₂₁ represents Glu, X₂₄ represents Lys or Orn, and X₂₇ represents Leu.

15. The glucagon peptide according to any one of the previous embodiments, wherein X₁₇ represents Lys, X₁₈ represents Lys, X₂₁ represents Glu and X₂₇ represents Leu.

16. The glucagon peptide according to any one of the previous embodiments, wherein X₁₇ represents Lys, X₂₁ represents Glu and X₂₇ represents Leu.

17. The glucagon peptide according to any one of the previous embodiments, wherein X₁₇ represents Lys and X₂₁ represents Glu.

18. The glucagon peptide according to any one of the previous embodiments, wherein X₂ represents Aib or D-Ser.

19. The glucagon peptide according to any one of the previous embodiments, wherein X₄ represents D-Phe.

20. The glucagon peptide according to any one of the previous embodiments, X₉ represents Glu.

21. The glucagon peptide according to any one of the previous embodiments, wherein X₁₀ represents Cys, Lys, Orn or (p)Tyr.

22. The glucagon peptide according to any one of the previous embodiments, wherein X₁₀ represents Cys.

23. The glucagon peptide according to any one of the previous embodiments, wherein X₁₀ represents Lys.

24. The glucagon peptide according to any one of the previous embodiments, wherein X₁₀ represents Orn.

25. The glucagon peptide according to any one of the previous embodiments, wherein X₁₂ represents Cys, Lys, Orn, Ile, His, Gln, Tyr, Leu or Arg.

26. The glucagon peptide according to any one of the previous embodiments, wherein X₁₂ represents Arg.

27. The glucagon peptide according to any one of the previous embodiments, wherein X₁₂ represents Cys, Lys or Orn.

28. The glucagon peptide according to any one of the previous embodiments, wherein X₁₂ represents Lys or Orn.

29. The glucagon peptide according to any one of the previous embodiments, wherein X₁₂ represents Cys.

30. The glucagon peptide according to any one of the previous embodiments, wherein X₁₂ represents Lys.

31. The glucagon peptide according to any one of the previous embodiments, wherein X₁₂ represents Orn.

32. The glucagon peptide according to any one of the previous embodiments, wherein X₁₆ represents Cys, Glu, Lys or Orn.

33. The glucagon peptide according to any one of the previous embodiments, wherein X₁₆ represents Cys, Lys or Orn.

34. The glucagon peptide according to any one of the previous embodiments, wherein X₁₆ represents Lys or Orn.

35. The glucagon peptide according to any one of the previous embodiments, wherein X₁₆ represents Lys.

36. The glucagon peptide according to any one of the previous embodiments, wherein X₁₆ represents Cys.

37. The glucagon peptide according to any one of the previous embodiments, wherein X₁₆ represents Orn.

38. The glucagon peptide according to any one of the previous embodiments, wherein X₁₇ represents Cys, Gln, Lys, His or Orn.

39. The glucagon peptide according to any one of the previous embodiments, wherein X₁₇ represents Lys.

40. The glucagon peptide according to any one of the previous embodiments, wherein X₁₇ represents Cys.

41. The glucagon peptide according to any one of the previous embodiments, wherein X₁₇ represents Orn.

42. The glucagon peptide according to any one of the previous embodiments, wherein X₁₈ represents Gln, Ala, Lys, His or Orn.

43. The glucagon peptide according to any one of the previous embodiments, wherein X₂₀ represents Cys, Arg, Lys, Glu, His or Orn.

44. The glucagon peptide according to any one of the previous embodiments, wherein X₂₀ represents Lys or Glu.

45. The glucagon peptide according to any one of the previous embodiments, wherein X₂₀ represents Lys.

46. The glucagon peptide according to any one of the previous embodiments, wherein X₂₀ represents Glu.

47. The glucagon peptide according to any one of the previous embodiments, wherein X₂₀ represents Cys.

48. The glucagon peptide according to any one of the previous embodiments, wherein X₂₀ represents Orn.

49. The glucagon peptide according to any one of the previous embodiments, wherein X₂₁ represents Cys, Orn, Glu, Arg, His or Lys.

50. The glucagon peptide according to any one of the previous embodiments, wherein X₂₁ represents Glu or Lys.

51. The glucagon peptide according to any one of the previous embodiments, wherein X₂₁ represents Glu.

52. The glucagon peptide according to any one of the previous embodiments, wherein X₂₁ represents Lys.

53. The glucagon peptide according to any one of the previous embodiments, wherein X₂₁ represents Cys.

54. The glucagon peptide according to any one of the previous embodiments, wherein X₂₁ represents Orn.

55. The glucagon peptide according to any one of the previous embodiments, wherein X₂₄ represents Cys, Lys, Arg, His, Glu, Asp, Gly, Ser or Orn.

56. The glucagon peptide according to any one of the previous embodiments, wherein X₂₄ represents Cys, Lys or Orn.

57. The glucagon peptide according to any one of the previous embodiments, wherein X₂₄ represents Lys or Orn.

58. The glucagon peptide according to any one of the previous embodiments, wherein X₂₄ represents Lys or His.

59. The glucagon peptide according to any one of the previous embodiments, wherein X₂₄ represents Lys.

60. The glucagon peptide according to any one of the previous embodiments, wherein X₂₄ represents His.

61. The glucagon peptide according to any one of the previous embodiments, wherein X₂₄ represents Cys.

62. The glucagon peptide according to any one of the previous embodiments, wherein X₂₄ represents Orn.

63. The glucagon peptide according to any one of the previous embodiments, wherein X₂₅ represents Arg, Lys, His, Glu, Asp, Gly, Phe, Ser, Tyr, (p)Tyr or Orn.

64. The glucagon peptide according to any one of the previous embodiments, wherein X₂₅ represents His, Lys, Ile, Leu, Ala, Met, Cys, Asn, Val, Ser, Gln, Asp, Glu, Thr or (p)Tyr.

65. The glucagon peptide according to any one of the previous embodiments, wherein X₂₅ represents His, Arg, Lys, or (p)Tyr.

66. The glucagon peptide according to any one of the previous embodiments, wherein X₂₅ represents Arg or Lys.

67. The glucagon peptide according to any one of the previous embodiments, wherein X₂₅ represents Arg.

68. The glucagon peptide according to any one of the previous embodiments, wherein X₂₅ represents Lys.

69. The glucagon peptide according to any one of the previous embodiments, wherein X₂₅ represents Cys.

70. The glucagon peptide according to any one of the previous embodiments, wherein X₂₅ represents Orn.

71. The glucagon peptide according to any one of the previous embodiments, wherein X₂₇ represents Cys, Met(O), Val, Ile, Leu, Arg, His, Lys, Glu, Gln or Orn

72. The glucagon peptide according to any one of the previous embodiments, wherein X₂₇ represents Leu, Lys, Glu or Gln.

73. The glucagon peptide according to any one of the previous embodiments, wherein X₂₇ represents Leu.

74. The glucagon peptide according to any one of the previous embodiments, wherein X₂₇ represents Lys.

75. The glucagon peptide according to any one of the previous embodiments, wherein X₂₇ represents Glu.

76. The glucagon peptide according to any one of the previous embodiments, wherein X₂₇ represents Gln.

77. The glucagon peptide according to any one of the previous embodiments, wherein X₂₇ represents Cys, Lys or Orn.

78. The glucagon peptide according to any one of the previous embodiments, wherein X₂₇ represents Lys or Orn.

79. The glucagon peptide according to any one of the previous embodiments, wherein X₂₇ represents Cys.

80. The glucagon peptide according to any one of the previous embodiments, wherein X₂₇ represents Orn.

81. The glucagon peptide according to any one of the previous embodiments, wherein X₂₈ represents Cys, Lys, His, Arg, Ser, Thr, Glu, Asp, Ala, Gln or Orn

82. The glucagon peptide according to any one of the previous embodiments, wherein X₂₈ represents Lys or Ser.

83. The glucagon peptide according to any one of the previous embodiments, wherein X₂₈ represents Cys, Lys or Orn.

84. The glucagon peptide according to any one of the previous embodiments, wherein X₂₈ represents Lys or Orn.

85. The glucagon peptide according to any one of the previous embodiments, wherein X₂₈ represents Cys.

86. The glucagon peptide according to any one of the previous embodiments, wherein X₂₈ represents Orn.

87. The glucagon peptide according to any one of the previous embodiments, wherein X₂₈ represents Lys.

88. The glucagon peptide according to any one of the previous embodiments, wherein X₂₉ represents Cys, Lys or Orn.

89. The glucagon peptide according to any one of the previous embodiments, wherein X₂₉ represents Lys or Orn.

90. The glucagon peptide according to any one of the previous embodiments, wherein X₂₉ represents Orn.

91. The glucagon peptide according to any one of the previous embodiments, wherein X₂₉ represents Lys or Pro.

92. The glucagon peptide according to any one of the previous embodiments, wherein X₂₉ represents Lys.

93. The glucagon peptide according to any one of the previous embodiments, wherein X₃₀ is absent or represents Cys, Lys, Arg, Glu, Gly, Pro or Orn.

94. The glucagon peptide according to any one of the previous embodiments, wherein X₃₀ is absent or represents Cys, Lys or Orn.

95. The glucagon peptide according to any one of the previous embodiments, wherein X₃₀ is absent or represents Lys or Orn.

96. The glucagon peptide according to any one of the previous embodiments, wherein X₃₀ is absent or represents Orn.

97. The glucagon peptide according to any one of the previous embodiments, wherein X₃₀ is absent or represents Cys.

98. The glucagon peptide according to any one of the previous embodiments, wherein X₃₀ is absent or represents Lys or Pro.

99. The glucagon peptide according to any one of the previous embodiments, wherein X₃₀ is absent or represents Lys.

100. The glucagon peptide according to any one of the previous embodiments, wherein X₃₀ is absent or represents Pro.

101. A glucagon peptide according to any of the previous embodiments, wherein said substituent has the formula II:

Z₁—Z₂—Z₃—Z₄ [II]

wherein,
Z₁ represents a structure according to one of the formulas IIa, IIb or IIc;

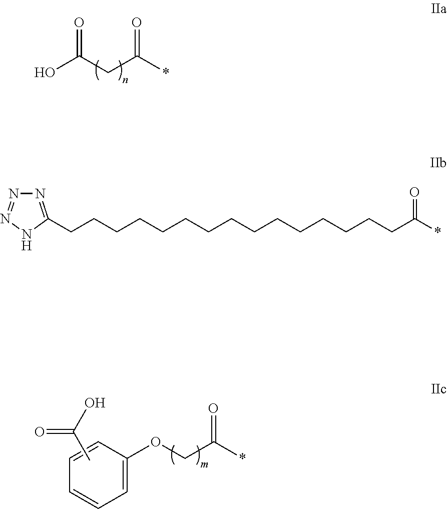

wherein n in formula IIa is 6-20,
m in formula IIc is 5-11
the COOH group in fomula IIc can be attached to position 2, 3 or 4 on the phenyl ring,
the symbol * in formula IIa, IIb and IIc represents the attachment point to the nitrogen in Z₂;
if Z₂ is absent, Z₁ is attached to the nitrogen on Z₃ at symbol * and if Z₂ and Z₃ are absent Z₁ is attached to the nitrogen on Z₄ at symbol *
Z₂ is absent or represents a structure according to one of the formulas IId, IIe, IIf, IIg, IIh, IIi, IIj or IIk;

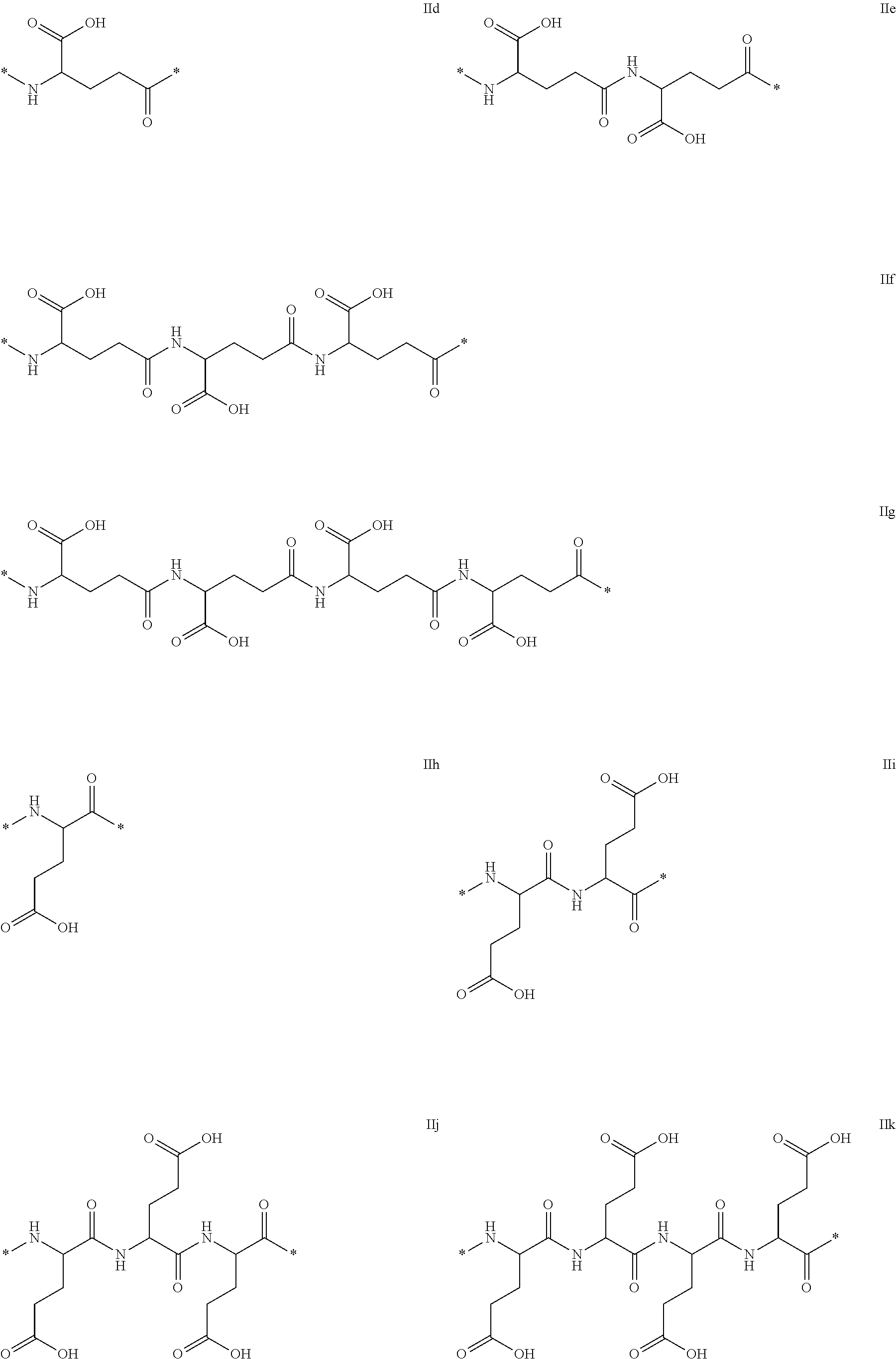

wherein each amino acid moiety independently has the stereochemistry L or D;
wherein Z₂ is connected via the carbon atom denoted * to the nitrogen of Z₃ denoted *;
if Z₃ is absent, Z₂ is connected via the carbon atom denoted * to the nitrogen of Z₄ denoted * and
if Z₃ and Z₄ are absent Z₂, is connected via the carbon denoted * to the epsilon nitrogen of a lysine or the delta nitrogen of an ornithine of the glucagon peptide.
Z₃ is absent or represents a structure according to one of the formulas IIm, IIn, IIo or IIp;

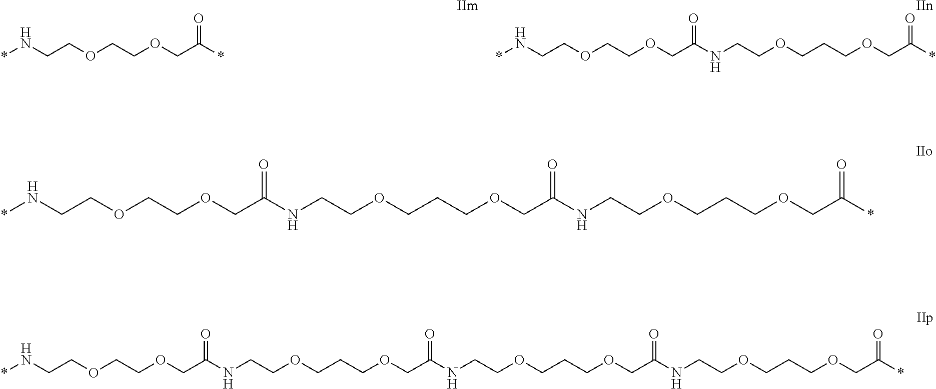

Z₃ is connected vi the carbon of Z₃ with symbol * to the nitrogen of Z₄ with symbol *, if Z₄ is absent Z₃ is connected via the carbon with symbol * to the epsilon nitrogen of a lysine or the delta nitrogen of an ornithine of the glucagon peptide
Z₄ is absent or represents a structure according to one of the formulas IId, IIe, IIf, IIg, IIh, IIi, IIj or IIk; wherein each amino acid moiety is independently either L or D, wherein Z₄ is connected via the carbon with symbol * to the epsilon nitrogen of a lysine or the delta nitrogen of an ornithine of the glucagon peptide.

102. The glucagon peptide according to any of the previous embodiments, wherein said substituent has the formula II:

Z₁—Z₂—Z₃—Z₄— [II]

wherein,
Z₁ represents a structure according to one of the formulas IIa, IIb or IIc;

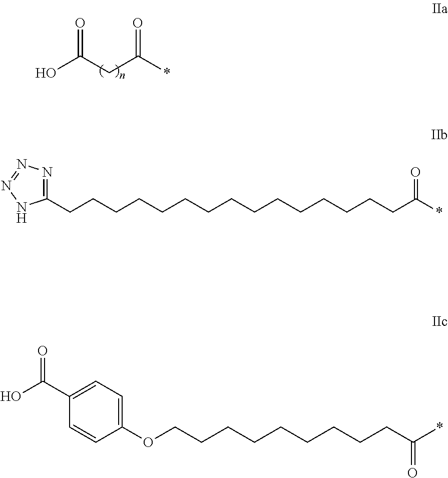

wherein n in formula IIa is 6-20,
Z₂ is absent or represents a structure according to one of the formulas IId, IIe, IIf, IIg, IIh, IIi, IIj or IIk;

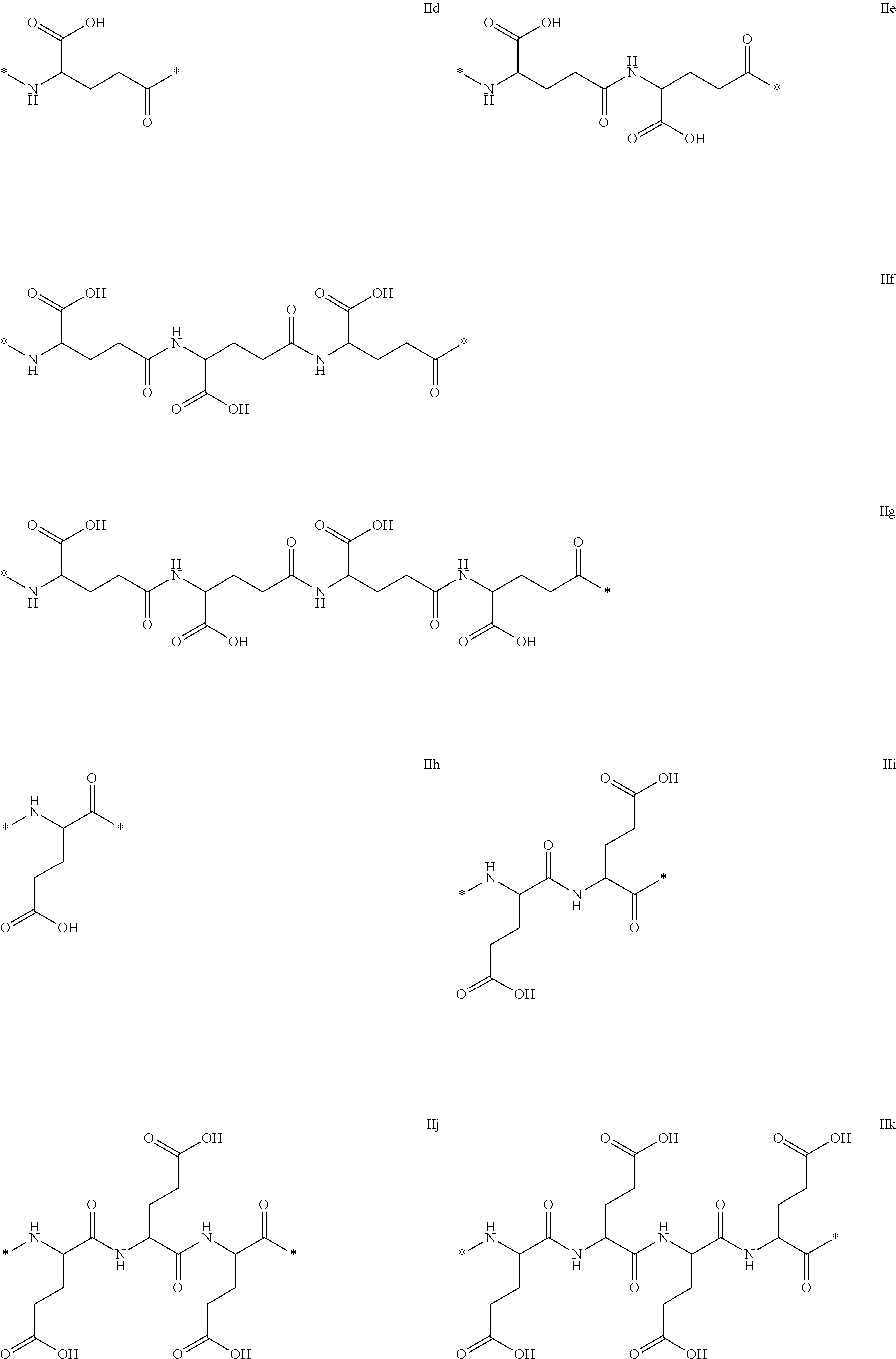

wherein each amino acid moiety independently has the stereochemistry L or D.
Z₃ is absent or represents a structure according to one of the formulas IIm, IIn, IIo or IIp;

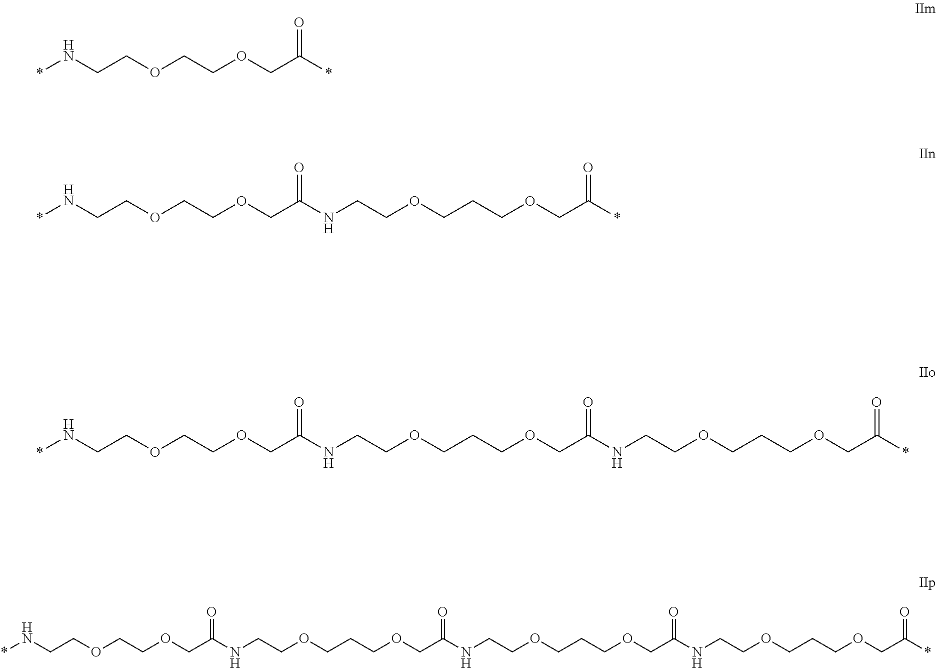 [SMILES: *NCCOCCOCC(*)=O ; *NCCOCCOCC(=O)NCCOCCCOCC(*)=O ; *NCCOCCOCC(=O)NCCOCCCOCC(=O)NCCOCCCOCC(*)=O ; *NCCOCCOCC(=O)NCCOCCCOCC(=O)NCCOCCCOCC(=O)NCCOCCCOCC(*)=O]

Z₄ is absent or represents a structure according to one of the formulas IId, IIe, IIf, IIg, IIh, IIi, IIj or IIk;
wherein each amino acid moiety independently has the stereochemistry L or D.

103. The glucagon peptide according to any one of the previous embodiments, wherein the structures of formulas IIa-IIp have the stereochemistry L.

104. The glucagon peptide according to any one of the previous embodiments, wherein the structures of formulas IIa-IIp have the stereochemistry D.

105. The glucagon peptide according to any of the previous embodiments, wherein Z₂ of said substituent of formula II is absent when Z₄ is present.

106. The glucagon peptide according to any of the previous embodiments, wherein Z₄ of said substituent of formula II is absent when Z₂ is present.

107. The glucagon peptide according to any of the previous embodiments, wherein said substituent represents a structure according to one of the formulas IIIa, IIIb, IIIc, IIId, IIIe, IIIf, IIIg, IIIh, IIIi, IIIj, IIIk, IIIl, IIIm, IIIn or IIIo:

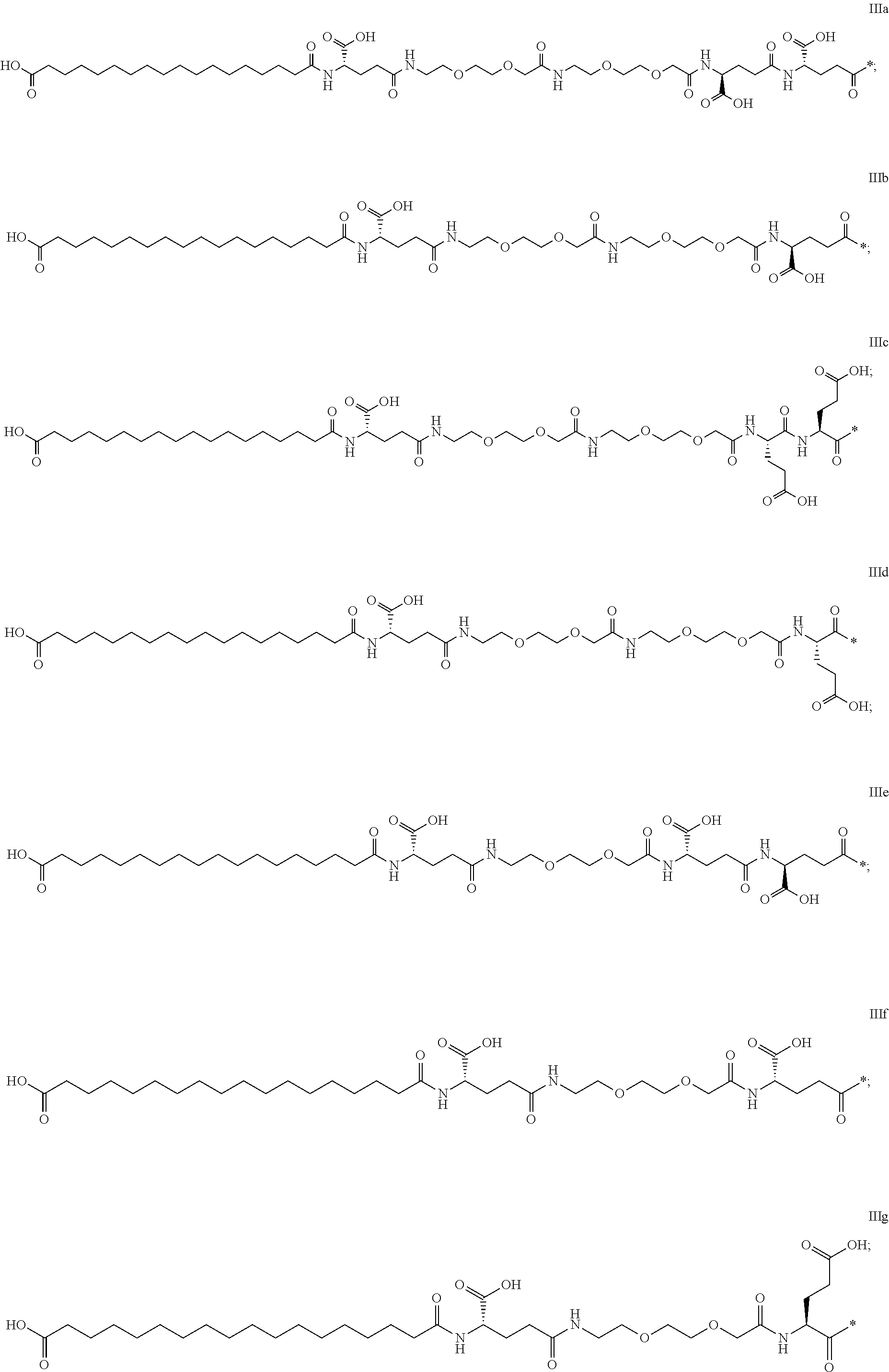

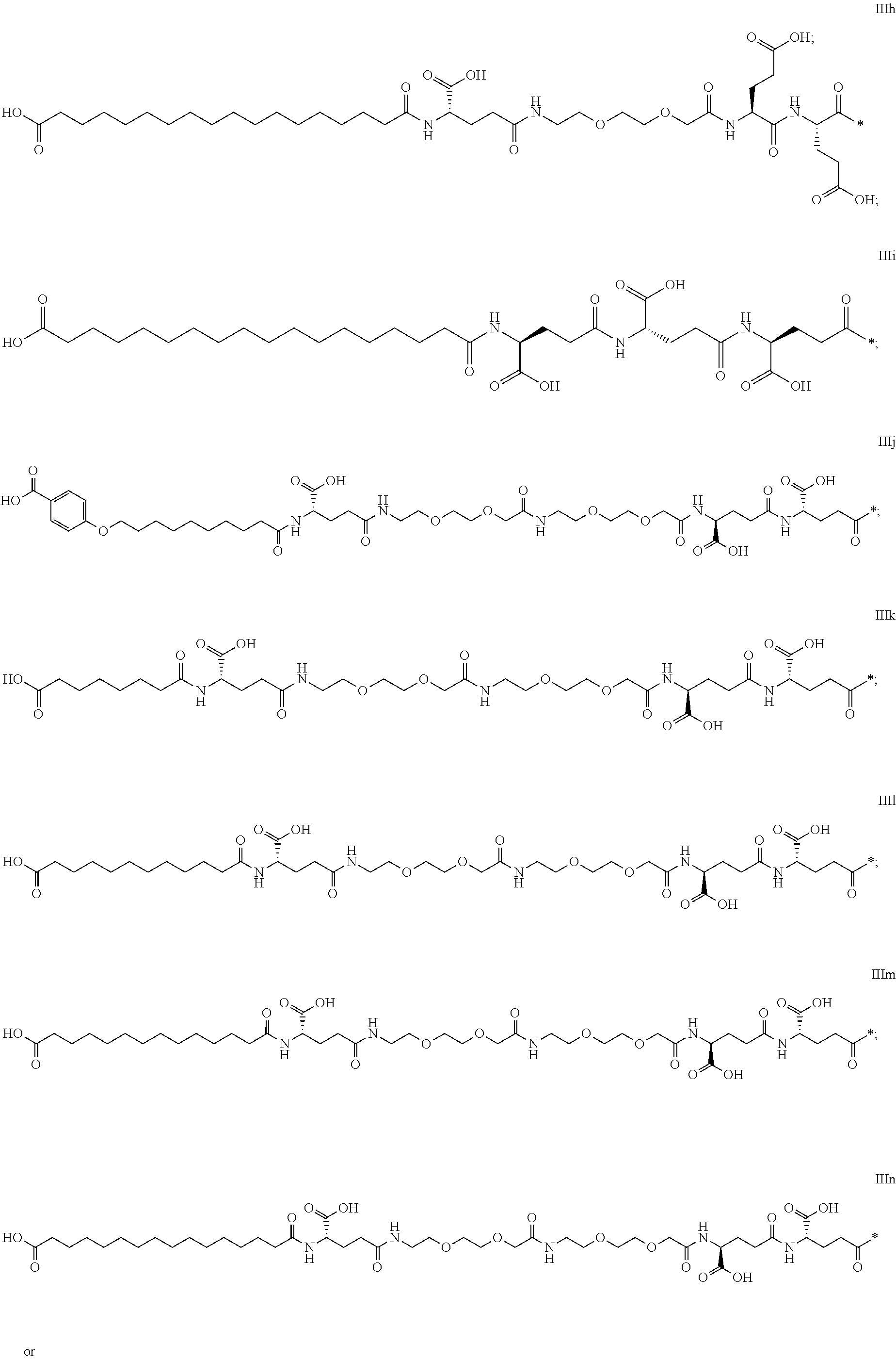

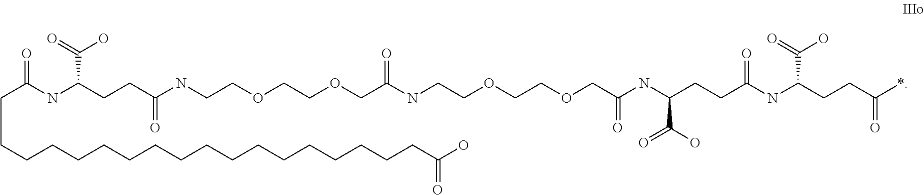

108. The glucagon peptide according to any of the previous embodiments, wherein Z₄ of said substituent is absent.

109. The glucagon peptide according to any of the previous embodiments, wherein Z₃ and Z₄ of said substituent are absent.

110. A glucagon peptide according to any of the previous embodiments, wherein Z₂ and Z₄ of said substituent are independently represented by negatively charged moieties such as γGlu, Glu and/or Asp.

111. A glucagon peptide according to any of the previous embodiments, wherein Z₂ and Z₄ of said substituent are independently represented by up to ten of said moieties.

112. A glucagon peptide according to any of the previous embodiments, wherein Z₂ and Z₄ of said substituent are independently represented by three of said moieties.

113. A glucagon peptide according to any of the previous embodiments, wherein Z₂ and Z₄ of said substituent are independently represented by four of said moieties.

114. A glucagon peptide according to any of the previous embodiments, wherein Z₂ and Z₄ of said substituent are independently represented by five of said moieties.

115. A glucagon peptide according to any of the previous embodiments, wherein Z₂ and Z₄ of said substituent are independently represented by Glu and/or γGlu moieties.

116. A glucagon peptide according to any of the previous embodiments, wherein Z₂ and Z₄ of said substituent are independently represented by γGlu, γGlu-Glu, γGlu-Glu-Glu, γGlu-Glu-Glu-Glu, γGlu-Glu-Glu-Glu-Glu.

117. A glucagon peptide according to any of the previous embodiments, wherein Z₂ and Z₄ of said substituent are independently represented by Glu and/or Asp moieties.

118. A glucagon peptide according to any of the previous embodiments, wherein Z₂ and Z₄ of said substituent are independently represented by γGlu and/or Asp moieties.

119. A glucagon peptide according to any of the previous embodiments, wherein Z₂ and Z₄ of said substituent are independently represented by Asp moieties

120. A glucagon peptide according to any of the previous embodiments, wherein Z₂ and Z₄ of said substituent are independently represented by Asp, Asp-Asp, Asp-Asp-Asp or Asp-Asp-Asp-Asp.

121. A glucagon peptide according to any of the previous embodiments, wherein Z₂ and Z₄ of said substituent are independently represented by Glu moieties.

122. A glucagon peptide according to any of the previous embodiments, wherein Z₂ and Z₄ of said substituent are independently represented by Glu, Glu-Glu, Glu-Glu-Glu, Glu-Glu-Glu-Glu, Glu-Glu-Glu-Glu-Glu.

123. A glucagon peptide according to any of the previous embodiments, wherein Z₂ and Z₄ of said substituent are independently represented by γGlu moieties.

124. A glucagon peptide according to any of the previous embodiments, wherein Z₂ and Z₄ of said substituent are independently represented by γGlu, γGlu-γGlu, γGlu-γGlu-γGlu, γGlu-γGlu-γGlu-γGlu, γGlu-γGlu-γGlu-γGlu-γGlu.

125. The glucagon peptide according to any of the previous embodiments, wherein said substituent comprises a lipophilic residue.

126. The glucagon peptide according to any of the previous embodiments, wherein said substituent comprises a straight chain alkyl group or a branched alkyl group.

127. The glucagon peptide according to any of the previous embodiments, wherein said substituent binds non-covalently to albumin.

128. The glucagon peptide according to any of the previous embodiments, wherein said substituent is negatively charged at physiological pH.

Further embodiments of the present invention relate to:

129. A glucagon peptide according to any of the previous embodiments, wherein said substituent is attached at the epsilon position of a Lys or at the delta position of an Orn, or at the sulphur of a Cys.

130. A glucagon peptide according to any of the previous embodiments, wherein said substituent is attached at the epsilon position of a Lys or at the delta position of an Orn.

131. A glucagon peptide according to any of the previous embodiments, wherein said substituent is attached at the epsilon position of a Lys.

132. A glucagon peptide according to any of the previous embodiments, wherein said substituent is attached at the delta position of an Orn.

133. A glucagon peptide according to any of the previous embodiments, wherein said substituent is attached at the sulphur position of a Cys.

134. The glucagon peptide according to any of the previous embodiments, wherein said substituent is attached in one or more of following amino acid positions of said glucagon peptide: X₁₀, X₁₂, X₁₆, X₁₇, X₁₈, X₂₀, X₂₁, X₂₄, X₂₅, X₂₇, X₂₈, X₂₉, and/or X₃₀.

135. The glucagon peptide according to any of the previous embodiments, wherein said substituent is in one or more of following amino acid positions of said glucagon peptide: X₁₂, X₁₆, X₂₀, X₂₄, X₂₅, X₂₈, X₂₉ and/or X₃₀.

136. The glucagon peptide according to any of the previous embodiments, wherein said substituent is in one or more of following amino acid positions of said glucagon peptide: X₁₆, X₂₄ and/or X₂₈.

137. The glucagon peptide according to any of the previous embodiments, wherein said substituent is at amino acid position X₁₂ of said glucagon peptide.

138. The glucagon peptide according to any of the previous embodiments, wherein said substituent is at amino acid position X₁₆ of said glucagon peptide.

139. The glucagon peptide according to any of the previous embodiments, wherein said substituent is at amino acid position X₂₀ of said glucagon peptide.

140. The glucagon peptide according to any of the previous embodiments, wherein said substituent is at amino acid position X₂₄ of said glucagon peptide.

141. The glucagon peptide according to any of the previous embodiments, wherein said substituent is at amino acid position X₂₈ of said glucagon peptide.

142. The glucagon peptide according to any of the previous embodiments, wherein said substituent is at amino acid position X₂₉ of said glucagon peptide.

143. The glucagon peptide according to any of the previous embodiments, wherein said substituent is at amino acid position X₃₀ of said glucagon peptide.

144. The glucagon peptide according to any of the previous embodiments, wherein said substituent is in up to five amino acid positions of said glucagon peptide.

145. The glucagon peptide according to any of the previous embodiments, wherein said substituent is in up to four amino acid positions of said glucagon peptide.

146. The glucagon peptide according to any of the previous embodiments, wherein said substituent is in up to three amino acid positions of said glucagon peptide.

147. The glucagon peptide according to any of the previous embodiments, wherein said substituent is in two amino acid position of said glucagon peptide.

148. The glucagon peptide according to any of the previous embodiments, wherein said substituent is in one amino acid position of said glucagon peptide.

Further embodiments of the present invention relate to:

The present invention relates to novel glucagon analogues with improved solubility, improved physical stability toward gel and fibril formation and with increased half life.

The inventors have found that the compounds of the present invention have a prolonged half life and that they show improved pharmacokinetic properties, i.e., they have prolonged exposure in vivo. Furthermore, the compounds of the present invention show a significant reduction in food intake when administered s.c. with a protracted effect up to 48 hours. This is to our best knowledge, the first demonstration of reduced food intake of a protracted glucagon analogue.

Protracted effect of the compounds of the present invention means that the period of time in which they exert a biological activity is prolonged. Effect is defined as being protracted when a compound significantly reduces food intake in the period from 24 hours to 48 hours in test animals compared to the food intake in the same time period in the vehicle-treated control group of animals in “Assay IV”. The protracted effect can be evaluated through different binding assays, for example the protracting effect may be evaluated in an indirect albumin-binding assay, in which Ki determined for binding in the presence of ovalbumin is compared with the EC₅₀ value determined in the presence of human serum albumin (HSA).

The inventors surprisingly found that the compounds of the present invention, show improved aqueous solubility at neutral pH or slightly basic pH. Furthermore, the present inventors have also surprisingly found that the glucagon analogues of the present invention have improved stability towards formation of gels and fibrils in aqueous solutions. The stability of the compounds of the present invention may be measured by a method as described in example 63.

A better control of blood glucose levels in Type 1 and 2 diabetes may be achieved by co-administration of glucagon with known antidiabetic agents such as insulin, GLP-1 agonists and GIP. The glucagon analogues of the invention show anorectic effects in rats when administered a single dose, and the effect at day two were observed to be as least as good as the effect at the day of dosing, clearly demonstrating the protracted effect of these analogues. Furthermore, the compounds of the present invention give a high reduction of body weight when administered to diet induced obese rats. An even more pronounced reduction of body weight can be obtained by co-administration with a protracted GLP-1 analogue, which in addition leads to a better control of blood glucose.

In one embodiment, the glucagon analogues of this invention can be co-formulated with GLP-1 analogues or insulin analogues, forming stable pharmaceutical compositions.

Combination of insulin and glucagon therapy may be advantageous compared to insulin-only therapy. Normally, in a postprandial situation when blood glucose levels become low the first hormonal response is reduction in the production of insulin. When blood glucose drop further the second line response is production of glucagon—resulting in increased glucose output from the liver. When diabetics receive an exogenous dose of insulin that is too high the natural response of raised glucagon is prevented by the presence of exogenous insulin, since insulin has an inhibiting effect on glucagon production. Consequently, slight overdosing of insulin may cause hypoglycaemia. Presently, many diabetic patients tend to prefer to use a little less insulin than optimal in fear of hypoglycaemic episodes which may be life-threatening.

The fact that the compounds of the present invention are soluble at neutral pH, may allow a co-formulation with insulin and allow for more stable blood glucose levels and a reduced number of hypoglycaemic episodes, as well as a reduced risk of diabetes related complications.

Further embodiments of the present invention relate to intramolecular bridges:

149. The glucagon peptide according to any of embodiments 1-148, further comprising an intramolecular bridge between the side chains of an amino acid at position Xi and an amino acid at position Xi+4.

150. The glucagon peptide according to any of embodiment 149, wherein the amino acid at position Xi and an amino acid at position Xi+4 are linked through a lactam bridge or a salt bridge.

151. The glucagon peptide according to any of embodiment 149, wherein the amino acid at position Xi and an amino acid at position Xi+4 are linked through a lactam bridge.

152. The glucagon peptide according to embodiment 149, wherein the amino acid at position Xi and an amino acid at position Xi+4 are linked through a salt bridge.

153. The glucagon peptide according to embodiments 149-152, wherein Xi is selected from positions X₁₂, X₁₆, X₁₇, X₂₀ or X₂₄.

154. The glucagon peptide according to any one of embodiments 149-153, wherein said intramolecular bridge is between the side chains of an amino acid at position 17 and an amino acid at position 21.

155. The glucagon peptide according to any one of embodiments 149-154, wherein one, two, three or more of positions X₁₆, X₁₇, X₂₀, X₂₁ or X₂₄ of said glucagon peptide, are substituted with an α amino acid and/or an α-disubstituted amino acid.

156. The glucagon peptide according to any one of the previous embodiments, wherein X₁₆ represents Glu and X₂₀ represents Lys.

157. A glucagon peptide according to any one of the previous embodiments, wherein said glucagon peptide comprises C-terminal extensions of up to three amino acid residues.

158. A glucagon peptide according to any one of the previous embodiments, wherein said glucagon peptide comprises C-terminal extensions of up to two amino acid residues.

159. A glucagon peptide according to any one of the previous embodiments, wherein said glucagon peptide comprises C-terminal extensions of one amino acid residue.

160. A glucagon peptide according to any one the previous embodiments, wherein the glucagon peptide is a C-terminal amide or a C-terminal carboxylic acid.

161. A glucagon peptide according to any one of the previous embodiments, wherein said glucagon peptide is a C-terminal amide.

162. A glucagon peptide according to any one of the previous embodiments, wherein said glucagon peptide is a C-terminal carboxylic acid.

163. The glucagon peptide according to any one of the previous embodiments, wherein said glucagon peptide is selected from glucagon (1-29), glucagon (1-29)-amide, or an analogue thereof.

164. The glucagon peptide according to any one of the previous embodiments, selected from the group consisting of:
N^{ε24}-([(4S)-5-hydroxy-4-[[(4S)-5-hydroxy-4-[[2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl]) [Lys²⁴,Leu²⁷] Glucagon

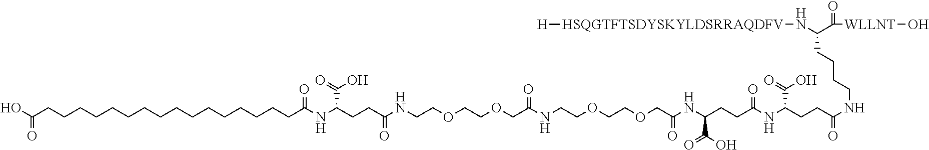

N^{ε28}-([(4S)-5-hydroxy-4-[[(4S)-5-hydroxy-4-[[2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl]) [Leu²⁷,Lys²⁸] Glucagon

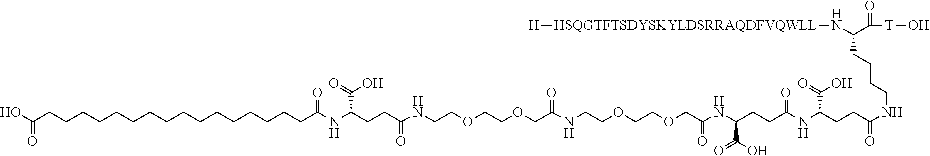

N^{ε29}-([(4S)-5-hydroxy-4-[[4S)-5-hydroxy-4-[[2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanyl]) [Leu²⁷,Lys²⁹] Glucagon

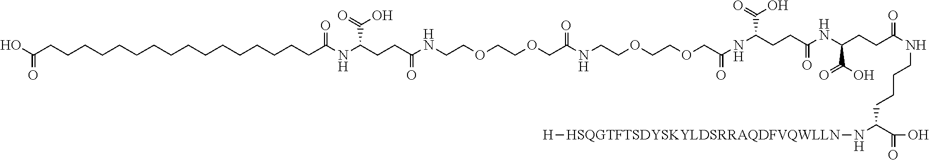

N^{α}-([Leu²⁷]Glucagonyl)N^{ε}-([(4S)-5-hydroxy-4-[[(4S)-5-hydroxy-4-[[2-[2-[2-[[2-[2-[2-[[(4S)- 5-hydroxy-4-[(18-hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl])Lysine

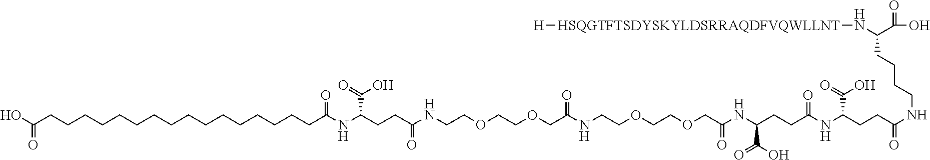

N^{ε28}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Leu²⁷,Lys²⁸]-Glucagon

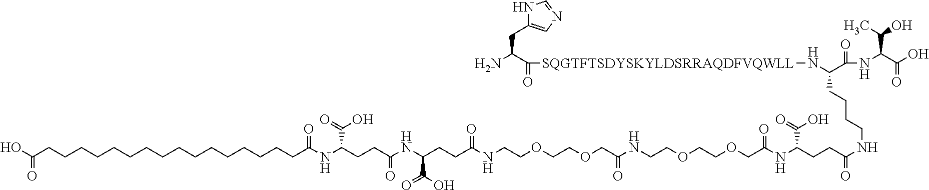

N^{ε28}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Leu²⁷,Lys²⁸]-Glucagon

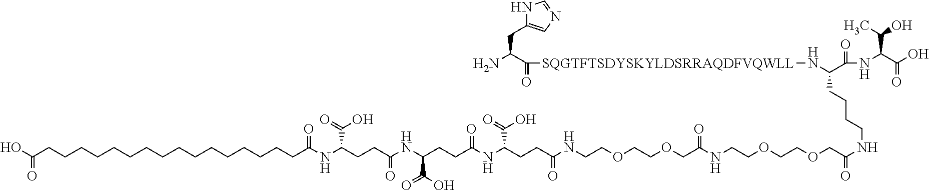

N^{ε24}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys²⁴,Leu²⁷,Ser²⁸]-Glucagon

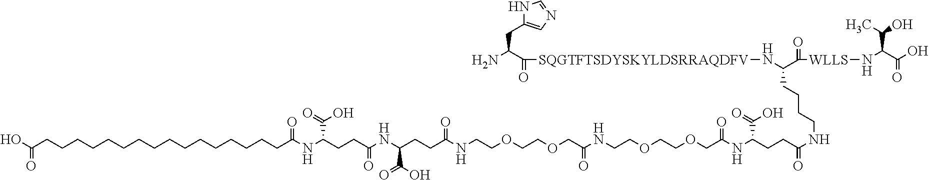

N^{ε24}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys²⁴,Leu²⁷,Ser²⁸]-Glucagon

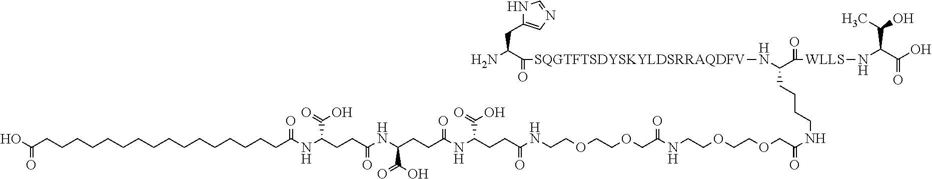

N^{ε16}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys¹⁶,Leu²⁷]-Glucagon

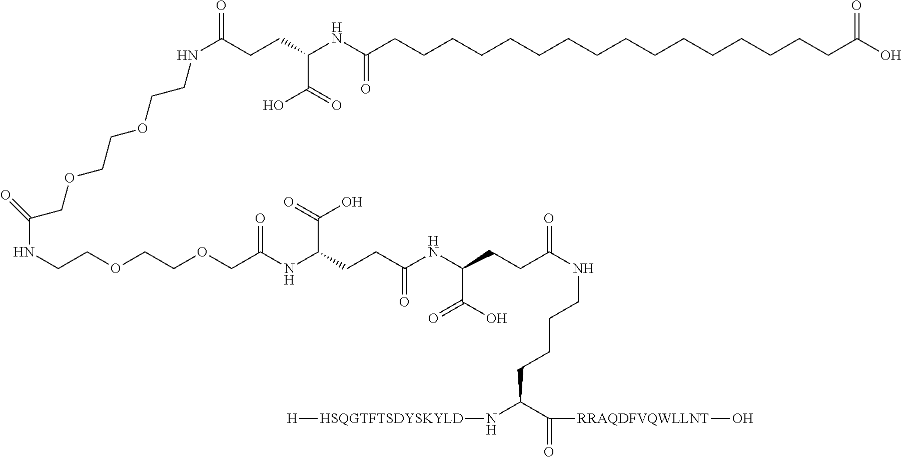

N^{ε24}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Lys²⁴,Leu²⁷,Ser²⁸]-Glucagon

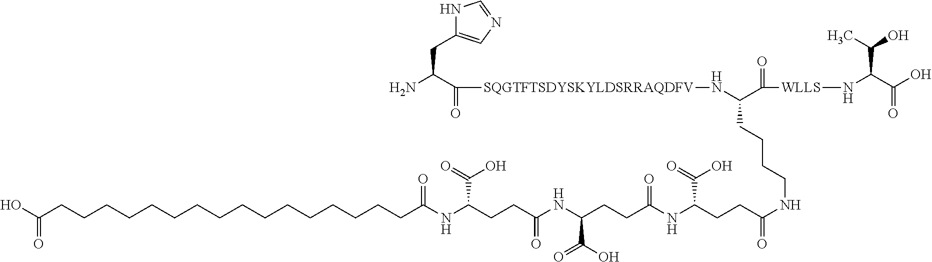

N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Arg¹²,Lys²⁴,Leu²⁷]-Glucagon

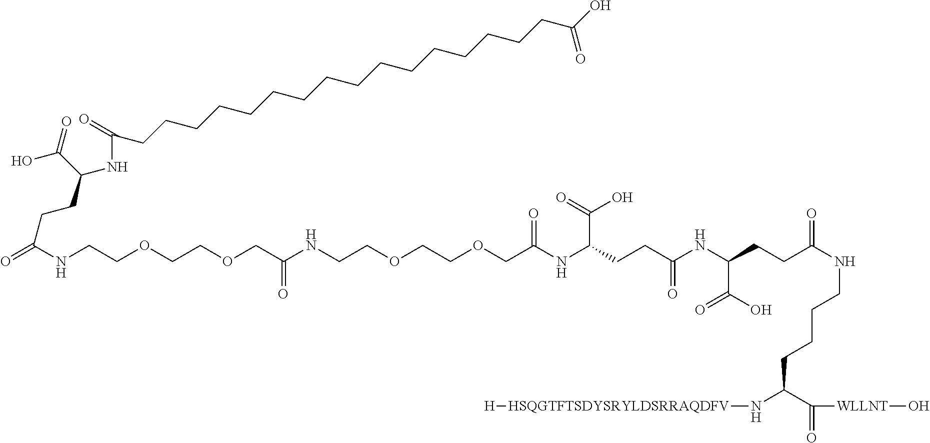

N^{ε24}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys²⁴,Leu²⁷]-Glucagon

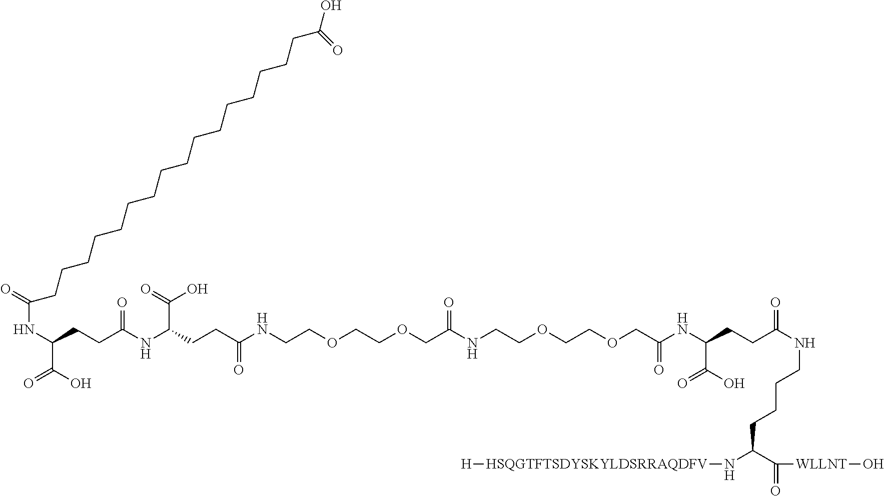

N^{ε24}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys²⁴,Leu²⁷]-Glucagon

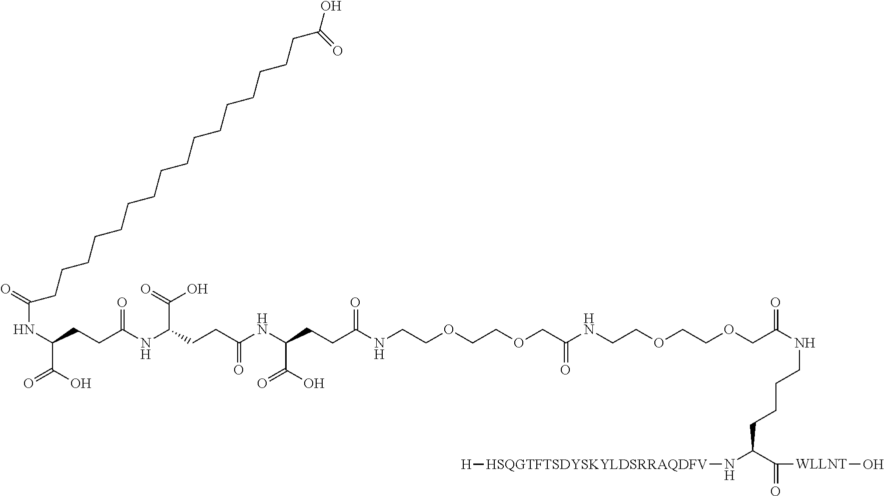

N^{ε24}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Lys²⁴,Leu²⁷]-Glucagon

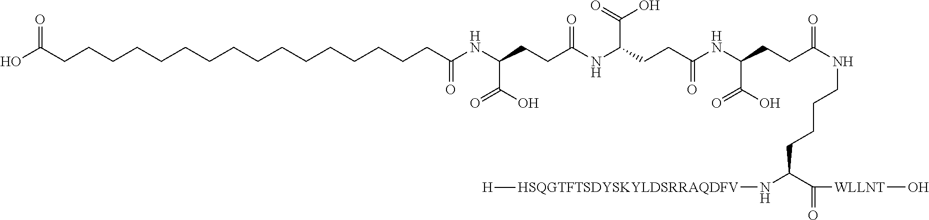

N^{ε25}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁵,Leu²⁷]-Glucagon

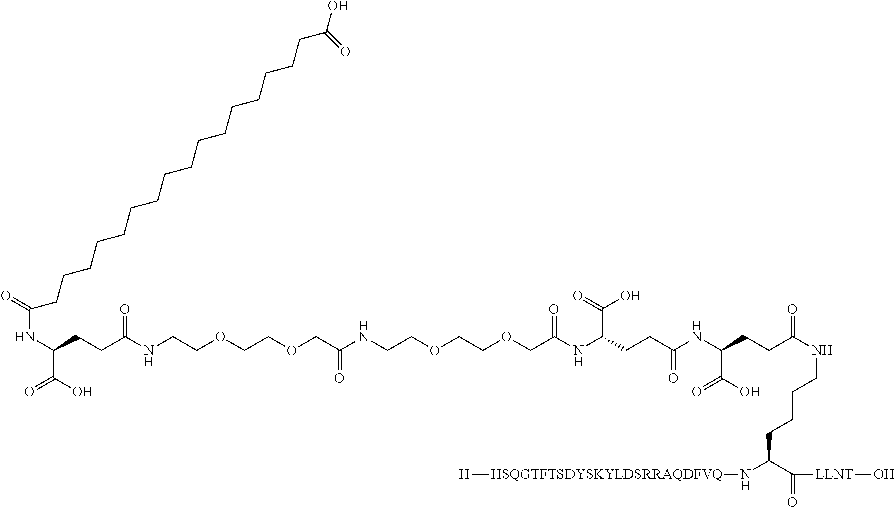

N^{ε16}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys¹⁶,Leu²⁷]-Glucagon

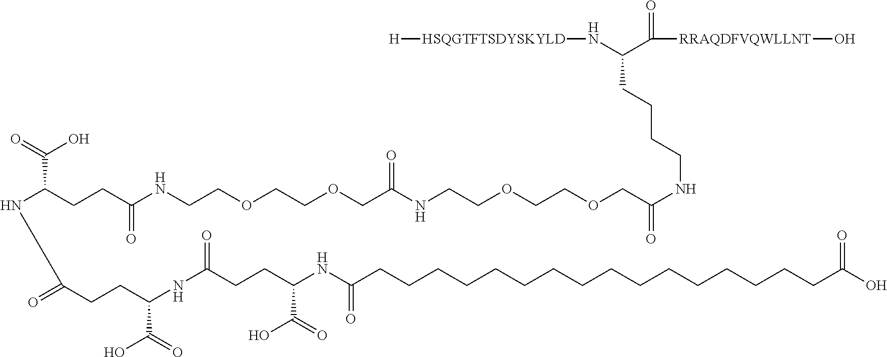

N^{ε16}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys¹⁶,Leu²⁷]-Glucagon

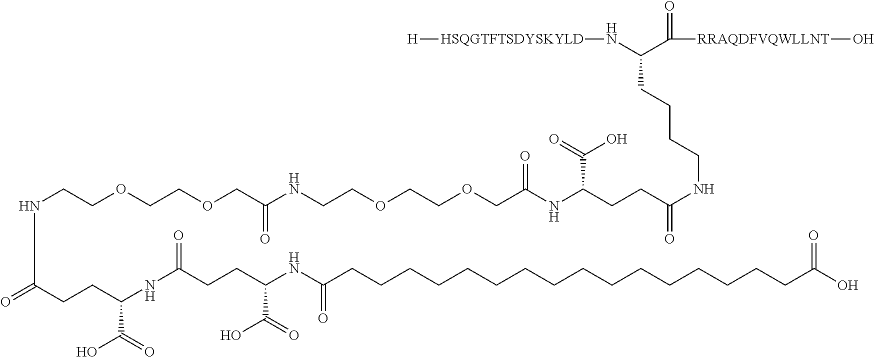

N^{ε28}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Leu²⁷,Lys²⁸]-Glucagon

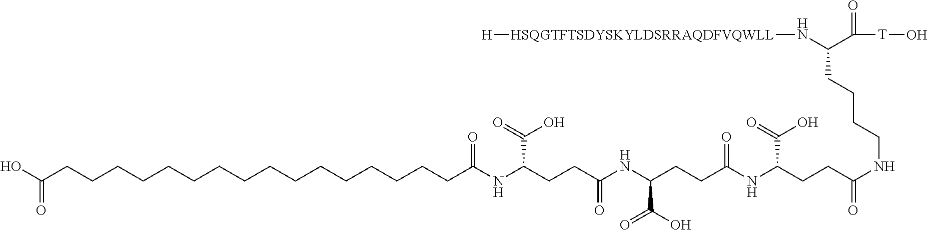

N^{ε12}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu²⁷,Pro²⁹]-Glucagon

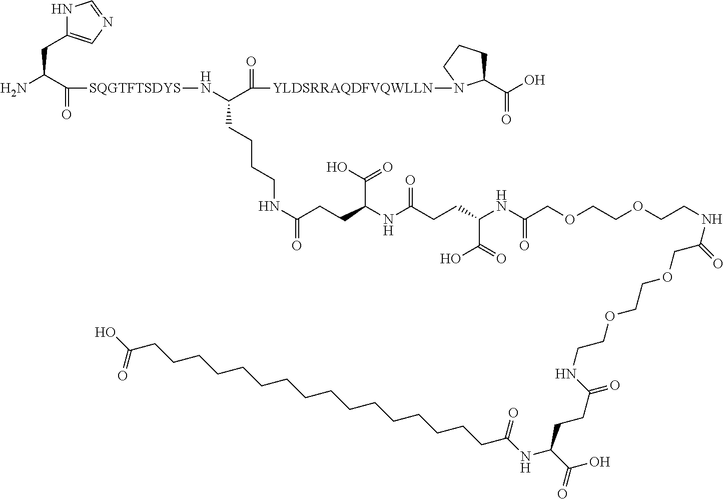

N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Leu²⁷,Pro²⁹]-Glucagon

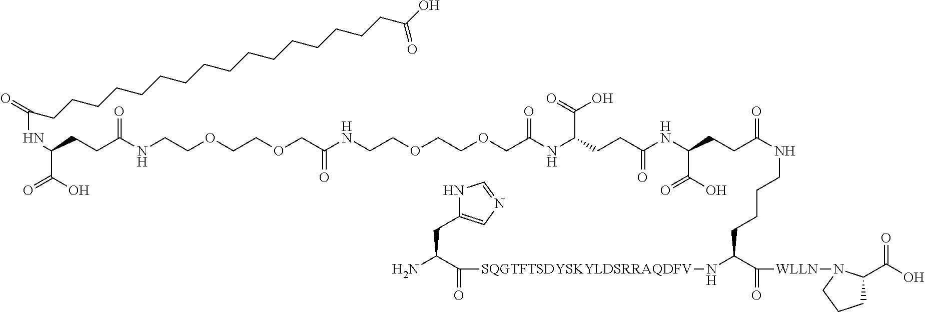

N^{ε28}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu²⁷,Lys²⁸]-Glucagonyl-Pro

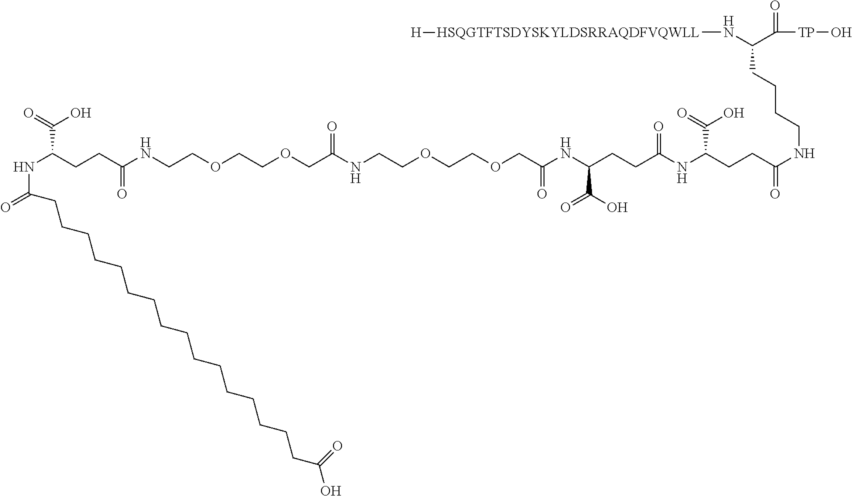

N^{ε12}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu²⁷]-Glucagon

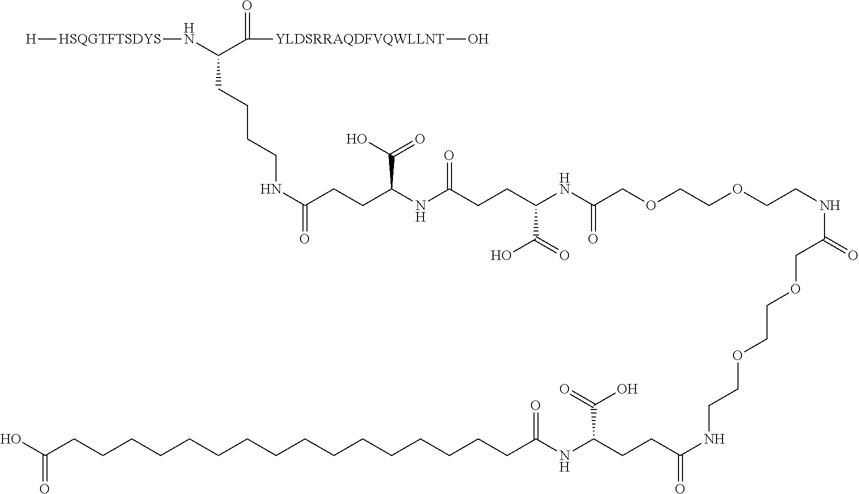

N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagonyl-Pro

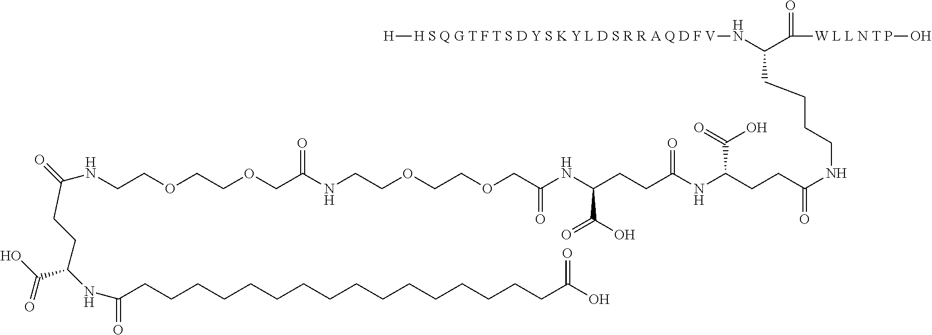

N^{ε27}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁷,Pro²⁹]-Glucagon

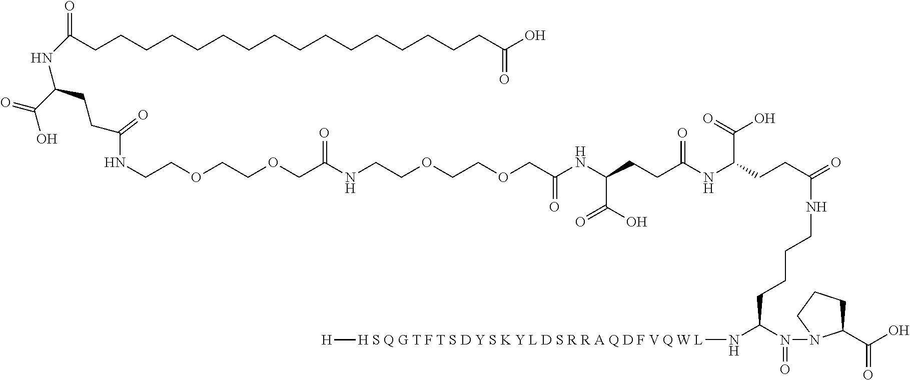

N^{ε28}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu²⁷,Lys²⁸,Pro²⁹]-Glucagon

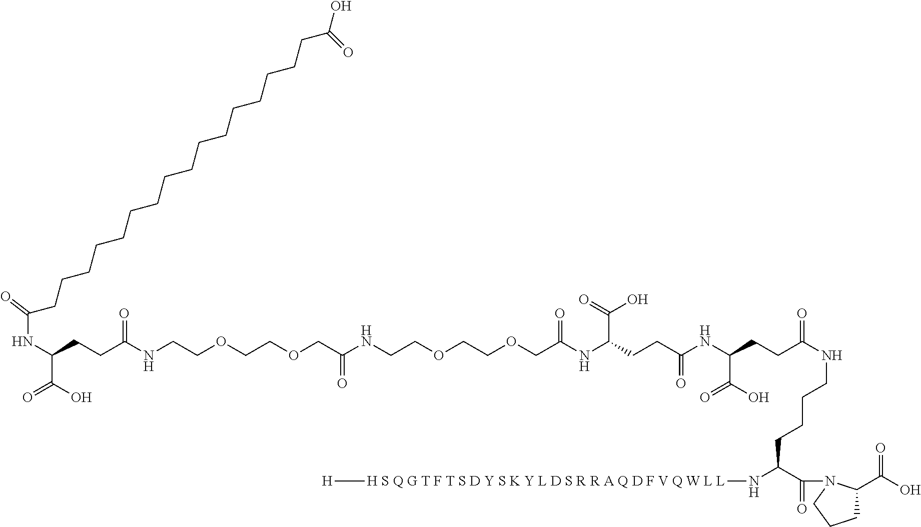

N^{ε27}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Arg¹², Lys²⁷,Pro²⁹]-Glucagon

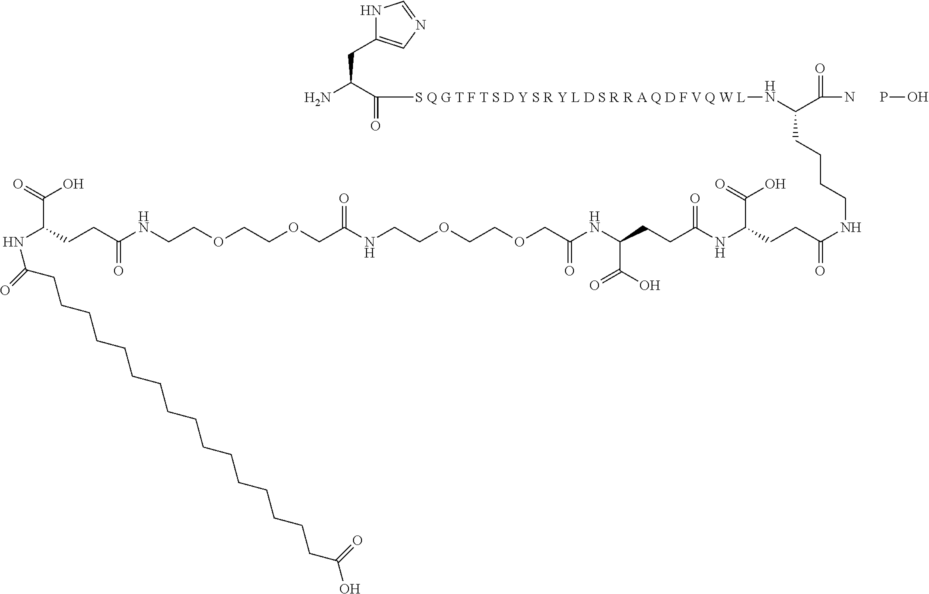

N^{ε24}-[(2S)-4-carboxy-2-[[(2S)-4-carboxy-2-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

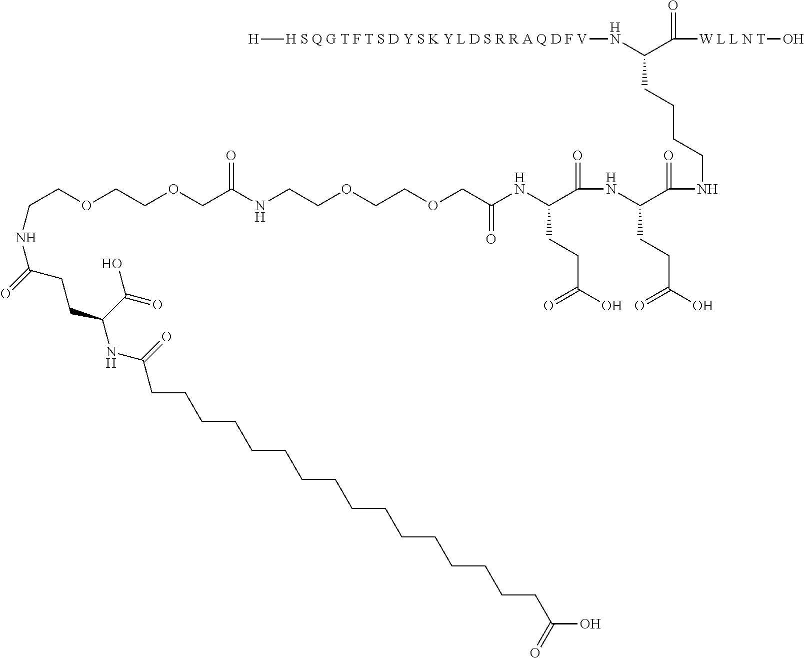

N^{ε24}-[(2S)-4-carboxy-2-[[(2S)-4-carboxy-2-[[2-[2-[2-[[2-[2-[2-[[(2S)-4-carboxy-2-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

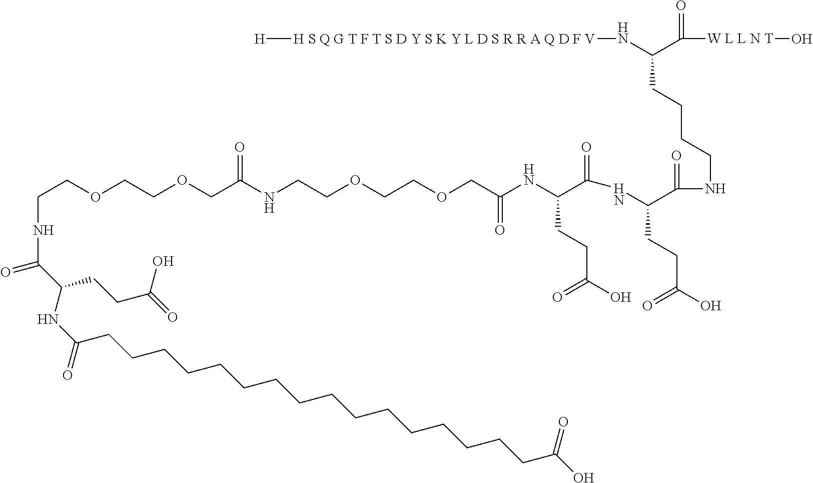

N^{ε24}-[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]-[Lys²⁴,Leu²⁷]-Glucagon

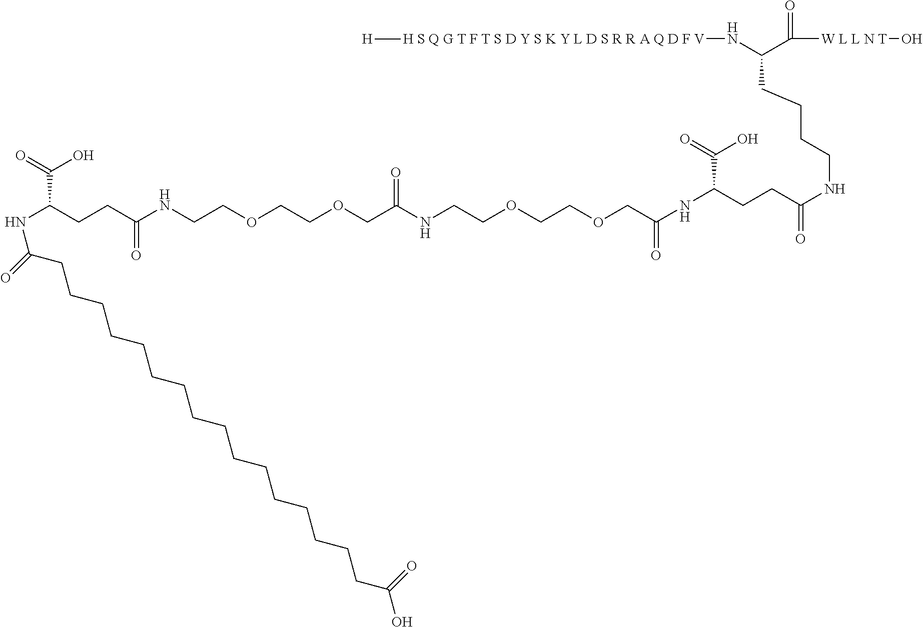

N^{ε24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu21,Lys24,Leu27,Ser28]-Glucagon

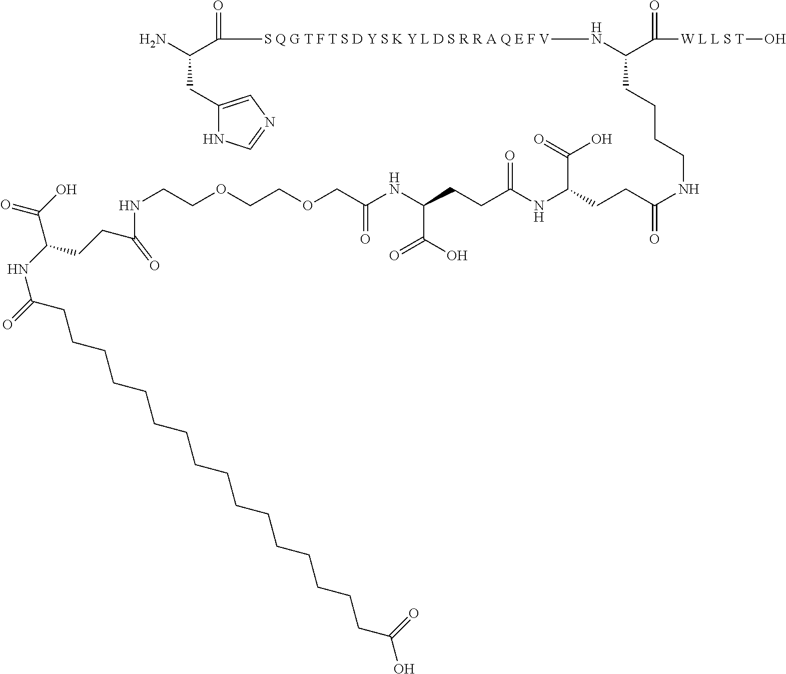

N^{ε24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu⁹,Lys²⁴,Leu²⁷,Ser²⁸]-Glucagon

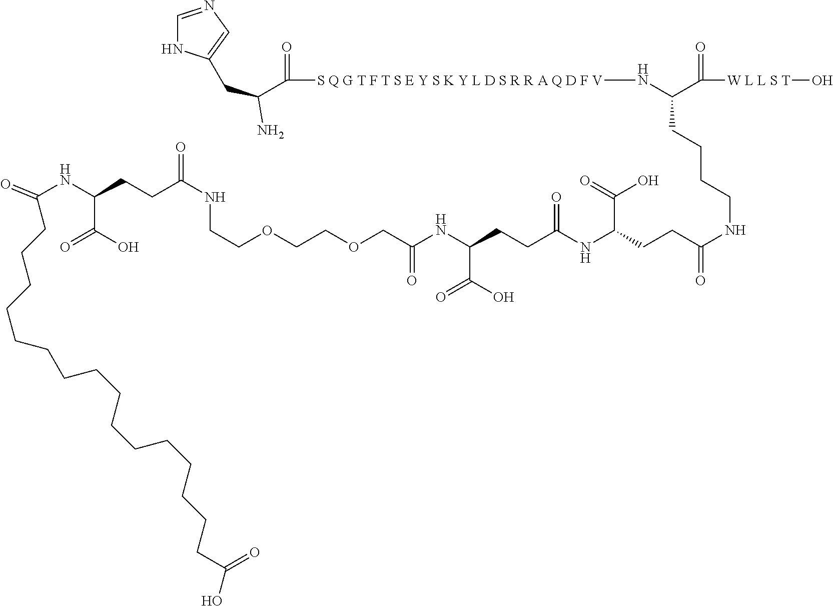

N^{ε24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu²⁰,Glu²¹,Lys²⁴,Leu²⁷,Ser²⁸]-Glucagon

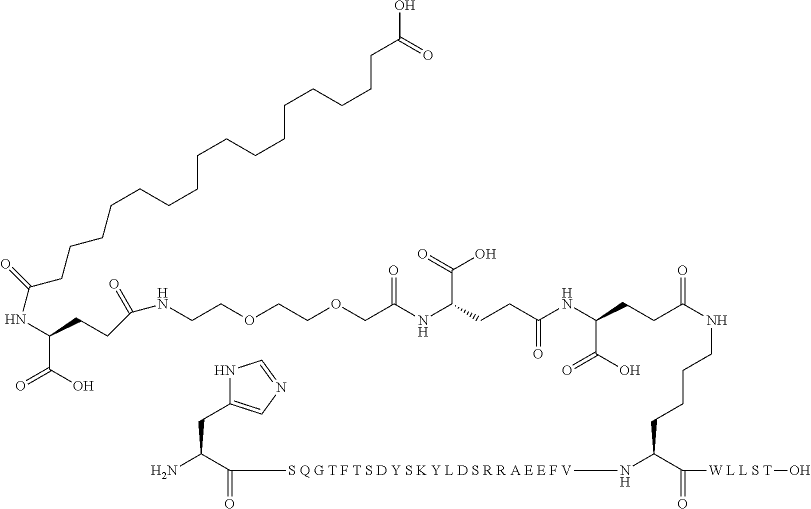

N^{ε24}-[(4S)-4-carboxy-4-[[(4S)-4-carboxy-4-[[2-[2-[2-[[2-[2-[2-[[(4S)-4-carboxy-4-(15-carboxypentadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

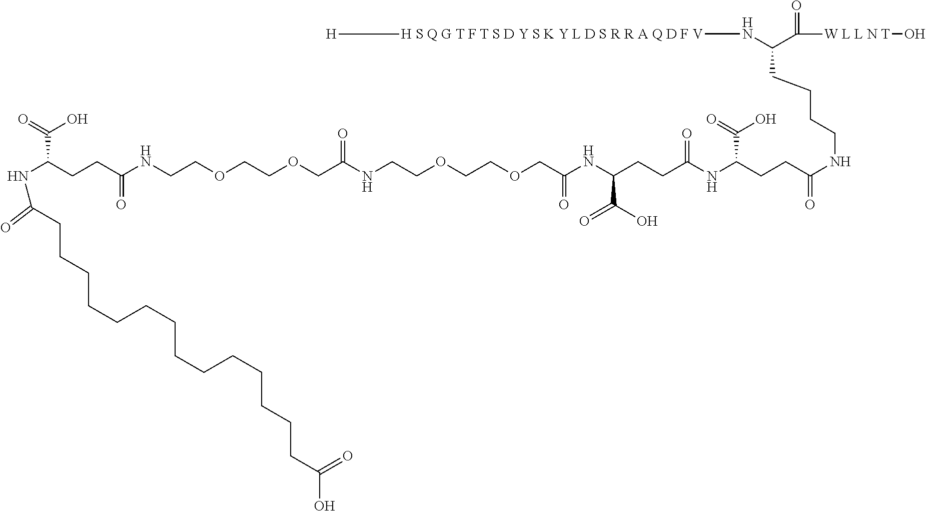

N^{ε24}-[(4S)-4-carboxy-4-[[(4S)-4-carboxy-4-[[2-[2-[2-[[2-[2-[2-[[(4S)-4-carboxy-4-(11-carboxyundecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

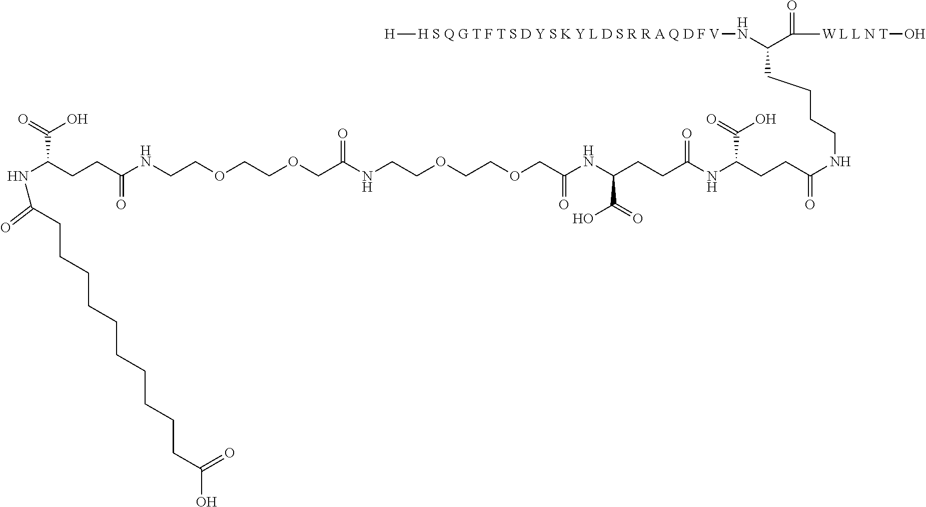

N^{ε24}-[(4S)-4-carboxy-4-[[(4S)-4-carboxy-4-[[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

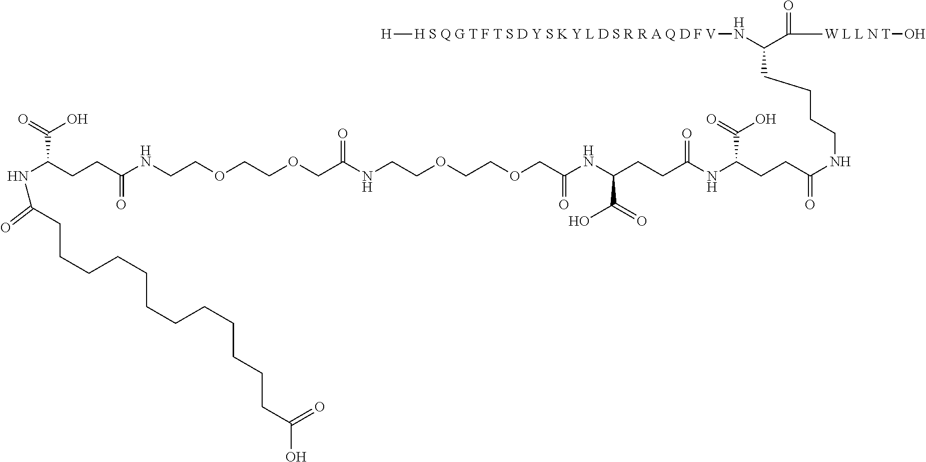

N^{ε24}-[(4S)-4-carboxy-4-[[(4S)-4-carboxy-4-[[2-[2-[2-[[2-[2-[2-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl]amino]ethoxy]ethoxy]acetyl]amino]butanoyl]amino]butanoyl]-[Lys²⁴,Leu²⁷]-Glucagon

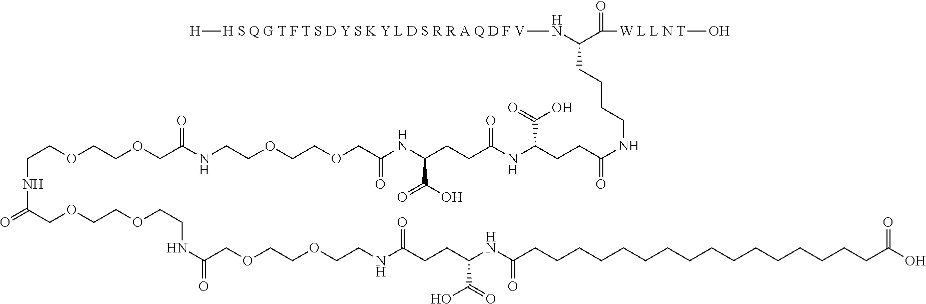

The N^{ε20}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁰,Leu²⁷]-Glucagon

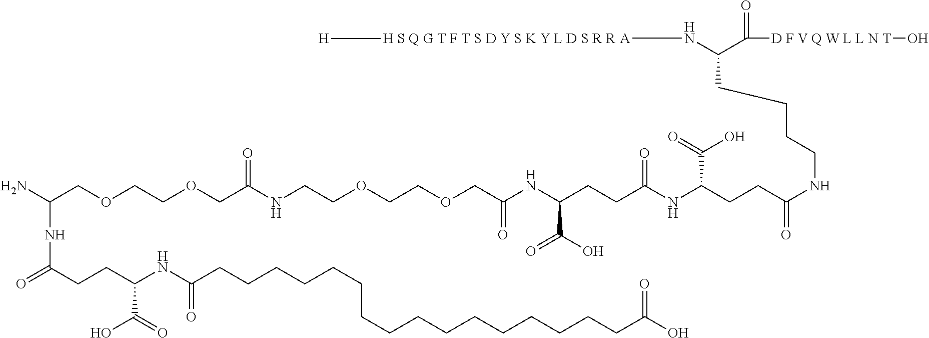

N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[D-Phe4,Lys24,Leu27,Ser28]-Glucagon

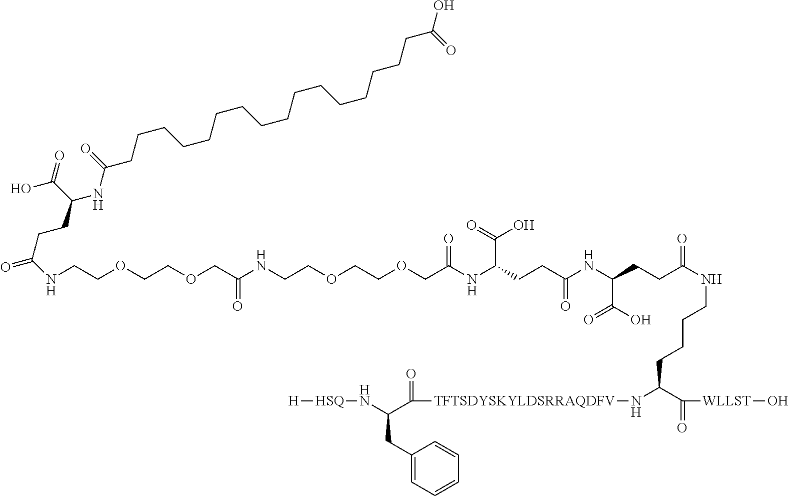

N^{ε16}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys¹⁶,Glu²¹,Arg²⁵,Leu²⁷]-Glucagon

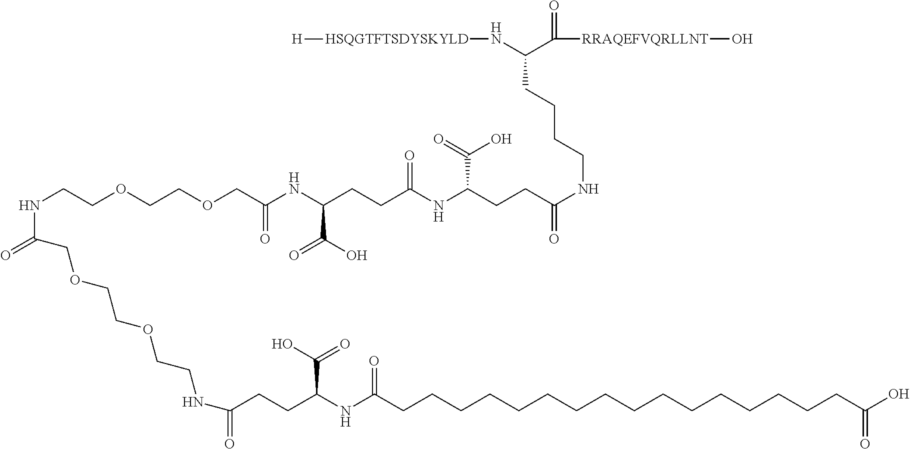

N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Glu²⁰,Lys²⁴,Leu²⁷,Ser²⁸]-Glucagon

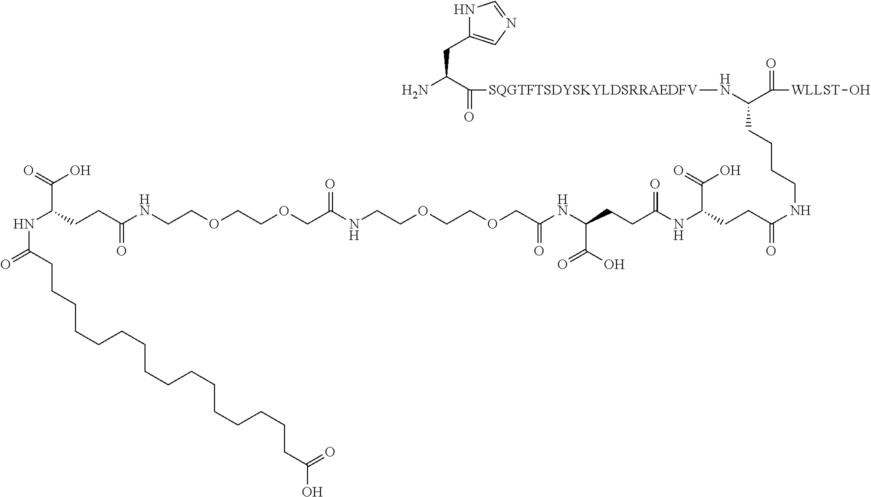

N^{ε24}-[(4S)-4-carboxy-4-[[(4S)-4-carboxy-4-[[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]amino]butanoyl]amino]butanoyl]-[Lys²⁴,Leu²⁷]-Glucagon

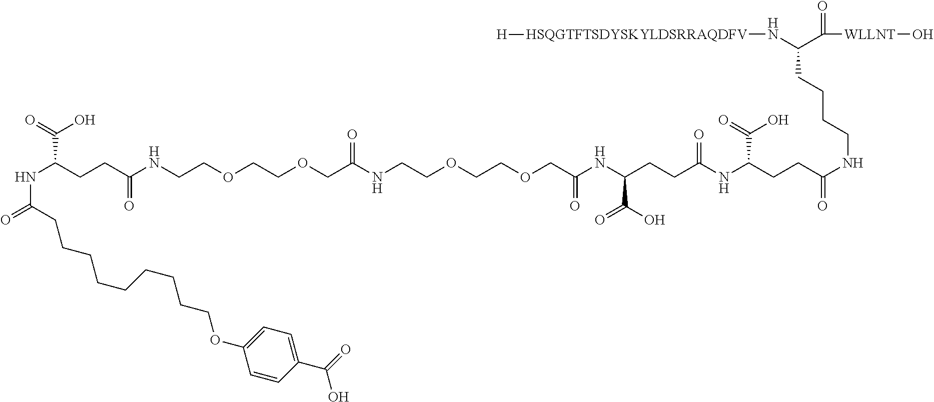

N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Gln²⁷]-Glucagon

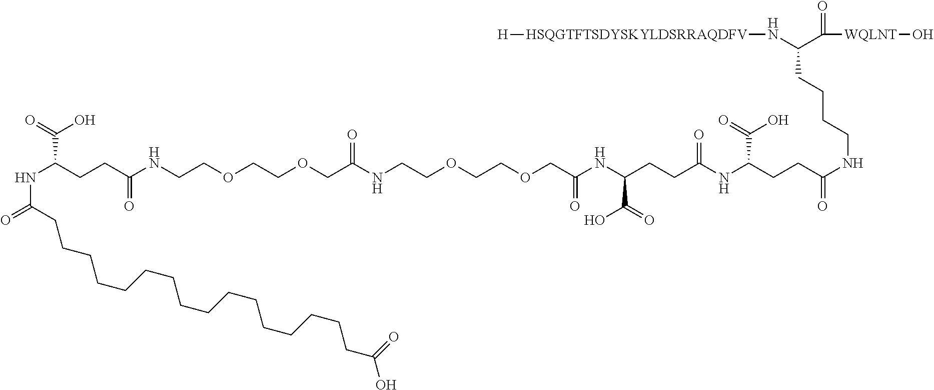

N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Glu²⁷]-Glucagon

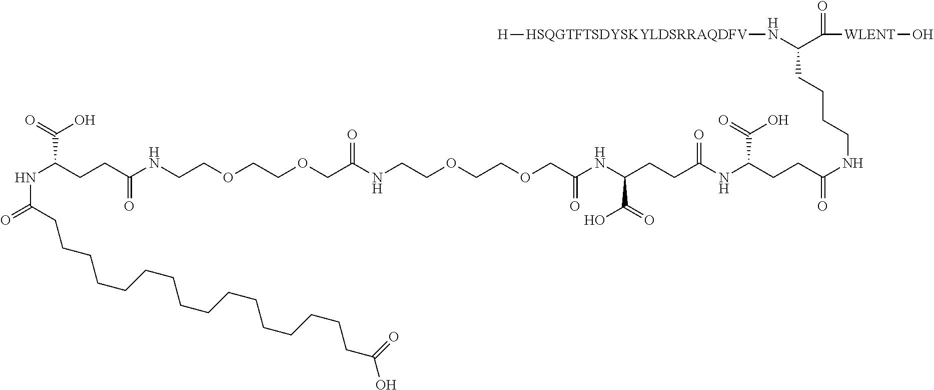

N^{α}([His²⁴,Leu²⁷]-Glucagonyl)-N^{ε}[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]Lys

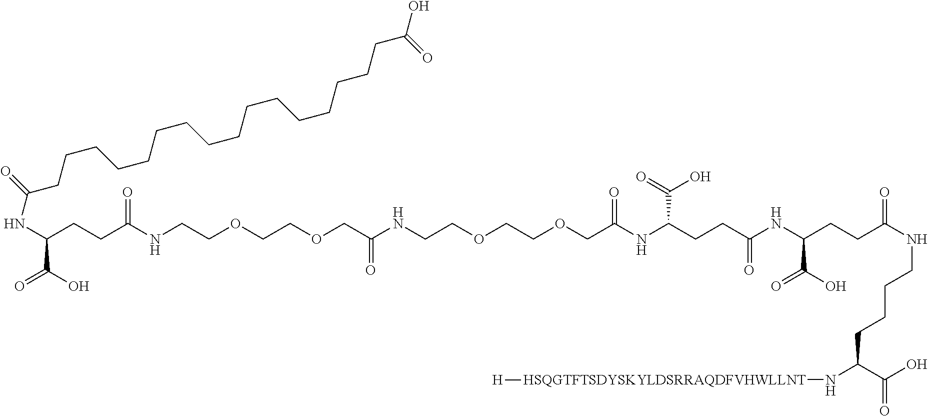

N^{ε24}-[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]-[Lys²⁴,Glu²⁷]-Glucagon

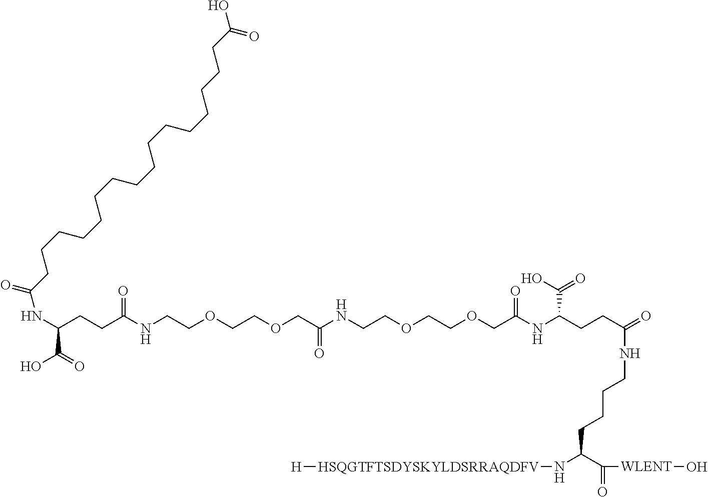

N^{ε24}-[(4S)-4-carboxy-4-[[(4S)-4-carboxy-4-[[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

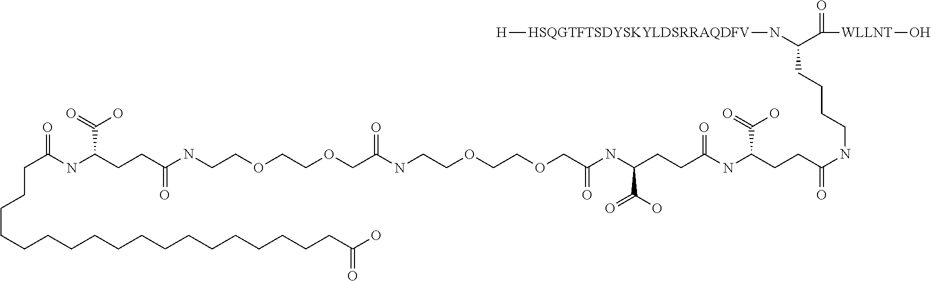

N^{ε24}-[(4S)-4-carboxy-4-[[(4S)-4-carboxy-4-[[2-[2-[2-[[2-[2-[2-[[(4S)-4-carboxy-4-(7-carboxyheptanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

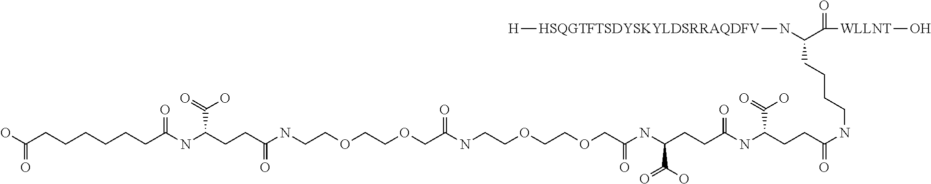

Further embodiments of the present invention relate to administration of the compounds of the present invention with antidiabetic agents or anti-obesity agents:

165. The glucagon peptide according to any one of the previous embodiments, in combination with a glucagon-like peptide 1 (GLP-1) compound.

166. The glucagon peptide according to any one of the previous embodiments, in combination with an insulinic compound.

167. The glucagon peptide according to any one of the previous embodiments, in combination with exendin-4.

168. The glucagon peptide according to any one of the previous embodiments, which is in a dual chamber, depository and/or micro-encapsulation formulation.

169. The glucagon peptide according to any one of the previous embodiments, in combination with a glucagon-like peptide 1 (GLP-1) compound, for the preparation of a medicament for the treatment of diabetes and/or obesity.

170. The glucagon peptide according to any one of the previous embodiments, in combination with an insulinic compound, for the preparation of a medicament for the treatment of diabetes and/or obesity.

171. The glucagon peptide according to any one of the previous embodiments, in combination with exendin-4, for the preparation of a medicament for the treatment of diabetes and/or obesity.

172. The glucagon peptide according to any one of the previous embodiments, wherein the GLP-1 compound and the insulinic compound are represented by formulas G1-G5:
N-epsilon26-((S)-4-Carboxy-4-hexadecanoylamino-butyryl)[Arg34]GLP-1-(7-37):

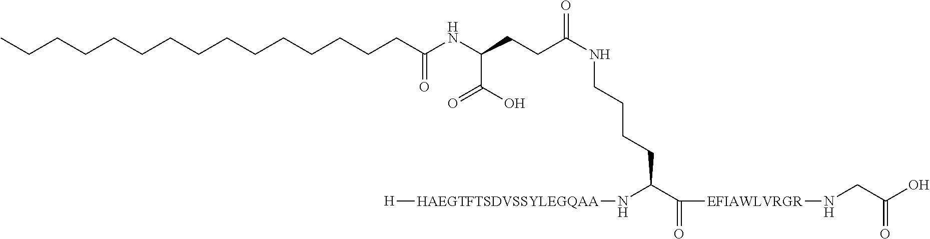

(compound G1);
N-epsilon37-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][DesaminoHis7,Glu22,Ara26,Ara34,Lys371]GLP-1-(7-37):

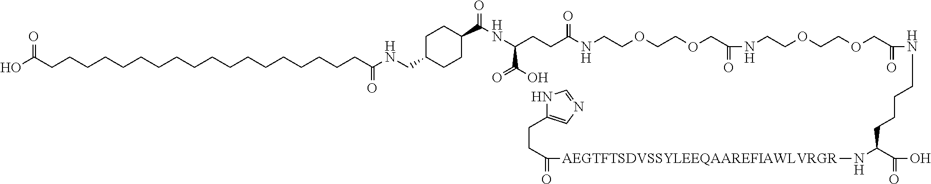

(compound G2);
N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Aib 8,Arg34]GLP-1-(7-37):

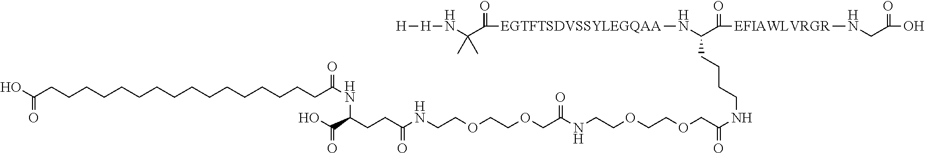

(compound G3);
N-epsilon37-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [Aib8,22,35,Lys³⁷]GLP-1-(7-37):

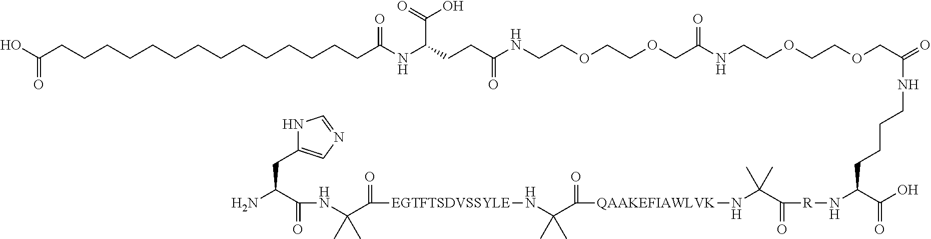

(compound G4) and
NεB29-hexadecandiyol-γ-Glu-(desB30) human insulin

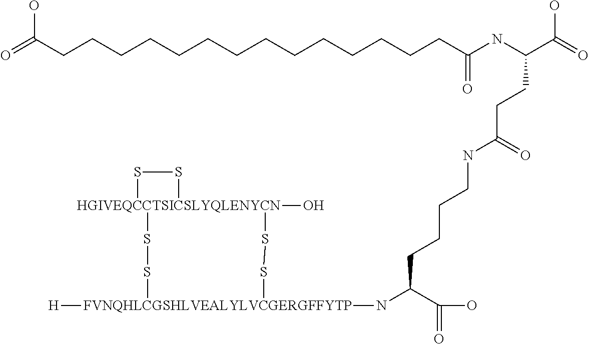

(compound G5).

GLP-1 is an incretin hormone produced by the endocrine cells of the intestine following ingestion of food. GLP-1 is a regulator of glucose metabolism, and the secretion of insulin from the beta cells of the islets of Langerhans in the pancreas. GLP-1 also causes insulin secretion in the diabetic state. The half-life in vivo of GLP-1 itself is, however, very short, thus, ways of prolonging the half-life of GLP-1 in vivo has attracted much attention.

WO 98/08871 discloses protracted GLP-1 analogues and derivatives based on human GLP-1(7-37) (amino acids 1-31 of SEQ ID NO:3) which have an extended half-life, including liraglutide, a GLP-1 derivative for once daily administration developed by Novo Nordisk A/S marketed for the treatment of type 2 diabetes.

Exenatide is a commercial incretin mimetic for the treatment of diabetes mellitus type 2 which is manufactured and marketed by Amylin Pharmaceuticals and Eli Lilly & Co. Exenatide is based on exendin-4, a hormone found in the saliva of the Gila monster. It displays biological properties similar to human GLP-1. U.S. Pat. No. 5,424,286 relates i.a. to a method of stimulating insulin release in a mammal by administration of exendin-4(7-45) (SEQ ID NO:1 in the US patent).

The term “GLP-1 compound” as used herein refers to human GLP-1(7-37) (amino acids 1-31 of SEQ ID NO:3), exendin-4(7-45) (amino acids 1-39 of SEQ ID NO:4), as well as analogues, fusion peptides, and derivatives thereof, which maintain GLP-1 activity.

As regards position numbering in GLP-1 compounds: for the present purposes any amino acid substitution, deletion, and/or addition is indicated relative to the sequences of SEQ ID NO:3, and/or 4. However, the numbering of the amino acid residues in the sequence listing always starts with no. 1, whereas for the present purpose we want, following the established practice in the art, to start with amino acid residue no. 7 and assign number 7 to it. Therefore, generally, any reference herein to a position number of the GLP-1 (7-37) or exendin-4 sequence is to the sequence starting with His at position 7 in both cases, and ending with Gly at position 37, or Ser at position 45, respectively.

GLP-1 compounds may be prepared as exemplified in example 65.

GLP-1 activity may be determined using any method known in the art, e.g. the assay (II) herein (stimulation of cAMP formation in a cell line expressing the human GLP-1 receptor).

Furthermore, the GLP-1 compound is a compound which may:

i) comprise at least one of the following: DesaminoHis7, Aib8, Aib22, Arg26, Aib35, and/or Lys37;

ii) be a GLP-1 derivative comprising an albumin binding moiety which comprises at least one, preferably at least two, more preferably two, free carboxylic acid groups; or a pharmaceutically acceptable salt thereof;

iii) be a GLP-1 derivative comprising an albumin binding moiety that comprises an acyl radical of a dicarboxylic acid, preferably comprising a total of from 12 to 24 carbon atoms, such as C12, C14, C16, C18, C20, C22, or C24, most preferably C16, C18, or C20; wherein preferably a) the acyl radical is attached to the epsilon amino group of a lysine residue of the GLP-1 peptide via a linker; b) the linker comprises at least one OEG radical, and/or at least one Trx radical, and, optionally, additionally at least one Glu; and/or

iv) be selected from the group consisting of compounds N-epsilon26-((S)-4-Carboxy-4-hexadecanoylamino-butyryl)[Arg34]GLP-1-(7-37):

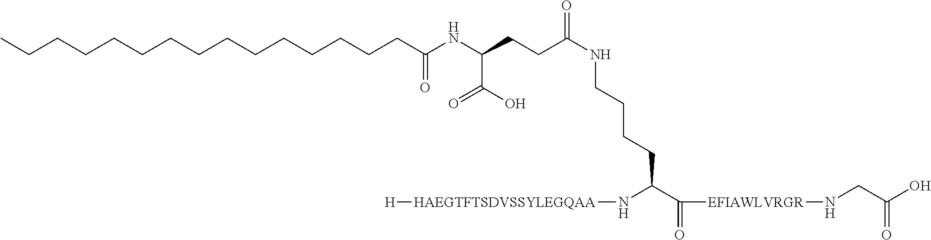

(compound G1);
N-epsilon37-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][DesaminoHis7,Glu22,Arg26,Arg34,Lys37]GLP-1-(7-37):

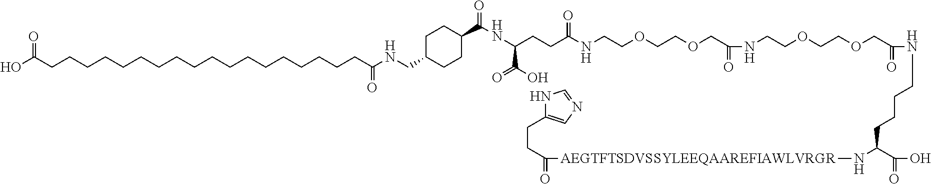

(compound G2);
N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Aib 8,Arg34]GLP-1-(7-37):

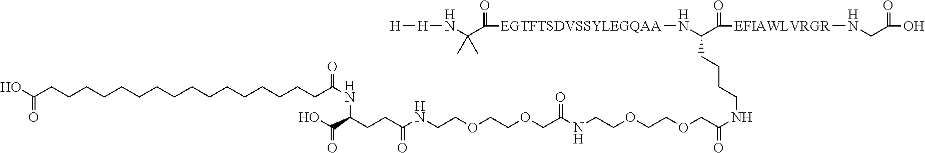

(compound G3);
N-epsilon37-[2-(2-{2-[2-(2-{2-[(S)-carboxy-4-(15-carboxy-pentadecanoylamino)-butyrylamino]-ethoxy}-ethoxy)-acetylamino]-ethoxy}-ethoxy)-acetyl][Aib8,22,35,Lys37]GLP-1-(7-37):

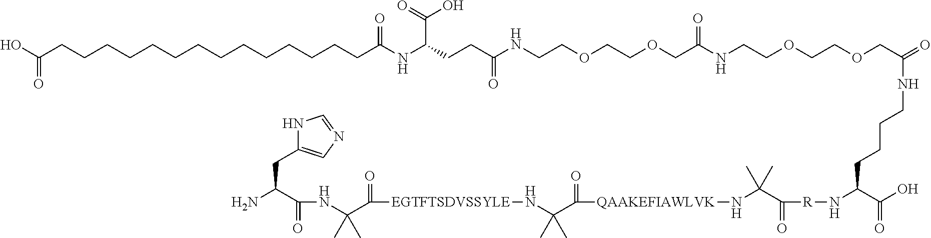

(compound G4);
and their pharmaceutically acceptable salts, amides, alkyls, or esters.

An “insulin” according to the invention is herein to be understood as human insulin, an insulin analogue or an insulin derivative.

The insulinic compound is a compound which may for example, be represented by:

NεB29-hexadecandiyol-γ-Glu-(desB30) human insulin

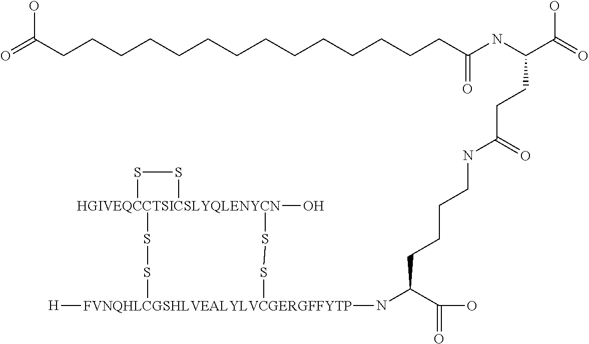

(compound G5);

The compounds of the present invention and anti-obesity or anti-diabetic agents as defined in the present specification, may be administered simultaneously or sequentially. The factors may be supplied in single-dosage form wherein the single-dosage form contains both compounds, or in the form of a kit-of-parts comprising a preparation of a compound of the present invention as a first unit dosage form and a preparation of a anti-obesity or anti-diabetic agents as a second unit dosage form. Whenever a first or second or third, etc., unit dose is mentioned throughout this specification this does not indicate the preferred order of administration, but is merely done for convenience purposes.

By “simultaneous” dosing of a preparation of a compound of the present invention and a preparation of anti-obesity or anti-diabetic agents is meant administration of the compounds in single-dosage form, or administration of a first agent followed by administration of a second agent with a time separation of no more than 15 minutes, preferably 10, more preferred 5, more preferred 2 minutes. Either factor may be administered first.

By “sequential” dosing is meant administration of a first agent followed by administration of a second agent with a time separation of more than 15 minutes. Either of the two unit dosage form may be administered first. Preferably, both products are injected through the same intravenous access.

As already indicated, in all of the therapeutic methods or indications disclosed above, a compound of the present invention may be administered alone. However, it may also be administered in combination with one or more additional therapeutically active agents, substances or compounds, either sequentially or concomitantly.

A typical dosage of a compound of the invention when employed in a method according to the present invention is in the range of from about 0.001 to about 100 mg/kg body weight per day, preferably from about 0.01 to about 10 mg/kg body weight, more preferably from about 0.01 to about 5 mg/kg body weight per day, e.g. from about 0.05 to about 10 mg/kg body weight per day or from about 0.03 to about 5 mg/kg body weight per day administered in one or more doses, such as from 1 to 3 doses. The exact dosage will depend upon the frequency and mode of administration, the sex, age, weight and general condition of the subject treated, the nature and severity of the condition treated, any concomitant diseases to be treated and other factors evident to those skilled in the art.

Compounds of the invention may conveniently be formulated in unit dosage form using techniques well known to those skilled in the art. A typical unit dosage form intended for oral administration one or more times per day, such as from one to three times per day, may suitably contain from about 0.05 to about 1000 mg, preferably from about 0.1 to about 500 mg, such as from about 0.5 to about 200 mg of a compound of the invention.

Compounds of the invention comprise compounds that are believed to be well-suited to administration with longer intervals than, for example, once daily, thus, appropriately formulated compounds of the invention may be suitable for, e.g., twice-weekly or once-weekly administration by a suitable route of administration, such as one of the routes disclosed herein.

As described above, compounds of the present invention may be administered or applied in combination with one or more additional therapeutically active compounds or substances, and suitable additional compounds or substances may be selected, for example, from antidiabetic agents, antihyperlipidemic agents, antiobesity agents, antihypertensive agents and agents for the treatment of complications resulting from, or associated with, diabetes.

Suitable antidiabetic agents include insulin, insulin derivatives or analogues, GLP-1 (glucagon like peptide-1) derivatives or analogues [such as those disclosed in WO 98/08871 (Novo Nordisk A/S), which is incorporated herein by reference, or other GLP-1 analogues such as exenatide (Byetta, Eli Lilly/Amylin; AVE0010, Sanofi-Aventis), taspoglutide (Roche), albiglutide (Syncria, GlaxoSmithKline), amylin, amylin analogues (e.g. Symlin™/Pramlintide) as well as orally active hypoglycemic agents.

Suitable orally active hypoglycemic agents include: metformin, imidazolines; sulfonylureas; biguanides; meglitinides; oxadiazolidinediones; thiazolidinediones; insulin sensitizers; α-glucosidase inhibitors; agents acting on the ATP-dependent potassium channel of the pancreatic β-cells, e.g. potassium channel openers such as those disclosed in WO 97/26265, WO 99/03861 and WO 00/37474 (Novo Nordisk A/S) which are incorporated herein by reference; potassium channel openers such as ormitiglinide; potassium channel blockers such as nateglinide or BTS-67582; glucagon receptor antagonists such as those disclosed in WO 99/01423 and WO 00/39088 (Novo Nordisk A/S and Agouron Pharmaceuticals, Inc.), all of which are incorporated herein by reference; GLP-1 receptor agonists such as those disclosed in WO 00/42026 (Novo Nordisk A/S and Agouron Pharmaceuticals, Inc.), which are incorporated herein by reference; amylin analogues (agonists on the amylin receptor); DPP-IV (dipeptidyl peptidase-IV) inhibitors; PTPase (protein tyrosine phosphatase) inhibitors; glucokinase activators, such as those described in WO 02/08209 to Hoffmann La Roche; inhibitors of hepatic enzymes involved in stimulation of gluconeogenesis and/or glycogenolysis; glucose uptake modulators; GSK-3 (glycogen synthase kinase-3) inhibitors; compounds modifying lipid metabolism, such as antihyperlipidemic agents and antilipidemic agents; compounds lowering food intake; as well as PPAR (peroxisome proliferator-activated receptor) agonists and RXR (retinoid X receptor) agonists such as ALRT-268, LG-1268 or LG-1069.

Other examples of suitable additional therapeutically active substances include insulin or insulin analogues; sulfonylureas, e.g. tolbutamide, chlorpropamide, tolazamide, glibenclamide, glipizide, glimepiride, glicazide or glyburide; biguanides, e.g. metformin; and meglitinides, e.g. repaglinide or senaglinide/nateglinide.

Further examples of suitable additional therapeutically active substances include thiazolidinedione insulin sensitizers, e.g. troglitazone, ciglitazone, pioglitazone, rosiglitazone, isaglitazone, darglitazone, englitazone, CS-011/CI-1037 or T 174, or the compounds disclosed in WO 97/41097 (DRF-2344), WO 97/41119, WO 97/41120, WO 00/41121 and WO 98/45292 (Dr. Reddy's Research Foundation), the contents of all of which are incorporated herein by reference.

Additional examples of suitable additional therapeutically active substances include insulin sensitizers, e.g. GI 262570, YM-440, MCC-555, JTT-501, AR-H039242, KRP-297, GW-409544, CRE-16336, AR-H049020, LY510929, MBX-102, CLX-0940, GW-501516 and the compounds disclosed in WO 99/19313 (NN622/DRF-2725), WO 00/50414, WO 00/63191, WO 00/63192 and WO 00/63193 (Dr. Reddy's Research Foundation), and in WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), the contents of all of which are incorporated herein by reference.

Still further examples of suitable additional therapeutically active substances include: α-glucosidase inhibitors, e.g. voglibose, emiglitate, miglitol or acarbose; glycogen phosphorylase inhibitors, e.g. the compounds described in WO 97/09040 (Novo Nordisk A/S); glucokinase activators; agents acting on the ATP-dependent potassium channel of the pancreatic β-cells, e.g. tolbutamide, glibenclamide, glipizide, glicazide, BTS-67582 or repaglinide;

Other suitable additional therapeutically active substances include antihyperlipidemic agents and antilipidemic agents, e.g. cholestyramine, colestipol, clofibrate, gemfibrozil, lovastatin, pravastatin, simvastatin, probucol or dextrothyroxine.

Further agents which are suitable as additional therapeutically active substances include antiobesity agents and appetite-regulating agents. Such substances may be selected from the group consisting of CART (cocaine amphetamine regulated transcript) agonists, NPY (neuropeptide Y receptor 1 and/or 5) antagonists, MC3 (melanocortin receptor 3) agonists, MC3 antagonists, MC4 (melanocortin receptor 4) agonists, orexin receptor antagonists, TNF (tumor necrosis factor) agonists, CRF (corticotropin releasing factor) agonists, CRF BP (corticotropin releasing factor binding protein) antagonists, urocortin agonists, neuromedin U analogues (agonists on the neuromedin U receptor subtypes 1 and 2), 33 adrenergic agonists such as CL-316243, AJ-9677, GW-0604, LY362884, LY377267 or AZ-40140, MC1 (melanocortin receptor 1) agonists, MCH (melanocyte-concentrating hormone) antagonists, CCK (cholecystokinin) agonists, serotonin reuptake inhibitors (e.g. fluoxetine, seroxat or citalopram), serotonin and norepinephrine reuptake inhibitors, 5HT (serotonin) agonists, 5HT6 agonists, 5HT2c agonists such as APD356 (U.S. Pat. No. 6,953,787), bombesin agonists, galanin antagonists, growth hormone, growth factors such as prolactin or placental lactogen, growth hormone releasing compounds, TRH (thyrotropin releasing hormone) agonists, UCP 2 or 3 (uncoupling protein 2 or 3) modulators, chemical uncouplers, leptin agonists, DA (dopamine) agonists (bromocriptin, doprexin), lipase/amylase inhibitors, PPAR modulators, RXR modulators, TR β agonists, adrenergic CNS stimulating agents, AGRP (agouti-related protein) inhibitors, histamine H3 receptor antagonists such as those disclosed in WO 00/42023, WO 00/63208 and WO 00/64884, the contents of all of which are incorporated herein by reference, exendin-4 analogues, GLP-1 analogues, ciliary neurotrophic factor, amylin analogues, peptide YY₃₋₃₆ (PYY3-36) (Batterham et al, Nature 418, 650-654 (2002)), PYY3-36 analogues, NPY Y2 receptor agonists, NPY Y4 receptor agonists and substances acting as combined NPY Y2 and NPY Y4 agonists, FGF21 and analogues thereof, μ-opioid receptor antagonists, oxyntomodulin or analogues thereof.

Further suitable antiobesity agents are bupropion (antidepressant), topiramate (anticonvulsant), ecopipam (dopamine D1/D5 antagonist) and naltrexone (opioid antagonist), and combinations thereof. Combinations of these antiobesity agents would be e.g.: phentermine+topiramate, bupropion sustained release (SR)+naltrexone SR, zonisamide SR and bupropion SR. Among embodiments of suitable antiobesity agents for use in a method of the invention as additional therapeutically active substances in combination with a compound of the invention are leptin and analogues or derivatives of leptin.

Additional embodiments of suitable antiobesity agents are serotonin and norepinephrine reuptake inhibitors, e.g. sibutramine.

Other embodiments of suitable antiobesity agents are lipase inhibitors, e.g. orlistat.

Still further embodiments of suitable antiobesity agents are adrenergic CNS stimulating agents, e.g. dexamphetamine, amphetamine, phentermine, mazindol, phendimetrazine, diethylpropion, fenfluramine or dexfenfluramine.

Other examples of suitable additional therapeutically active compounds include antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin.

The compounds of the present invention have higher glucagon receptor selectivity in relation to previously disclosed peptides in the art. The peptides of the present invention also have prolonged in vivo half-life. The compounds of the present invention can be a soluble glucagon receptor agonist, for example with solubility of at least 0.2 mmol/l, at least 0.5 mmol/l, at least 2 mmol/l, at least 4 mmol/l, at least 8 mmol/l, at least 10 mmol/l, or at least 15 mmol/l.

In the present context, if not stated otherwise, the terms “soluble”, “solubility”, “soluble in aquous solution”, “aqueous solubility”, “water soluble”, “water-soluble”, “water solubility” and “water-solubility”, refer to the solubility of a compound in water or in an aqueous salt or aqueous buffer solution, for example a 10 mM phosphate solution, or in an aqueous solution containing other compounds, but no organic solvents.

The term “polypeptide” and “peptide” as used herein means a compound composed of at least five constituent amino acids connected by peptide bonds. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (α-aminoisobutyric acid), Abu (α-aminobutyric acid), Tle (tert-butylglycine), β-alanine, 3-aminomethyl benzoic acid, anthranilic acid.

The term “analogue” as used herein referring to a polypeptide means a modified peptide wherein one or more amino acid residues of the peptide have been substituted by other amino acid residues and/or wherein one or more amino acid residues have been deleted from the peptide and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide. A simple system is used to describe analogues. Formulae of peptide analogs and derivatives thereof are drawn using standard single letter or three letter abbreviations for amino acids used according to IUPAC-IUB nomenclature.

The term “derivative” as used herein in relation to a peptide means a chemically modified peptide or an analogue thereof, wherein at least one substituent is not present in the unmodified peptide or an analogue thereof, i.e. a peptide which has been covalently modified. Typical modifications are amides, carbohydrates, alkyl groups, acyl groups, esters and the like.

All amino acids for which the optical isomer is not stated is to be understood to mean the L-isomer.

The term “glucagon peptide” as used herein means glucagon peptide, glucagon compound, compound according to the present invention, compound of the present invention, compound of formula I, a glucagon analogue, a glucagon derivative or a derivative of a glucagon analogue human glucagon, human glucagon(1-29), glucagon(1-30), glucagon(1-31), glucagon(1-32) as well as analogues, fusion peptides, and derivatives thereof, which maintain glucagon activity.

As regards position numbering in glucagon compounds: for the present purposes any amino acid substitution, deletion, and/or addition is indicated relative to the sequences of native human glucagon (1-29) (SEQ ID 1). Human glucagon amino acids positions 1-29 are herein to be the same as amino acid positions X₁ to X₂₉. The human glucagon (1-29) sequence is His-Ser-Gln-Gly-Thr-Phe-Thr-Ser-Asp-Tyr-Ser-Lys-Tyr-Leu-Asp-Ser-Arg-Arg-Ala-GI n-Asp-Phe-Val-Gln-Trp-Leu-Met-Asn-Thr (SEQ ID 1).

Glucagon(1-30) means human glucagon with an extension of one amino acid in the C-terminal, glucagon(1-31) means human glucagon with an extension of two amino acid in the C-terminal and glucagon(1-32) means human glucagon with an extension of three amino acid in the C-terminal.

The term “distal” as used herein, means most remote (terminal) from the point of attachment.

The term “negative charged moiety” as used herein, means a negatively chargeable chemical moiety such as, but not limited to a carboxylic acid, sulphonic acid or a tetrazole moiety.

The term “lipophilic moiety” as used herein, means an alkyl chain —(CH₂)n- where n=5-20.

The term “substituent” as used herein, means a chemical moiety or group replacing a hydrogen.

In embodiments of the invention a maximum of 17 amino acids in the glucagon analogue have been modified (substituted, deleted, added or any combination thereof) relative to human glucagon(1-29). In embodiments of the invention a maximum of 15 amino acids in the glucagon analogue have been modified. In embodiments of the invention a maximum of 10 amino acids in the glucagon analogue have been modified. In embodiments of the invention a maximum of 8 amino acids in the glucagon analogue have been modified. In embodiments of the invention a maximum of 7 amino acids in the glucagon analogue have been modified. In embodiments of the invention a maximum of 6 amino acids in the glucagon analogue have been modified. In embodiments of the invention a maximum of 5 amino acids in the glucagon analogue have been modified. In embodiments of the invention a maximum of 4 amino acids in the glucagon analogue have been modified. In embodiments of the invention a maximum of 3 amino acids in the glucagon analogue have been modified. In embodiments of the invention a maximum of 2 amino acids in the glucagon analogue have been modified. In embodiments of the invention 1 amino acid in the glucagon analogue has been modified.

Further embodiments of the present invention relate to:

173. A glucagon peptide according to any of the previous embodiments, wherein said glucagon peptide is a DPPIV protected compound.

174. A glucagon peptide according to any of the previous embodiments, wherein said glucagon peptide is DPPIV stabilised.

175. A glucagon peptide according to any of the previous embodiments, wherein said glucagon peptide is an agonist of the glucagon receptor.

176. A glucagon peptide according to any of the previous embodiments, wherein said glucagon peptide is an agonist of the glucagon receptor, with an EC₅₀<1 nM.

The term “DPP-IV protected” as used herein referring to a polypeptide means a polypeptide which has been chemically modified in order to render said compound resistant to the plasma peptidase dipeptidyl aminopeptidase-4 (DPP-IV). The DPP-IV enzyme in plasma is known to be involved in the degradation of several peptide hormones, e.g. glucagon, GLP-1, GLP-2, oxyntomodulin etc. Thus, a considerable effort is being made to develop analogues and derivatives of the polypeptides susceptible to DPP-IV mediated hydrolysis in order to reduce the rate of degradation by DPP-IV.

Furthermore, the compounds of the present invention are stabilized against DPP-IV cleavage in an albumin free assay as described in Assay VI.

The term “glucagon agonist” as used herein refers to any glucagon peptide which fully or partially activates the human glucagon receptor. In a preferred embodiment, the “glucagon agonist” is any glucagon peptide that binds to a glucagon receptor, preferably with an affinity constant (KD) or a potency (EC₅₀) of below 1 μM, e.g., below 100 nM or below 1 nM, as measured by methods known in the art and exhibits insulinotropic activity, where insulinotropic activity may be measured in vivo or in vitro assays known to those of ordinary skill in the art. For example, the glucagon agonist may be administered to an animal and the insulin concentration measured over time.

In the present context, the term “agonist” is intended to indicate a substance (ligand) that activates the receptor type in question.

In the present context, the term “antagonist” is intended to indicate a substance (ligand) that blocks, neutralizes or counteracts the effect of an agonist.

More specifically, receptor ligands may be classified as follows:

Receptor agonists, which activate the receptor; partial agonists also activate the receptor, but with lower efficacy than full agonists. A partial agonist will behave as a receptor partial antagonist, partially inhibiting the effect of a full agonist.

Receptor neutral antagonists, which block the action of an agonist, but do not affect the receptor-constitutive activity.

Receptor inverse agonists, which block the action of an agonist and at the same time attenuate the receptor-constitutive activity. A full inverse agonist will attenuate the receptor-constitutive activity completely; a partial inverse agonist will attenuate the receptor-constitutive activity to a lesser extent.

As used herein the term “antagonist” includes neutral antagonists and partial antagonists, as well as inverse agonists. The term “agonist” includes full agonists as well as partial agonists.

In the present context, the term “pharmaceutically acceptable salt” is intended to indicate a salt which is not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric and nitric acids, and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene-salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. (1977) 66, 2, which is incorporated herein by reference. Examples of relevant metal salts include lithium, sodium, potassium and magnesium salts, and the like. Examples of alkylated ammonium salts include methylammonium, dimethylammonium, trimethylammonium, ethylammonium, hydroxyethylammonium, diethylammonium, butylammonium and tetramethylammonium salts, and the like.

As use herein, the term “therapeutically effective amount” of a compound refers to an amount sufficient to cure, alleviate or partially arrest the clinical manifestations of a given disease and/or its complications. An amount adequate to accomplish this is defined as a “therapeutically effective amount”. Effective amounts for each purpose will depend on the severity of the disease or injury, as well as on the weight and general state of the subject. It will be understood that determination of an appropriate dosage may be achieved using routine experimentation, by constructing a matrix of values and testing different points in the matrix, all of which is within the level of ordinary skill of a trained physician or veterinarian.

The terms “treatment”, “treating” and other variants thereof as used herein refer to the management and care of a patient for the purpose of combating a condition, such as a disease or a disorder. The terms are intended to include the full spectrum of treatments for a given condition from which the patient is suffering, such as administration of the active compound(s) in question to alleviate symptoms or complications thereof, to delay the progression of the disease, disorder or condition, to cure or eliminate the disease, disorder or condition, and/or to prevent the condition, in that prevention is to be understood as the management and care of a patient for the purpose of combating the disease, condition, or disorder, and includes the administration of the active compound(s) in question to prevent the onset of symptoms or complications. The patient to be treated is preferably a mammal, in particular a human being, but treatment of other animals, such as dogs, cats, cows, horses, sheep, goats or pigs, is within the scope of the invention.

As used herein, the term “solvate” refers to a complex of defined stoichiometry formed between a solute (in casu, a compound according to the present invention) and a solvent. Solvents may include, by way of example, water, ethanol, or acetic acid.

The present invention also relates to substituents, which may have the general formula II:

Z₁—Z₂-Z₃—Z₄ [II],

wherein
Z1 may be a lipophillic hydrocarbon chain with a negatively charged group such as a carboxylic acid or a 5-yl tetrazole in the terminus,
Z₂ and Z₄ may comprise one or more moieties of gamma-glutamic acid or glutamic acid and, Z₃ may comprise one or more units of Ado. An example of a substituent of the present invention, in which moiety Z₄ is absent, may be:

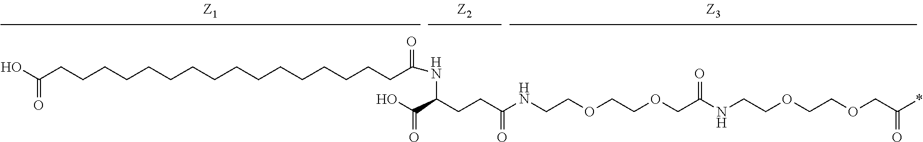 [SMILES: *C(=O)COCCOCCNC(=O)COCCOCCNC(=O)CC[C@H](NC(=O)CCCCCCCCCCCCCCCCC(=O)O)C(=O)O]

Where the symbol * indicates attachment point to the peptide.

In one embodiment, the substituent is attached via the epsilon position of a lysine or via the delta position of an ornithine and can reside on one or more of the following positions of peptide of formula I: X₁₀, X₁₂, X₁₆, X₁₇, X₁₈, X₂₀, X₂₁, X₂₄, X₂₅, X₂₇, X₂₈, X₂₉, and/or X₃₀.

In another embodiment, the substituent is attached via the epsilon position of a lysine or via the delta position of an ornithine and can reside on one or more of the following positions of peptide of formula I: X₁₂, X₁₆, X₂₄, X₂₅, X₂₇, X₂₈, X₂₉, and/or X₃₀.

In another embodiment, the substituent is attached via the epsilon position of a lysine or via the delta position of an ornithine and can reside on one or more of the following positions of peptide of formula I: X₂₄, X₂₈, X₂₉, and/or X₃₀.

In another embodiment, the substituent is attached via the epsilon position of a lysine or via the delta position of an ornithine and can reside on one or more of the following positions of peptide of formula I: X₂₄, X₂₈, X₂₉, and/or X₃₀.

Further embodiment of the present invention relate to a substituent:

177. A substituent with the formula II:

Z₁—Z₂—Z₃—Z₄ [11]

wherein,
Z₁ represents a structure according to one of the formulas IIa, IIb or IIc;

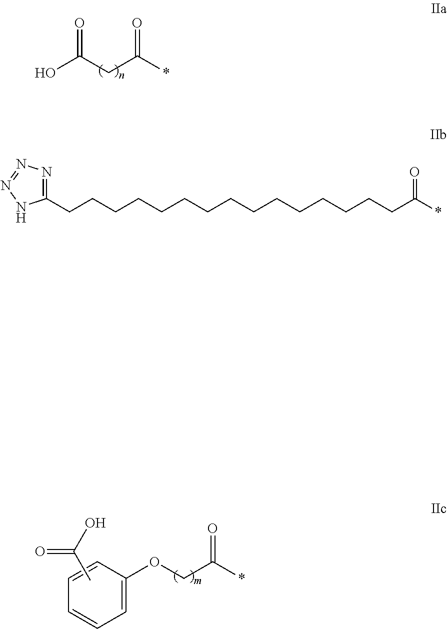

wherein n in formula IIa is 6-20,
m in formula IIc is 5-11,
the COOH group in fomula IIc can reside on position 2, 3 or 4 on the phenyl ring,
the symbol * in formula IIa, IIb and IIc represents the attachment point to the nitrogen in Z₂;
if Z₂ is absent, Z₁ is attached to the nitrogen on Z₃ at symbol * and if Z₂ and Z₃ are absent Z₁ is attached to the nitrogen on Z₄ at symbol *,
Z₂ is absent or represents a structure according to one of the formulas IId, IIe, IIf, IIg, IIh, IIi, IIj or IIk;

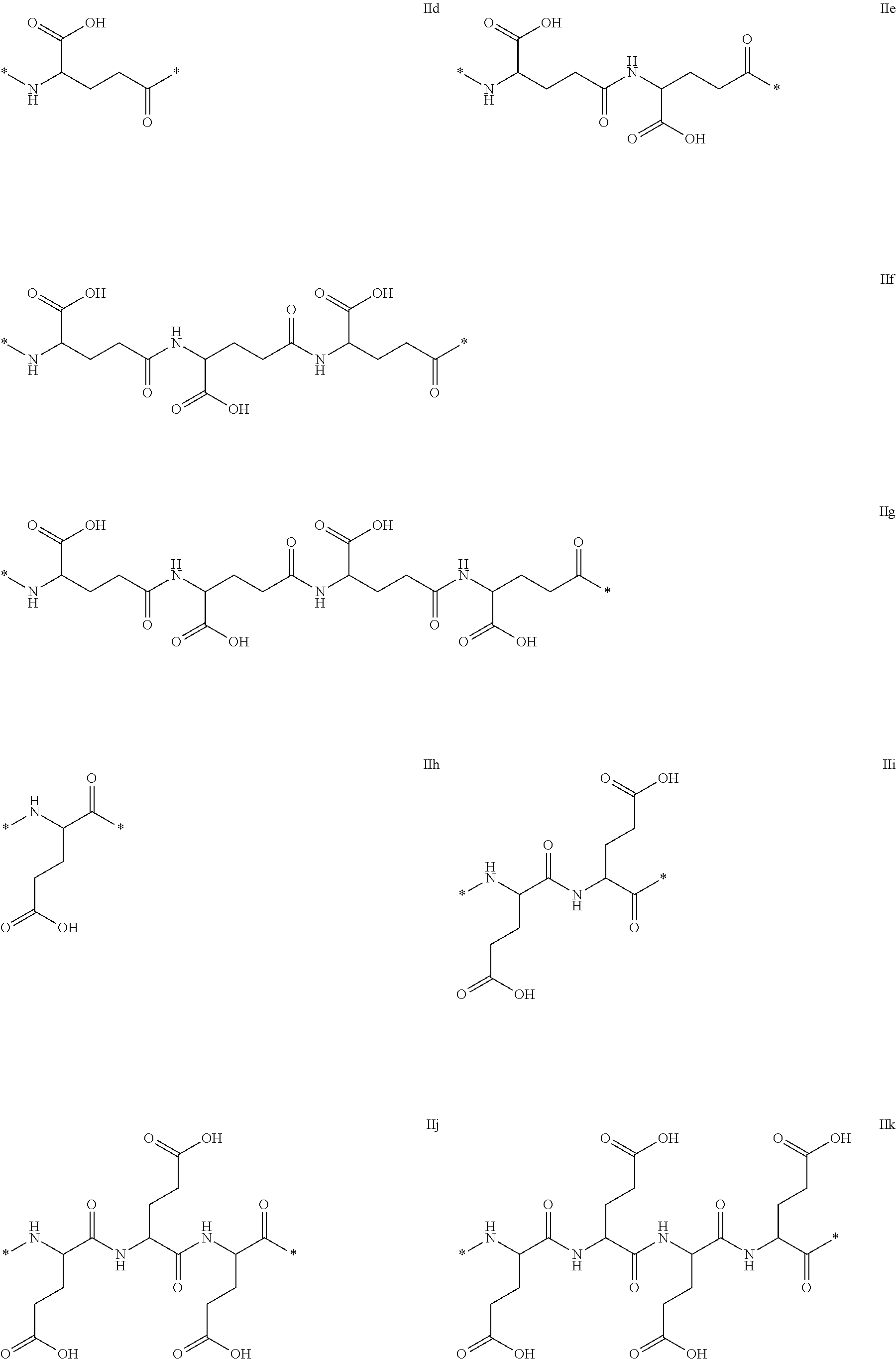

wherein each amino acid has the stereochemistry L or D;
wherein Z₂ is connected via the carbon atom denoted * to the nitrogen of Z₃ denoted *;
if Z₃ is absent, Z₂ is connected via the carbon atom denoted * to the nitrogen of Z₄ denoted * and
if Z₃ and Z₄ are absent Z₂, is connected via the carbon denoted * to the epsilon nitrogen of a lysine or the delta nitrogen of an ornithine of the glucagon peptide;
Z₃ is absent or represents a structure according to one of the formulas IIm, IIn, IIo or IIp;

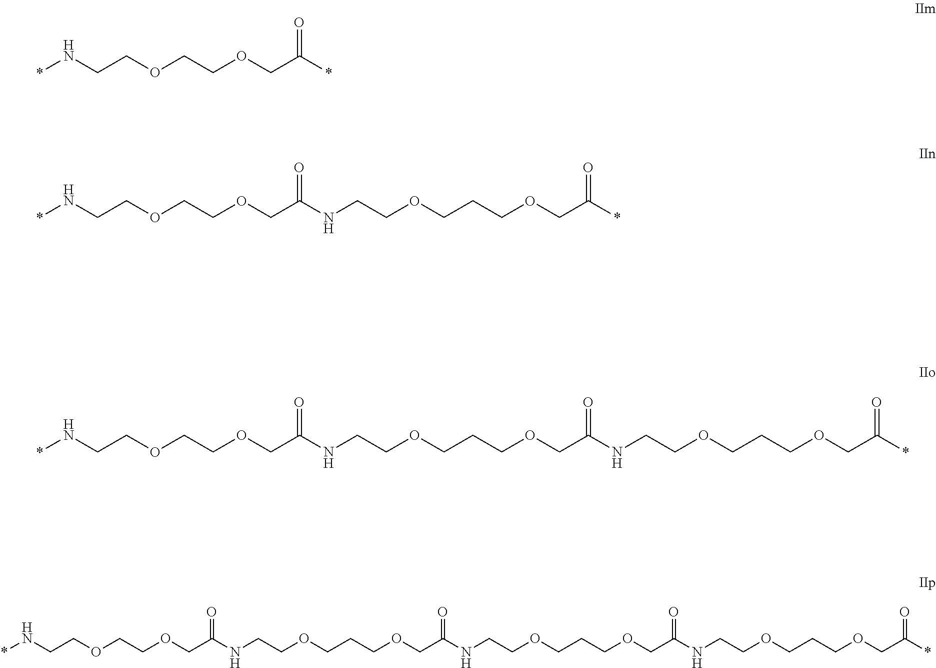 [SMILES: *NCCOCCOCC(*)=O ; *NCCOCCOCC(=O)NCCOCCCOCC(*)=O ; *NCCOCCOCC(=O)NCCOCCCOCC(=O)NCCOCCCOCC(*)=O ; *NCCOCCOCC(=O)NCCOCCCOCC(=O)NCCOCCCOCC(=O)NCCOCCCOCC(*)=O]

Z₃ is connected vi the carbon of Z₃ with symbol * to the nitrogen of Z₄ with symbol *, if Z₄ is absent Z₃ is connected via the carbon with symbol * to the epsilon nitrogen of a lysine or the delta nitrogen of an ornithine of the glucagon peptide;
Z₄ is absent or represents a structure according to one of the formulas IId, IIe, IIf, IIg, IIh, IIi, IIj or IIk; wherein each amino acid moiety is independently either L or D, wherein Z₄ is connected via the carbon with symbol * to the epsilon nitrogen of a lysine or the delta nitrogen of an ornithine of the glucagon peptide.

178. The substituent according to embodiment 177, wherein

Z₁ represents a structure according to one of the formulas IIa, IIb or IIc;

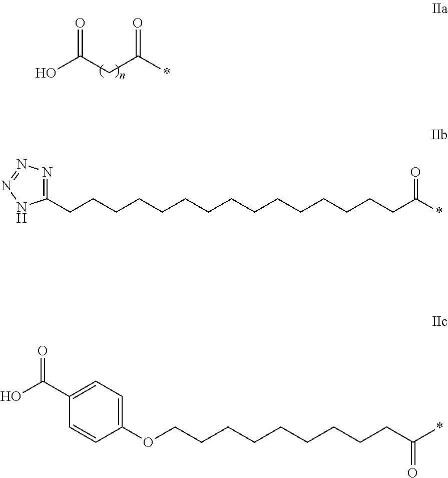

wherein n in formula IIa is 6-20,
Z₂ is absent or represents a structure according to one of the formulas IId, IIe, IIf, IIg, IIh, IIi, IIj or IIk;

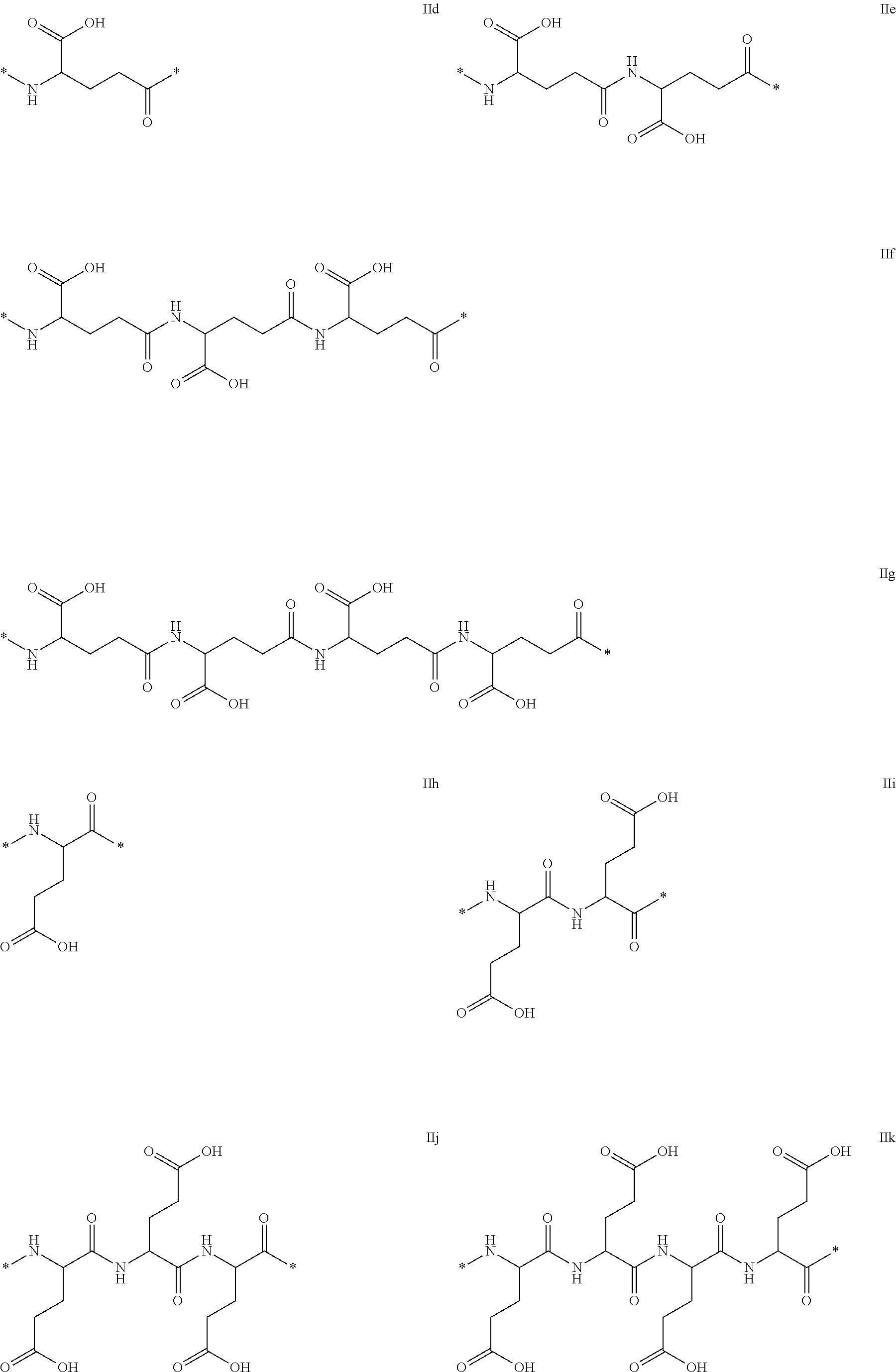

wherein each amino acid moiety is independently either L or D.

Z₃ is absent or represents a structure according to one of the formulas IIm, IIn, IIo or IIp;

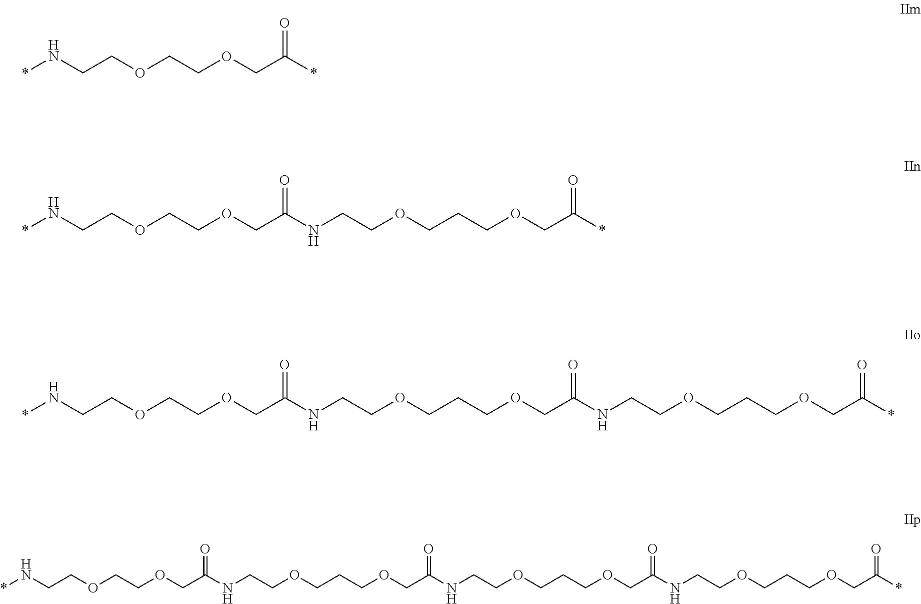 [SMILES: *NCCOCCOCC(*)=O ; *NCCOCCOCC(=O)NCCOCCCOCC(*)=O ; *NCCOCCOCC(=O)NCCOCCCOCC(=O)NCCOCCCOCC(*)=O ; *NCCOCCOCC(=O)NCCOCCCOCC(=O)NCCOCCCOCC(=O)NCCOCCCOCC(*)=O]

Z₄ is absent or represents a structure according to one of the formulas IId, IIe, IIf, IIg, IIh, IIi, IIj or IIk;
wherein each amino acid moiety is independently either L or D.

179. The substituent according to any one of embodiments 177-178, wherein Z₂ is absent when Z₄ is present.

180. The substituent according to any one of embodiments 177-178, wherein Z₄ is absent when Z₂ is present.

181. The substituent according to any one of embodiments 177-180, which is selected from the structures according to one of the formulas IIIa, IIIb, a, IIIb, IIIc, IIId, IIIe, IIIf, IIIg, IIIh, IIIi, IIIj, IIIk, IIIl, IIIm, IIIn or IIIo:

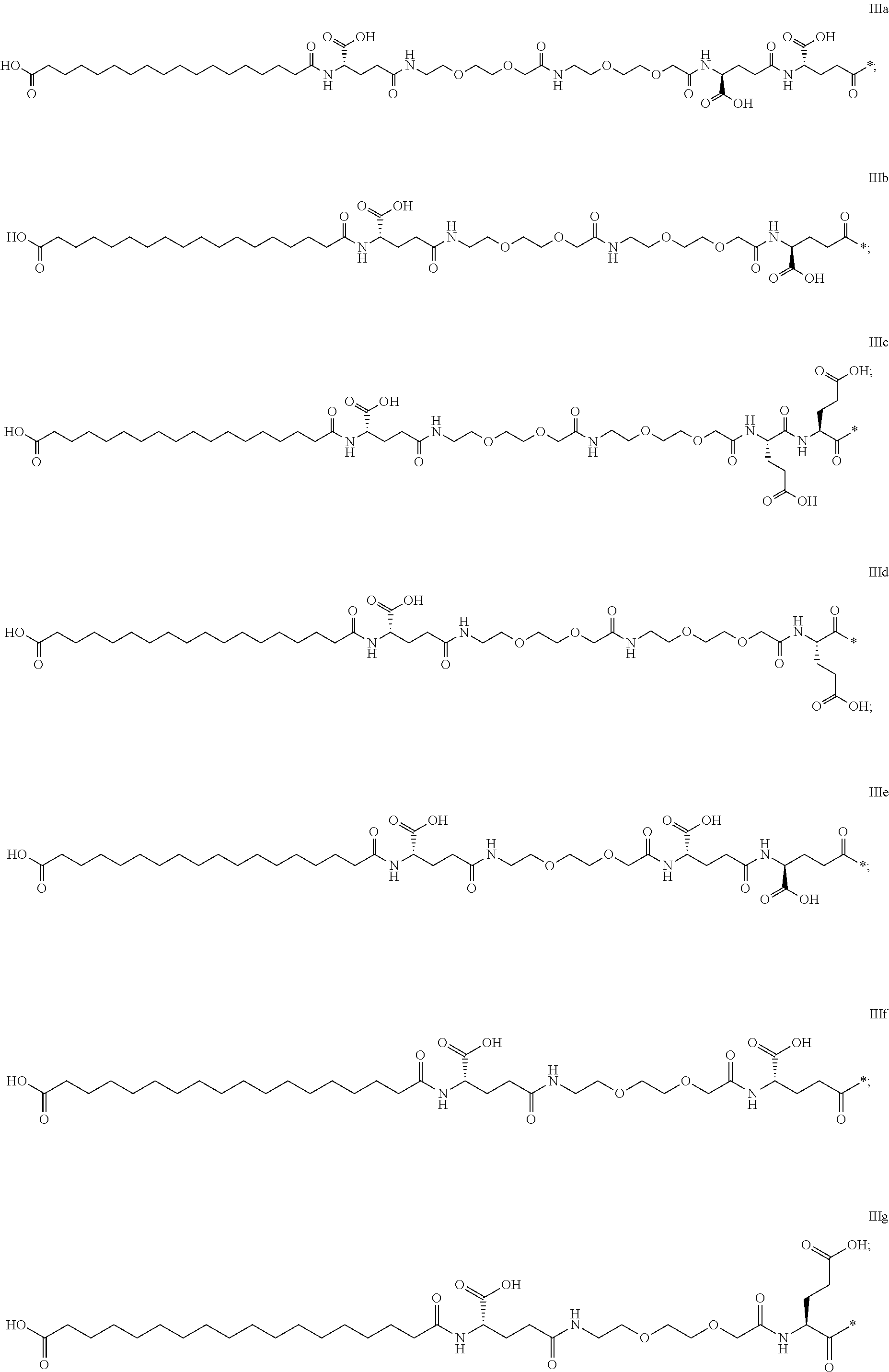

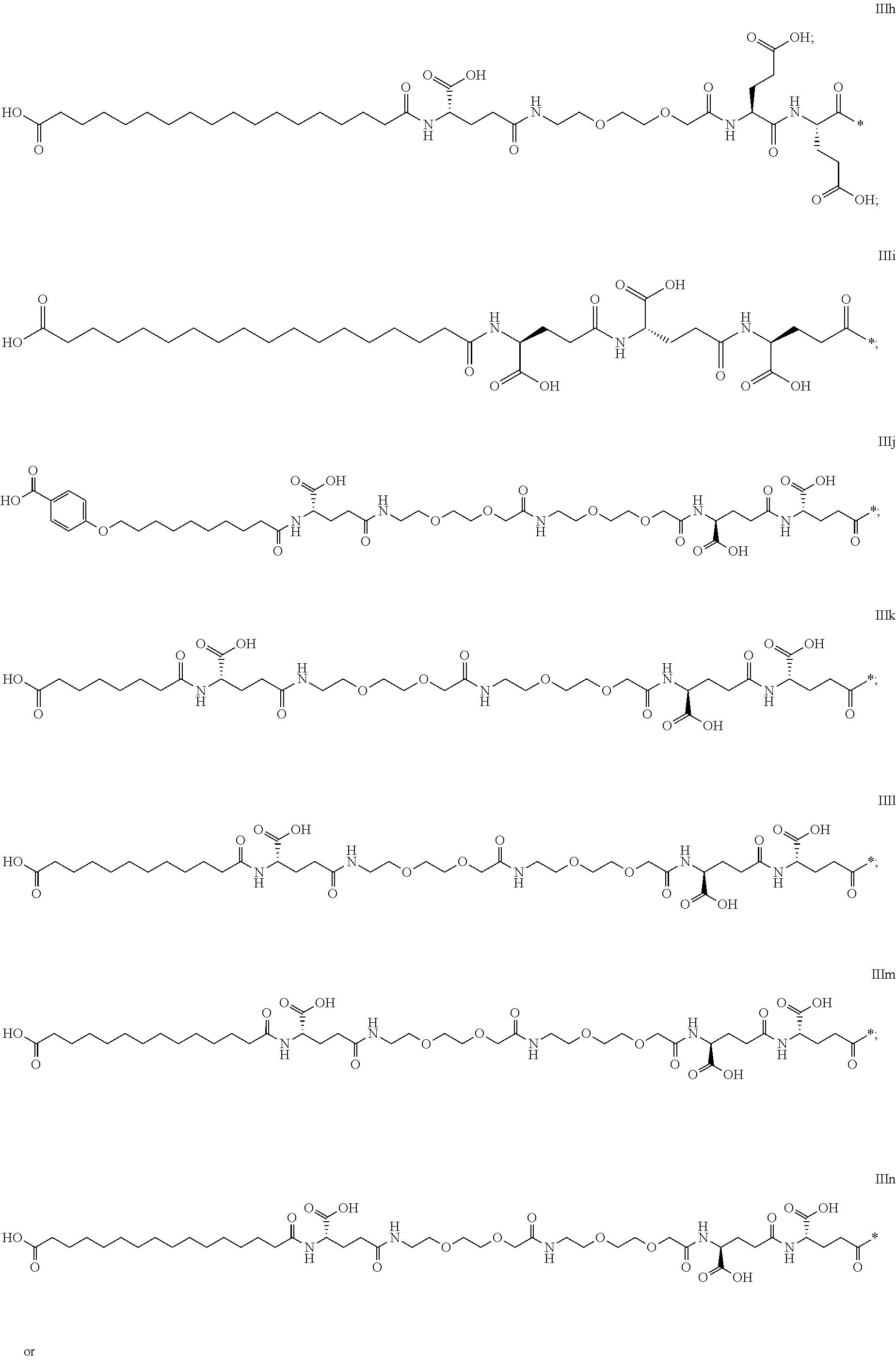

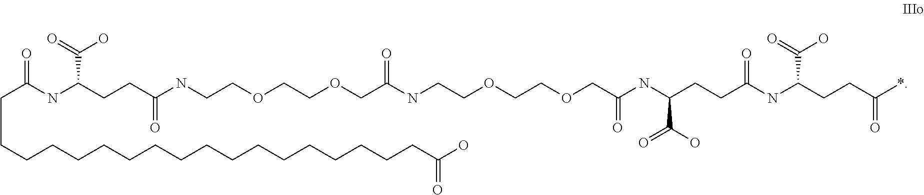

182. The substituent according to any one of embodiments 177-180, which represents a structure of formula IIIa:

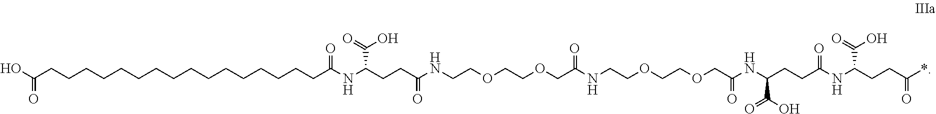

183. The substituent according to any one of embodiments 177-182, wherein Z₄ is absent.

184. The substituent according to any one of embodiments 177-182, wherein Z₃ and Z₄ are absent.

The term “albumin binding residue” as used herein means a residue which binds non-covalently to human serum albumin. The albumin binding residue attached to the therapeutic polypeptide typically has an affinity below 10 μM to human serum albumin and preferably below 1 μM. A range of albumin binding residues are known among linear and branched lipohophillic moieties containing 4-40 carbon atoms.

Other embodiments of the present relates to pharmaceutical compositions:

185. A pharmaceutical composition comprising a glucagon peptide according to any one of embodiments 1-176.

186. The pharmaceutical composition according to embodiment 185, further comprising one or more additional therapeutically active compounds or substances.

187. The pharmaceutical composition according to any one of embodiments 185-186, further comprising a GLP-1 compound.

188. The pharmaceutical composition according to any one of embodiments 185-186, wherein the GLP-1 compound is selected from the group consisting of:
N-epsilon26-((S)-4-Carboxy-4-hexadecanoylamino-butyryl)[Arg34]GLP-1-(7-37):

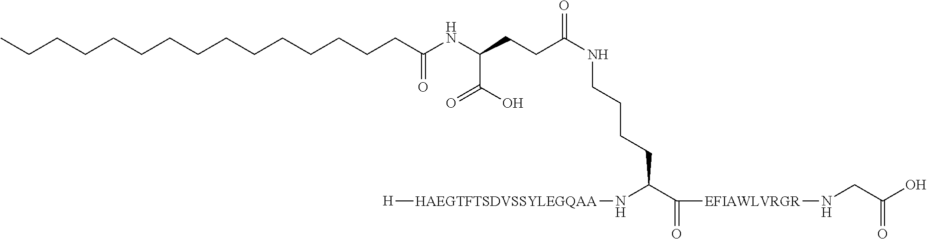

(compound G1);
N-epsilon37-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][DesaminoHis7,Glu22,Arg26,Arg34,Lys37]GLP-1-(7-37):

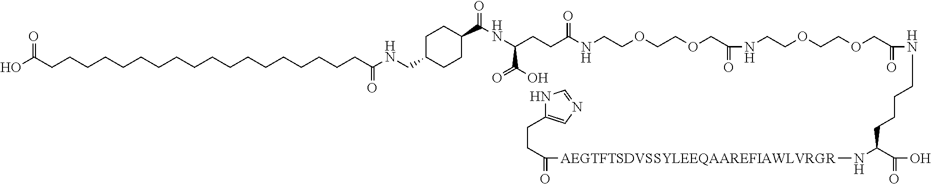

(compound G2);
N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Aib 8,Arg34]GLP-1-(7-37):

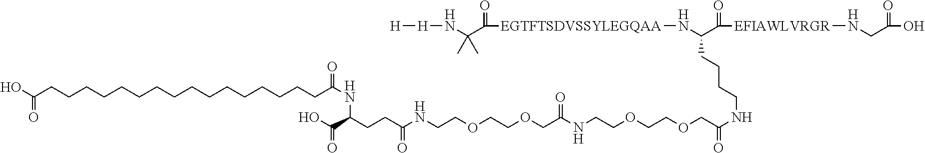

(compound G3);
N-epsilon37-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [Aib8,22,35,Lys37]GLP-1-(7-37):

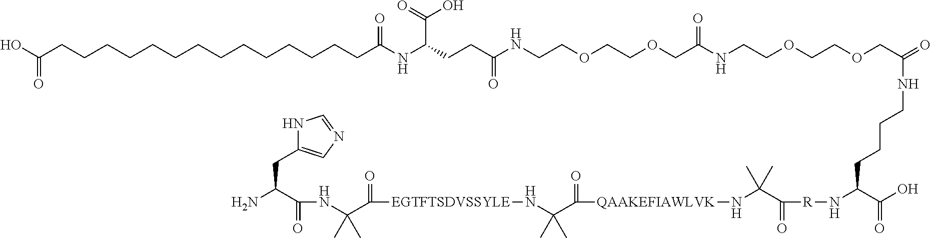

(compound G4);
and their pharmaceutically acceptable salts, amides, alkyls, or esters.

189. The pharmaceutical composition according to embodiments 185-188, further comprising an insulinic compound.

190. The pharmaceutical composition according to embodiment 189, wherein the insulin compound is:
NεB29-hexadecandiyol-γ-Glu-(desB30) human insulin

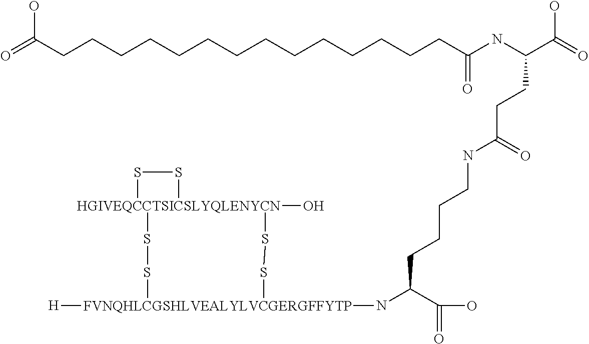

(compound G5);

191. The pharmaceutical composition according to any one of embodiments 185-190, in unit dosage form comprising from about 0.05 mg to about 1000 mg, such as from about 0.1 mg to about 500 mg, from about 2 mg to about 5 mg, e.g. from about 0.5 mg to about 200 mg, of a glucagon peptide according to any of embodiments 1-177.

192. The pharmaceutical composition according to any one of embodiments 185-190, which is suited for parenteral administration.

193. A glucagon peptide according to any of any one of embodiments 1-177, for use in therapy.

Further embodiments of the present invention relate to the following:

194. A glucagon peptide according to any of embodiments 1-177, optionally in combination with one or more additional therapeutically active compounds, for use in treatment or prevention of hyperglycemia, type 2 diabetes, impaired glucose tolerance, type 1 diabetes and obesity

195. A glucagon peptide according to any of embodiments 1-177, optionally in combination with one or more additional therapeutically active compounds, for use in delaying or preventing disease progression in type 2 diabetes.

196. A glucagon peptide according to any of embodiments 1-177, optionally in combination with one or more additional therapeutically active compounds, for use treating obesity or preventing overweight.

197. A glucagon peptide according to any of embodiments 1-177, optionally in combination with one or more additional therapeutically active compounds, for use in for decreasing food intake.

198. A glucagon peptide according to any of embodiments 1-177, optionally in combination with one or more additional therapeutically active compounds, for use in increasing energy expenditure.

199. A glucagon peptide according to any of embodiments 1-177, optionally in combination with one or more additional therapeutically active compounds, for use in reducing body weight.

200. A glucagon peptide according to any of embodiments 1-177, optionally in combination with one or more additional therapeutically active compounds, for use in delaying the progression from impaired glucose tolerance (IGT) to type 2 diabetes.

201. A glucagon peptide according to any of embodiments 1-177, optionally in combination with one or more additional therapeutically active compounds, for use in delaying the progression from type 2 diabetes to insulin-requiring diabetes.

202. A glucagon peptide according to any of embodiments 1-177, optionally in combination with one or more additional therapeutically active compounds, for use regulating appetite.

203. A glucagon peptide according to any of embodiments 1-177, optionally in combination with one or more additional therapeutically active compounds, for use inducing satiety.

204. A glucagon peptide according to any of embodiments 1-177, optionally in combination with one or more additional therapeutically active compounds, for use in preventing weight regain after successful weight loss.

205. A glucagon peptide according to any of embodiments 1-177, optionally in combination with one or more additional therapeutically active compounds, for use in treating a disease or state related to overweight or obesity.

206. A glucagon peptide according to any of embodiments 1-177, optionally in combination with one or more additional therapeutically active compounds, for use in treating bulimia.

207. A glucagon peptide according to any of embodiments 1-177, optionally in combination with one or more additional therapeutically active compounds, for use in treating binge-eating.

208. A glucagon peptide according to any of embodiments 1-177, optionally in combination with one or more additional therapeutically active compounds, for use in treating atherosclerosis.

209. A glucagon peptide according to any of embodiments 1-177, optionally in combination with one or more additional therapeutically active compounds, for use in treating hypertension.

210. A glucagon peptide according to any of embodiments 1-177, optionally in combination with one or more additional therapeutically active compounds, for use in treating type 2 diabetes.

211. A glucagon peptide according to any of embodiments 1-177, optionally in combination with one or more additional therapeutically active compounds, for use in treating impaired glucose tolerance.

212. A glucagon peptide according to any of embodiments 1-177, optionally in combination with one or more additional therapeutically active compounds, for use in treating dyslipidemia.

213. A glucagon peptide according to any of embodiments 1-177, optionally in combination with one or more additional therapeutically active compounds, for use in treating coronary heart disease.

214. A glucagon peptide according to any of embodiments 1-177, optionally in combination with one or more additional therapeutically active compounds, for use in treating hepatic steatosis.

215. A glucagon peptide according to any of embodiments 1-177, optionally in combination with one or more additional therapeutically active compounds, for use in treating hepatic steatosis.

216. A glucagon peptide according to any of embodiments 1-177, optionally in combination with one or more additional therapeutically active compounds, for use in treating beta-blocker poisoning.

217. A glucagon peptide according to any of embodiments 1-177, optionally in combination with one or more additional therapeutically active compounds, for use in inhibition of the motility of the gastrointestinal tract, useful in connection with investigations of the gastrointestinal tract using techniques such as x-ray, CT- and NMR-scanning.

218. A glucagon peptide according to any of embodiments 1-177, optionally in combination with one or more additional therapeutically active compounds, for use in treatment or prevention of hypoglycaemia.

219. A glucagon peptide according to any of embodiments 1-177, optionally in combination with one or more additional therapeutically active compounds, for use in treatment or prevention of insulin induced hypoglycaemia.

220. A glucagon peptide according to any of embodiments 1-177, optionally in combination with one or more additional therapeutically active compounds, for use in treatment or prevention of reactive hypoglycaemia.

221. A glucagon peptide according to any of embodiments 1-177, optionally in combination with one or more additional therapeutically active compounds, for use in treatment or prevention of diabetic hypoglycaemia.

222. A glucagon peptide according to any of embodiments 1-177, optionally in combination with one or more additional therapeutically active compounds, for use in treatment or prevention of non-diabetic hypoglycaemia.

223. A glucagon peptide according to any of embodiments 1-177, optionally in combination with one or more additional therapeutically active compounds, for use in treatment or prevention of fasting hypoglycaemia.

224. A glucagon peptide according to any of embodiments 1-177, optionally in combination with one or more additional therapeutically active compounds, for use in treatment or prevention of drug-induced hypoglycaemia.

225. A glucagon peptide according to any of embodiments 1-177, optionally in combination with one or more additional therapeutically active compounds, for use in treatment or prevention of gastric by-pass induced hypoglycaemia.

226. A glucagon peptide according to any of embodiments 1-177, optionally in combination with one or more additional therapeutically active compounds, for use in treatment or prevention of hypoglycemia in pregnancy.

227. A glucagon peptide according to any of embodiments 1-177, optionally in combination with one or more additional therapeutically active compounds, for use in treatment or prevention of alcohol-induced hypoglycaemia.

228. A glucagon peptide according to any of embodiments 1-177, optionally in combination with one or more additional therapeutically active compounds, for use in treatment or prevention of insulinoma.

229. A glucagon peptide according to any of embodiments 1-177, optionally in combination with one or more additional therapeutically active compounds, for use in treatment or prevention of Von Girkes disease.

Further embodiments of the present invention relate to the following methods:

230. A method for treating or preventing hyperglycemia, type 2 diabetes, impaired glucose tolerance, type 1 diabetes and obesity, comprising administering to a patient in need thereof, an effective amount of a glucagon peptide according to any of embodiments 1-177, optionally in combination with one or more additional therapeutically active compounds.

231. A method for delaying or preventing disease progression in type 2 diabetes, comprising administering to a patient in need thereof, an effective amount of a glucagon peptide according to any of embodiments 1-177, optionally in combination with one or more additional therapeutically active compounds.

232. A method for treating obesity or preventing overweight, comprising administering to a patient in need thereof, an effective amount of a glucagon peptide according to any of embodiments 1-177, optionally in combination with one or more additional therapeutically active compounds.

233. A method for decreasing food intake, comprising administering to a patient in need thereof, an effective amount of a glucagon peptide according to any of embodiments 1-177, optionally in combination with one or more additional therapeutically active compounds.

234. A method for use in increasing energy expenditure, comprising administering to a patient in need thereof, an effective amount of a glucagon peptide according to any of embodiments 1-177, optionally in combination with one or more additional therapeutically active compounds.

235. A method for use in reducing body weight, comprising administering to a patient in need thereof, an effective amount of a glucagon peptide according to any of embodiments 1-177, optionally in combination with one or more additional therapeutically active compounds.

236. A method for use in delaying the progression from impaired glucose tolerance (IGT) to type 2 diabetes, comprising administering to a patient in need thereof, an effective amount of a glucagon peptide according to any of embodiments 1-177, optionally in combination with one or more additional therapeutically active compounds.

237. A method for use in delaying the progression from type 2 diabetes to insulin-requiring diabetes, comprising administering to a patient in need thereof, an effective amount of a glucagon peptide according to any of embodiments 1-177, optionally in combination with one or more additional therapeutically active compounds.

238. A method for use in regulating appetite, comprising administering to a patient in need thereof, an effective amount of a glucagon peptide according to any of embodiments 1-177, optionally in combination with one or more additional therapeutically active compounds.

239. A method for use in inducing satiety, comprising administering to a patient in need thereof, an effective amount of a glucagon peptide according to any of embodiments 1-177, optionally in combination with one or more additional therapeutically active compounds.

240. A method for use in preventing weight regain after successful weight loss, comprising administering to a patient in need thereof, an effective amount of a glucagon peptide according to any of embodiments 1-177, optionally in combination with one or more additional therapeutically active compounds.

241. A method for use in treating a disease or state related to overweight or obesity, comprising administering to a patient in need thereof, an effective amount of a glucagon peptide according to any of embodiments 1-177, optionally in combination with one or more additional therapeutically active compounds.

242. A method for use in treating bulimia, comprising administering to a patient in need thereof, an effective amount of a glucagon peptide according to any of embodiments 1-177, optionally in combination with one or more additional therapeutically active compounds.

243. A method for use in treating binge-eating, comprising administering to a patient in need thereof, an effective amount of a glucagon peptide according to any of embodiments 1-177, optionally in combination with one or more additional therapeutically active compounds.

244. A method for use in treating atherosclerosis, comprising administering to a patient in need thereof, an effective amount of a glucagon peptide according to any of embodiments 1-177, optionally in combination with one or more additional therapeutically active compounds.

245. A method for use in treating hypertension, comprising administering to a patient in need thereof, an effective amount of a glucagon peptide according to any of embodiments 1-177, optionally in combination with one or more additional therapeutically active compounds.

246. A method for use in treating type 2 diabetes, comprising administering to a patient in need thereof, an effective amount of a glucagon peptide according to any of embodiments 1-177, optionally in combination with one or more additional therapeutically active compounds.

247. A method for use in treating impaired glucose tolerance, comprising administering to a patient in need thereof, an effective amount of a glucagon peptide according to any of embodiments 1-177, optionally in combination with one or more additional therapeutically active compounds.

248. A method for use in treating dyslipidemia, comprising administering to a patient in need thereof, an effective amount of a glucagon peptide according to any of embodiments 1-177, optionally in combination with one or more additional therapeutically active compounds.

249. A method for use in treating coronary heart disease, comprising administering to a patient in need thereof, an effective amount of a glucagon peptide according to any of embodiments 1-177, optionally in combination with one or more additional therapeutically active compounds.

250. A method for use in treating hepatic steatosis, comprising administering to a patient in need thereof, an effective amount of a glucagon peptide according to any of embodiments 1-177, optionally in combination with one or more additional therapeutically active compounds.

251. A method for use in treating beta-blocker poisoning, comprising administering to a patient in need thereof, an effective amount of a glucagon peptide according to any of embodiments 1-177, optionally in combination with one or more additional therapeutically active compounds.

252. A method for use in inhibition of the motility of the gastrointestinal tract, useful in connection with investigations of the gastrointestinal tract using techniques such as x-ray, CT- and NMR-scanning, comprising administering to a patient in need thereof, an effective amount of a glucagon peptide according to any of embodiments 1-177, optionally in combination with one or more additional therapeutically active compounds.

253. A method for use in treatment or prevention of hypoglycaemia, comprising administering to a patient in need thereof, an effective amount of a glucagon peptide according to any of embodiments 1-177, optionally in combination with one or more additional therapeutically active compounds.

254. A method for use in treatment or prevention of insulin induced hypoglycaemia, comprising administering to a patient in need thereof, an effective amount of a glucagon peptide according to any of embodiments 1-177, optionally in combination with one or more additional therapeutically active compounds.

255. A method for use in treatment or prevention of reactive hypoglycaemia, comprising administering to a patient in need thereof, an effective amount of a glucagon peptide according to any of embodiments 1-177, optionally in combination with one or more additional therapeutically active compounds.

256. A method for use in treatment or prevention of diabetic hypoglycaemia, comprising administering to a patient in need thereof, an effective amount of a glucagon peptide according to any of embodiments 1-177, optionally in combination with one or more additional therapeutically active compounds.

257. A method for use in treatment or prevention of non-diabetic hypoglycaemia, comprising administering to a patient in need thereof, an effective amount of a glucagon peptide according to any of embodiments 1-177, optionally in combination with one or more additional therapeutically active compounds.

258. A method for use in treatment or prevention of fasting hypoglycaemia, comprising administering to a patient in need thereof, an effective amount of a glucagon peptide according to any of embodiments 1-177, optionally in combination with one or more additional therapeutically active compounds.

259. A method for use in treatment or prevention of drug-induced hypoglycaemia, comprising administering to a patient in need thereof, an effective amount of a glucagon peptide according to any of embodiments 1-177, optionally in combination with one or more additional therapeutically active compounds.

260. A method for use in treatment or prevention of gastric by-pass induced hypoglycaemia, comprising administering to a patient in need thereof, an effective amount of a glucagon peptide according to any of embodiments 1-177, optionally in combination with one or more additional therapeutically active compounds.

261. A method for use in treatment or prevention of hypoglycemia in pregnancy, comprising administering to a patient in need thereof, an effective amount of a glucagon peptide according to any of embodiments 1-177, optionally in combination with one or more additional therapeutically active compounds.

262. A method for use in treatment or prevention of alcohol-induced hypoglycaemia, comprising administering to a patient in need thereof, an effective amount of a glucagon peptide according to any of embodiments 1-177, optionally in combination with one or more additional therapeutically active compounds.

263. A method for use in treatment or prevention of insulinoma, comprising administering to a patient in need thereof, an effective amount of a glucagon peptide according to any of embodiments 1-177, optionally in combination with one or more additional therapeutically active compounds.

264. A method for use in treatment or prevention of Von Girkes disease, comprising administering to a patient in need thereof, an effective amount of a glucagon peptide according to any of embodiments 1-177, optionally in combination with one or more additional therapeutically active compounds.

Further embodiments of the present invention relate to the following uses:

265. Use of a glucagon peptide according to any one of the embodiments 1-177, for the preparation of a medicament.

266. Use of a glucagon peptide according to any one of embodiments 1-177, for the preparation of a medicament for the treatment or prevention of hyperglycemia, type 2 diabetes, impaired glucose tolerance, type 1 diabetes and obesity.

267. Use of a glucagon peptide according to any one of the embodiments 1-177, for the preparation of a medicament for delaying or preventing disease progression in type 2 diabetes, treating obesity or preventing overweight, for decreasing food intake, increase energy expenditure, reducing body weight, delaying the progression from impaired glucose tolerance (IGT) to type 2 diabetes; delaying the progression from type 2 diabetes to insulin-requiring diabetes; regulating appetite; inducing satiety; preventing weight regain after successful weight loss; treating a disease or state related to overweight or obesity; treating bulimia; treating binge-eating; treating atherosclerosis, hypertension, type 2 diabetes, IGT, dyslipidemia, coronary heart disease, hepatic steatosis, treatment of beta-blocker poisoning, use for inhibition of the motility of the gastrointestinal tract, useful in connection with investigations of the gastrointestinal tract using techniques such as x-ray, CT- and NMR-scanning.

268. Use of a glucagon peptide according to any one of the embodiments 1-177, for the preparation of a medicament for reatment or prevention of hypoglycemia, insulin induced hypoglycemia, reactive hypoglycemia, diabetic hypoglycemia, non-diabetic hypoglycemia, fasting hypoglycemia, drug-induced hypoglycemia, gastric by-pass induced hypoglycemia, hypoglycemia in pregnancy, alcohol induced hypoglycemia, insulinoma and Von Girkes disease.

Further embodiments of the present invention relate to the following:

269. A glucagon peptide according to any of the previous embodiments, wherein said glucagon peptide has more than 70% recovery in the ThT fibrillation assay.

270. A glucagon peptide according to any of the previous embodiments, wherein said glucagon peptide has more than 90% recovery in the ThT fibrillation assay.

271. A glucagon peptide according to any of the previous embodiments, wherein said glucagon peptide has about 100% recovery in the ThT fibrillation assay.

272. A glucagon peptide according to any of the previous embodiments, wherein said glucagon peptide has more than 7 hours lag time in the ThT fibrillation assay.

273. A glucagon peptide according to any of the previous embodiments, wherein said glucagon peptide has more than 20 hours lag time in the ThT fibrillation assay.

274. A glucagon peptide according to any of the previous embodiments, wherein said glucagon peptide has 45 hours lag time or more in the ThT fibrillation assay.

In certain embodiments of the uses and methods of the present invention, the glucagon peptide of the present invention may be administered or applied in combination with more than one of the above-mentioned, suitable additional therapeutically active compounds or substances, e.g. in combination with: metformin and a sulfonylurea such as glyburide; a sulfonylurea and acarbose; nateglinide and metformin; acarbose and metformin; a sulfonylurea, metformin and troglitazone; insulin and a sulfonylurea; insulin and metformin; insulin, metformin and a sulfonylurea; insulin and troglitazone; insulin and lovastatin; etc.

In the case, in particular, of administration of a glucagon peptide of the invention, optionally in combination with one or more additional therapeutically active compounds or substances as disclosed above, for a purpose related to treatment or prevention of obesity or overweight, i.e. related to reduction or prevention of excess adiposity, it may be of relevance to employ such administration in combination with surgical intervention for the purpose of achieving weight loss or preventing weight gain, e.g. in combination with bariatric surgical intervention. Examples of frequently used bariatric surgical techniques include, but are not limited to, the following: vertical banded gastroplasty (also known as “stomach stapling”), wherein a part of the stomach is stapled to create a smaller pre-stomach pouch which serves as a new stomach; gastric banding, e.g. using an adjustable gastric band system (such as the Swedish Adjustable Gastric Band (SAGB), the LAP-BAND™ or the MIDband™), wherein a small pre-stomach pouch which is to serve as a new stomach is created using an elastomeric (e.g. silicone) band which can be adjusted in size by the patient; and gastric bypass surgery, e.g. “Roux-en-Y” bypass wherein a small stomach pouch is created using a stapler device and is connected to the distal small intestine, the upper part of the small intestine being reattached in a Y-shaped configuration.

The administration of a glucagon peptide of the invention (optionally in combination with one or more additional therapeutically active compounds or substances as disclosed above) may take place for a period prior to carrying out the bariatric surgical intervention in question and/or for a period of time subsequent thereto. In many cases it may be preferable to begin administration of a compound of the invention after bariatric surgical intervention has taken place.

The term “obesity” implies an excess of adipose tissue. When energy intake exceeds energy expenditure, the excess calories are stored in adipose tissue, and if this net positive balance is prolonged, obesity results, i.e. there are two components to weight balance, and an abnormality on either side (intake or expenditure) can lead to obesity. In this context, obesity is best viewed as any degree of excess adipose tissue that imparts a health risk. The distinction between normal and obese individuals can only be approximated, but the health risk imparted by obesity is probably a continuum with increasing adipose tissue. However, in the context of the present invention, individuals with a body mass index (BMI=body weight in kilograms divided by the square of the height in meters) above 25 are to be regarded as obese.

Further embodiments of the present invention relate to the following:

275. A compound according to formula I:

His-X₂-Gln-Gly-Thr-X₆-X₇-Ser-Asp-X₁₀-Ser-X₁₂-Tyr-Leu-Asp-X₁₆-X₁₇-X₁₈-Ala-X₂₀-X₂₁-Phe-Val-X₂₄-X₂₅-Leu-X₂₇-X₂₈-X₂₉-X₃₀ [I]

wherein
X₂ represents Ser, Aib or D-Ser;
X₆ represents Phe or Gln;
X₇ represents Thr, Lys or Orn;
X₁₀ represents Tyr, Lys, Orn or (p)Tyr;
X₁₂ represents Lys, Orn or Arg;
X₁₆ represents Ser, Glu, Thr, Lys or Orn;
X₁₇ represents Arg, Gln, Lys or Orn;
X₁₈ represents Arg, Gln, Ala, Lys or Orn;
X₂₀ represents Arg, Gln, Lys or Orn;
X₂₁ represents Asp, Glu or Lys;
X₂₄ represents Gln, Lys, Arg, His, Glu, Asp, Gly, Pro, Ser or Orn;
X₂₅ represents Trp, Arg, Lys, His, Glu, Asp, Gly, Pro, Phe, Ser, Tyr, (p)Tyr or Orn;
X₂₇ represents Met, Met(O), Val, Pro, Leu, Arg, Lys or Orn;
X₂₈ represents Asn, Lys, Arg, Ser, Thr, Glu, Asp, Ala, Gln, Pro or Orn;
X₂₉ represents Thr, Glu, Asp, Lys, Arg, Pro or Orn and
X₃₀ is absent or represents Lys, Gly, Pro or Orn,
and an albumin binding residue comprising two or more negatively charged groups, wherein one of the said negatively charged groups is terminal of the said albumin binding residue and where the albumin binding residue is attached at the epsilon position of a Lys or at the delta position of an Orn, in one or more of the following amino acid positions of the compound of formula I: X₇, X₁₀, X₁₂, X₁₆, X₁₇, X₁₈, X₂₀, X₂₁, X₂₄, X₂₅, X₂₇, X₂₈, X₂₉, and/or X₃₀.
or a pharmaceutically acceptable salt, amide, acid or prodrug thereof.

276. A compound according to embodiment 181, selected from the group consisting of the glucagon peptides of the examples.

277. A compound according to any of embodiment 275-276, wherein said albumin binding residue has the formula II:

Z₁—Z₂—Z₃—Z₄— [II]

wherein,
Z₁ represents a structure according to one of the formulas IIa, IIb or IIc;

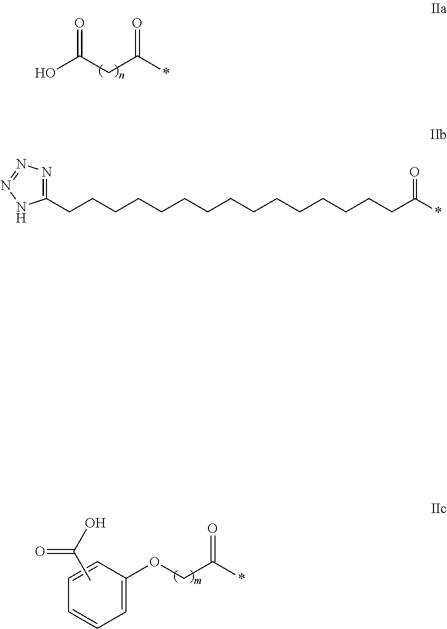

wherein n in formula IIa is 6-20,
m in formula IIc is 5-9
the COOH group in fomula IIc can reside on position 2, 3 or 4 on the phenyl ring,
the symbol * in formula IIa, IIb and IIc represents the attachment point to the nitrogen in Z₂, Z₃ or Z₄;
Z₂ is absent or represents a structure according to one of the formulas IId, IIe, IIf, IIg, IIh, IIi, IIj or IIk;

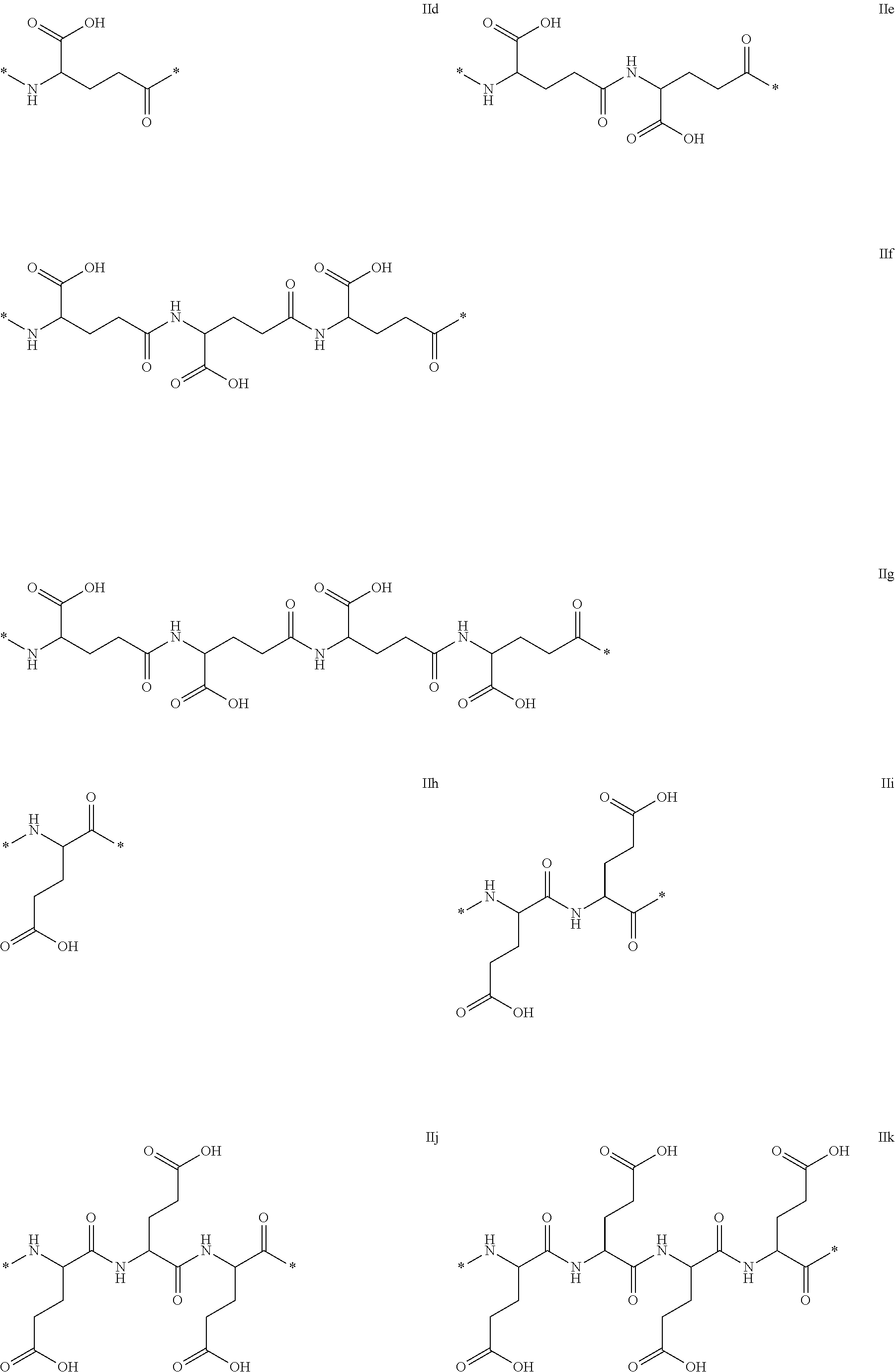

wherein each amino acid moiety is independently either L or D;
wherein Z₂ is connected via the carbon atom with symbol * to the nitrogen of Z₃, Z₄ or to the epsilon nitrogen of a lysine or the delta nitrogen of an ornithine of the glucagon peptide;
Z₃ is absent or represents a structure according to one of the formulas IIm, IIn, IIo or IIp;

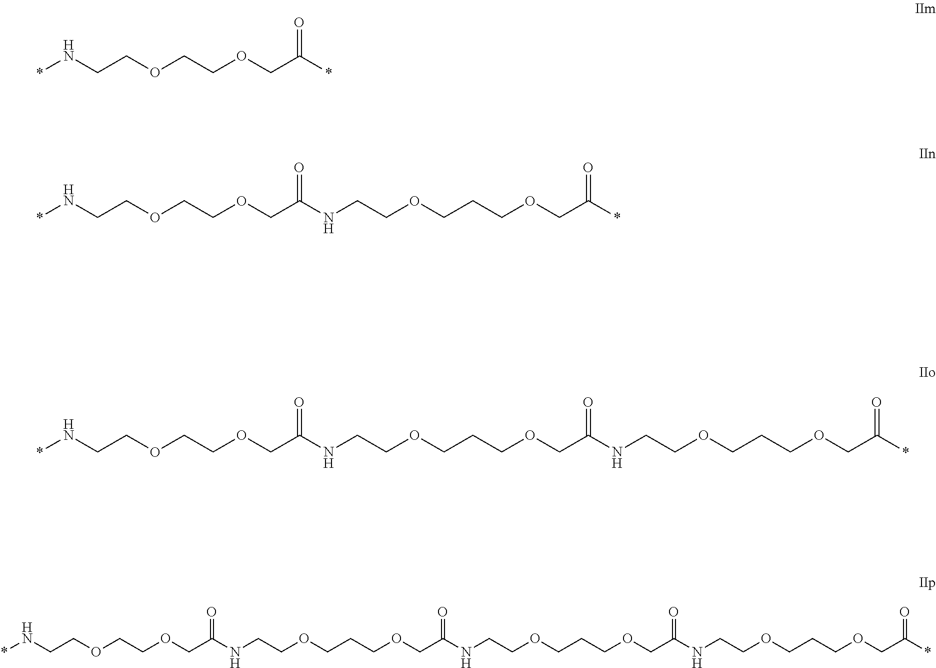 [SMILES: *NCCOCCOCC(*)=O ; *NCCOCCOCC(=O)NCCOCCCOCC(*)=O ; *NCCOCCOCC(=O)NCCOCCCOCC(=O)NCCOCCCOCC(*)=O ; *NCCOCCOCC(=O)NCCOCCCOCC(=O)NCCOCCCOCC(=O)NCCOCCCOCC(*)=O]

Z₃ is connected vi the carbon of Z₃ with symbol * to the nitrogen of Z₄ with symbol * or to the epsilon nitrogen of a lysine or the delta nitrogen of an ornithine of the glucagon peptide;
Z₄ is absent or represents a structure according to one of the formulas IId, IIe, IIf, IIg, IIh, IIi, IIj or IIk; wherein each amino acid moiety is independently either L or D, wherein Z₄ is connected via the carbon with symbol * to the epsilon nitrogen of a lysine or the delta nitrogen of an ornithine of the glucagon peptide.

278. An albumin binding residue according to embodiment 277, which is selected from the structures according to one of the formulas IIIa, IIIb, IIIc, IIId, IIIe, IIIf or IIIg:

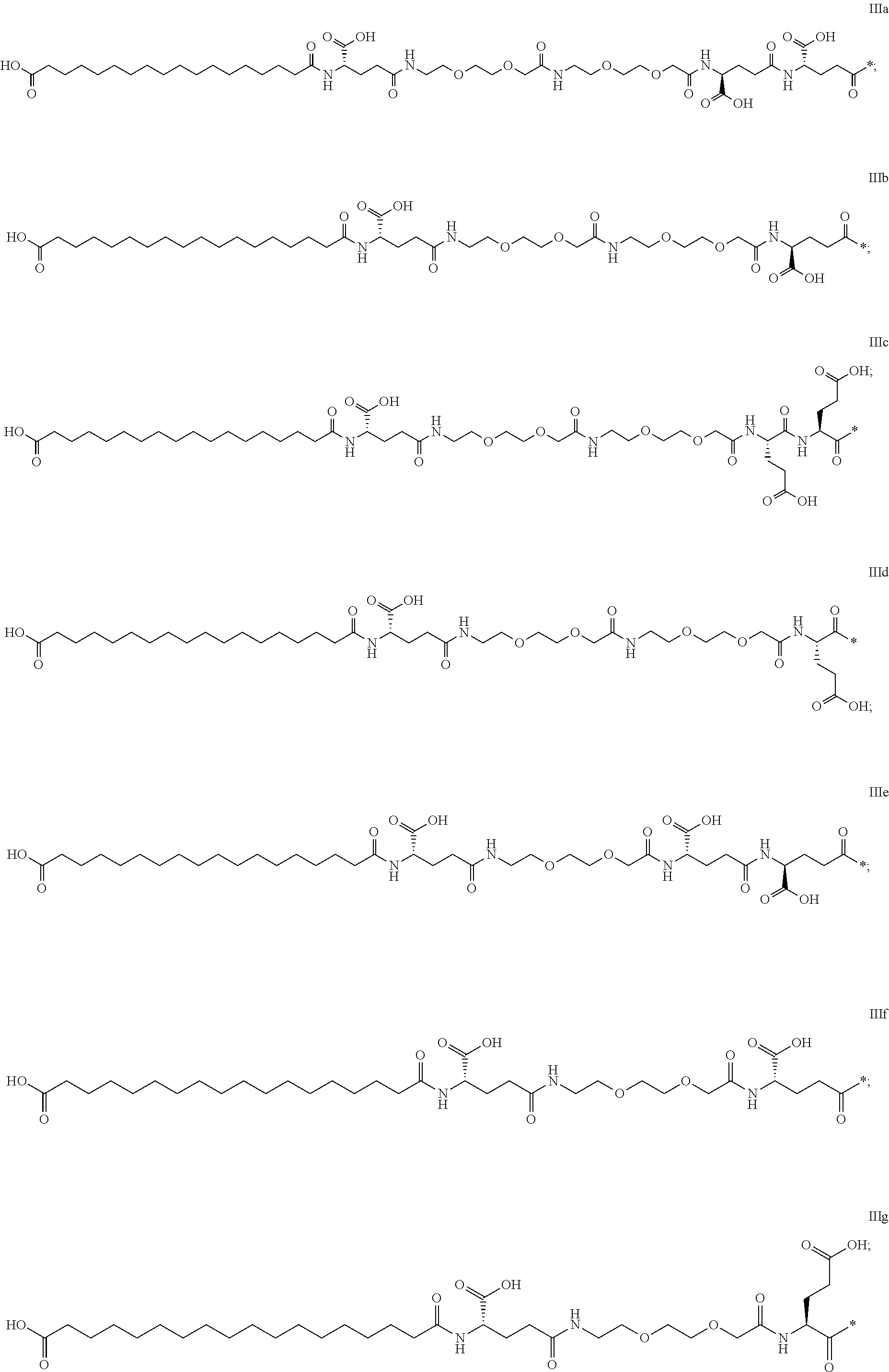

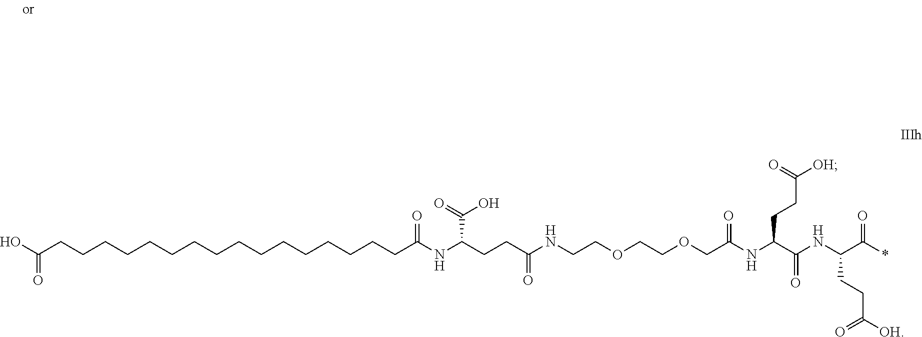 [SMILES: *C(=O)[C@H](CCC(=O)O)NC(=O)[C@H](CCC(=O)O)NC(=O)COCCOCCNC(=O)CC[C@H](NC(=O)CCCCCCCCCCCCCCCCC(=O)O)C(=O)O]

279. An albumin binding residue according to embodiments 276-278, selected from a structure according to one of the formulas Iva, IVb, IVc or IVd:

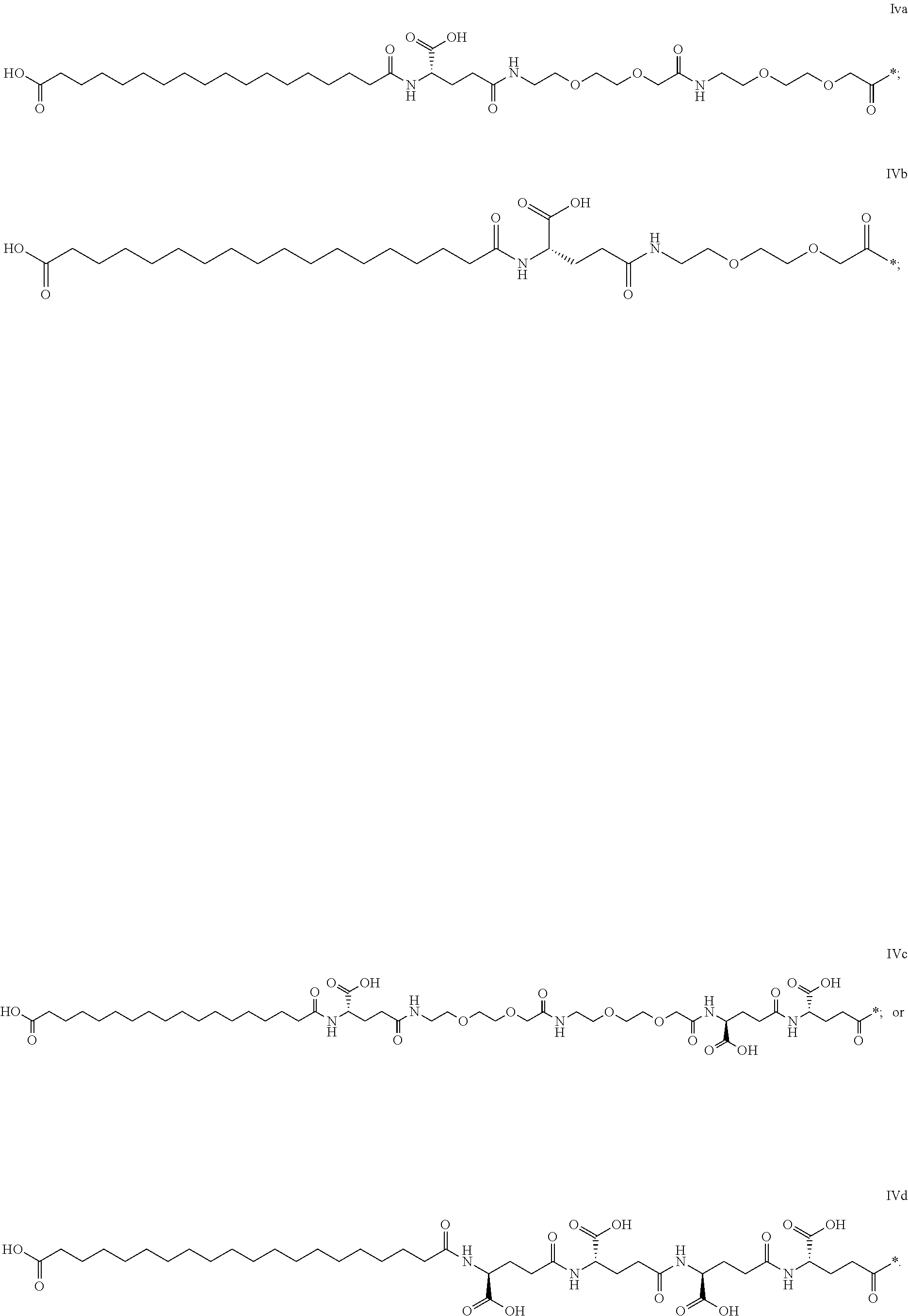

280. A pharmaceutical composition comprising a compound according to any one of embodiments 275-277.

281. A pharmaceutical composition according to any one of embodiments 275-277, further comprising one or more additional therapeutically active compounds or substances.

282. A pharmaceutical composition according to any one of embodiments, further comprising a GLP-1 compound.

283. A pharmaceutical composition according to any one of embodiments, further comprising an insulinic compound.

284. The pharmaceutical composition according to any one of embodiments, which is suited for parenteral administration.

285. A compound according to any one of embodiments, for use in therapy.

286. Use of a compound according to any one of embodiments, for the preparation of a medicament.

287. Use of a compound according to any one of embodiments, for the preparation of a medicament for the treatment or prevention of hyperglycemia, type 2 diabetes, impaired glucose tolerance, type 1 diabetes and obesity.

288. Use of a compound according to any one of embodiments, for the preparation of a medicament for delaying or preventing disease progression in type 2 diabetes, treating obesity or preventing overweight, for decreasing food intake, increase energy expenditure, reducing body weight, delaying the progression from impaired glucose tolerance (IGT) to type 2 diabetes; delaying the progression from type 2 diabetes to insulin-requiring diabetes; regulating appetite; inducing satiety; preventing weight regain after successful weight loss; treating a disease or state related to overweight or obesity; treating bulimia; treating binge-eating; treating atherosclerosis, hypertension, type 2 diabetes, IGT, dyslipidemia, coronary heart disease, hepatic steatosis, treatment of beta-blocker poisoning, use for inhibition of the motility of the gastrointestinal tract, useful in connection with investigations of the gastrointestinal tract using techniques such as x-ray, CT- and NMR-scanning.

289. Use of a compound according to any one of embodiments, for the preparation of a medicament for reatment or prevention of hypoglycemia, insulin induced hypoglycemia, reactive hypoglycemia, diabetic hypoglycemia, non-diabetic hypoglycemia, fasting hypoglycemia, drug-induced hypoglycemia, gastric by-pass induced hypoglycemia, hypoglycemia in pregnancy, alcohol induced hypoglycemia, insulinoma and Von Girkes disease.

The amino acid abbreviations used in the present context have the following meanings:

| | |
|---|---|
| Ado | 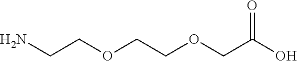 [SMILES: NCCOCCOCC(=O)O] |
| Aib | 2-Aminoisobutyric acid |
| Ala | Alanine |
| Asn | Asparagine |
| Asp | Aspartic acid |
| Arg | Arginine |
| Cit | Citrulline |
| Cys | Cysteine |
| Gln | Glutamine |
| Glu | Glutamic acid |
| γ-Glu | 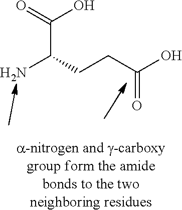 |
| Gly | Glycine |
| His | Histidine |
| Hyp | 4-hydroxyproline |
| Ile | Isoleucine |
| Leu | Leucine |
| Lys | Lysine |
| Met | Methionine |
| Met(O) | 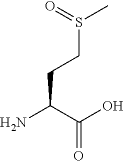 |
| Orn | Ornithine |
| Phe | Phenylalanine |
| Pro | Proline |
| Ser | Serine |
| Thr | Threonine |
| Tyr | Tyrosine |
| p(Tyr) | 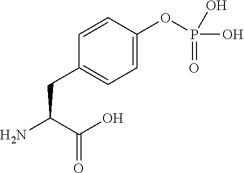 [SMILES: N[C@@H](Cc1ccc(OP(=O)(O)O)cc1)C(=O)O] |
| Trp | Tryptophan |
| Val | Valine |

Amino acid abbreviations beginning with D- followed by a three letter code, such as D-Ser, D-His and so on, refer to the D-enantiomer of the corresponding amino acid, for example D-serine, D-histidine and so on.

### PHARMACEUTICAL COMPOSITIONS

Pharmaceutical compositions containing a compound according to the present invention may be prepared by conventional techniques, e.g. as described in Remington's Pharmaceutical Sciences, 1985 or in Remington: The Science and Practice of Pharmacy, 19^{th} edition, 1995.

As already mentioned, one aspect of the present invention is to provide a pharmaceutical formulation comprising a compound according to the present invention which is present in a concentration from about 0.01 mg/mL to about 25 mg/mL, such as from about 0.1 mg/mL to about 5 mg/mL and from about 2 mg/mL to about 5 mg/mL, and wherein said formulation has a pH from 2.0 to 10.0. The pharmaceutical formulation may comprise a compound according to the present invention which is present in a concentration from about 0.1 mg/ml to about 50 mg/ml, and wherein said formulation has a pH from 2.0 to 10.0. The formulation may further comprise a buffer system, preservative(s), isotonicity agent(s), chelating agent(s), stabilizers and surfactants. In one embodiment of the invention the pharmaceutical formulation is an aqueous formulation, i.e. formulation comprising water. Such formulation is typically a solution or a suspension. In a further embodiment of the invention the pharmaceutical formulation is an aqueous solution. The term “aqueous formulation” is defined as a formulation comprising at least 50% w/w water. Likewise, the term “aqueous solution” is defined as a solution comprising at least 50% w/w water, and the term “aqueous suspension” is defined as a suspension comprising at least 50% w/w water.

In another embodiment the pharmaceutical formulation is a freeze-dried formulation, whereto the physician or the patient adds solvents and/or diluents prior to use.

In another embodiment the pharmaceutical formulation is a dried formulation (e.g. freeze-dried or spray-dried) ready for use without any prior dissolution.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 2.0 to about 10.0.

In a further aspect the invention relates to a pharmaceutical formulation comprising an aqueous solution of a compound according to the present invention, and a buffer, wherein said compound is present in a concentration from 0.1 mg/ml or above, and wherein said formulation has a pH from about 7.0 to about 8.5.

In a another embodiment of the invention the pH of the formulation is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0. Preferably, the pH of the formulation is at least 1 pH unit from the isoelectric point of the compound according to the present invention, even more preferable the pH of the formulation is at least 2 pH unit from the isoelectric point of the compound according to the present invention.

In a further embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethane, hepes, bicine, tricine, malic acid, succinate, maleic acid, fumaric acid, tartaric acid, aspartic acid or mixtures thereof. Each one of these specific buffers constitutes an alternative embodiment of the invention.

In a further embodiment of the invention the formulation further comprises a pharmaceutically acceptable preservative. In a further embodiment of the invention the preservative is selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, ethanol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 30 mg/ml. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19^{th} edition, 1995.

In a further embodiment of the invention the formulation further comprises an isotonic agent. In a further embodiment of the invention the isotonic agent is selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galacititol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19^{th} edition, 1995.

In a further embodiment of the invention the formulation further comprises a chelating agent. In a further embodiment of the invention the chelating agent is selected from salts of ethylenediaminetetraacetic acid (EDTA), citric acid, and aspartic acid, and mixtures thereof. In a further embodiment of the invention the chelating agent is present in a concentration from 0.1 mg/ml to 5 mg/ml. In a further embodiment of the invention the chelating agent is present in a concentration from 0.1 mg/ml to 2 mg/ml. In a further embodiment of the invention the chelating agent is present in a concentration from 2 mg/ml to 5 mg/ml. Each one of these specific chelating agents constitutes an alternative embodiment of the invention. The use of a chelating agent in pharmaceutical compositions is well-known to the skilled person. For convenience reference is made to Remington: The Science and Practice of Pharmacy, 19^{th} edition, 1995.

In a further embodiment of the invention the formulation further comprises a stabiliser. The use of a stabilizer in pharmaceutical compositions is well-known to the skilled person. For convenience reference is made to Remington: The Science and Practice of Pharmacy, 19^{th} edition, 1995.

More particularly, compositions of the invention are stabilized liquid pharmaceutical compositions whose therapeutically active components include a polypeptide that possibly exhibits aggregate formation during storage in liquid pharmaceutical formulations. By “aggregate formation” is intended a physical interaction between the polypeptide molecules that results in formation of oligomers, which may remain soluble, or large visible aggregates that precipitate from the solution. By “during storage” is intended a liquid pharmaceutical composition or formulation once prepared, is not immediately administered to a subject. Rather, following preparation, it is packaged for storage, either in a liquid form, in a frozen state, or in a dried form for later reconstitution into a liquid form or other form suitable for administration to a subject. By “dried form” is intended the liquid pharmaceutical composition or formulation is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53). Aggregate formation by a polypeptide during storage of a liquid pharmaceutical composition can adversely affect biological activity of that polypeptide, resulting in loss of therapeutic efficacy of the pharmaceutical composition. Furthermore, aggregate formation may cause other problems such as blockage of tubing, membranes, or pumps when the polypeptide-containing pharmaceutical composition is administered using an infusion system.

The pharmaceutical compositions of the invention may further comprise an amount of an amino acid base sufficient to decrease aggregate formation by the polypeptide during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids used for preparing the compositions of the invention are those carrying a charged side chain, such as arginine, lysine, aspartic acid, and glutamic acid. In one embodiment, the amino acid used for preparing the compositions of the invention is glycine. Any stereoisomer (i.e. L or D) of a particular amino acid (e.g. methionine, histidine, imidazole, arginine, lysine, isoleucine, aspartic acid, tryptophan, threonine and mixtures thereof) or combinations of these stereoisomers, may be present in the pharmaceutical compositions of the invention so long as the particular amino acid is present either in its free base form or its salt form. In one embodiment the L-stereoisomer is used. Compositions of the invention may also be formulated with analogues of these amino acids. By “amino acid analogue” is intended a derivative of the naturally occurring amino acid that brings about the desired effect of decreasing aggregate formation by the polypeptide during storage of the liquid pharmaceutical compositions of the invention. Suitable arginine analogues include, for example, aminoguanidine, ornithine and N-monoethyl L-arginine, suitable methionine analogues include ethionine and buthionine and suitable cystein analogues include S-methyl-L cystein. As with the other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In a further embodiment of the invention methionine (or other sulphuric amino acids or amino acid analogous) may be added to inhibit oxidation of methionine residues to methionine sulfoxide when the polypeptide acting as the therapeutic agent is a polypeptide comprising at least one methionine residue susceptible to such oxidation. By “inhibit” is intended minimal accumulation of methionine oxidized species over time. Inhibiting methionine oxidation results in greater retention of the polypeptide in its proper molecular form. Any stereoisomer of methionine (L, D or a mixture thereof) can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be achieved by adding methionine such that the ratio of methionine added to methionine residues ranges from about 1:1 to about 1000:1, such as 10:1 to about 100:1.

In a further embodiment of the invention the formulation further comprises a stabiliser selected from the group of high molecular weight polymers or low molecular compounds. In a further embodiment of the invention the stabilizer is selected from polyethylene glycol (e.g. PEG 3350), polyvinylalcohol (PVA), polyvinylpyrrolidone, carboxy-/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active polypeptide therein. Stabilizing agents of particular interest to the present invention include, but are not limited to, methionine and EDTA, which protect the polypeptide against methionine oxidation, and a nonionic surfactant, which protects the polypeptide against aggregation associated with freeze-thawing or mechanical shearing.

In a further embodiment of the invention the formulation further comprises a surfactant. In a further embodiment of the invention the surfactant is selected from a detergent, ethoxylated castor oil, polyglycolyzed glycerides, acetylated monoglycerides,

sorbitan fatty acid esters, polyoxypropylene-polyoxyethylene block polymers (eg. poloxamers such as Pluronic® F68, poloxamer 188 and 407, Triton X-100), polyoxyethylene sorbitan fatty acid esters, starshaped PEO, polyoxyethylene and polyethylene derivatives such as alkylated and alkoxylated derivatives (tweens, e.g. Tween-20, Tween-40, Tween-80 and Brij-35), polyoxyethylene hydroxystearate, monoglycerides or ethoxylated derivatives thereof, diglycerides or polyoxyethylene derivatives thereof, alcohols, glycerol, lecitins and phospholipids (eg. phosphatidyl serine, phosphatidyl choline, phosphatidyl ethanolamine, phosphatidyl inositol, diphosphatidyl glycerol and sphingomyelin), derivates of phospholipids (eg. dipalmitoyl phosphatidic acid) and lysophospholipids (eg. palmitoyl lysophosphatidyl-L-serine and 1-acyl-sn-glycero-3-phosphate esters of ethanolamine, choline, serine or threonine) and alkyl, alkoxyl (alkyl ester), alkoxy (alkyl ether)-derivatives of lysophosphatidyl and phosphatidylcholines, e.g. lauroyl and myristoyl derivatives of lysophosphatidylcholine, dipalmitoylphosphatidylcholine, and modifications of the polar head group, that is cholines, ethanolamines, phosphatidic acid, serines, threonines, glycerol, inositol, and the positively charged DODAC, DOTMA, DCP, BISHOP, lysophosphatidylserine and lysophosphatidylthreonine, and glycerophospholipids (eg. cephalins), glyceroglycolipids (eg. galactopyransoide), sphingoglycolipids (eg. ceramides, gangliosides), dodecylphosphocholine, hen egg lysolecithin, fusidic acid derivatives—(e.g. sodium tauro-dihydrofusidate etc.), long-chain fatty acids and salts thereof C6-C12 (eg. oleic acid and caprylic acid), acylcarnitines and derivatives, N^{α}-acylated derivatives of lysine, arginine or histidine, or side-chain acylated derivatives of lysine or arginine, N^{α}-acylated derivatives of dipeptides comprising any combination of lysine, arginine or histidine and a neutral or acidic amino acid, N^{α}-acylated derivative of a tripeptide comprising any combination of a neutral amino acid and two charged amino acids, DSS (docusate sodium, CAS registry no [577-11-7]), docusate calcium, CAS registry no [128-49-4]), docusate potassium, CAS registry no [7491-09-0]), SDS (sodium dodecyl sulfate or sodium lauryl sulfate), sodium caprylate, cholic acid or derivatives thereof, bile acids and salts thereof and glycine or taurine conjugates, ursodeoxycholic acid, sodium cholate, sodium deoxycholate, sodium taurocholate, sodium glycocholate, N-Hexadecyl-N,N-dimethyl-3-ammonio-1-propanesulfonate, anionic (alkyl-aryl-sulphonates) monovalent surfactants, zwitterionic surfactants (e.g. N-alkyl-N,N-dimethylammonio-1-propanesulfonates,3-cholamido-1-propyldimethylammonio-1-propanesulfonate, cationic surfactants (quarternary ammonium bases) (e.g. cetyl-trimethylammonium bromide, cetylpyridinium chloride), non-ionic surfactants (eg. Dodecyl 13-D-glucopyranoside), poloxamines (eg. Tetronic's), which are tetrafunctional block copolymers derived from sequential addition of propylene oxide and ethylene oxide to ethylenediamine, or the surfactant may be selected from the group of imidazoline derivatives, or mixtures thereof. Each one of these specific surfactants constitutes an alternative embodiment of the invention.

The use of a surfactant in pharmaceutical compositions is well-known to the skilled person. For convenience reference is made to Remington: The Science and Practice of Pharmacy, 19^{th} edition, 1995.

Additional ingredients may also be present in the pharmaceutical formulation of the present invention. Such additional ingredients may include wetting agents, emulsifiers, antioxidants, bulking agents, tonicity modifiers, chelating agents, metal ions, oleaginous vehicles, proteins (e.g., human serum albumin, gelatin or proteins) and a zwitterion (e.g., an amino acid such as betaine, taurine, arginine, glycine, lysine and histidine). Such additional ingredients, of course, should not adversely affect the overall stability of the pharmaceutical formulation of the present invention.

Pharmaceutical compositions containing a compound according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, lingual, sublingual, buccal, in the mouth, oral, in the stomach and intestine, nasal, pulmonary, for example, through the bronchioles and alveoli or a combination thereof, epidermal, dermal, transdermal, vaginal, rectal, ocular, for examples through the conjunctiva, uretal, and parenteral to patients in need of such a treatment.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the compound, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block copolymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the formulation of solids, semisolids, powder and solutions for pulmonary administration of the compound, using, for example a metered dose inhaler, dry powder inhaler and a nebulizer, all being devices well known to those skilled in the art.

Compositions of the current invention are specifically useful in the formulation of controlled, sustained, protracting, retarded, and slow release drug delivery systems. More specifically, but not limited to, compositions are useful in formulation of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles,

Methods to produce controlled release systems useful for compositions of the current invention include, but are not limited to, crystallization, condensation, co-cystallization, precipitation, co-precipitation, emulsification, dispersion, high pressure homogenization, encapsulation, spray drying, microencapsulation, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Formulation and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the compound according to the present invention in the form of a nasal or pulmonal spray. As a still further option, the pharmaceutical compositions containing the compound of the invention can also be adapted to transdermal administration, e.g. by needle-free injection or from a patch, optionally an iontophoretic patch, or transmucosal, e.g. buccal, administration.

The term “stabilized formulation” refers to a formulation with increased physical stability, increased chemical stability or increased physical and chemical stability.

The term “physical stability” of the protein formulation as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces. Physical stability of the aqueous protein formulations is evaluated by means of visual inspection and/or turbidity measurements after exposing the formulation filled in suitable containers (e.g. cartridges or vials) to mechanical/physical stress (e.g. agitation) at different temperatures for various time periods. Visual inspection of the formulations is performed in a sharp focused light with a dark background. The turbidity of the formulation is characterized by a visual score ranking the degree of turbidity for instance on a scale from 0 to 3 (a formulation showing no turbidity corresponds to a visual score 0, and a formulation showing visual turbidity in daylight corresponds to visual score 3). A formulation is classified physical unstable with respect to protein aggregation, when it shows visual turbidity in daylight. Alternatively, the turbidity of the formulation can be evaluated by simple turbidity measurements well-known to the skilled person. Physical stability of the aqueous protein formulations can also be evaluated by using a spectroscopic agent or probe of the conformational status of the protein. The probe is preferably a small molecule that preferentially binds to a non-native conformer of the protein. One example of a small molecular spectroscopic probe of protein structure is Thioflavin T. Thioflavin T is a fluorescent dye that has been widely used for the detection of amyloid fibrils. In the presence of fibrils, and perhaps other protein configurations as well, Thioflavin T gives rise to a new excitation maximum at about 450 nm and enhanced emission at about 482 nm when bound to a fibril protein form. Unbound Thioflavin T is essentially non-fluorescent at the wavelengths.

Other small molecules can be used as probes of the changes in protein structure from native to non-native states. For instance the “hydrophobic patch” probes that bind preferentially to exposed hydrophobic patches of a protein. The hydrophobic patches are generally buried within the tertiary structure of a protein in its native state, but become exposed as a protein begins to unfold or denature. Examples of these small molecular, spectroscopic probes are aromatic, hydrophobic dyes, such as antrhacene, acridine, phenanthroline or the like. Other spectroscopic probes are metal-amino acid complexes, such as cobalt metal complexes of hydrophobic amino acids, such as phenylalanine, leucine, isoleucine, methionine, and valine, or the like.

The term “chemical stability” of the protein formulation as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed. Elimination of chemical degradation can most probably not be completely avoided and increasing amounts of chemical degradation products is often seen during storage and use of the protein formulation as well-known by the person skilled in the art. Most proteins are prone to deamidation, a process in which the side chain amide group in glutaminyl or asparaginyl residues is hydrolysed to form a free carboxylic acid. Other degradations pathways involves formation of high molecular weight transformation products where two or more protein molecules are covalently bound to each other through transamidation and/or disulfide interactions leading to formation of covalently bound dimer, oligomer and polymer degradation products (Stability of Protein Pharmaceuticals, Ahern. T. J. & Manning M. C., Plenum Press, New York 1992). Oxidation (of for instance methionine residues) can be mentioned as another variant of chemical degradation. The chemical stability of the protein formulation can be evaluated by measuring the amount of the chemical degradation products at various time-points after exposure to different environmental conditions (the formation of degradation products can often be accelerated by for instance increasing temperature). The amount of each individual degradation product is often determined by separation of the degradation products depending on molecule size and/or charge using various chromatography techniques (e.g. SEC-HPLC and/or RP-HPLC).

Hence, as outlined above, a “stabilized formulation” refers to a formulation with increased physical stability, increased chemical stability or increased physical and chemical stability. In general, a formulation must be stable during use and storage (in compliance with recommended use and storage conditions) until the expiration date is reached.

In one embodiment of the invention the pharmaceutical formulation comprising the compound according to the present invention is stable for more than 6 weeks of usage and for more than 3 years of storage.

In another embodiment of the invention the pharmaceutical formulation comprising the compound according to the present invention is stable for more than 4 weeks of usage and for more than 3 years of storage.

In a further embodiment of the invention the pharmaceutical formulation comprising the compound according to the present invention is stable for more than 4 weeks of usage and for more than two years of storage.

In an even further embodiment of the invention the pharmaceutical formulation comprising the compound is stable for more than 2 weeks of usage and for more than two years of storage.

Pharmaceutical compositions containing a glucagon peptide according to the present invention may be administered parenterally to patients in need of such a treatment. Parenteral administration may be performed by subcutaneous, intramuscular or intravenous injection by means of a syringe, optionally a pen-like syringe. Alternatively, parenteral administration can be performed by means of an infusion pump. A further option is a composition which may be a powder or a liquid for the administration of the glucagon peptide in the form of a nasal or pulmonal spray. As a still further option, the glucagon peptides of the invention can also be administered transdermally, e.g. from a patch, optionally a iontophoretic patch, or transmucosally, e.g. bucally.

Thus, the injectable compositions of the glucagon peptide of the present invention can be prepared using the conventional techniques of the pharmaceutical industry which involves dissolving and mixing the ingredients as appropriate to give the desired end product.

According to one embodiment of the present invention, the glucagon peptide is provided in the form of a composition suitable for administration by injection. Such a composition can either be an injectable solution ready for use or it can be an amount of a solid composition, e.g. a lyophilised product, which has to be dissolved in a solvent before it can be injected. The injectable solution preferably contains not less than about 2 mg/ml, preferably not less than about 5 mg/ml, more preferred not less than about 10 mg/ml of the glucagon peptide and, preferably, not more than about 100 mg/ml of the glucagon peptide.

The glucagon peptides of this invention can be used in the treatment of various diseases. The particular glucagon peptide to be used and the optimal dose level for any patient will depend on the disease to be treated and on a variety of factors including the efficacy of the specific peptide derivative employed, the age, body weight, physical activity, and diet of the patient, on a possible combination with other drugs, and on the severity of the case. It is recommended that the dosage of the glucagon peptide of this invention be determined for each individual patient by those skilled in the art.

In particular, it is envisaged that the glucagon peptide will be useful for the preparation of a medicament with a protracted profile of action for the treatment of non-insulin dependent diabetes mellitus and/or for the treatment of obesity.

In another aspect the present invention relates to the use of a compound according to the invention for the preparation of a medicament.

In one embodiment the present invention relates to the use of a compound according to the invention for the preparation of a medicament for the treatment of hyperglycemia, type 2 diabetes, impaired glucose tolerance, type 1 diabetes, obesity, hypertension, syndrome X, dyslipidemia, β-cell apoptosis, β-cell deficiency, myocardial infarction, inflammatory bowel syndrome, dyspepsia, cognitive disorders, e.g. cognitive enhancing, neuroprotection, atheroschlerosis, coronary heart disease and other cardiovascular disorders.

In another embodiment the present invention relates to the use of a compound according to the invention for the preparation of a medicament for the treatment of small bowel syndrome, inflammatory bowel syndrome or Crohns disease.

In another embodiment the present invention relates to the use of a compound according to the invention for the preparation of a medicament for the treatment of hyperglycemia, type 1 diabetes, type 2 diabetes or β-cell deficiency.

The treatment with a compound according to the present invention may also be combined with combined with a second or more pharmacologically active substances, e.g. selected from antidiabetic agents, antiobesity agents, appetite regulating agents, antihypertensive agents, agents for the treatment and/or prevention of complications resulting from or associated with diabetes and agents for the treatment and/or prevention of complications and disorders resulting from or associated with obesity. In the present context the expression “antidiabetic agent” includes compounds for the treatment and/or prophylaxis of insulin resistance and diseases wherein insulin resistance is the pathophysiological mechanism.

Examples of these pharmacologically active substances are: Insulin, GLP-1 agonists, sulphonylureas (e.g. tolbutamide, glibenclamide, glipizide and gliclazide), biguanides e.g. metformin, meglitinides, glucosidase inhibitors (e.g. acorbose), glucagon antagonists, DPP-IV (dipeptidyl peptidase-IV) inhibitors, inhibitors of hepatic enzymes involved in stimulation of gluconeogenesis and/or glycogenolysis, glucose uptake modulators, thiazolidinediones such as troglitazone and ciglitazone, compounds modifying the lipid metabolism such as antihyperlipidemic agents as HMG CoA inhibitors (statins), compounds lowering food intake, RXR agonists and agents acting on the ATP-dependent potassium channel of the β-cells, e.g. glibenclamide, glipizide, gliclazide and repaglinide; Cholestyramine, colestipol, clofibrate, gemfibrozil, lovastatin, pravastatin, simvastatin, probucol, dextrothyroxine, neteglinide, repaglinide; β-blockers such as alprenolol, atenolol, timolol, pindolol, propranolol and metoprolol, ACE (angiotensin converting enzyme) inhibitors such as benazepril, captopril, enalapril, fosinopril, lisinopril, alatriopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin; CART (cocaine amphetamine regulated transcript) agonists, NPY (neuropeptide Y) antagonists, MC4 (melanocortin 4) agonists, orexin antagonists, TNF (tumor necrosis factor) agonists, CRF (corticotropin releasing factor) agonists, CRF BP (corticotropin releasing factor binding protein) antagonists, urocortin agonists, β3 agonists, MSH (melanocyte-stimulating hormone) agonists, MCH (melanocyte-concentrating hormone) antagonists, CCK (cholecystokinin) agonists, serotonin re-uptake inhibitors, serotonin and noradrenaline re-uptake inhibitors, mixed serotonin and noradrenergic compounds, 5HT (serotonin) agonists, bombesin agonists, galanin antagonists, growth hormone, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists; histamine H3 antagonists.

It should be understood that any suitable combination of the compounds according to the invention with one or more of the above-mentioned compounds and optionally one or more further pharmacologically active substances are considered to be within the scope of the present invention.

The present invention is further illustrated by the following examples which, however, are not to be construed as limiting the scope of protection. The features disclosed in the foregoing description and in the following examples may, both separately and in any combination thereof, be material for realizing the invention in diverse forms thereof.

### EXAMPLES

### List of Abbreviations Used

DCM: dichloromethane
Dde: 1-(4,4-dimethyl-2,6-dioxocyclohexylidene)ethyl
DIC: diisopropylcarbodiimide
DIPEA: diisopropylethylamine
Fmoc: 9-fluorenylmethyloxycarbonyl
HATU: (O-(7-azabenzotriazol-1-yl)-1,1,3,3-tetramethyluronium hexafluorophosphate)
HBTU: (2-(1H-benzotriazol-1-yl-)-1,1,3,3 tetramethyluronium hexafluorophosphate)
HFIP 1,1,1,3,3,3-hexafluoro-2-propanol or hexafluoroisopropanol
HOAt: 1-hydroxy-7-azabenzotriazole
HOBt: 1-hydroxybenzotriazole

### HPLC: High Performance Liquid Chromatography

ivDde: 1-(4,4-dimethyl-2,6-dioxocyclohexylidene)-3-methylbutyl

### LCMS: Liquid Chromatography Mass Spectroscopy

MeOH: methanol
Mmt: 4-methoxytrityl
Mtt: 4-methyltrityl
NMP: N-methyl pyrrolidone
OEG: 8-amino-3,6-dioxaoctanic acid
OtBu: tert butyl ester

### PBS: Phosphate Buffered Saline

### RP: Reverse Phase

### RP-HPLC: Reverse Phase High Performance Liquid Chromatography

### RT: Room Temperature

Rt: Retention time

### SPPS: Solid Phase Peptide Synthesis

TFA: trifluoroacetic acid
TIPS: triisopropylsilane
Trt: triphenylmethyl or trityl

### UPLC: Ultra High Performance Liquid Chromatography

### General Methods

This section relates to methods for synthesising resin bound peptide (SPPS methods, including methods for de-protection of amino acids, methods for cleaving the peptide from the resin, and for its purification), as well as methods for detecting and characterising the resulting peptide (LCMS and UPLC methods).

### Synthesis of Resin Bound Peptide

### SPPS Method A

SPPS method A refers to peptide synthesis by Fmoc chemistry on a Prelude Solid Phase Peptide Synthesizer from Protein Technologies (Tucson, Ariz. 85714 U.S.A.).

The Fmoc-protected amino acid derivatives used were the standard recommended: Fmoc-Ala-OH, Fmoc-Arg(Pbf)-OH, Fmoc-Asn(Trt)-OH, Fmoc-Asp(OtBu)-OH, Fmoc-Cys(Trt)-OH, Fmoc-Gln(Trt)-OH, Fmoc-Glu(OtBu)-OH, Fmoc-Gly-OH, Fmoc-His(Trt)-OH, Fmoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH and Fmoc-Val-OH, supplied from e.g. Anaspec, Bachem, Iris Biotech, or Novabiochem.

When the albumin binding residue was present on a lysine sidechain the epsilon amino group of lysine to be acylated was protected with Mtt (e.g. Fmoc-Lys(Mtt)-OH) and the N-terminal alpha amino group was protected with Boc. Likewise when the albumin binding residue was present on an ornithine sidechain the delta aminogroup of the ornithine to be acylated was protected with Mtt (e.g. Fmoc-Orn(Mtt)-OH.

A suitable resin for the synthesis of a glucagon analogues with a C-terminal carboxylic acid is a pre-loaded, low-load Wang resin available from Novabiochem (e.g. low load fmoc-Thr(tBu)-Wang resin, LL, 0.27 mmol/g). A suitable resin for the synthesis of glucagon analogues with a C-terminal amide is PAL-ChemMatrix resin available from Matrix-Innovation. Fmoc-deprotection was achieved with 20% piperidine in NMP for 2×3 min. The coupling chemistry was DIC/HOAt/collidine in NMP. Amino acid/HOAt solutions (0.3 M/0.3 M in NMP at a molar excess of 3-10 fold) were added to the resin followed by the same molar equivalent of DIC (3 M in NMP) followed by collidine (3 M in NMP). For example, the following amounts of 0.3 M amino acid/HOAt solution were used per coupling for the following scale reactions: Scale/ml, 0.05 mmol/1.5 mL, 0.10 mmol/3.0 mL, 0.25 mmol/7.5 mL. Coupling time were in general 30 min. All couplings were repeated to ensure complete couplings.

Deprotection of the Mtt protected lysine was performed on a Prelude Solid Phase Peptide Synthesizer or by manual synthesis.

Manual synthesis; the Mtt group was removed by washing the resin with DCM and suspending the resin in HFIP/DCM/TIPS (70:28:2) (2×20 min) and subsequently washed in sequence with DCM (3×), 5% DIPEA in DCM (1×), DCM 4×) and NMP-DCM (4:1).

Prelude Synthesizer; the Mtt group was removed by washing the resin with HFIP/DCM (75:25) (2×2 min), washed with DCM and suspending the resin in HFIP/DCM (75:25)(2×20 min) and subsequently washed in sequence with Piperidine/NMP (20:80), DCM(1×), NMP(1×), DCM(1×), NMP(1×)

### SPPS Method B—Attachment of the Preformed Albumin Binding Moiety

A solution the carboxylic acid of the preformed albumin binding moiety such as 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid. (4 eq.), HOAt (4 eq.) and DIC (4 eq.) in NMP-DCM (4:1) was stirred for 30 min before it was added to the resin. The resin was agitated for 30 min in the mixture before collidine (4 eq.) was added. The resin was agitated for 16 h. before it was washed with NMP (5×) and DCM (5×).

### SPPS Method C—Attachment of the Albumin Binding Moiety—Stepwise Procedure

The albumin binding moiety can be introduced in a stepwise procedure by the Prelude peptide synthesizer as described above (SPPC method A) using suitably protected building blocks, with the modification that the amino acids and fatty acid derivatives including Fmoc-Ado-OH, Fmoc-Glu-OtBu, and octadecanedioic acid mono-tert-butyl ester (or the analogous C8, C10, C12-, C14-C16-, C20-diacid mono tert-butyl esters) were coupled for 6 hrs in each step. After each coupling step, unreacted peptide intermediate was capped using acetic acid anhydride and collidine in excess (>10 eq.).

Cleavage from the Resin

After synthesis the resin was washed with DCM, and the peptide was cleaved from the resin by a 2-3 hour treatment with TFA/TIS/water (95/2.5/2.5) followed by precipitation with diethylether. The precipitate was washed with diethylether.

### Purification and Quantification

The crude peptide is dissolved in a suitable mixture of water and MeCN such as water/MeCN (4:1) and purified by reversed-phase preparative HPLC (Waters Deltaprep 4000 or Gilson) on a column containing C18-silica gel. Elution is performed with an increasing gradient of MeCN in water containing 0.1% TFA. Relevant fractions are checked by analytical HPLC or UPLC. Fractions containing the pure target peptide are mixed and concentrated under reduced pressure. The resulting solution is analyzed (UPLC, HPLC and LCMS) and the product is quantified using a chemiluminescent nitrogen specific HPLC detector (Antek 8060 HPLC-CLND) or by measuring UV-absorption at 280 nm. The product is dispensed into glass vials. The vials are capped with Millipore glassfibre prefilters. Freeze-drying affords the peptide trifluoroacetate as a white solid.

### Methods for Detection and Characterization

### LCMS Methods

### LCMS

### Method: LCMS_2

A Perkin Elmer Sciex API 3000 mass spectrometer was used to identify the mass of the sample after elution from a Perkin Elmer Series 200 HPLC system. Eluents: A: 0.05% Trifluoro acetic acid in water; B: 0.05% Trifluoro acetic acid in acetonitrile. Column: Waters Xterra MS C-18×3 mm id 5 μm. Gradient: 5%-90% B over 7.5 min at 1.5 ml/min.

### Method: LCMS_4

LCMS_4 was performed on a setup consisting of Waters Acquity UPLC system and LCT Premier XE mass spectrometer from Micromass. Eluents: A: 0.1% Formic acid in water

B: 0.1% Formic acid in acetonitrile The analysis was performed at RT by injecting an appropriate volume of the sample (preferably 2-10 μl) onto the column which was eluted with a gradient of A and B. The UPLC conditions, detector settings and mass spectrometer settings were: Column: Waters Acquity UPLC BEH, C-18, 1.7 μm, 2.1 mm×50 mm. Gradient: Linear 5%-95% acetonitrile during 4.0 min (alternatively 8.0 min) at 0.4 ml/min. Detection: 214 nm (analogue output from TUV (Tunable UV detector)) MS ionisation mode: API-ES

Scan: 100-2000 amu (alternatively 500-2000 amu), step 0.1 amu.

### Method: LCMS_AP

A Micromass Quatro micro API mass spectrometer was used to identify the mass of the sample after elution from a HPLC system composed of Waters2525 binary gradient modul, Waters2767 sample manager, Waters 2996 Photodiode Array Detector and Waters 2420 ELS Detector. Eluents: A: 0.1% Trifluoro acetic acid in water; B: 0.1% Trifluoro acetic acid in acetonitrile. Column: Phenomenex Synergi MAXRP, 4 um, 75×4.6 mm. Gradient: 5%-95% B over 7 min at 1.0 ml/min.

### UPLC Methods

Method 04_A3_1

UPLC (method 04_A3_1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 90% H2O, 10% CH3CN, 0.25 M ammonium bicarbonate

B: 70% CH3CN, 30% H2O

The following linear gradient was used: 75% A, 25% B to 45% A, 55% B over 16 minutes at a flow-rate of 0.35 ml/min.
Method 04_A4_1

UPLC (method 04_A4_1): The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 90% H2O, 10% CH3CN, 0.25 M ammonium bicarbonate

B: 70% CH3CN, 30% H2O

The following linear gradient was used: 65% A, 35% B to 25% A, 65% B over 16 minutes at a flow-rate of 0.35 ml/min.
Method: 04_A2_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 90% H2O, 10% CH3CN, 0.25 M ammonium bicarbonate; B: 70% CH3CN, 30% H2O. The following linear gradient was used: 90% A, 10% B to 60% A, 40% B over 16 minutes at a flow-rate of 0.40 ml/min.

Method: 04_A6_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 10 mM TRIS, 15 mM ammonium sulphate, 80% H2O, 20%, pH 7.3; B: 80% CH3CN, 20% H2O. The following linear gradient was used: 95% A, 5% B to 10% A, 90% B over 16 minutes at a flow-rate of 0.35 ml/min.

Method: 04_A7_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 10 mM TRIS, 15 mM ammonium sulphate, 80% H2O, 20%, pH 7.3; B: 80% CH3CN, 20% H2O. The following linear gradient was used: 95% A, 5% B to 40% A, 60% B over 16 minutes at a flow-rate of 0.40 ml/min.

Method: 04_A9_1

The RP-analysis was performed using a Waters UPLC system fitted with a dual band detector. UV detections at 214 nm and 254 nm were collected using an ACQUITY UPLC BEH Shield RP18, C18, 1.7 um, 2.1 mm×150 mm column, 60° C. The UPLC system was connected to two eluent reservoirs containing: A: 200 mM Na2SO4+20 mM Na2HPO4+20 mM NaH2PO4 in 90% H₂O/10% CH3CN, pH 7.2; B: 70% CH₃CN, 30% H₂O. The following step gradient was used: 90% A, 10% B to 80% A, 20% B over 3 minutes, 80% A, 20% B to 50% A, 50% B over 17 minutes at a flow-rate of 0.40 ml/min.

Method 05_B5_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 0.2 M Na2SO4, 0.04 M H3PO4, 10% CH3CN (pH 3.5)

B: 70% CH3CN, 30% H2O

The following linear gradient was used: 60% A, 40% B to 30% A, 70% B over 8 minutes at a flow-rate of 0.35 ml/min.
Method: 05_B7_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 0.2 M Na2SO4, 0.04 M H3PO4, 10% CH3CN (pH 3.5); B: 70% CH3CN, 30% H2O. The following linear gradient was used: 80% A, 20% B to 40% A, 60% B over 8 minutes at a flow-rate of 0.40 ml/min.

Method: 05_B8_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 0.2 M Na2SO4, 0.04 M H3PO4, 10% CH3CN (pH 3.5); B: 70% CH3CN, 30% H2O. The following linear gradient was used: 50% A, 50% B to 20% A, 80% B over 8 minutes at a flow-rate of 0.40 ml/min.

Method: 05_B9_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 0.2 M Na2SO4, 0.04 M H3PO4, 10% CH3CN (pH 3.5); B: 70% CH3CN, 30% H2O. The following linear gradient was used: 70% A, 30% B to 20% A, 80% B over 8 minutes at a flow-rate of 0.40 ml/min.

Method: 05_B10_1

The RP-analyses was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 0.2 M Na2SO4, 0.04 M H3PO4, 10% CH3CN (pH 3.5); B: 70% CH3CN, 30% H2O. The following linear gradient was used: 40% A, 60% B to 20% A, 80% B over 8 minutes at a flow-rate of 0.40 ml/min.

Method: 07_B4_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing: A: 99.95% H2O, 0.05% TFA; B: 99.95% CH3CN, 0.05% TFA. The following linear gradient was used: 95% A, 5% B to 5% A, 95% B over 16 minutes at a flow-rate of 0.40 ml/min.

Method: 09_B2_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 99.95% H2O, 0.05% TFA; B: 99.95% CH3CN, 0.05% TFA. The following linear gradient was used: 95% A, 5% B to 40% A, 60% B over 16 minutes at a flow-rate of 0.40 ml/min.

Method: 09_B4_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing: A: 99.95% H2O, 0.05% TFA; B: 99.95% CH3CN, 0.05% TFA. The following linear gradient was used: 95% A, 5% B to 5% A, 95% B over 16 minutes at a flow-rate of 0.40 ml/min.

Method 08_B2_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C. The UPLC system was connected to two eluent reservoirs containing:

A: 99.95% H2O, 0.05% TFA

B: 99.95% CH3CN, 0.05% TFA

The following linear gradient was used: 95% A, 5% B to 40% A, 60% B over 16 minutes at a flow-rate of 0.40 ml/min.
Method 08_B4_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 40° C.

The UPLC system was connected to two eluent reservoirs containing:

A: 99.95% H2O, 0.05% TFA

B: 99.95% CH3CN, 0.05% TFA

The following linear gradient was used: 95% A, 5% B to 5% A, 95% B over 16 minutes at a flow-rate of 0.40 ml/min.
Method 10_B4_2

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 50° C.

The UPLC system was connected to two eluent reservoirs containing:

A: 99.95% H2O, 0.05% TFA

B: 99.95% CH3CN, 0.05% TFA

The following linear gradient was used: 95% A, 5% B to 5% A, 95% B over 12 minutes at a flow-rate of 0.40 ml/min.
Method 10_B5_2

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 50° C.

The UPLC system was connected to two eluent reservoirs containing:

A: 70% MeCN, 30% Water

B: 0.2M Na2SO4, 0.04 M H3PO4, 10% MeCN, pH 2.25

The following linear gradient was used: 40% A in 1 min, 40-->70% A in 7 min at a flow-rate of 0.40 ml/min.
Method: 10_B14_1

The RP-analyses was performed using a Waters UPLC system fitted with a dual band detector. UV detections at 214 nm and 254 nm were collected using an ACQUITY UPLC BEH ShieldRP18, 1.7 um, 2.1 mm×150 mm column, 50° C. The UPLC system was connected to two eluent reservoirs containing: A: 99.95% H2O, 0.05% TFA; B: 99.95% CH3CN, 0.05% TFA. The following linear gradient was used: 70% A, 30% B to 40% A, 60% B over 12 minutes at a flow-rate of 0.40 ml/min.

Method: AP_B4_1

The RP-analysis was performed using a Waters UPLC system fitted with a dual band detector. UV detections at 214 nm and 254 nm were collected using an ACQUITY UPLC BEH130, C18, 130 Å, 1.7 um, 2.1 mm×150 mm column, 30° C.

The UPLC system was connected to two eluent reservoirs containing: A: 99.95% H2O, 0.05% TFA; B: 99.95% CH3CN, 0.05% TFA. The following linear gradient was used: 95% A, 5% B to 5% A, 95% B over 16 minutes at a flow-rate of 0.30 ml/min.

### Example 1

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[D-Ser²,Lys²⁴,Leu²⁷]Glucagon

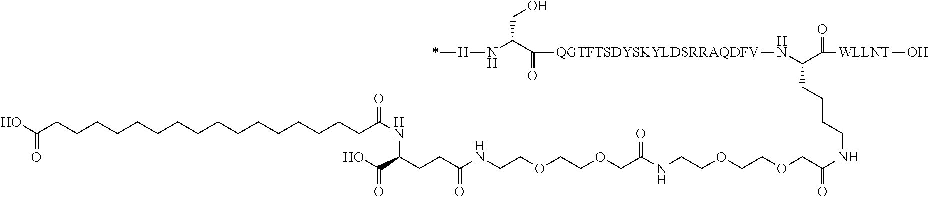

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1:8.3 min

UPLC 04_A4_1:6.3 min

UPLC 05_B5_1:5.8 min

LCMS: m/z 1494.8 (M+3H)3+, 1046.6 (M+4H)4+, 837.5 (M+5)5+

### Preparation of building block 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid

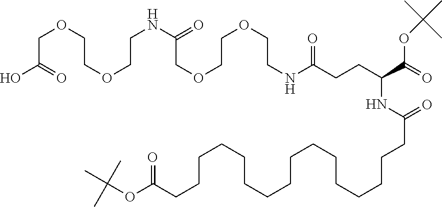 [SMILES: CC(C)(C)OC(=O)CCCCCCCCCCCCCCCCC(=O)N[C@@H](CCC(=O)NCCOCCOCC(=O)NCCOCCOCC(=O)O)C(=O)OC(C)(C)C]

2-Chlorotrityl resin 100-200 mesh (42.6 g, 42.6 mmol) was left to swell in dry dichloromethane (205 mL) for 20 min. A solution of {2-[2-(9H-fluoren-9-ylmethoxycarbonylamino)-ethoxy]-ethoxy}-acetic acid (13.7 g, 35.5 mmol) and N,N-diisopropylethylamine (23.5 mL, 135 mmol) in dry dichloromethane (30 mL) was added to resin and the mixture was shaken for 3 hrs. Resin was filtered and treated with a solution of N,N-diisopropylethylamine (12.4 mL, 70.9 mmol) in methanol/dichloromethane mixture (4:1, 250 mL, 2×5 min). Then resin was washed with N,N-dimethylformamide (2×150 mL), dichloromethane (3×150 mL) and N,N-dimethylformamide (3×150 mL). Fmoc group was removed by treatment with 20% piperidine in dimethylformamide (1×5 min, 1×30 min, 2×150 mL). Resin was washed with N,N-dimethylformamide (3×150 mL), 2-propanol (2×150 mL) and dichloromethane (200 mL, 2×150 mL). Solution of {2-[2-(9H-fluoren-9-ylmethoxycarbonylamino)-ethoxy]-ethoxy}-acetic acid (20.5 g, 53.2 mmol), O-(6-chloro-benzotriazol-1-yl)-N,N,N′,N′-tetramethyluronium tetrafluoroborate (TCTU, 18.9 g, 53.2 mmol) and N,N-diisopropylethylamine (16.7 mL, 95.7 mmol) in N,N-dimethylformamide (100 mL) and dichloromethane (50 mL) was added to resin and mixture was shaken for 1 hr. Resin was filtered and washed with N,N-dimethylformamide (2×150 mL), dichloromethane (3×150 mL) and N,N-dimethylformamide (155 mL). Fmoc group was removed by treatment with 20% piperidine in dimethylformamide (1×5 min, 1×30 min, 2×150 mL). Resin was washed with N,N-dimethylformamide (3×150 mL), 2-propanol (2×150 mL) and dichloromethane (200 mL, 2×150 mL). Solution of Fmoc-Glu-OtBu (22.6 g, 53.2 mmol), O-(6-chloro-benzotriazol-1-yl)-N,N,N′,N′-tetramethyluronium tetrafluoroborate (TCTU, 18.9 g, 53.2 mmol) and N,N-diisopropylethylamine (16.7 mL, 95.7 mmol) in N,N-dimethylformamide (155 mL) was added to resin and mixture was shaken for 1 hr. Resin was filtered and washed with N,N-dimethylformamide (2×150 mL), dichloromethane (2×150 mL) and N,N-dimethylformamide (150 mL). Fmoc group was removed by treatment with 20% piperidine in dimethylformamide (1×5 min, 1×30 min, 2×150 mL). Resin was washed with N,N-dimethylformamide (3×150 mL), 2-propanol (2×150 mL) and dichloromethane (200 mL, 2×150 mL). Solution of octadecanedioic acid mono-tert-butyl ester (19.7 g, 53.2 mmol), O-(6-chloro-benzotriazol-1-yl)-N,N,N′,N′-tetramethyluronium tetrafluoroborate (TCTU, 18.9 g, 53.2 mmol) and N,N-diisopropylethylamine (16.7 mL, 95.7 mmol) in N,N-dimethylformamide/dichloromethane mixture (1:4, 200 mL) was added to resin. Resin was shaken for 2 hrs, filtered and washed with N,N-dimethylformamide (3×150 mL), dichloromethane (2×150 mL), methanol (2×150 mL) and dichloromethane (300 mL, 6×150 mL). The product was cleaved from resin by treatment with 2,2,2-trifluoethanol (200 mL) for 19 hrs. Resin was filtered off and washed with dichloromethane (2×150 mL), 2-propanol/dichloromethane mixture (1:1, 2×150 mL), 2-propanol (150 mL) and dichloromethane (2×150 mL). Solutions were combined; solvent evaporated and crude product was purified by flash column chromatography (Silicagel 60, 0.040-0.060 mm; eluent: dichloromethane/methanol 1:0-9:1). Pure product was dried in vacuo and obtained as yellow oil.

Yield: 25.85 g (86%).

R_{F} (SiO₂, chloroform/methanol 85:15): 0.25.

¹H NMR spectrum (300 MHz, CDCl₃, δ_{H}): 7.38 (bs, 1H); 7.08 (bs, 1H); 6.61 (d, J=7.5 Hz, 1H); 4.43 (m, 1H); 4.15 (s, 2H); 4.01 (s, 2H); 3.78-3.39 (m, 16H); 2.31 (t, J=6.9 Hz, 2H); 2.27-2.09 (m, 5H); 2.01-1.84 (m, 1H); 1.69-1.50 (m, 4H); 1.46 (s, 9H); 1.43 (s, 9H); 1.24 (bs, 24H).

LC-MS purity: 100%.

LC-MS Rt (Sunfire 4.6 mm×100 mm, acetonitrile/water 60:40 to 0:100+0.1% FA): 7.89 min. LC-MS m/z: 846.6 (M+H)⁺.

### Example 2

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[DSer²,Glu¹⁶,Lys²⁴,Leu²⁷]

### Glucagon

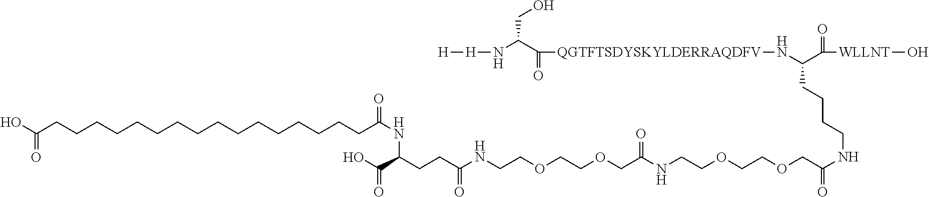

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: 8.4 min

UPLC 08_B2_1:12.6 min

UPLC 05_B5_1: 6.2 min

UPLC 04_A3_1: 9.3 min

LCMS: m/z 1408.08 (M+3H)3+, 1056.08 (M+4H)4+, 845.10 (M+5)5+

### Example 3

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys¹⁷,Lys¹⁸,Glu²¹,Lys²⁴,Leu²⁷]Glucagon

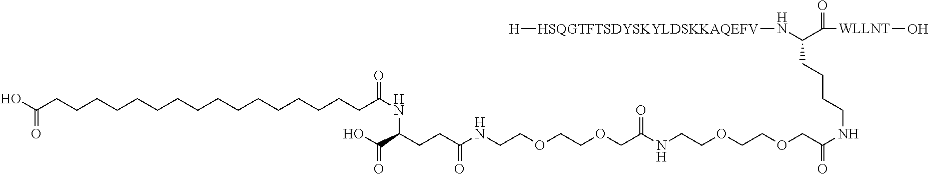

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: 8.2 min

UPLC 08_B2_1:12.5 min

UPLC 05_B5_1: 6.1 min

UPLC 04_A3_1:11.0 min

LCMS METHOD: LCMS_4: m/z 1380.09 (M+3H)3+, 1035.10 (M+4H)4+, 828.31 (M+5)5+

### Example 4

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys¹⁷,Glu²¹,Lys²⁴,Leu²⁷] Glucagon

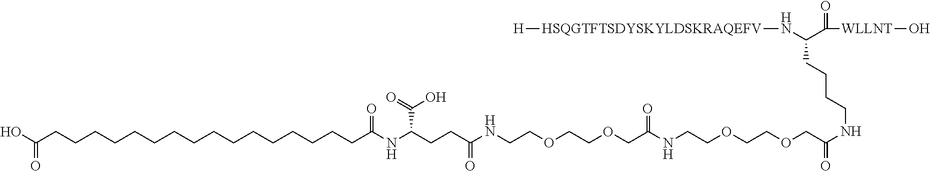

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1:8.5 min

UPLC 08_B2_1: 12.9 min

UPLC 05_B5_1:5.8 min

LCMS METHOD: LCMS 4: m/z 1389.32 (M+3H)3+, 1042.24 (M+4H)4+, 833.99 (M+5)5+

### Example 5

### N^{ε16}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys¹⁶,Lys¹⁷,Glu²¹,Leu²⁷] Glucagon

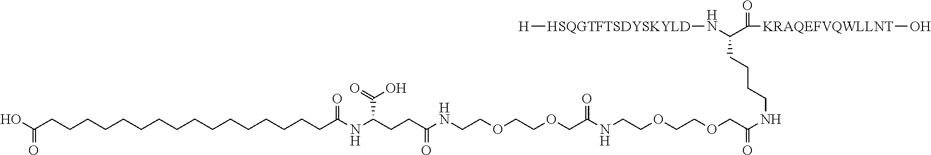

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1:8.6 min

UPLC 08_B2_1: 13.0 min

UPLC 05_B5_1: 6.0 min

LCMS METHOD: LCMS_4: m/z 1402.99 (M+3H)3+, 1052.5 (M+4H)4+, 842.21 (M+5)5+

### Example 6

### N^{ε16}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]]) [Lys¹⁶,Lys¹⁷,Lys¹⁸,Glu²¹,Leu²⁷]Glucagon

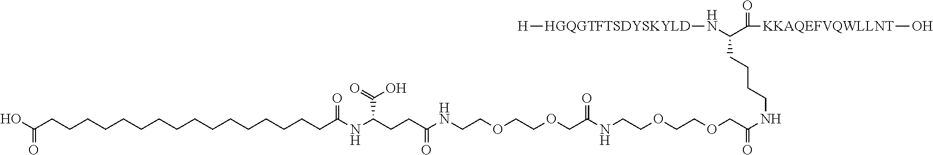

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1:8.5 min

UPLC 08_B2_1: 12.9 min

UPLC 05_B5_1: 6.0 min

LCMS METHOD: LCMS_4: m/z 1393.67 (M+3H)3+, 1045.50 (M+4H)4+, 836.61 (M+5)5+

### Example 7

### N^{ε25}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]]) [Lys²⁵,Leu²⁷] Glucagon

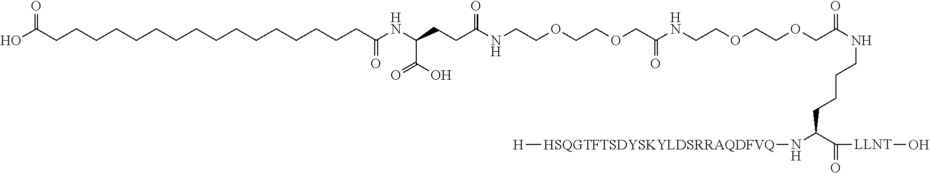

The peptide was prepared essentially as described in SPPS method A and C

UPLC 10_B5_2: 7.0 min

LCMS METHOD: LCMS_4: m/z 1374.65 (M+3H)3+, 1031.24 (M+4H)4+, 825.02 (M+5)5+

### Example 8

### N^{ε28}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]]) [Leu²⁷,Lys²⁸] Glucagon

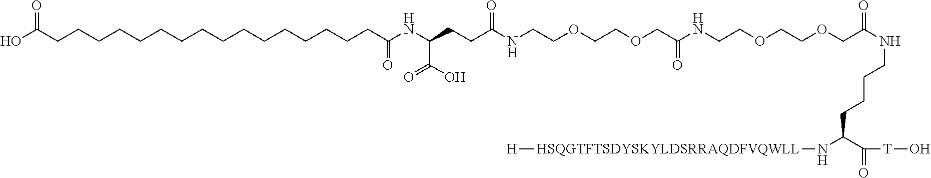

The peptide was prepared essentially as described in SPPS method A and C

UPLC 10_B5_2: 7.8 min

LCMS METHOD: LCMS_4: m/z 1399.34 (M+3H)3+, 1049.76 (M+4H)4+, 840.01 (M+5)5+

### Example 9

### N^{ε27}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys²⁷]Glucagon

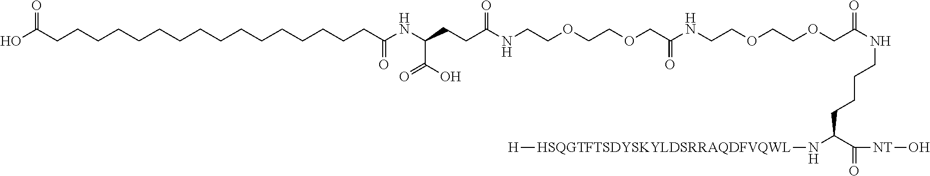

The peptide was prepared essentially as described in SPPS method A and C

UPLC 10_B5_2: 6.8 min

LCMS METHOD: LCMS_4: m/z 1399.4 (M+3H)3+

### Example 10

### N^{ε29}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Leu²⁷,Lys²⁹] Glucagon

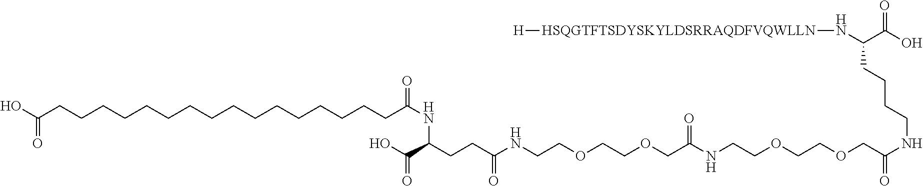

The peptide was prepared essentially as described in SPPS method A and C

UPLC 10_B4_2: 8.5 min

UPLC 10_B5_2: 8.1 min

LCMS METHOD: LCMS_4: m/z 1403.32 (M+3H)3+, 1052.50 (M+4H)4+, 842.19 (M+5)5+

### Example 11

### N^{α} ([Leu²⁷] Glucagonyl) N^{ε}[(4S)-5-hydroxy-4-[[(4S)-5-hydroxy-4-[[(4S)-5-hydroxy-4-[[(4S)-5-hydroxy-4-[(20-hydroxy-20-oxo-icosanoyl)amino]-5-oxo-pentanoyl]amino]-5-oxo-pentanoyl]amino]-5-oxo-pentanoyl]amino]-5-oxo-pentanoyl]Lysine

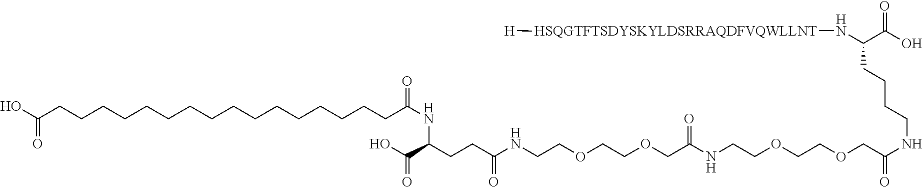

The peptide was prepared essentially as described in SPPS method A and C

UPLC 10_B4_2: 8.5 min

UPLC 10_B5_2: 7.9 min

LCMS METHOD: LCMS_4: m/z 1437.02 (M+3H)3+, 1078.01 (M+4H)4+, 862.41 (M+5)5+

### Example 12

### N^{ε12}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys¹²,Leu²⁷]Glucagon

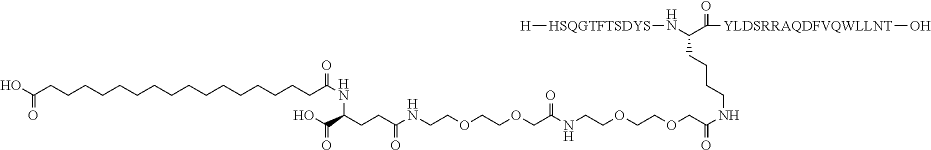

The peptide was prepared essentially as described in SPPS method A and C

UPLC 10_B4_2: 8.7 min

UPLC 10_B5_2: 8.4 min

UPLC 05_B5_1: min

UPLC 04_A3_1: min

LCMS METHOD: LCMS_4: m/z 1394.35 (M+3H)3+, 1045.99 (M+4H)4+

### Example 13

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Thr¹⁶,Lys²⁴,Leu²⁷,Ser²⁸] Glucagon

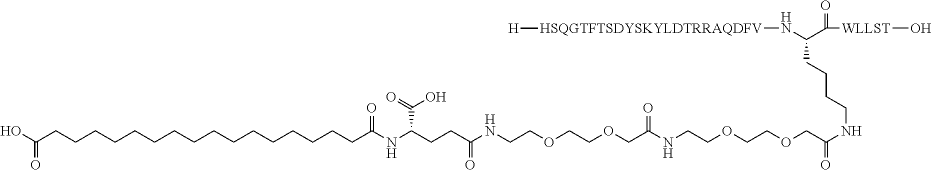

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: 5.1 min

UPLC 04_A3_1: 12.6 min

LCMS METHOD: LCMS_4: m/z 1389.79 (M+3H)3+, 1042.58 (M+4H)4+, 834.28 (M+5)5+

### Example 14

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys²⁴,Leu²⁷,Ser²⁸] Glucagon

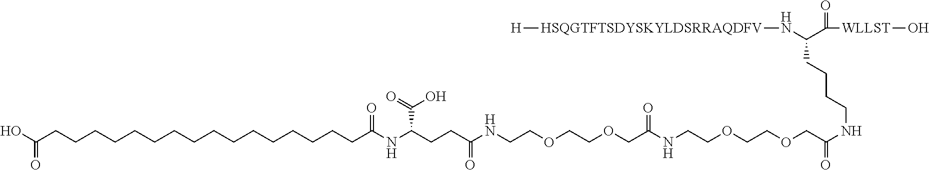

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 04_A4_1:6.7 min

UPLC 05_B5_1: 4.9 min

UPLC 04_A3_1: 12.0 min

LCMS METHOD: LCMS_4: m/z 1385.41 (M+3H)3+, 1039.06 (M+4H)4+, 831.45 (M+5)5+

### Example 15

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys²⁴,Leu²⁷,Thr²⁸] Glucagon

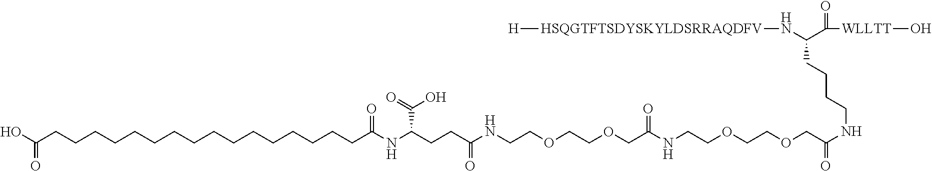

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 04_A4_1:6.4 min

UPLC 05_B5_1: 4.8 min

UPLC 04_A3_1: 11.7 min

LCMS METHOD: LCMS_4: m/z 1389.77 (M+3H)3+, 1042.58 (M+4H)4+, 834.27 (M+5) 5+

### Example 16

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys²⁴,Leu²⁷]Glucagon

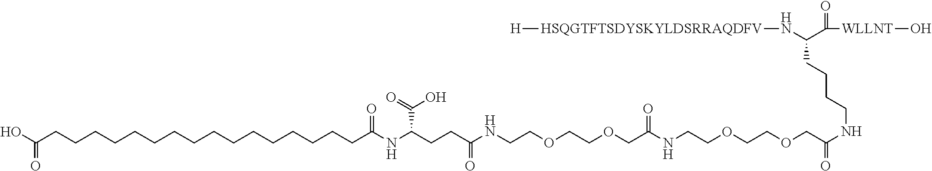

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 04_A4_1: 6.3 min

UPLC 05_B5_1:4.6 min

UPLC 04_A3_1: 11.6 min

LCMS METHOD: LCMS_4: m/z 1394.46 (M+3H)3+, 1045.84 (M+4H)4+, 836.88 (M+5)5+

### Example 17

### N^{ε16}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys¹⁶,Leu²⁷]Glucagon

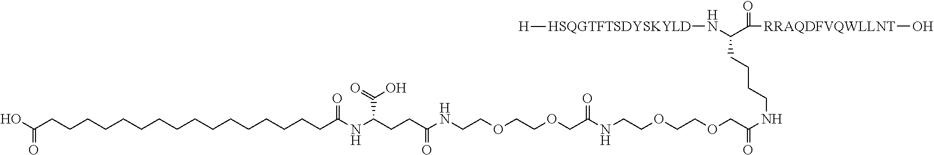

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1:8.5 min

UPLC 08_B2_1: 12.9 min

UPLC 05_B5_1: 4.8 min

UPLC 04_A3_1: 11.9 min

LCMS METHOD: LCMS_4: m/z 1407.65 (M+3H)3+, 1055.97 (M+4H)4+, 845.2 (M+5)5+

### Example 18

### N^{ε18}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys¹⁸,Leu²⁷] Glucagon

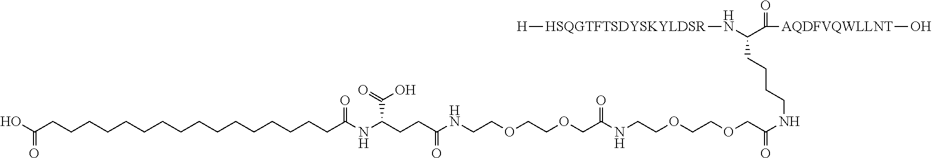

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

LCT Premier UPLC-MS: Rt 2.11 min. m/z: 1384.58 ((M/3)+3); 1038.69 ((M/4)+4).

UPLC 08_B4_1: 8.9 min

UPLC 08_B2_1: 13.5 min

UPLC 05_B5_1:5.1 min

UPLC 04_A3_1: 11.5 min

LCMS METHOD: LCMS_4: m/z 1384.58 (M+3H)3+, 1038.69 (M+4H)4+

### Example 19

### N^{ε17}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Lys¹⁷,Leu²⁷]Glucagon

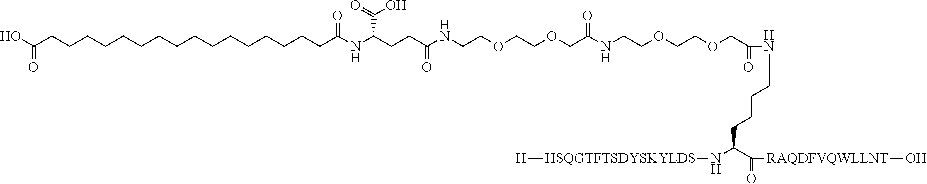

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

LCT Premier UPLC-MS: Rt 2.06 min. m/z: 1384.81 ((M/3)+3); 1038.62 ((M/4)+4).

UPLC 08_B4_1: 8.7 min

UPLC 08_B2_1: 13.2 min

UPLC 05_B5_1: 4.9 min

LCMS METHOD: LCMS_4: m/z 1384.81 (M+3H)3+, 1038.62 (M+4H)4+

### Example 20

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Arg¹², Lys²⁴,Leu²⁷] Glucagon

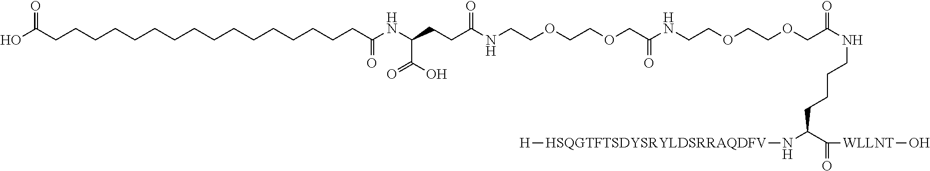

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: 8.74 min

UPLC 05_B5_1: 5.25 min

LCMS METHOD: LCMS_4: 4208.0

### Example 21

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Glu²¹,Lys²⁴,Leu²⁷] Glucagon

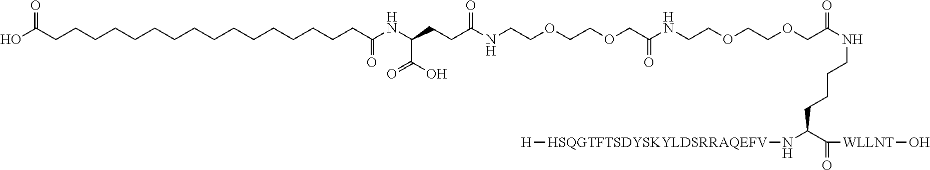

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: 8.50 min

LCMS METHOD: LCMS_4: 4193

### Example 22

### N^{α}-Glucagonyl-N^{ε}[(4S)-5-hydroxy-4-[[(4S)-5-hydroxy-4-[[(4S)-5-hydroxy-4-[[(4S)-5-hydroxy-4-[(20-hydroxy-20-oxo-icosanoyl)amino]-5-oxo-pentanoyl]amino]-5-oxo-pentanoyl]amino]-5-oxo-pentanoyl]amino]-5-oxo-pentanoyl]lysinyl amide

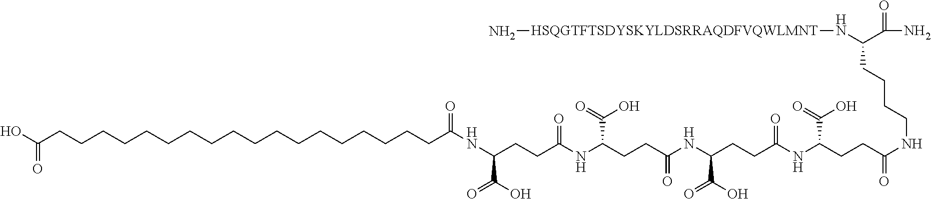

The peptide was prepared essentially as described in SPPS method A and C.

UPLC 08_B4_1:8.7 min

LCMS METHOD: LCMS_4: m/z 4450

### Example 23

### N^{α}—(N^{ε24}[2-[2-[2-[[(4S)-5-hydroxy-4-[(18-hydroxy-18-oxo-octadecanoyl)amino]-5-oxo-pentanoyl]amino]ethoxy]ethoxy]acetyl] [D-Ser², Lys²⁰] Glucagonyl) Lysinyl amide

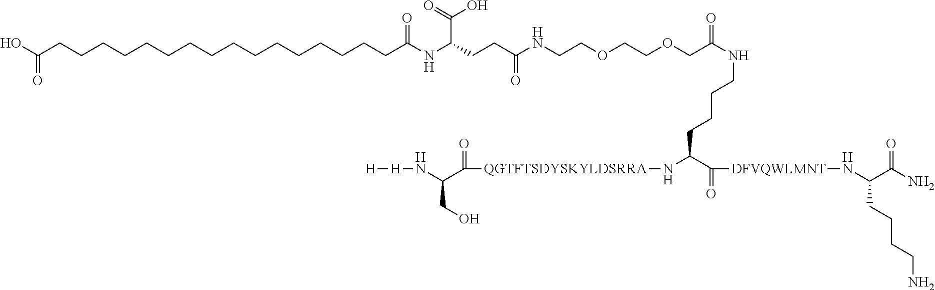

The peptide was prepared essentially as described in SPPS method A and C

UPLC 08_B4_1: 7.87 min

LCMS METHOD: LCMS_4: m/z 4181

### Example 24

### N^{ε24}-[2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl][Glu¹⁶,Lys²⁴]Glucagon peptide amide

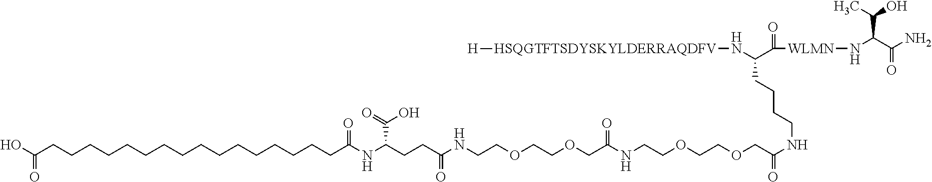

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: Rt=6.2 min

UPLC 04_A3_1: Rt=11.7 min

LCMS METHOD: LCMS_4: m/z 1413.8 (M+3H)3+, 1060.7 (M+4H)4+, 848.8 (M+5)5+

### Example 25

### N^{α}([Glu¹⁶]Glucagonyl)N^{ε}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])Lysinyl amide

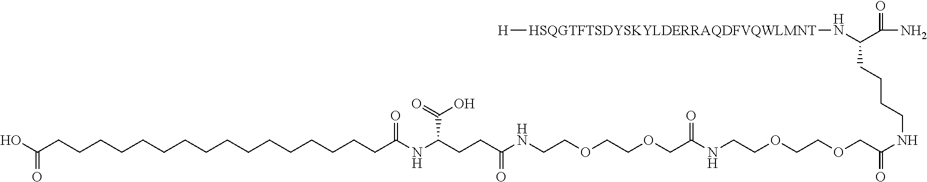

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.3

UPLC 08_B4_1: Rt=8.2

UPLC 05_B5_1: Rt=5.0

UPLC 04_A3_1: Rt=10.9

LCMS METHOD: LCMS_4: m/z 1457 (M+3H)3+, 1093 (M+4H)4+, 874 (M+5)5+

### Example 26

### N^{α}([Glu¹⁶,Gln¹⁷,Arg²⁰]Glucagonyl)N^{ε}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])Lysinyl amide

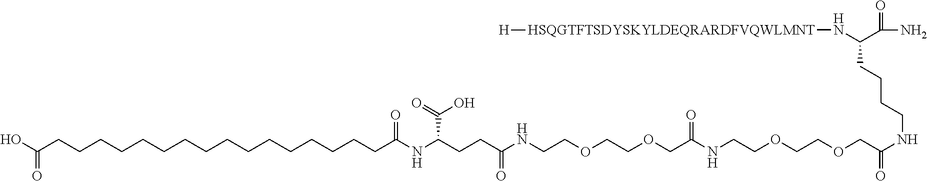

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.2

UPLC 08_B4_1: Rt=8.1

UPLC 05_B5_1: Rt=4.8

UPLC 04_A3_1: Rt=11.1

LCMS METHOD: LCMS_4: m/z 1457 (M+3H)3+, 1092 (M+4H)4+, 874 (M+5)5+

### Example 27

### N^{α}([Glu¹⁶,Gln¹⁷,Ala¹⁸,Arg²⁰]Glucagonyl)N^{ε}-([2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]) Lysinyl amide

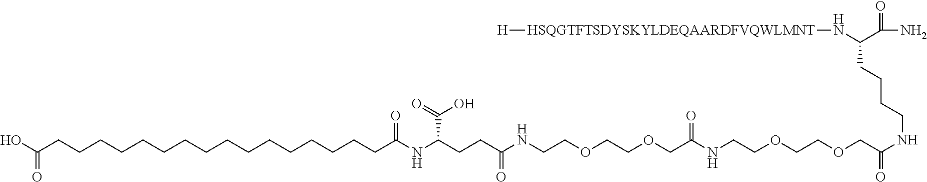

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.9

UPLC 08_B4_1: Rt=8.6

UPLC 05_B5_1: Rt=5.7

UPLC 04_A3_1: Rt=11.3

LCMS METHOD: LCMS_4: m/z 1428 (M+3H)3+, 1071 (M+4H)4+, 857 (M+5)5+

### Example 28

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Glu¹⁶,Lys²⁴,Met(O)²⁷] Glucagon peptide amide

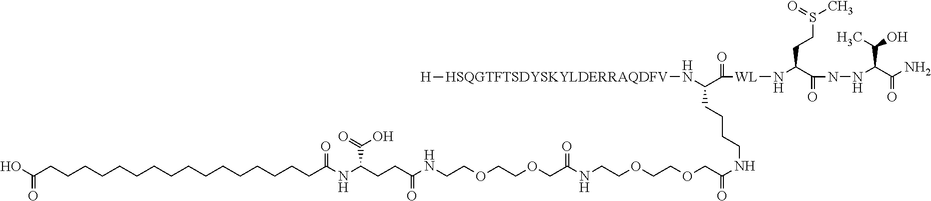

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: Rt=4.7

UPLC 04_A4_1: Rt=4.1

LCMS METHOD: LCMS_4: m/z 1419.2 (M+3H)3+, 1064.7 (M+4H)4+, 852.0 (M+5)5+

### Example 29

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl]) [Aib²,Glu¹⁶,Lys²⁴,Leu²⁷]Glucagon peptide amide

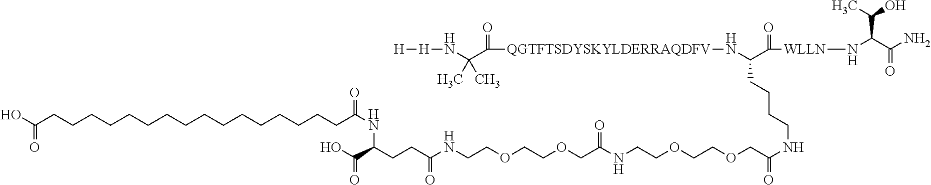

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: Rt=8.4

UPLC 04_A4_1: Rt=7.2

LCMS METHOD: LCMS_4: m/z 1407.8 (M+3H)3+, 1056.4 (M+4H)4+, 845.6 (M+5)5+

### Example 30

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[D-Ser²,Glu¹⁶,Gln¹⁷,Ala¹⁸,Arg²⁰,Lys²⁴,Leu²⁷] Glucagon peptide amide

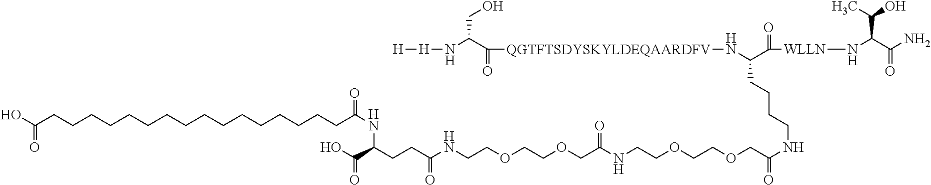

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: Rt=7.1

UPLC 04_A4_1: Rt=7.7

LCMS METHOD: LCMS_4: m/z 1380.4 (M+3H)3+, 1035.6 (M+4H)4+, 828.7 (M+5)5+

### Example 31

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Glu²¹,Lys²⁴,Arg²⁵ Leu²⁷,]Glucagon peptide amide

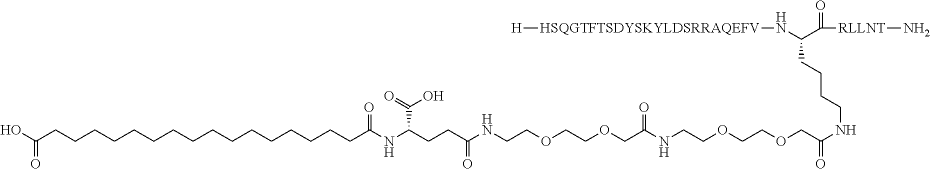

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: Rt=5.8

UPLC 08_B4_1: Rt=7.6

LCMS METHOD: LCMS_4: m/z 1388.7 (M+3H)3+, 1041.8 (M+4H)4+, 833.7 (M+5)5+

### Example 32

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Glu¹⁸, Lys²⁴, Leu²⁷, Ala²⁸] Glucagon peptide amide

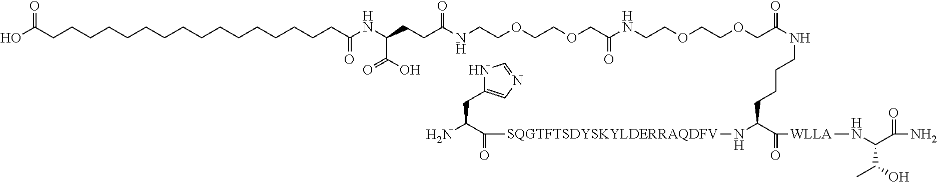

The peptide was prepared essentially as described in SPPS method A and C.

UPLC 05_B9_1: Rt=8.2

UPLC 08_B4_1: Rt=8.5

LCMS METHOD: LCMS_4: m/z 1393.7 (M+3H)3+

### Example 33

### (N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Gln¹⁷,Lys²⁴,Val²⁷,Lys²⁸] Glucagonyl)-Gly-Pro amide

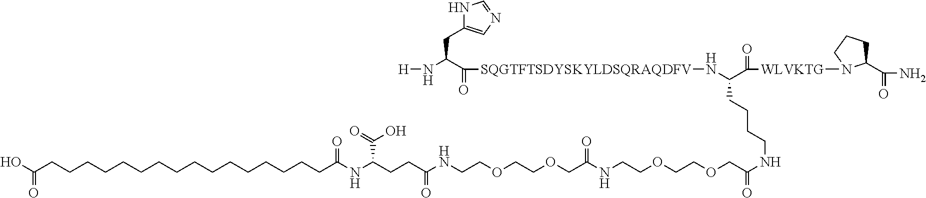

The peptide was prepared essentially as described in SPPS method A and C.

UPLC 08_B4_1: Rt=8.0

LCMS METHOD: LCMS_4: m/z 1436.3 (M+3H)3+

### Example 34

### N^{ε16}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁶,Lys¹⁷,Glu²¹,Leu²⁷] Glucagon peptide amide

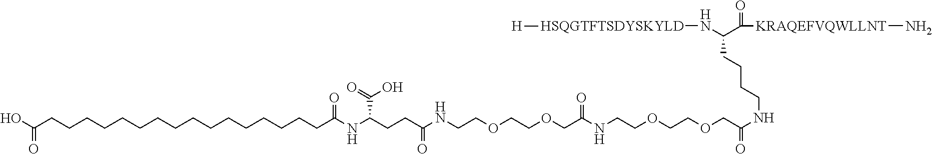

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.9

UPLC 08_B4_1: Rt=8.5

UPLC 05_B5_1: Rt=6.4

LCMS METHOD: LCMS_4: m/z 1402.7 (M+3H)3+, 1052.3 (M+4H)4+, 842.2 (M+5)5+

### Example 35

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁷,Glu²¹,Lys²⁴,Leu²⁷] Glucagon peptide amide

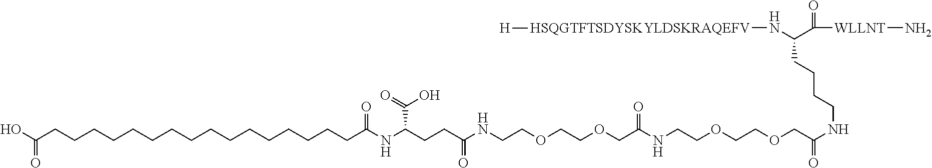

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.8

UPLC 08_B4_1: Rt=8.5

UPLC 05_B5_1: Rt=6.2

LCMS METHOD: LCMS_4: m/z 1389.3 (M+3H)3+, 1042.0 (M+4H)4+, 833.1 (M+5)5+

### Example 36

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Glu¹⁶,Lys¹⁷,Ala¹⁸,Glu²¹, Lys²⁴,Leu²⁷]Glucagon peptide amide

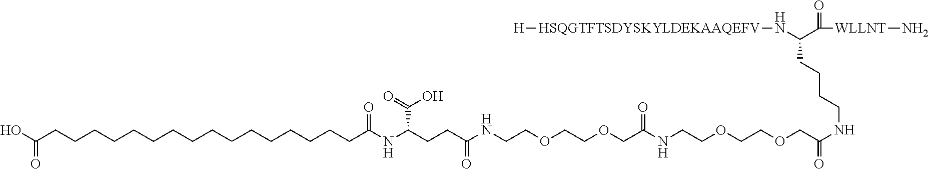

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=13.7

UPLC 08_B4_1: Rt=9.0

UPLC 05_B5_1: Rt=7.1

LCMS METHOD: LCMS_4: m/z 1374.7 (M+3H)3+, 1031.2 (M+4H)4+

### Example 37

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁷,Ala¹⁸,Glu²¹,Lys²⁴, Leu²⁷]Glucagon peptide amide

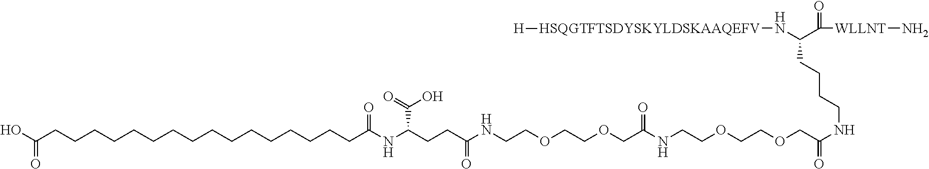

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=13.6

UPLC 08_B4_1: Rt=8.9

UPLC 05_B5_1: Rt=7.1

LCMS METHOD: LCMS_4: m/z 1361.0 (M+3H)3+, 1020.75 (M+4H)4+

### Example 38

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Glu¹⁶,Lys¹⁷,Glu²¹, Lys²⁴,Leu²⁷] Glucagon peptide amide

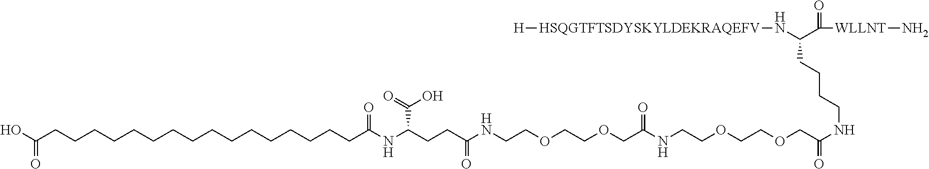

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.9

UPLC 08_B4_1: Rt=8.5

UPLC 05_B5_1: Rt=6.1

LCMS METHOD: LCMS_4: m/z 1403.3 (M+3H)3+, 1052.5 (M+4H)4+, 842.2 (M+5)5+

### Example 39

### N^{ε16}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Aib²,Lys¹⁶,Lys¹⁷, Glu²¹,Leu²⁷]Glucagon peptide amide

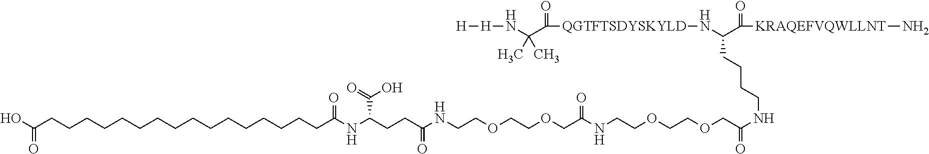

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: Rt=5.0

UPLC 04_A3_1: Rt=14.5

UPLC 04_A4_1: Rt=9.2

LCMS METHOD: LCMS_4: m/z 1402.5 (M+3H)3+, 1051.85 (M+4H)4+, 841.7 (M+5)5+

### Example 40

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Glu²¹,Lys²⁴,Leu²⁷,Ser²⁸]Glucagon peptide amide

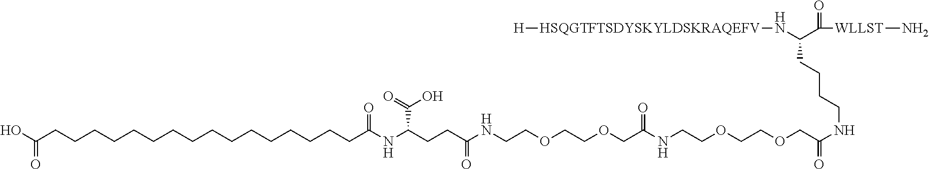

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 09_B2_1: Rt=12.8

UPLC 09_B4_1: Rt=8.5

UPLC 05_B5_1: Rt=5.6

LCMS METHOD: LCMS_4: m/z 1380.2 (M+3H)3+, 1035.1 (M+4H)4+, 828.3 (M+5)5+

### Example 41

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁷, Glu²¹,Lys²⁴,Leu²⁷,Glu²⁸,]Glucagon peptide amide

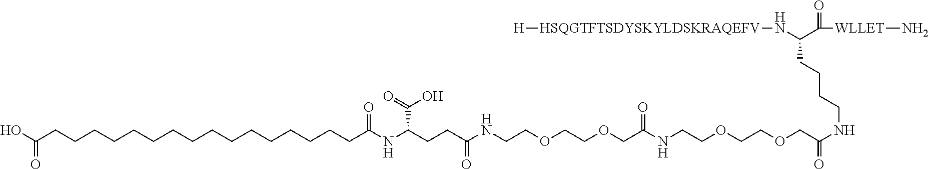

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.8

UPLC 08_B4_1: Rt=8.5

UPLC 05_B5_1: Rt=5.4

LCMS METHOD: LCMS_4: m/z 1394.1 (M+3H)3+, 1045.6 (M+4H)4+, 836.7 (M+5)5+

### Example 42

### N^{α}-([Lys¹⁷, Glu²¹, Leu²⁷] Glucagonyl) N^{ε}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl]) Lysinyl amide

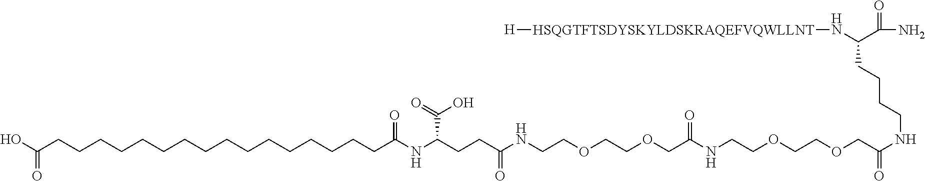

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.4

UPLC 08_B4_1: Rt=8.2

UPLC 05_B5_1: Rt=4.6

LCMS METHOD: LCMS_4: m/z 1431.9 (M+3H)3+, 1074.2 (M+4H)4+, 859.4 (M+5)5+

### Example 43

### N^{ε28}([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁷,Glu²¹,Leu²⁷,Lys²⁸] Glucagon peptide amide

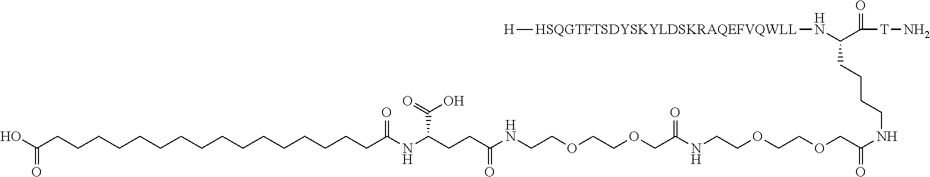

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B2_1: Rt=12.7

UPLC 08_B4_1: Rt=8.5

UPLC 05_B5_1: Rt=5.2

LCMS METHOD: LCMS_4: m/z 1393.9 (M+3H)3+, 1045.7 (M+4H)4+, 836.6 (M+5)5+

### Example 44

### N^{ε25}([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁷,Glu²¹,Lys²⁵,Leu²⁷] Glucagon peptide amide

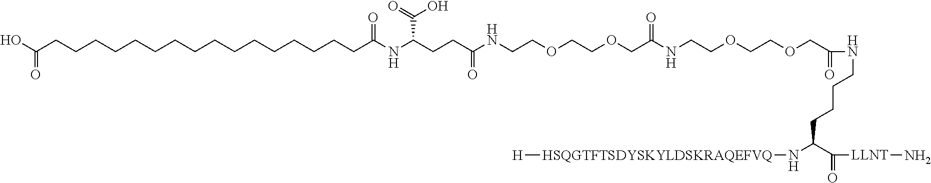

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: Rt=4.5

LCMS METHOD: LCMS_4: m/z 1369.5 (M+3H)3+, 1027.4 (M+4H)4+, 822.1 (M+5)5+

### Example 45

### N^{ε27}([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁷,Glu²¹,Lys²⁷] Glucagon peptide amide

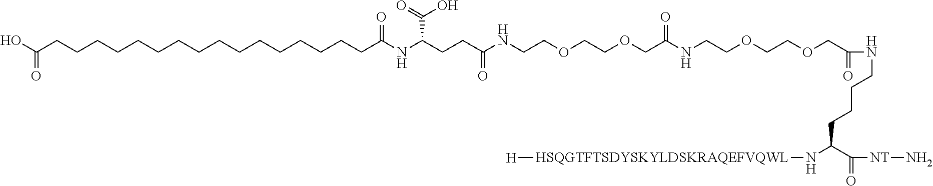

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: Rt=4.2

LCMS METHOD: LCMS_4: m/z 1394.2 (M+3H)3+, 1045.6 (M+4H)4+, 836.7 (M+5)5+

### Example 46

### N^{ε29}([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁷,Glu²¹,Leu²⁷,Lys²⁹] Glucagon peptide amide

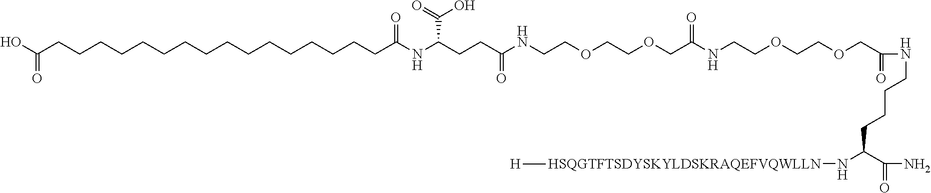

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 05_B5_1: Rt=4.930 min; 93% purity.

LCMS METHOD: LCMS_4: m/z 1398.2 (M+3H)3+, 1048.6 (M+4H)4+, 839.1 (M+5)5+

### Example 47

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Arg¹², Lys²⁴,Leu²⁷] Glucagon

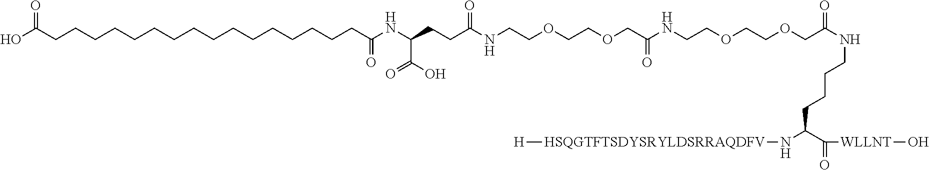

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: Rt=8.7

UPLC 05_B5_1: Rt=5.2

LCMS METHOD: LCMS_4: m/z 4208

### Example 48

### N^{ε24}-([2-[2-[2-[[(4S)-5-hydroxy-4-[(18-hydroxy-18-oxooctadecanoyl)amino]5-oxopentanoyl]amino]ethoxy]ethoxy]acetyl]amino]ethoxy]ethoxy]acetyl]])[Glu²¹,Lys²⁴,Leu²⁷] Glucagon

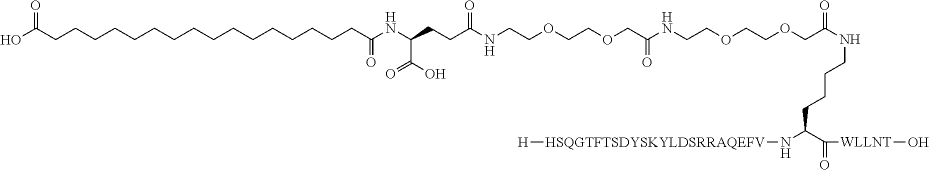

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: Rt=8.5

LCMS METHOD: LCMS_4: m/z 4193

### Example 49

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Gln¹⁸,Glu²¹,Lys²⁴,Leu²⁷] Glucagon

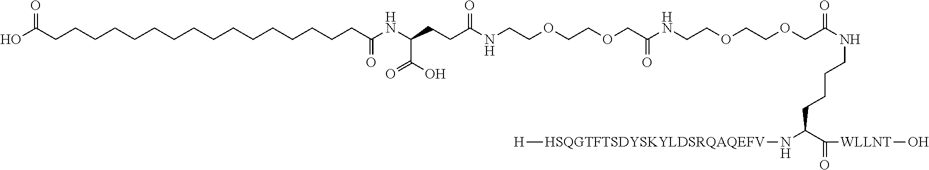

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: Rt=8.7

UPLC 05_B5_1: Rt=5.6

LCMS METHOD: LCMS_4: m/z 4166

### Example 50

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys²⁴,His²⁵,Leu²⁷] Glucagon

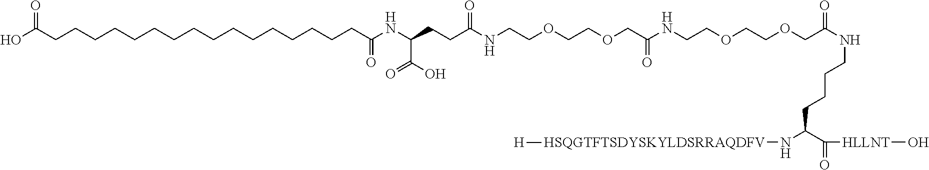

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: Rt=7.8

UPLC 05_B5_1: Rt=4.3

LCMS METHOD: LCMS_4: m/z 4131

### Example 51

### N^{ε24}-([(4S)-5-hydroxy-4-[[(4S)-5-hydroxy-4-[[2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl]) [Lys²⁴,Leu²⁷] Glucagon

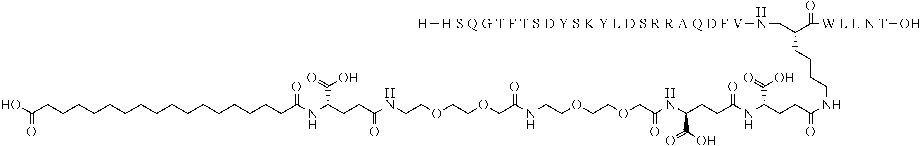

The peptide was prepared essentially as described in SPPS method A and C

UPLC 09_B2_1: Rt=12.7

UPLC 09_B4_1: Rt=8.4

LCMS METHOD: LCMS_4 m/z: 4439.00 (M)+; 1480.15 ((M/3)+3); 1110.11 ((M/4)+4); 888.29 ((M/5)+5)

### Example 52

### N^{ε28}-([(4S)-5-hydroxy-4-[[(4S)-5-hydroxy-4-[[2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl]) [Leu²⁷,Lys²⁸] Glucagon

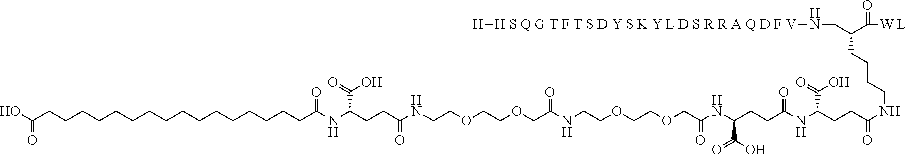

The peptide was prepared essentially as described in SPPS method A and C

UPLC 08_B2_1: Rt=12.7

UPLC 08_B4_1: Rt=8.4

LCMS METHOD: LCMS_4: m/z 4452.50 (M)+; 1484.79 ((M/3)+3); 1113.59 ((M/4)+4); 891.08 ((M/5)+5).

### Example 53

### N^{ε29}-([(4S)-5-hydroxy-4-[[(4S)-5-hydroxy-4-[[2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanyl]) [Leu²⁷,Lys²⁹] Glucagon

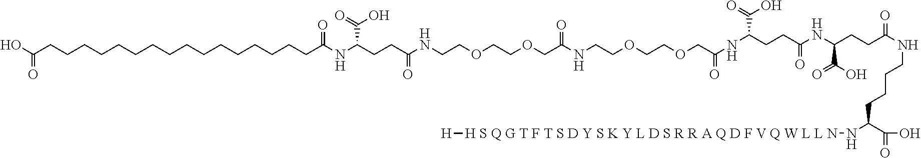

The peptide was prepared essentially as described in SPPS method A and C

UPLC 08_B2_1: Rt=12.6

UPLC 08_B4_1: Rt=8.4

LCMS METHOD: LCMS_4 m/z: 4465.50 (M)+; 1489.12 ((M/3)+3); 1117.09 ((M/4)+4); 893.67 (M/5)+5)

### Example 54

### N^{α}-([Leu²⁷]Glucagonyl)N^{ε}-([(4S)-5-hydroxy-4-[[(4S)-5-hydroxy-4-[[2-[2-[2-[[2-[2-[2-[[(4S)- 5-hydroxy-4-[(18-hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl])Lysine

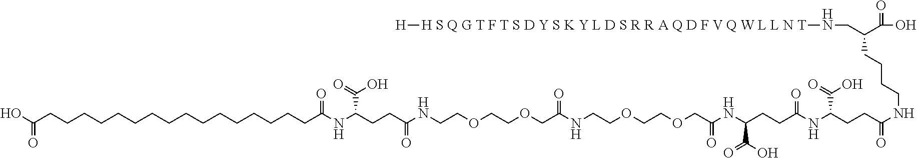

The peptide was prepared essentially as described in SPPS method A and C

UPLC 08_B2_1: Rt=12.6

UPLC 08_B4_1: Rt=8.4

LCMS METHOD: LCMS_4 m/z: 4465.50 (M)+; 1489.12 ((M/3)+3); 1117.09 ((M/4)+4); 893.67 (M/5)+5).

### Example 55

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl]) [Lys¹⁷,Lys¹⁸,Glu²¹,Lys²⁴,Leu²⁷, Ser²⁸] Glucagon

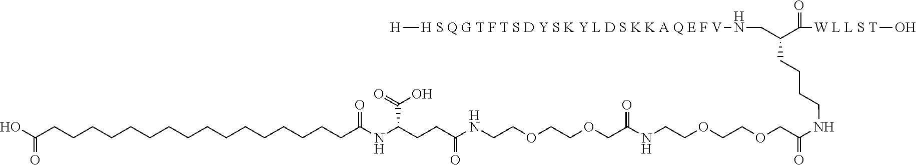

The peptide was prepared essentially as described in SPPS method A and C

UPLC 08_B2_1: Rt=12.9

UPLC 08_B4_1: Rt=8.5

LCMS METHOD: LCMS_4 m/z: 4110.50 (M)+; 1370.92 ((M/3)+3); 1028.19 ((M/4)+4); 822.75 ((M/5)+5).

### Example 56

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys²⁴,(p)Tyr²⁵,Leu²⁷] Glucagon

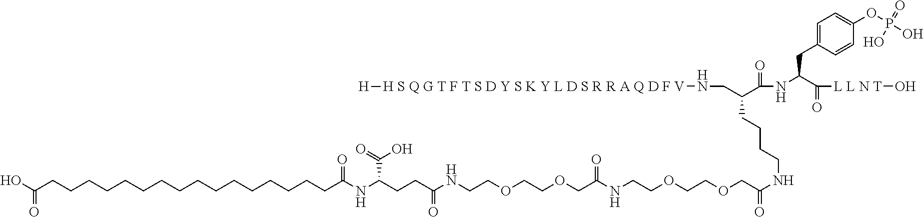

The peptide was prepared essentially as described in SPPS method A and B using Fmoc-Tyr(PO(NMe₂)₂)-OH in the synthesis of the peptide and 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid. The protected phosphotyrosine was deprotected by adding water to a total of 10% (V/V) after cleavage from the resin. The TFA-water mixture was kept for 16 hours to ensure deprotection of the phosphotyrosine.

UPLC 09_B2_1: Rt=12.7

UPLC 09_B4_1: Rt=8.4

LCMS METHOD: LCMS_4 m/z: 4237.00 (M)+; 1413.04 ((M/3)+3); 1059.78 ((M/4)+4); 848.26 ((M/5)+5).

### Example 57

### N^{ε10}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Lys¹⁰,Leu²⁷]Glucagon

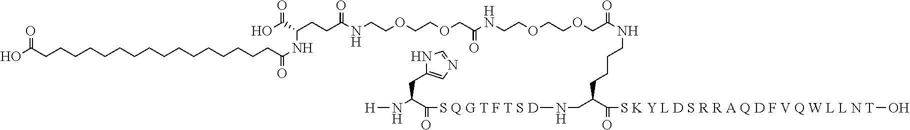

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: Rt=8.3

UPLC 05_B5_1: Rt=5.0

LCMS METHOD: LCMS_4 m/z: 1382.18 ((M/3)+3); 1036.89 ((M/4)+4); 829.72 ((M/5)+5).

### Example 58

### N^{ε24}-([2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl])[Glu²¹,Lys²⁴,Arg²⁵,Leu²⁷] Glucagon

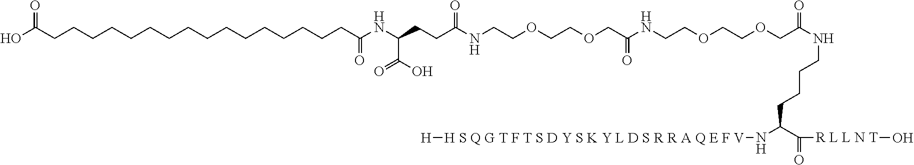

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: Rt=8.55

LCMS METHOD: LCMS_4: 4164.8

### Example 59

### N^{α}-([Lys¹⁷,Lys¹⁸,Glu²¹,Leu²⁷] Glucagonyl) N^{ε}-([2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]-acetyl]) Lysin

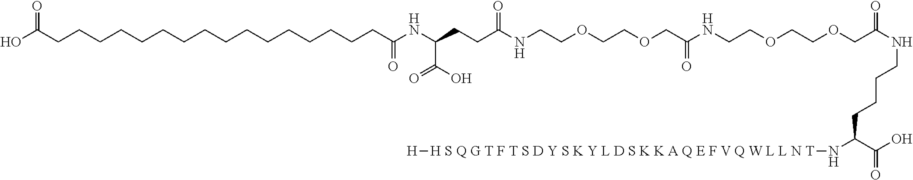

The peptide was prepared essentially as described in SPPS method A and B using 2-[2-[2-[[2-[2-[2-[[(4S)-5-tert-butoxy-4-[(18-tert-butoxy-18-oxo-octadecanoyl)amino]-5-oxo-pentanoyl]amino]ethoxy]ethoxy]acetyl]amino]ethoxy]ethoxy]acetic acid.

UPLC 08_B4_1: Rt=8.45

LCMS METHOD: LCMS_4: 4266.5

### Example 60

### ThT Fibrillation Assays for the Assessment of Physical Stability of Protein Formulations

Low physical stability of a peptide may lead to amyloid fibril formation, which is observed as well-ordered, thread-like macromolecular structures in the sample eventually resulting in gel formation. This has traditionally been measured by visual inspection of the sample. However, that kind of measurement is very subjective and depending on the observer. Therefore, the application of a small molecule indicator probe is much more advantageous. Thioflavin T (ThT) is such a probe and has a distinct fluorescence signature when binding to fibrils [Naiki et al. (1989) Anal. Biochem. 177, 244-249; LeVine (1999) Methods. Enzymol. 309, 274-284].

The time course for fibril formation can be described by a sigmoidal curve with the following expression [Nielsen et al. (2001) Biochemistry 40, 6036-6046]:

$$F = {f}_{i} + {m}_{i} t + \frac{{f}_{f} + {m}_{f} t}{1 + {e}^{- [ ( t - {t}_{0} ) / τ ]}} \qquad Eq . ( 1 )$$

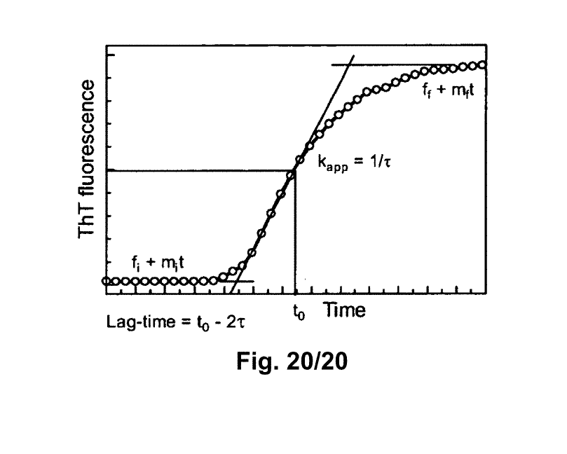

Here, F is the ThT fluorescence at the time t. The constant t0 is the time needed to reach 50% of maximum fluorescence, as depicted in FIG. 20. The two important parameters describing fibril formation are the lag-time calculated by t0-2τ and the apparent rate constant kapp 1/τ.

Formation of a partially folded intermediate of the peptide is suggested as a general initiating mechanism for fibrillation. Few of those intermediates nucleate to form a template onto which further intermediates may assembly and the fibrillation proceeds. The lag-time corresponds to the interval in which the critical mass of nucleus is built up and the apparent rate constant is the rate with which the fibril itself is formed.

Samples were prepared freshly before each assay. Each sample composition is described in the legends. The pH of the sample was adjusted to the desired value using appropriate amounts of concentrated NaOH and HCl. Thioflavin T was added to the samples from a stock solution in H₂O to a final concentration of 1 μM.

Sample aliquots of 200 μl were placed in a 96 well microtiter plate (Packard OptiPlate™-96, white polystyrene). Usually, four or eight replica of each sample (corresponding to one test condition) were placed in one column of wells. The plate was sealed with Scotch Pad (Qiagen).

Incubation at given temperature, shaking and measurement of the ThT fluorescence emission were done in a Fluoroskan Ascent FL fluorescence platereader (Thermo Labsystems). The temperature was adjusted to the desired value, typically 30° C. or 37° C. The plate was either incubated without shaking (no external physical stress) or with orbital shaking adjusted to 960 rpm with an amplitude of 1 mm. Fluorescence measurement was done using excitation through a 444 nm filter and measurement of emission through a 485 nm filter.

Each run was initiated by incubating the plate at the assay temperature for 10 min. The plate was measured every 20 minutes for a desired period of time. Between each measurement, the plate was shaken and heated as described.

After completion of the ThT assay the four or eight replica of each sample was pooled and centrifuged at 20000 rpm for 30 minutes at 18° C. The supernatant was filtered through a 0.22 μm filter and an aliquot was transferred to a HPLC vial.

The concentration of peptide in the initial sample and in the filtered supernatant was determined by reverse phase HPLC using an appropriate standard as reference. The percentage fraction the concentration of the filtered sample constituted of the initial sample concentration was reported as the recovery.

The measurement points were saved in Microsoft Excel format for further processing and curve drawing and fitting was performed using GraphPad Prism. The background emission from ThT in the absence of fibrils was negligible. The data points are typically a mean of four or eight samples and shown with standard deviation error bars. Only data obtained in the same experiment (i.e. samples on the same plate) are presented in the same graph ensuring a relative measure of fibrillation between experiments.

The data set may be fitted to Eq. (1). However, the lag time before fibrillation may be assessed by visual inspection of the curve identifying the time point at which ThT fluorescence increases significantly above the background level.

### Example 61

### Peptide Solubility

The solubility of peptides and proteins depends on the pH of the solution. Often a protein or peptide precipitates at or close to its isoelectric point (pI), at which its net charge is zero. At low pH (i.e. lower than the pI) proteins and peptides are typically positively charged, at pH higher than the pI they are negatively charged.

It is advantageous for a therapeutic peptide if it is soluble in a sufficient concentration at a given pH, which is suitable for both formulating a stable drug product and for administrating the drug product to the patient e.g. by subcutaneous injection.

Solubility versus pH curves were measured as described: A formulation or a peptide solution in water was prepared and aliquots were adjusted to pH values in the desired range by adding HCl and NaOH. These samples were left equilibrating at room temperature for 2-3 days. Then the samples were centrifuged. A small aliquot of each sample was withdrawn for reverse HPLC analysis for determination of the concentration of the proteins in solution. The pH of each sample was measured after the centrifugation, and the concentration of each protein was depicted versus the measured pH.

### Example 62

### Peptide Solubility at pH 7.5

A solubility test at pH 7.5 of native glucagon and glucagon analogues was performed in order to establish if the solubility of the glucagon analogues near physiological pH was improved compared to native glucagon.

A sample of native glucagon or glucagon analogue (typical 250 nmol) was added HEPES buffer (typical 1 mL) to a nominal concentration of 250 μM. The mixture was kept for 1 h at room temperature and was occasionally shaken whereupon a sample of 200 μL was taken from the solution. The sample was centrifuged (6000 rpm, 5 min) whereupon the supernatant was quantified using a chemiluminescent nitrogen specific HPLC detector (Antek 8060 HPLC-CLND).

### Example 63

### Peptide Solubility/Stability

A stability test of glucagon analogues was performed in order to establish if the stability of the solutions were improved compared to solutions of native glucagon.

A sample of glucagon analogue (typical 250 nmol) was added HEPES buffer (typical 1 mL) to a nominal concentration of 250 μM. The mixture was kept for 1 h at room temperature and was occasionally shaken whereupon a sample of 200 μL was taken from the solution. The sample was centrifuged (6000 rpm, 5 min) and the supernatant was analyzed on a UPLC and the area under the peak (UV absorption at 214 nm) was measured as t=0. Due to the poor solubility of glucagon at pH 7.5 a sample of glucagon (GlucaGen® hypokit, Novo Nordisk in water, 250 μM, pH 2-3)) was included for comparison. The solutions were kept at 30° C. for 6 days whereupon the solution was filtered (Millex®-GV, 0.22 μm filter unit, Durapore® Membrane) and analyzed on a UPLC. The area under the peak (UV absorption at 214 nm) was measured as t=6 days.

### Example 64

Co-Formulation of a Glucagon Analogue (Example 3) with GLP-1 Analogue G1, GLP-1 Analogue G3 and Insulin Analogue G5

Co-formulation of the glucagon analogue (Example 3) was investigated with a number of peptides with potential for treatment of obesity and diabetes. The following formulations were prepared:

1. 250 μM glucagon analogue (Example 3), 10 mM Hepes pH 7.5

2. 250 μM glucagon analogue (Example 3), 0.6 mM insulin analogue G5, 0.5 mM Zn(Ac)2, 16 mM m-cresol, 16 mM phenol, 213 mM glycerol, pH 7.6

3. 250 μM glucagon analogue (Example 3), 1.6 mM GLP-1 analogue G1, 58 mM phenol, 10 mM phosphate pH 8.15

4. 250 μM glucagon analogue (Example 3), 1.2 mM GLP-1 analogue G3, 58 mM phenol, 10 mM phosphate pH 7.4

5. 0.6 mM insulin analogue G5, 0.5 mM Zn(Ac)2, 16 mM m-cresol, 16 mM phenol, 213 mM glycerol, pH 7.6

6. 1.6 mM GLP-1 analogue G1, 58 mM phenol, 10 mM phosphate pH 8.15

Formulation 2 was prepared by diluting an appropriate insulin analogue G5 stock solution in water, adding m-cresol and phenol, and then adding zinc acetate. The glucagon analogue was added as the last component. Formulation 5 was prepared in a similar fashion.

These 6 formulations were subjected to the ThT fibrillation assay. Samples were incubated at 37° C. for 45 hours and with vigorously shaking (960 rpm). Under these conditions no samples exhibited any ThT fluorescence signal and full recovery of both the glucagon analogue and the combined peptides (GLP-1 analogue G3 was not analysed due to technical reasons) were found in formulations. Thus co-formulating glucagon analogue (Example 3) with other peptides did not result in less stable formulations compared to the individual peptides (Formulations 1, 5, and 6).

### Example 65: Preparation of GLP-1 Derivatives

The following GLP-1 compounds were prepared (all being derivatives of analogues of GLP-1(7-37)):

### Compound G1

### N-epsilon26-((S)-4-Carboxy-4-hexadecanoylamino-butyryl)[Arg34]GLP-1-(7-37), which may also be designated Arg³⁴Lys²⁶(Nε-(γ-glutamyl(Nα-hexadecanoyl)))-GLP-1(7-37)-OH

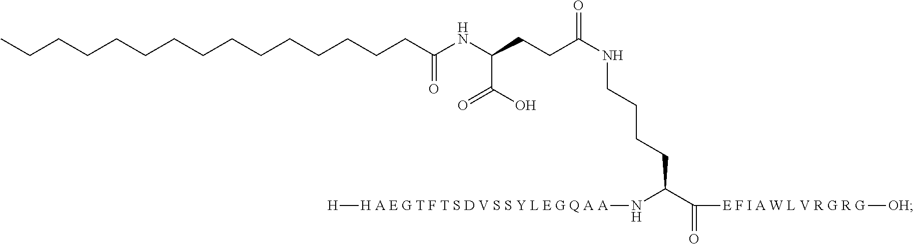

### Compound G2

### N-epsilon37-[2-(2-{2-[2-(2-{2-[(S)-4-Carboxy-4-({trans-4-[(19-carboxynonadecanoylamino)methyl]cyclohexanecarbonyl}amino)butyrylamino]ethoxy}ethoxy)acetylamino]ethoxy}ethoxy)acetyl][DesaminoHis7,Glu22,Arg26,Arg34,Lys37]GLP-1-(7-37)

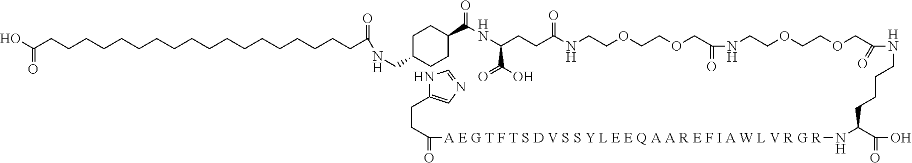

### Compound G3

### N-epsilon26-[2-(2-{2-[2-(2-{2-[(S)-4-Carboxy-4-(17-carboxyheptadecanoylamino)butyrylamino]ethoxy}ethoxy)acetylamino]ethoxy}ethoxy)acetyl][Aib 8,Arg34]GLP-1-(7-37)

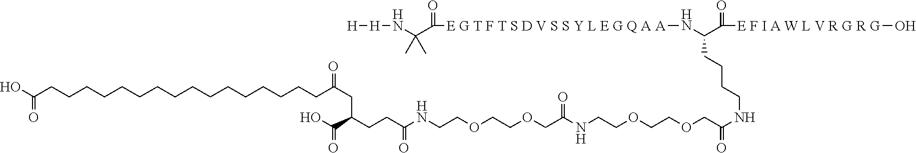

### Compound G4

### N-epsilon37-[2-(2-{2-[2-(2-{2-[(S)-4-carboxy-4-(15-carboxy-pentadecanoylamino)-butyrylamino]-ethoxy}-ethoxy)-acetylamino]-ethoxy}-ethoxy)-acetyl] [Aib8,22,35,Lys37]GLP-1-(7-37)

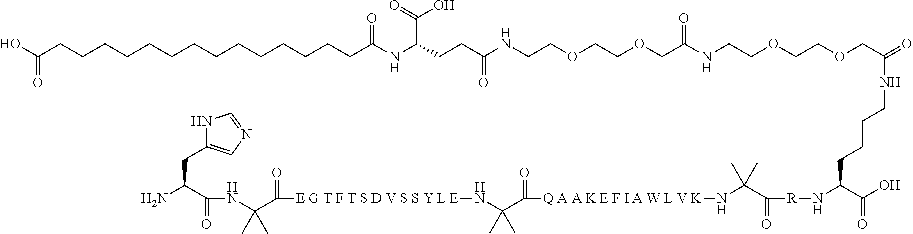

Compound G1 was prepared as described in Example 37 of WO 98/08871. Compound G2 was prepared as described in Example 26 of WO 09030771. Compound G3 was prepared as described in Example 4 of WO 2006/097537.

Novel compound G4 was prepared in similar fashion to the methods described in WO 09/030771, using a CEM Liberty peptide synthesizer.

LCMS METHOD: LCMS_4: m/z=1046 (M/4)

Calculated (M)=4184.8

### Example 66

### N^{ε28}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Leu²⁷,Lys²⁸]-Glucagon

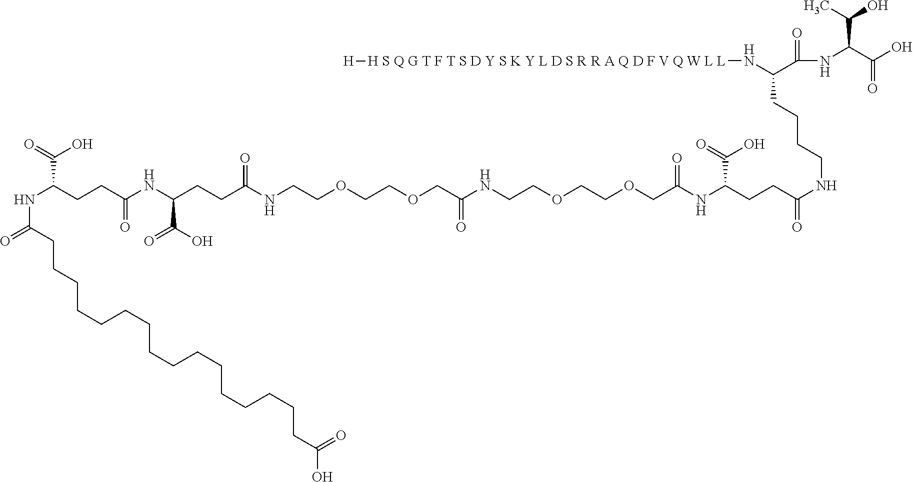

The peptide was prepared essentially as described in SPPS method A and C

UPLC Method: 04_A6_1: Rt=5.2 min

UPLC Method: 09_B4_1: Rt=8.3 min

LCMS Method: LCMS_4: Rt=2.0 min, m/3=1485; m/4=1114; m/5=891

### Example 67

### N^{ε28}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Leu²⁷,Lys²⁸]-Glucagon

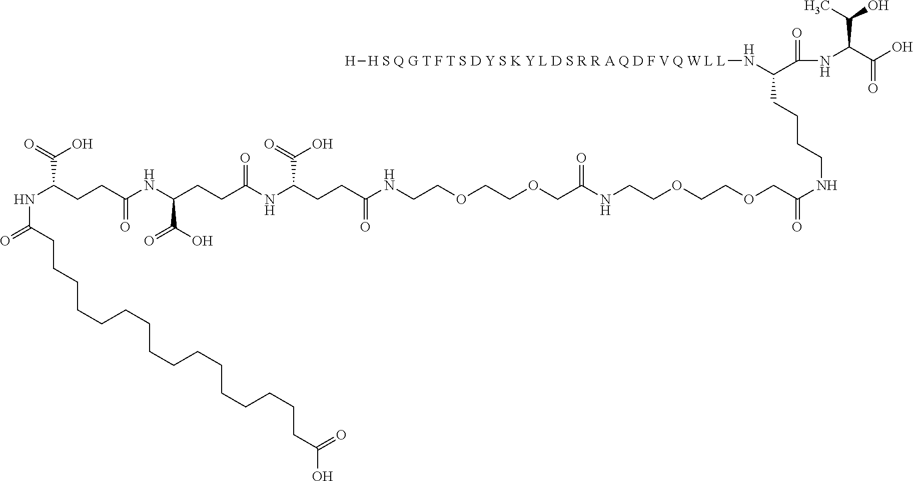

The peptide was prepared essentially as described in SPPS method A and C

UPLC Method: 04_A6_1: Rt=5.2 min

UPLC Method: 09_B4_1: Rt=8.3 min

LCMS Method: LCMS_4: Rt=2.1 min, m/3=1485; m/4=1114; m/5=891

### Example 68

### N^{ε24}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys²⁴,Leu²⁷,Ser²⁸]-Glucagon

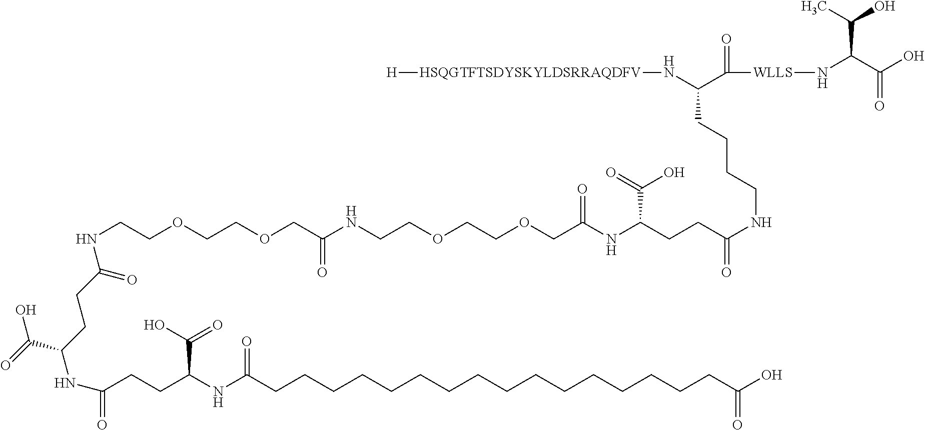

The peptide was prepared essentially as described in SPPS method A and C

UPLC Method: 04_A6_1: Rt=5.8 min

UPLC Method: 09_B2_1: Rt=12.6 min

LCMS Method: LCMS_4: Rt=2.1 min, m/3=1471; m/4=1103; m/5=883

### Example 69

### N^{ε24}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys²⁴,Leu²⁷,Ser²⁸]-Glucagon

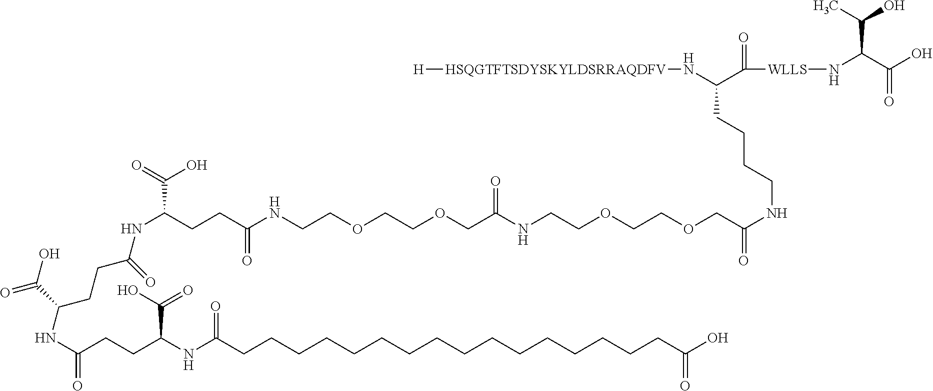

The peptide was prepared essentially as described in SPPS method A and C

UPLC Method: 04_A6_1: Rt=5.8 min

UPLC Method: 09_B2_1: Rt=12.6 min

LCMS Method: LCMS_4: Rt=2.0 min, m/3=1470; m/4=1103; m/5=883

### Example 70

### N^{ε16}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys¹⁶,Leu²⁷]-Glucagon

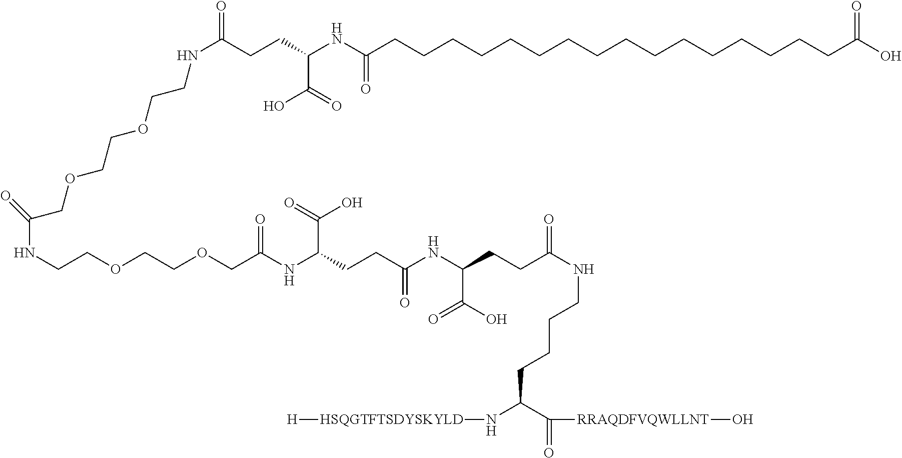

The peptide was prepared essentially as described in SPPS method A and C

UPLC Method: 04_A6_1: Rt=6.41 min

LCMS Method: LCMS_4: Rt=1.9 min, m/3=1494; m/4=1121; m/5=897

### Example 71

### N^{ε24}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Lys²⁴,Leu²⁷,Ser²⁸]-Glucagon

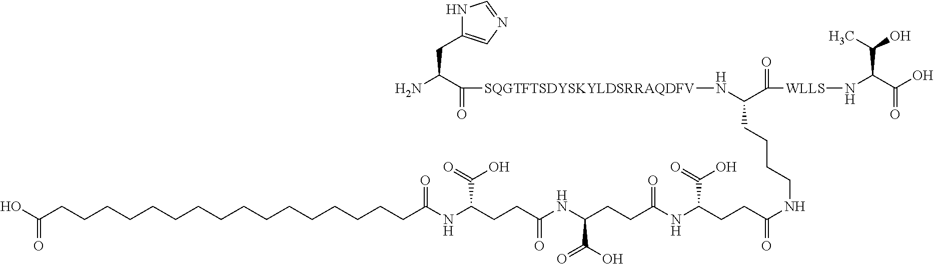

The peptide was prepared essentially as described in SPPS method A and C

UPLC Method: 04_A6_1: Rt=6.1 min

UPLC Method: 09_B4_1: Rt=8.5 min

LCMS Method: LCMS_4: Rt=2.1 min, m/3=1374; m/4=1030; m/5=824

### Example 72

### N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Arg¹²,Lys²⁴,Leu²⁷]-Glucagon

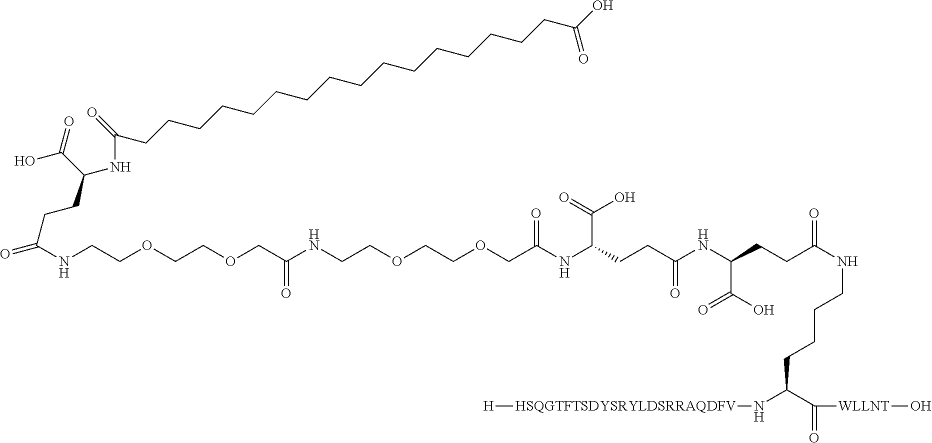

The peptide was prepared essentially as described in SPPS method A and C

UPLC Method: 04_A6_1: Rt=5.9 min

UPLC Method: 09_B4_1: Rt=8.4 min

LCMS Method: LCMS_4: Rt=2.1 min, m/3=1490; m/4=1118; m/5=894

### Example 73

### N^{ε24}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys²⁴,Leu²⁷]-Glucagon

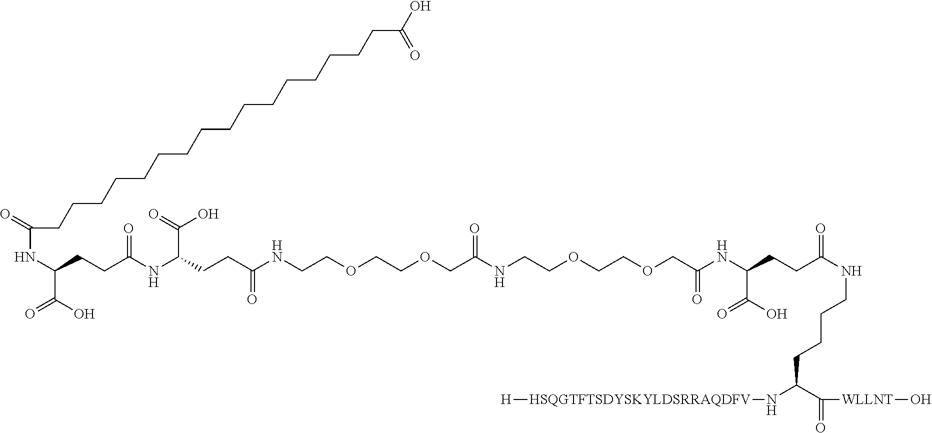

The peptide was prepared essentially as described in SPPS method A and C

UPLC Method: 04_A9_1: Rt=12.4 min

UPLC Method: 08_B2_1: Rt=12.7 min

UPLC Method: 04_B4_1: Rt=8.4 min

UPLC Method: 05_B5_1: Rt=4.7 min

LCMS Method: LCMS_4: Rt=2.1 min, m/3=1480; m/4=1110; m/5=888

### Example 74

### N^{ε24}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys²⁴,Leu²⁷]-Glucagon

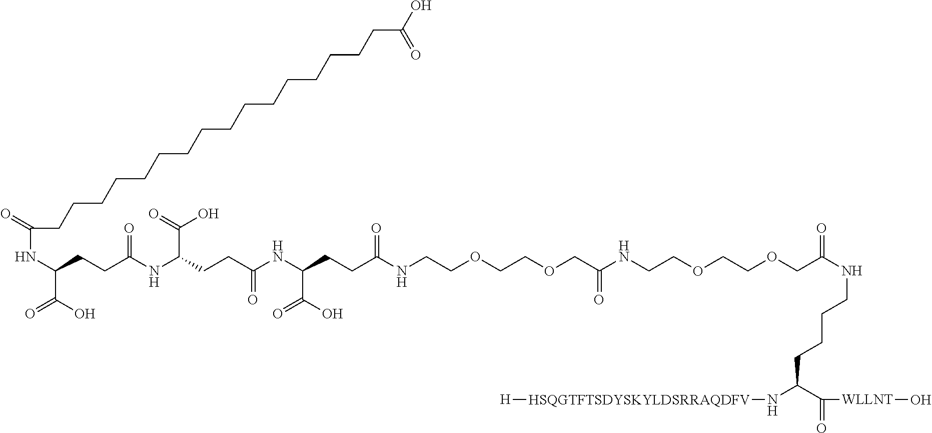

The peptide was prepared essentially as described in SPPS method A and C

UPLC Method: 04_A9_1: Rt=11.7 min

UPLC Method: 08_B2_1: Rt=12.6 min

UPLC Method: 08_B4_1: Rt=8.3 min

UPLC Method: 05_B5_1: Rt=4.6 min

LCMS Method: LCMS_4: Rt=2.1 min, m/3=1780; m/4=1110; m/5=888

### Example 75

### N^{ε24}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Lys²⁴,Leu²⁷]-Glucagon

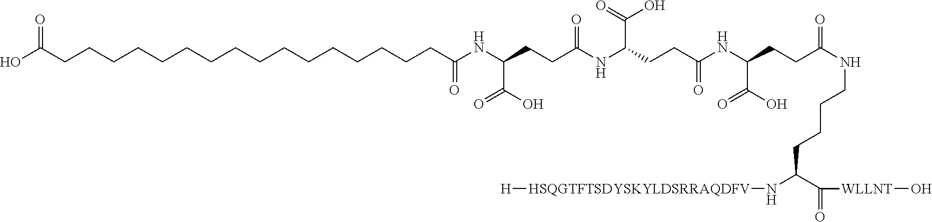

The peptide was prepared essentially as described in SPPS method A and C

UPLC Method: 04_A9_1: Rt=11.3 min

UPLC Method: 09_B4_1: Rt=8.4 min

LCMS Method: LCMS_4: Rt=2.1 min, m/3=1383; m/4=1038; m/5=830

### Example 76

### N^{ε25}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁵,Leu²⁷]-Glucagon

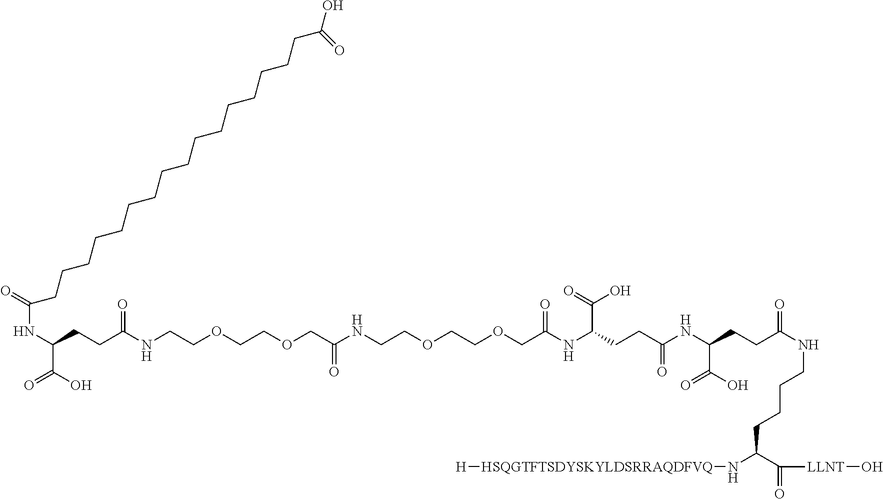

The peptide was prepared essentially as described in SPPS method A and C

UPLC Method: 04_A9_1: Rt=10.1 min

UPLC Method: 09_B4_1: Rt=8.0 min

LCMS Method: LCMS_4: Rt=2.1 min, m/4=1096; m/5=877

### Example 77

### N^{ε16}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys¹⁶,Leu²⁷]-Glucagon

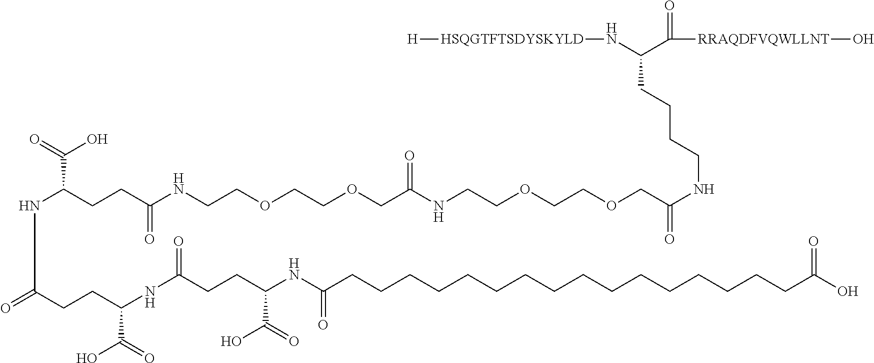

The peptide was prepared essentially as described in SPPS method A and C

UPLC Method: 04_A9_1: Rt=11.6 min

UPLC Method: 09_B2_1: Rt=12.5 min

UPLC Method: 09_B4_1: Rt=8.3 min

UPLC Method: 05_B5_1: Rt=4.3 min

LCMS Method: LCMS_4: Rt=2.0 min, m/3=1494; m/4=1120; m/5=896

### Example 78

### N^{ε16}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys¹⁶,Leu²⁷]-Glucagon

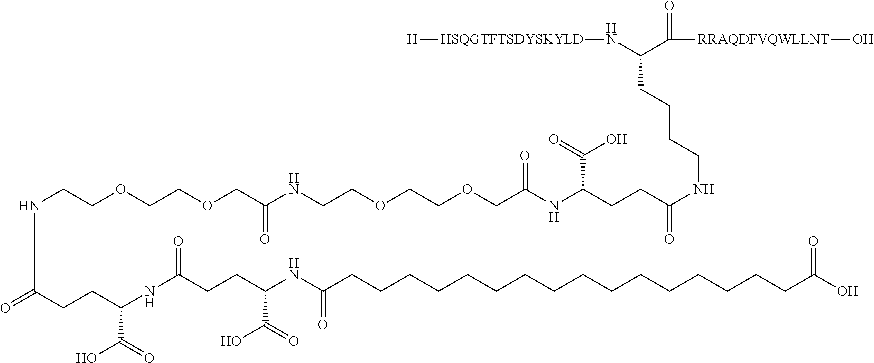

The peptide was prepared essentially as described in SPPS method A and C

UPLC Method: 04_A9_1: Rt=10.9 min

UPLC Method: 09_B2_1: Rt=12.5 min

UPLC Method: 09_B4_1: Rt=8.3 min

UPLC Method: 05_B5_1: Rt=4.3 min

LCMS Method: LCMS_4: Rt=2.0 min, m/3=1494; m/7=1120; m/5=896

### Example 79

### N^{ε28}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Leu²⁷,Lys²⁸]-Glucagon

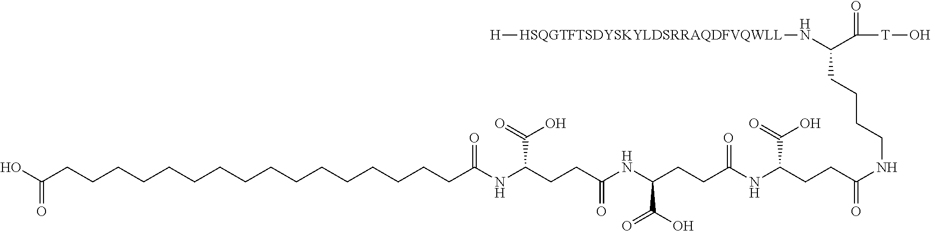

The peptide was prepared essentially as described in SPPS method A and C

UPLC Method: 04_A9_1: Rt=10.8 min

UPLC Method: 09_B2_1: Rt=12.7 min

UPLC Method: 09_B4_1: Rt=8.4 min

UPLC Method: 05_B5_1: Rt=4.6 min

LCMS Method: LCMS_4: Rt=2.1 min, m/3=1387; m/4=1040; m/5=832

### Example 80

### N^{ε12}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu²⁷,Pro²⁹]-Glucagon

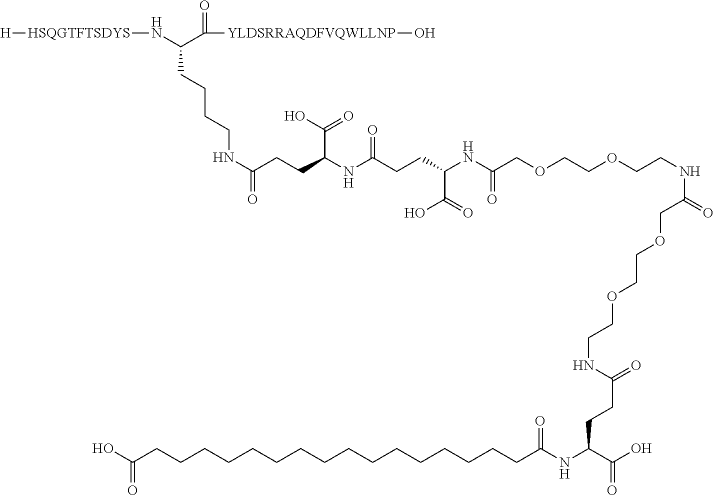

The peptide was prepared essentially as described in SPPS method A and C

UPLC Method: 04_A9_1: Rt=12.9 min

UPLC Method: 09_B4_1: Rt=8.6 min

LCMS Method: LCMS_4: Rt=2.2 min, m/3=1479; m/4=1110; m/5=888

### Example 81

### N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Leu²⁷,Pro²⁹]-Glucagon

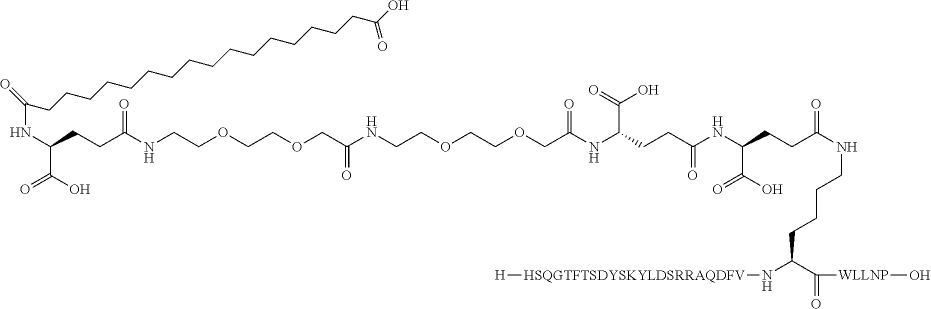

The peptide was prepared essentially as described in SPPS method A and C

UPLC Method: 04_A9_1: Rt=12.6 min

UPLC Method: 09_B4_1: Rt=8.4 min

LCMS Method: LCMS_4: Rt=2.1 min, m/3=1479; m/4=1109; m/5=888

### Example 82

### N^{ε28}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu²⁷,Lys²⁸]-Glucagonyl-Pro

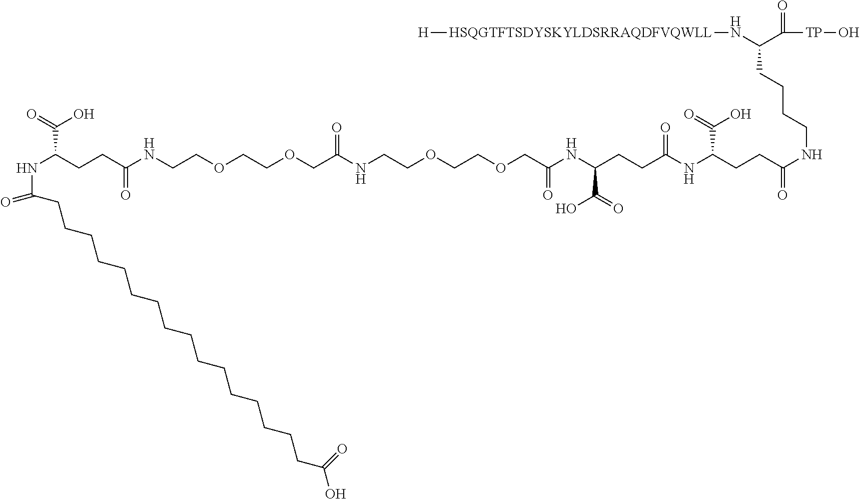

The peptide was prepared essentially as described in SPPS method A and C

UPLC Method: 04_A9_1: Rt=12.4 min

UPLC Method: 09_B2_1: Rt=12.6 min

UPLC Method: 09_B4_1: Rt=8.4 min

UPLC Method: 05_B5_1: Rt=4.9 min

LCMS Method: LCMS_4: Rt=2.0 min, m/3=1517; m/4=1138; m/5=910

### Example 83

### N^{ε12}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu²⁷]-Glucagon

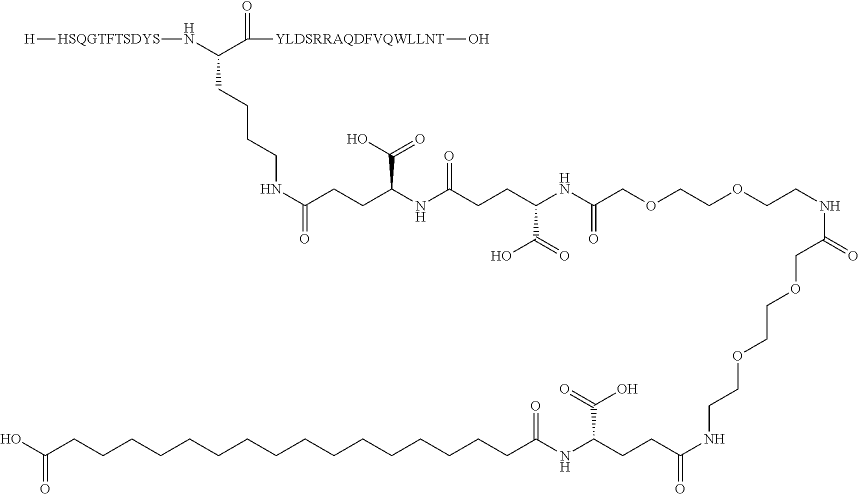

The peptide was prepared essentially as described in SPPS method A and C

UPLC Method: 04_A9_1: Rt=12.7 min

UPLC Method: 09_B2_1: Rt=13.0 min

UPLC Method: 09_B4_1: Rt=8.6 min

UPLC Method: 05_B5_1: Rt=5.1 min

LCMS Method: LCMS_4: Rt=2.1 min, m/3=1480; m/4=1110

### Example 84

### N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagonyl-Pro

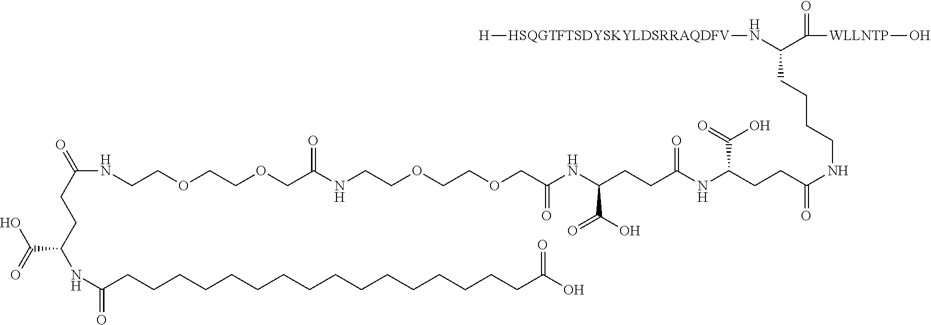

The peptide was prepared essentially as described in SPPS method A and C

UPLC Method: 04_A9_1: Rt=12.7 min

UPLC Method: 09_B2_1: Rt=12.6 min

UPLC Method: 09_B4_1: Rt=8.3 min

UPLC Method: 05_B5_1: Rt=5.1 min

LCMS Method: LCMS_4: Rt=2.0 min, m/3=1512; m/4=1134; m/5=907

### Example 85

### N^{ε27}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁷,Pro²⁹]-Glucagon

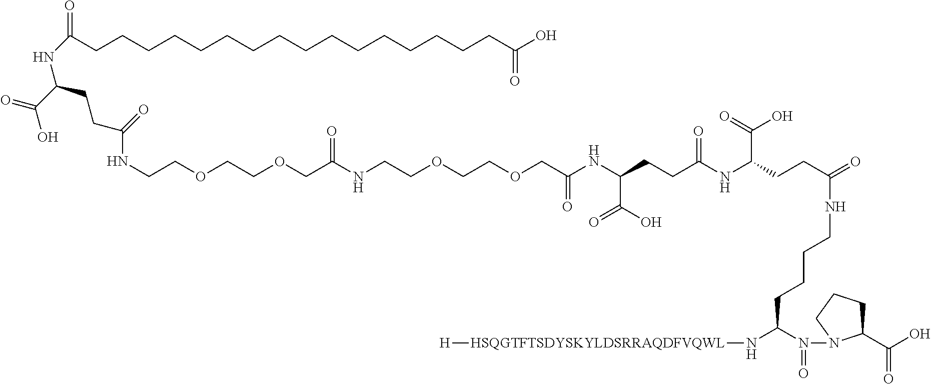

The peptide was prepared essentially as described in SPPS method A and C

UPLC Method: 04_A9_1: Rt=11.1 min

UPLC Method: 09_B4_1: Rt=8.2 min

LCMS Method: LCMS_2: Rt=4.4 min, m/3=1485; m/4=1114; m/5=891

### Example 86

### N^{ε28}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Leu²⁷,Lys²⁸,Pro²⁹]-Glucagon

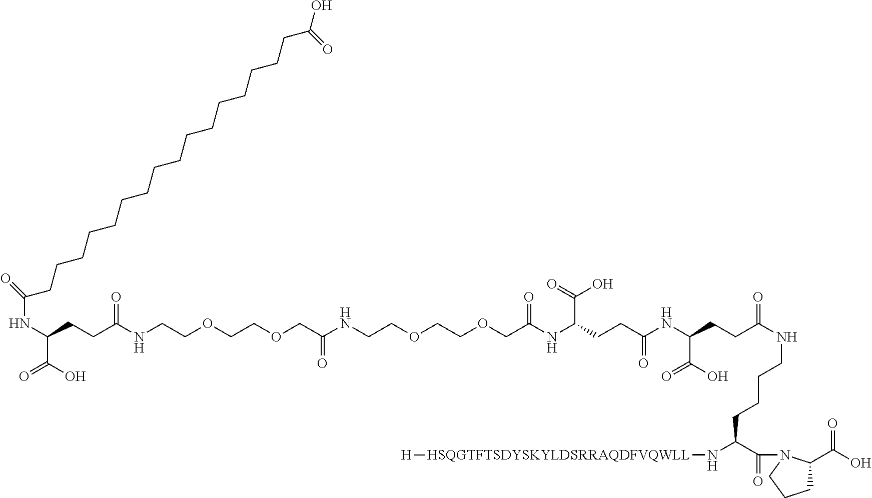

The peptide was prepared essentially as described in SPPS method A and C

UPLC Method: 04_A9_1: Rt=12.0 min

UPLC Method: 09_B4_1: Rt=8.6 min

LCMS Method: LCMS_2: Rt=4.4 min, m/3=1484; m/4=1113; m/5=891

### Example 87

### N^{ε27}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Arg¹²,Lys²⁷,Pro²⁹]-Glucagon

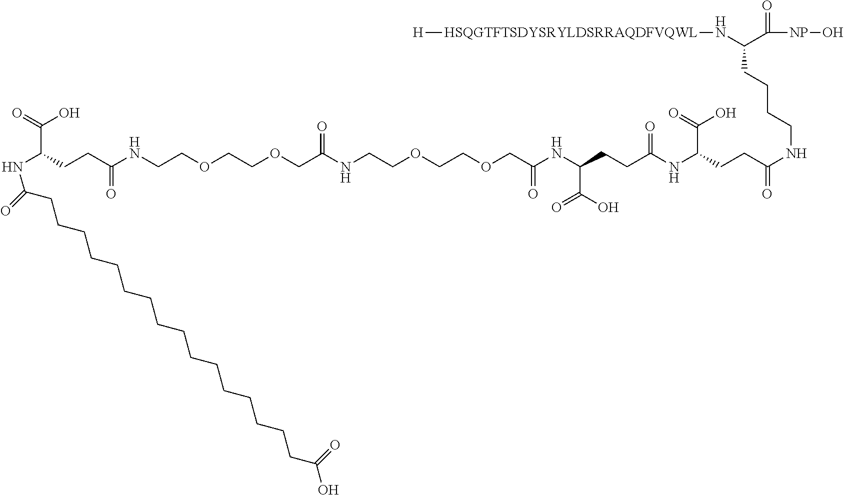

The peptide was prepared essentially as described in SPPS method A and C

UPLC Method: 04_A9_1: Rt=9.9 min

UPLC Method: 09_B4_1: Rt=8.2 min

LCMS Method: LCMS_2: Rt=4.2 min, m/3=1494; m/4=1121; m/5=897

### Example 88

### N^{ε24}-[(2S)-4-carboxy-2-[[(2S)-4-carboxy-2-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

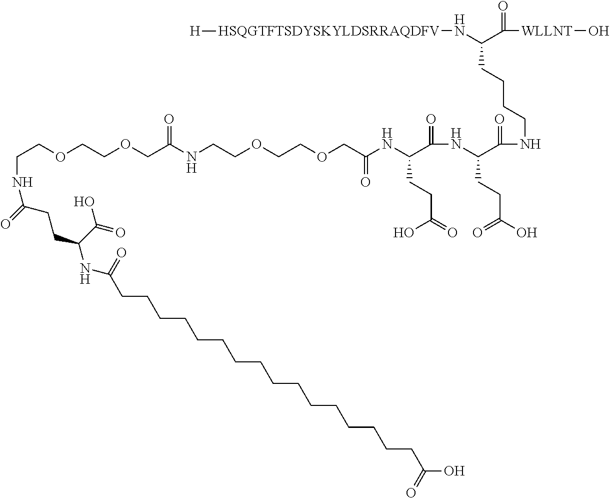

The peptide was prepared essentially as described in SPPS method A and C

UPLC Method: AP_B4_1: Rt=9.0 min

LCMS Method: LCMS_AP: Rt=9.0 min, m/3=1480; m/4=1110

### Example 89

### N^{ε24}-[(2S)-4-carboxy-2-[[(2S)-4-carboxy-2-[[2-[2-[2-[[2-[2-[2-[[(2S)-4-carboxy-2-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

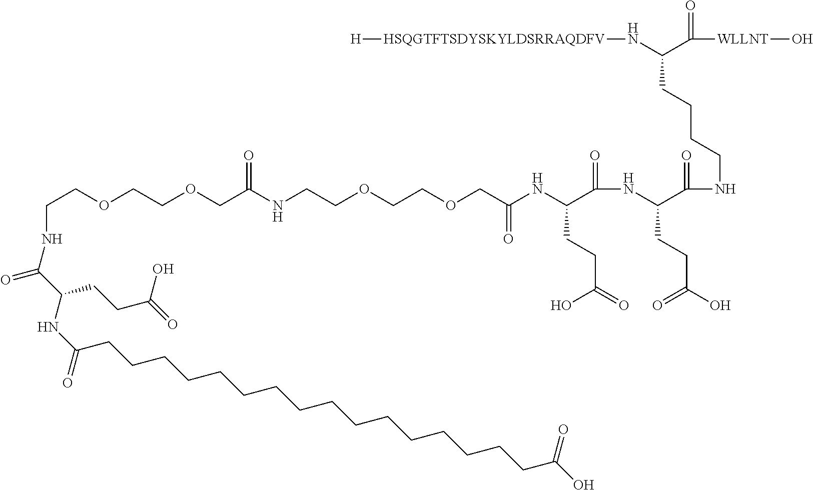

The peptide was prepared essentially as described in SPPS method A and C

UPLC Method: AP_B4_1: Rt=9.1 min 9204-0000-0163

LCMS Method: LCMS_AP: Rt=9.0 min, m/3=1480; m/4=1111

### Example 90

### N^{ε24}-[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]-[Lys²⁴,Leu²⁷]-Glucagon

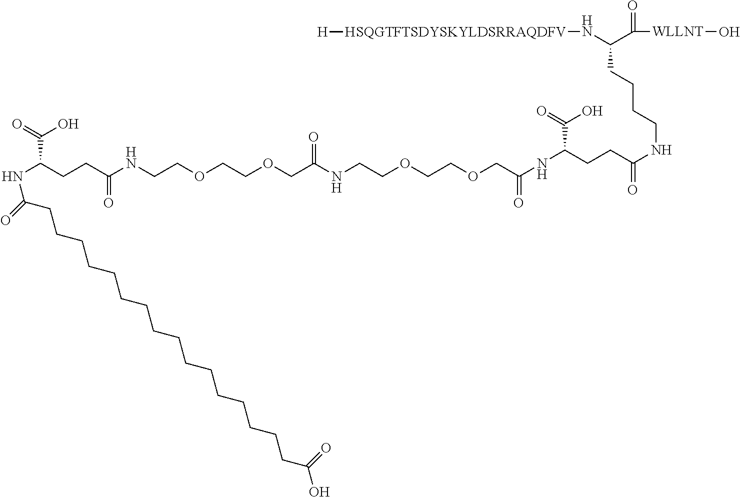

The peptide was prepared essentially as described in SPPS method A and C

UPLC Method: AP_B4_1: Rt=9.1 min

LCMS Method: LCMS_AP: Rt=8.9 min, m/3=1437; m/4=1078

### Example 91

### N^{ε24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu21,Lys24,Leu27,Ser28]-Glucagon

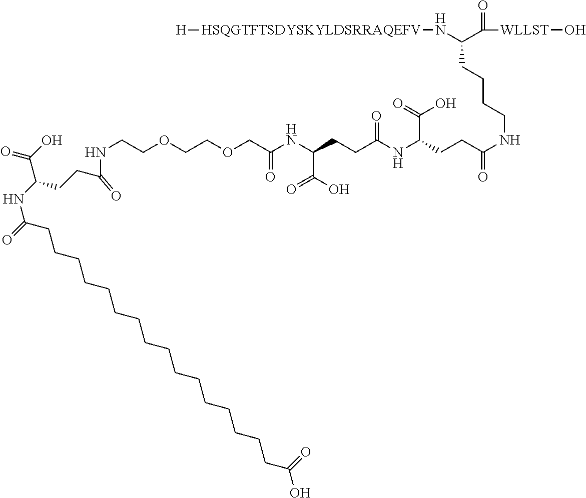

The peptide was prepared essentially as described in SPPS method A and C

UPLC Method: 04_A9_1: Rt=13.6 min

UPLC Method: 09_B4_1: Rt=8.6 min

LCMS Method: LCMS_4: Rt=2.2 min, m/3=1428; m/4=1071; m/5=857

### Example 92

### N^{ε24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu⁹,Lys²⁴,Leu²⁷,Ser²⁸]-Glucagon

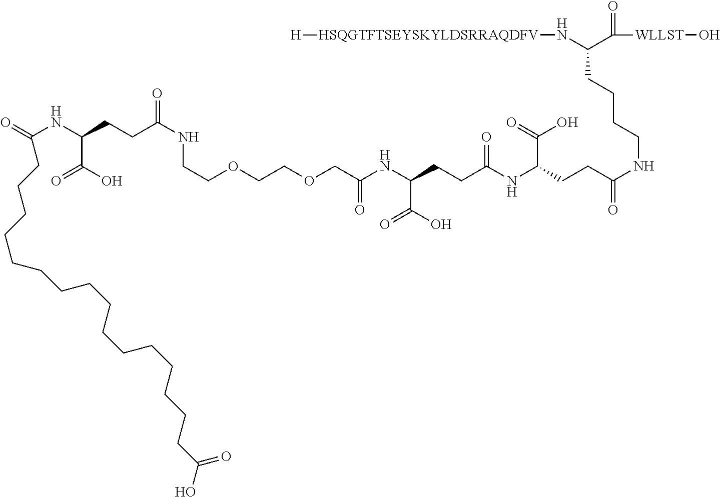

The peptide was prepared essentially as described in SPPS method A and C

UPLC Method: 04_A9_1: Rt=13.2 min

UPLC Method: 09_B4_1: Rt=8.6 min

LCMS Method: LCMS_4: Rt=3.7 min, m/3=1428; m/4=1071; m/5=857

### Example 93

### N^{ε24}-[(4S)-4-carboxy-4-[[(4S)-4-carboxy-4-[[2-[2-[2-[[(4S)-4-carboxy-4-(17-carboxyheptadecanoylamino)butanoyl]amino]ethoxy]ethoxy]acetyl]amino]butanoyl]amino]butanoyl]-[Glu²⁰,Glu²¹,Lys²⁴,Leu²⁷,Ser²⁸]-Glucagon

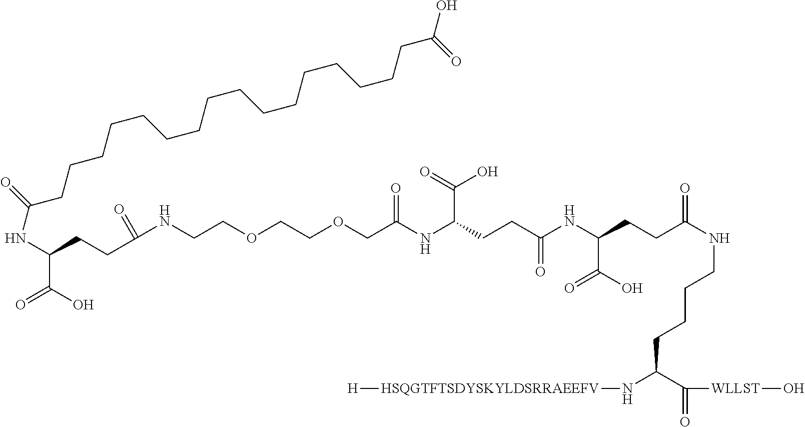

The peptide was prepared essentially as described in SPPS method A and C

UPLC Method: 04_A9_1: Rt=12.5 min

UPLC Method: 09_B4_1: Rt=8.6 min

LCMS Method: LCMS_4: Rt=3.7 min, m/3=1428; m/4=1071; m/5=857

### Example 94

### N^{ε24}-[(4S)-4-carboxy-4-[[(4S)-4-carboxy-4-[[2-[2-[2-[[2-[2-[2-[[(4S)-4-carboxy-4-(15-carboxypentadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

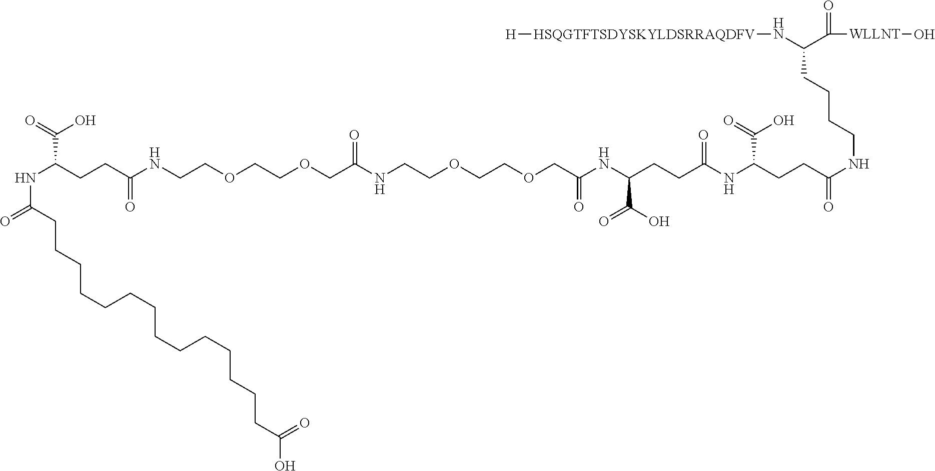

The peptide was prepared essentially as described in SPPS method A and C

UPLC Method: 04_A9_1: Rt=12.3 min

UPLC Method: 08_B2_1: Rt=11.8 min

UPLC Method: 08_B4_1: Rt=7.8 min

UPLC Method: 05_B5_1: Rt=4.2 min

LCMS Method: LCMS_4: Rt=2.0 min, m/3=1471; m/4=1103; m/5=882

### Example 95

### N^{ε24}-[(4S)-4-carboxy-4-[[(4S)-4-carboxy-4-[[2-[2-[2-[[2-[2-[2-[[(4S)-4-carboxy-4-(11-carboxyundecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

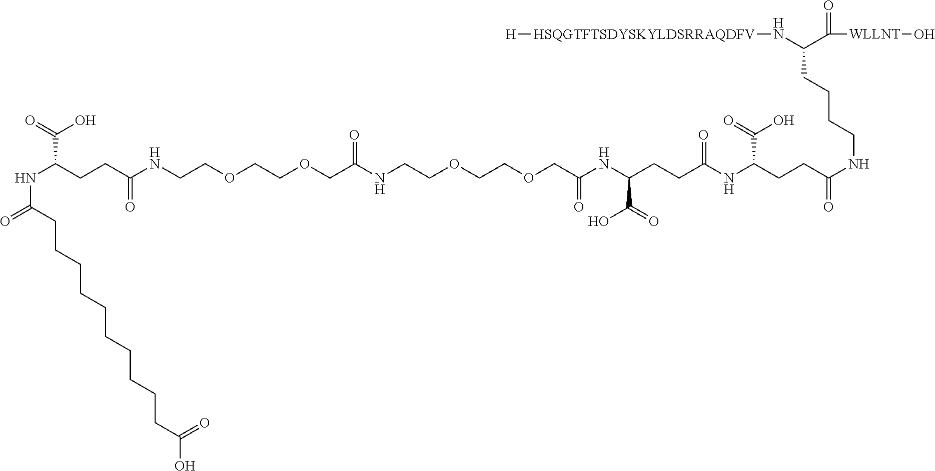

The peptide was prepared essentially as described in SPPS method A and C

UPLC Method: 04_A9_1: Rt=10.6 min

UPLC Method: 08_B2_1: Rt=10.6 min

UPLC Method: 08_B4_1: Rt=7.0 min

UPLC Method: 05_B7_1: Rt=6.7 min

LCMS Method: LCMS_4: Rt=1.8 min, m/3=1452; m/4=1089; m/5=871

### Example 96

### N^{ε24}-[(4S)-4-carboxy-4-[[(4S)-4-carboxy-4-[[2-[2-[2-[[2-[2-[2-[[(4S)-4-carboxy-4-(13-carboxytridecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

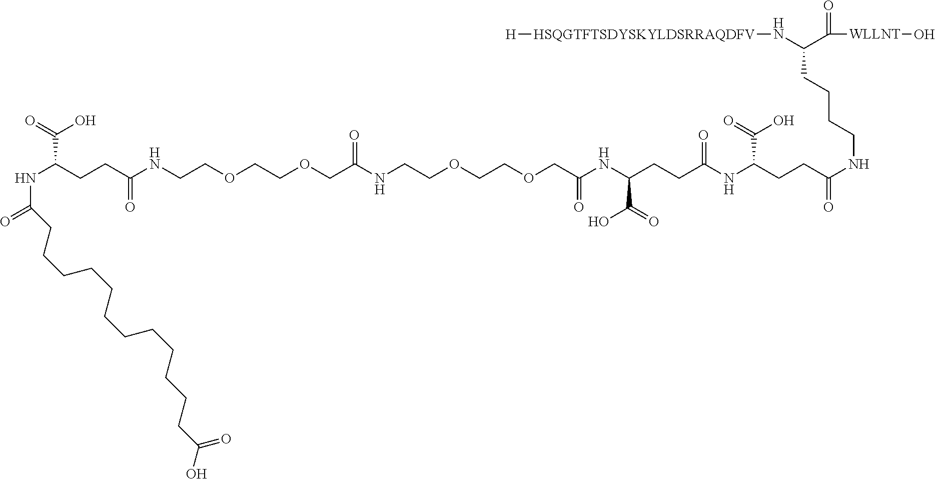

The peptide was prepared essentially as described in SPPS method A and C

UPLC Method: 04_A9_1: Rt=11.2 min

UPLC Method: 09_B2_1: Rt=11.2 min

UPLC Method: 09_B4_1: Rt=7.4 min

UPLC Method: 05_B7_1: Rt=7.2 min

LCMS Method: LCMS_4: Rt=1.9 min, m/3=1461; m/4=1096; m/5=877

### Example 97

### N^{ε24}-[(4S)-4-carboxy-4-[[(4S)-4-carboxy-4-[[2-[2-[2-[[2-[2-[2-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]ethoxy]ethoxy]acetyl]amino]ethoxy]ethoxy]acetyl]amino]butanoyl]amino]butanoyl]-[Lys²⁴,Leu²⁷]-Glucagon

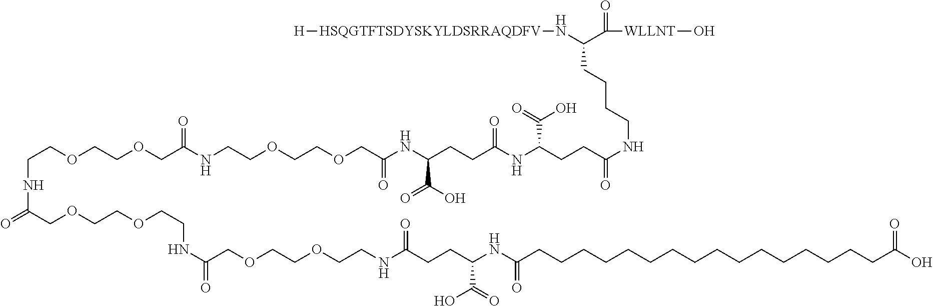

The peptide was prepared essentially as described in SPPS method A and C

UPLC Method: 04_A9_1: Rt=13.6 min

UPLC Method: 09_B2_1: Rt=12.7 min

UPLC Method: 09_B4_1: Rt=8.4 min

UPLC Method: 05_B5_1: Rt=5.1 min

LCMS Method: LCMS_4: Rt=2.1 min, m/3=1576; m/4=1182; m/5=946

### Example 98

### N^{ε20}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁰,Leu²⁷]-Glucagon

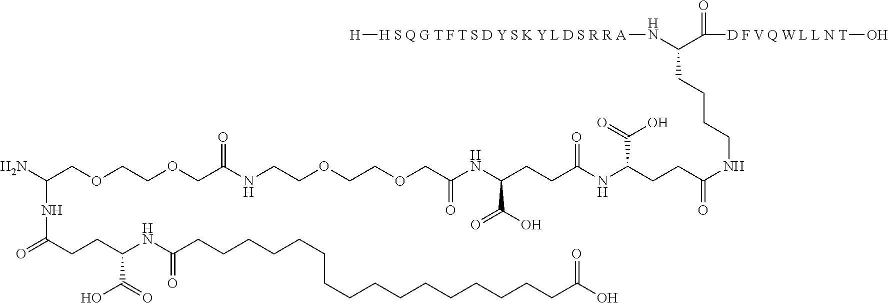

The peptide was prepared essentially as described in SPPS method A and C

UPLC Method: 04_A9_1: Rt=13.9 min

UPLC Method: 09_B2_1: Rt=13.1 min

UPLC Method: 09_B4_1: Rt=8.7 min

UPLC Method: 05_B5_1: Rt=5.3 min

LCMS Method: LCMS_4: Rt=2.2 min, m/3=1480; m/4=1110; m/5=888

### Example 99

### N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[D-Phe4,Lys24,Leu27,Ser28]-Glucagon

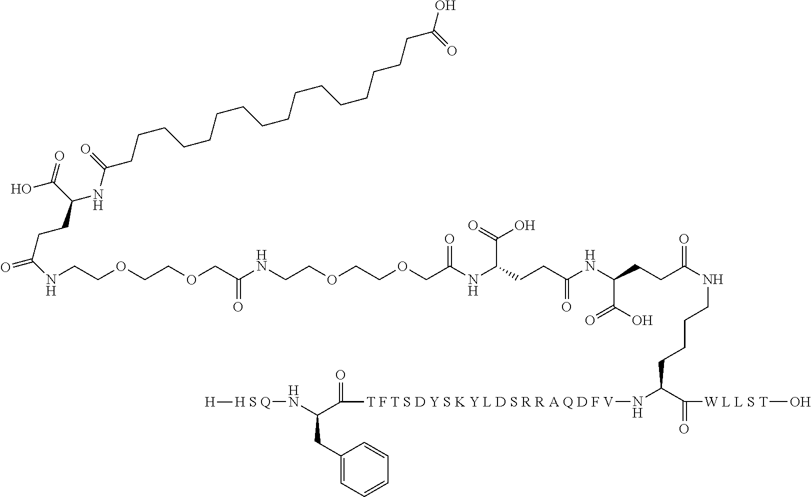

The peptide was prepared essentially as described in SPPS method A and C

UPLC Method: 04_A9_1: Rt=13.4 min

UPLC Method: 09_B4_1: Rt=8.7 min

LCMS Method: LCMS_4: Rt=2.3 min, m/3=1501; m/4=1126; m/5=901

### Example 100

### N^{ε16}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys¹⁶,Glu²¹,Arg²⁵,Leu²⁷]-Glucagon

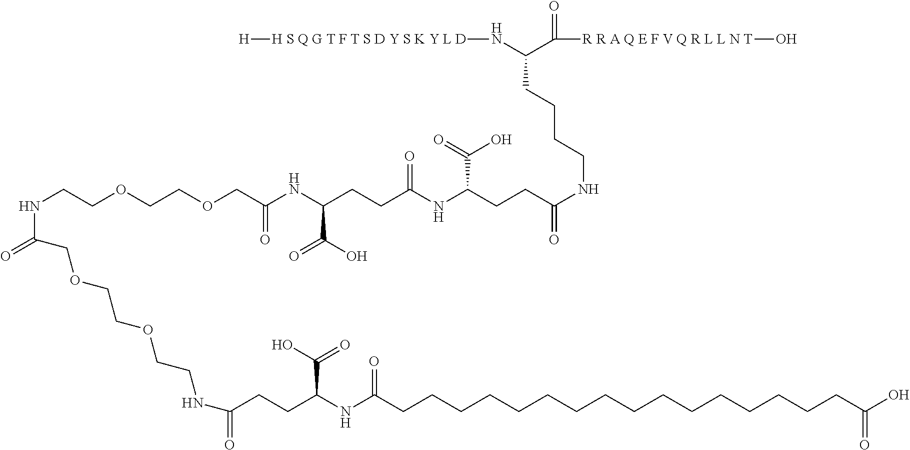

The peptide was prepared essentially as described in SPPS method A and C

UPLC Method: 04_A9_1: Rt=11.7 min

UPLC Method: 08_B2_1: Rt=11.5 min

UPLC Method: 08_B4_1: Rt=7.6 min

UPLC Method: 05_B5_1: Rt=4.2 min

LCMS Method: LCMS_4: Rt=2.2 min, m/3=1488; m/4=1116; m/5=893

### Example 101

### N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Glu²⁰,Lys²⁴,Leu²⁷,Ser²⁸]-Glucagon

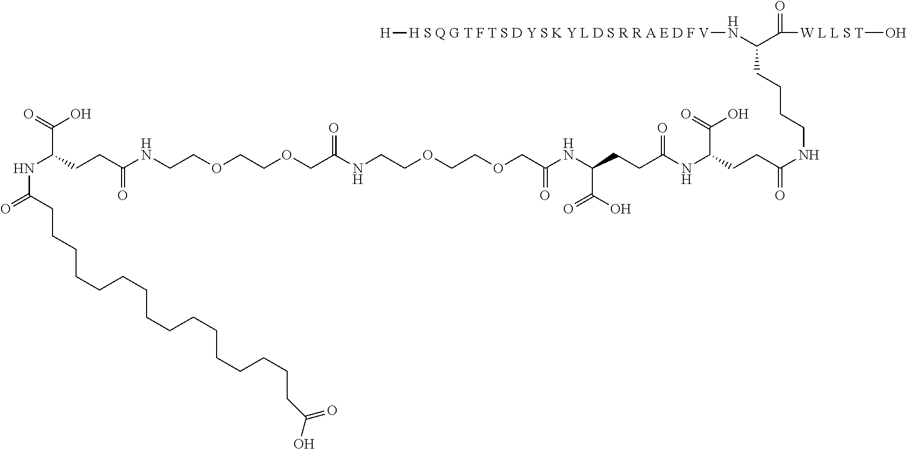

The peptide was prepared essentially as described in SPPS method A and C

UPLC Method: 04_A9_1: Rt=11.5 min

UPLC Method: 09_B4_1: Rt=8.6 min

LCMS Method: LCMS_4: Rt=3.8 min, m/3=1472; m/4=1104; m/5=884

### Example 102

### N^{ε24}-[(4S)-4-carboxy-4-[[(4S)-4-carboxy-4-[[2-[2-[2-[[2-[2-[2-[[(4S)-4-carboxy-4-[10-(4-carboxyphenoxy)decanoylamino]butanoyl]amino]ethoxy]ethoxy]acetyl]amino]ethoxy]ethoxy]acetyl]amino]butanoyl]amino]butanoyl]-[Lys²⁴,Leu²⁷]-Glucagon

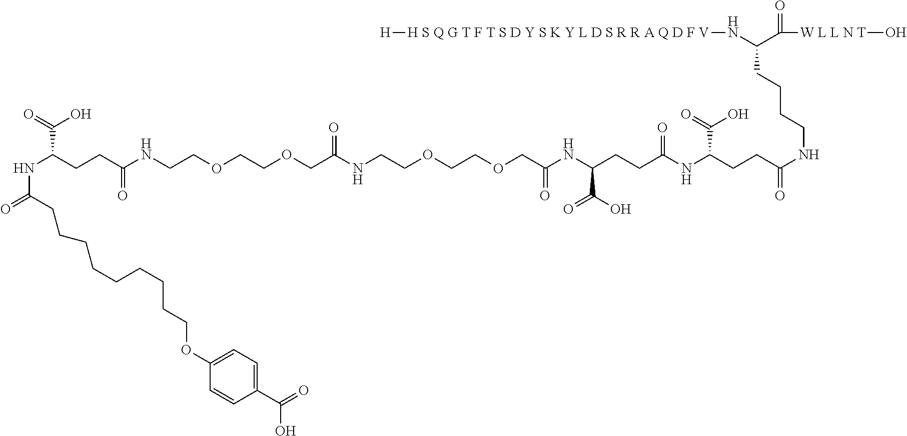

The peptide was prepared essentially as described in SPPS method A and C

UPLC Method: 04_A9_1: Rt=11.1 min

UPLC Method: 09_B2_1: Rt=11.1 min

LCMS Method: LCMS_4: Rt=1.9 min, m/3=1478; m/4=1109; m/5=888

### Example 103

### N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Gln²⁷]-Glucagon

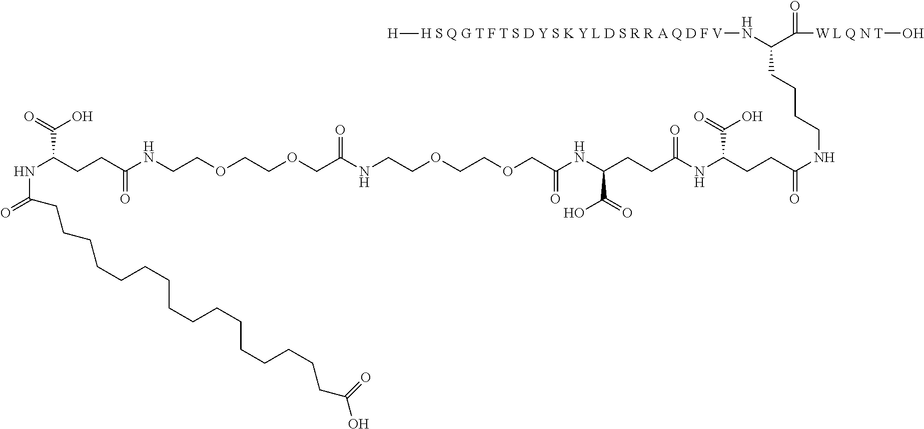

The peptide was prepared essentially as described in SPPS method A and C

UPLC Method: 04_A9_1: Rt=11.4 min

UPLC Method: 09_B2_1: Rt=12.1 min

UPLC Method: 09_B4_1: Rt=8.0 min

UPLC Method: 05_B5_1: Rt=3.5 min

LCMS Method: LCMS_4: Rt=1.9 min, m/3=1485; m/4=1114; m/5=891

### Example 104

### N^{ε24}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]-[Lys²⁴,Glu²⁷]-Glucagon

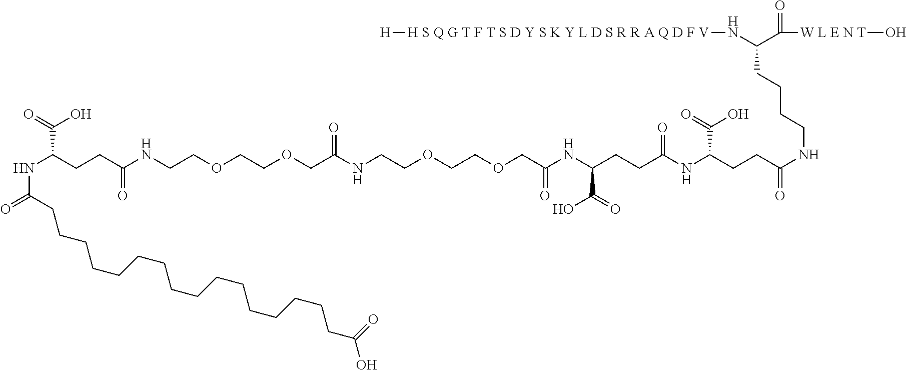

The peptide was prepared essentially as described in SPPS method A and C

UPLC Method: 04_A9_1: Rt=8.9 min

UPLC Method: 09_B2_1: Rt=12.3 min

UPLC Method: 09_B4_1: Rt=8.2 min

UPLC Method: 05_B5_1: Rt=3.8 min

LCMS Method: LCMS_4: Rt=2.0 min, m/3=1486; m/4=1114; m/5=892

### Example 105

### N^{α}([His²⁴,Leu²⁷]-Glucagonyl)-N^{ε}[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]amino]butanoyl]Lys

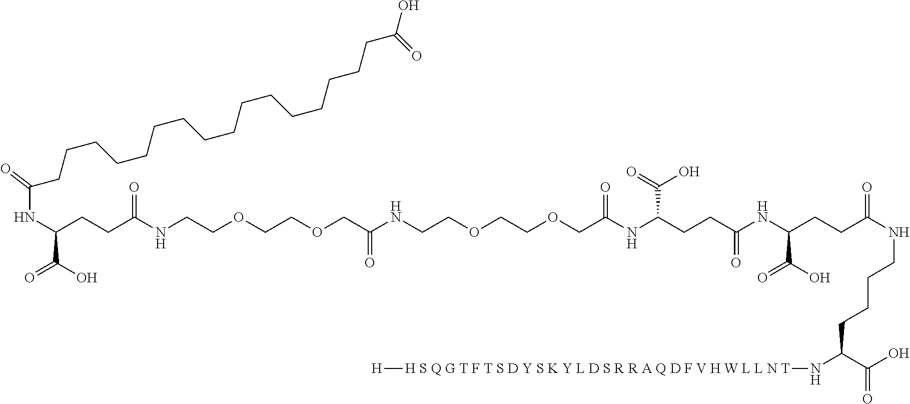

The peptide was prepared essentially as described in SPPS method A and C

UPLC: Method: 04_A6_1: Rt=6.0 min

UPLC: Method: 09_B4_1_214 nm: Rt=8.1 min

LC-MS Method: LCMS_4: Rt=2.7 min, m/3=1526, m/4=1145, m/5=763

### Example 106

### N^{ε24}-[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino]butanoyl]-[Lys²⁴,Glu²⁷]-Glucagon

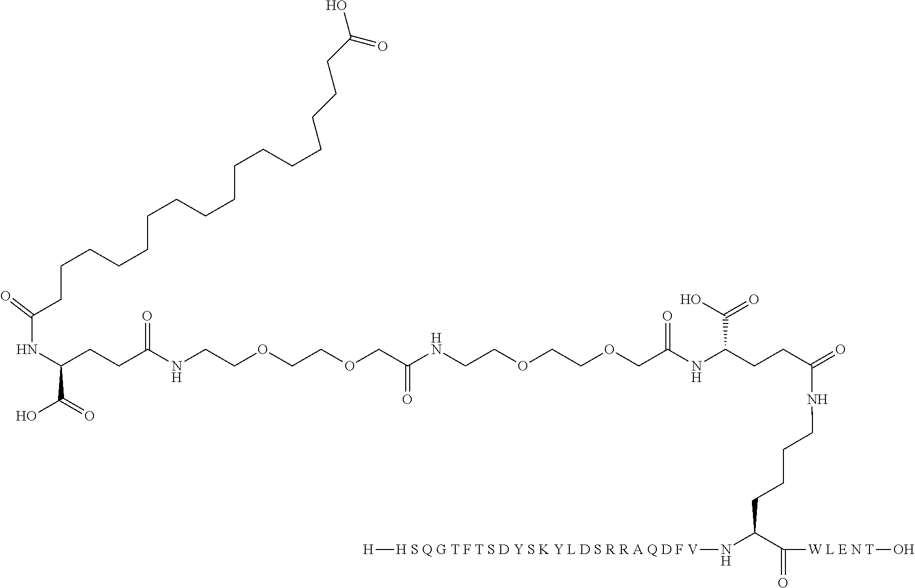

The peptide was prepared essentially as described in SPPS method A and C

UPLC Method: 04_A9_1: Rt=7.7 min

UPLC Method: 09_B2_1: Rt=12.3 min

UPLC Method: 09_B4_1: Rt=8.2 min

LCMS Method: LCMS_4: Rt=3.9 min, m/3=1443; m/4=1082; m/5

### Example 107

### N^{ε24}-[(4S)-4-carboxy-4-[[(4S)-4-carboxy-4-[[2-[2-[2-[[2-[2-[2-[[(4S)-4-carboxy-4-(19-carboxynonadecanoylamino)butanoyl]amino]ethoxy]ethoxy]acetyl]amino]ethoxy]ethoxy]acetyl]amino]butanoyl]amino]butanoyl]-[Lys²⁴,Leu²⁷]-Glucagon

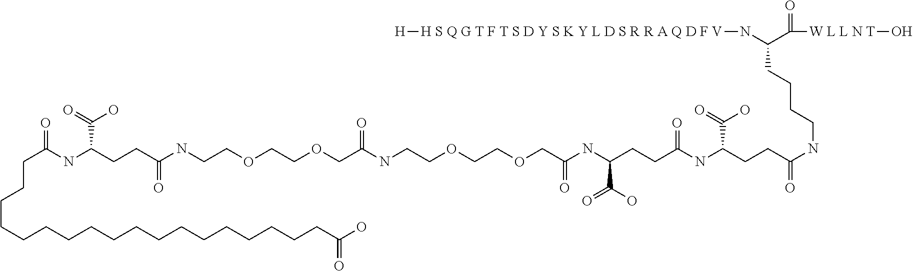

The peptide was prepared essentially as described in SPPS method A and C

UPLC Method: 09_B2_1: Rt=13.7 min

UPLC Method: 09_B4_1: Rt=9.1 min

UPLC Method: 09_A9_1: Rt=13.1 min

LCMS Method: LCMS_4: Rt=2.3 min, m/3=1489.7; m/4=1117.3; m/5=894.2

### Example 108

### N^{ε24}-[(4S)-4-carboxy-4-[[(4S)-4-carboxy-4-[[2-[2-[2-[[2-[2-[2-[[(4S)-4-carboxy-4-(7-carboxyheptanoylamino)butanoyl]amino]ethoxy]ethoxy]acetyl]amino]ethoxy]ethoxy]acetyl]amino]butanoyl]amino]butanoyl]-[Lys24,Leu27]-Glucagon

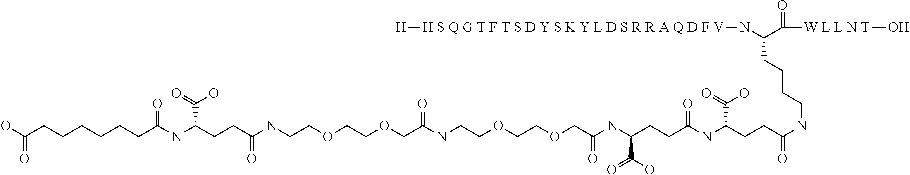

The peptide was prepared essentially as described in SPPS method A and C

UPLC Method: 09_B2_1: Rt=9.7

UPLC Method: 09_B4_1: Rt=6.5

UPLC Method: 04_A9_1: Rt=8.4

LCMS Method: LCMS_4: Rt=1.8 min, m/3=1434; m/4=1075.5; m/5=860.8

### Pharmacological Methods

### Assay (I)

### Glucagon Activity

The glucagon receptor was cloned into HEK-293 cells having a membrane bound cAMP biosensor (ACTOne™). The cells (14000 per well) were incubated (37° C., 5% CO2) overnight in 384-well plates. Next day the cells were loaded with a calcium responsive dye that only distributed into the cytoplasm. Probenecid, an inhibitor of the organic anion transporter, was added to prevent the dye from leaving the cell. A PDE inhibitor was added to prevent formatted cAMP from being degraded. The plates were placed into a FLIPRTETRA and the glucagon analogues were added. End point data were collected after 6 minutes. An increase in intracellular cAMP was proportional to an increased in calcium concentrations in the cytoplasm. When calcium was bound the dry a fluorescence signal was generated. EC50-values were calculated in Prism5.

TABLE 1
In vitro data on receptor binding

| | | Assay |
|---|---|---|
| | | (I) |
| | | Gluc- |
| | | agon |
| Exam- | | [EC- |
| ple | | 50] |
| Nr. | Structure | (nM) |
| hGluc- | H—H S Q G T F T S D Y S K Y L D S R R A Q D F V Q W L M N T—OH | 0.003 |
| agon | | |
| Exam- ple 1 | 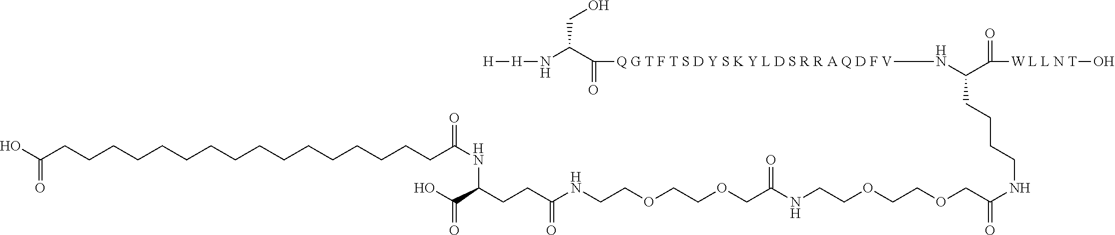 | 0.093 |
| Exam- ple 2 | 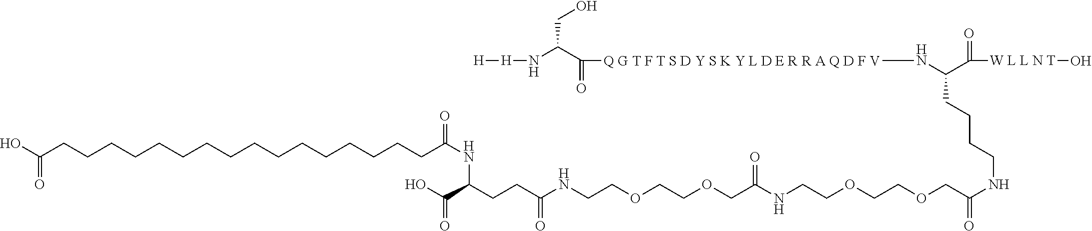 | 0.149 |
| Exam- ple 3 | 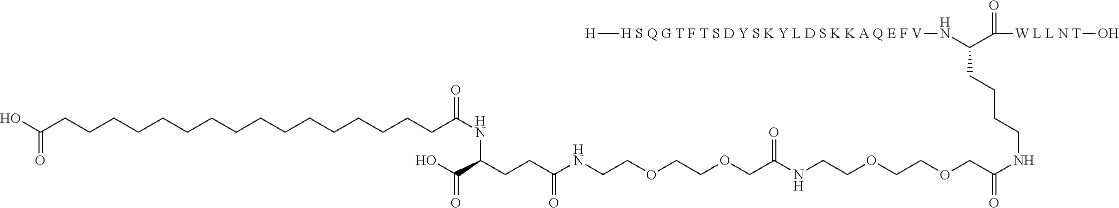 | 0.019 |
| Exam- ple 4 | 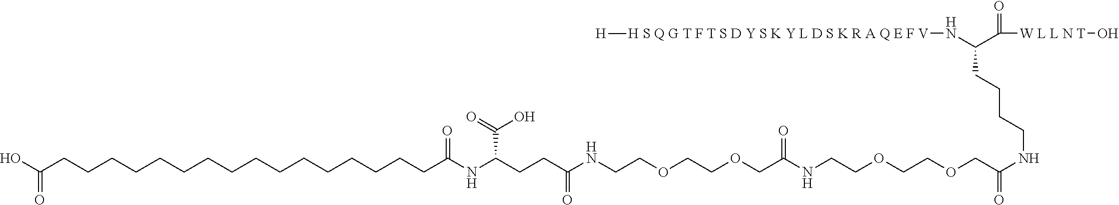 | 0.022 |
| Exam- ple 5 | 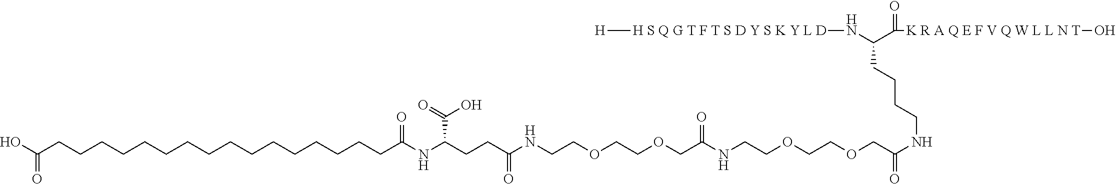 | 0.020 |
| Exam- ple 6 | 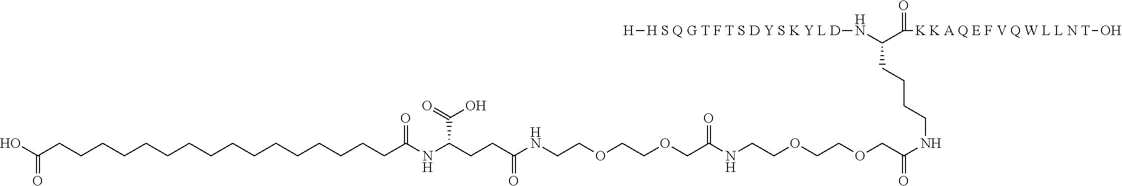 | 0.020 |
| Exam- ple 7 | 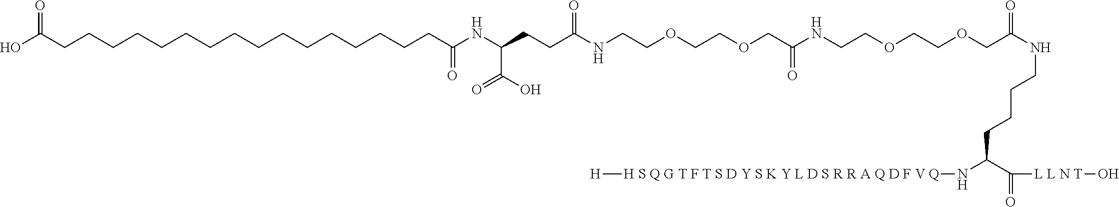 | 0.155 |
| Exam- ple 8 | 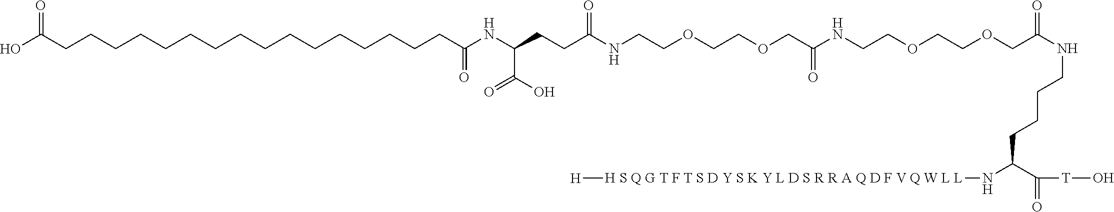 | 0.022 |
| Exam- ple 9 | 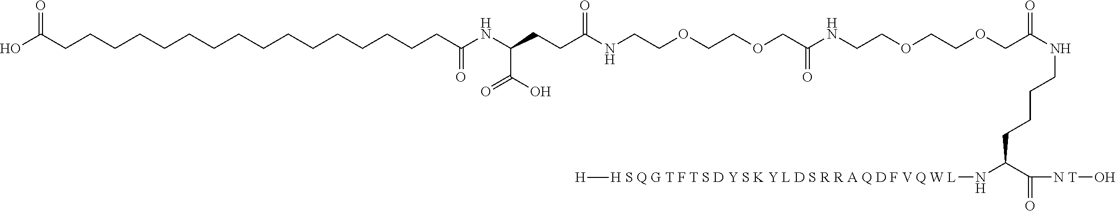 | 0.128 |
| Exam- ple 10 | 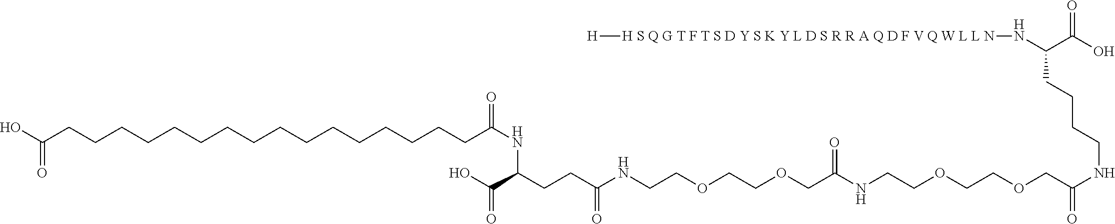 | 0.046 |
| Exam- ple 11 | 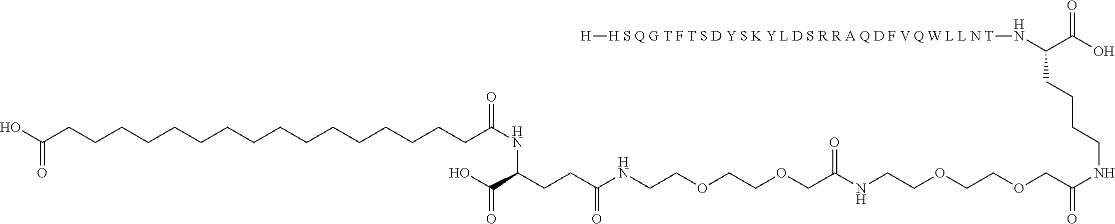 | 0.019 |
| Exam- ple 12 | 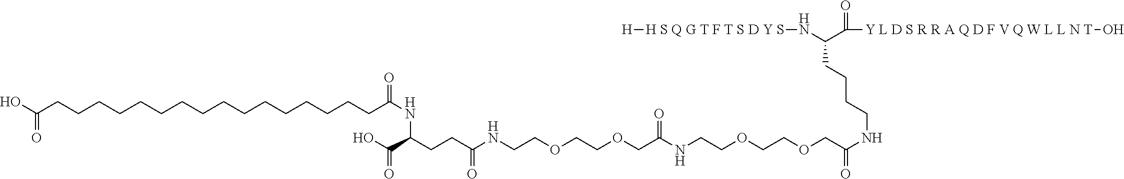 | 0.034 |
| Exam- ple 13 | 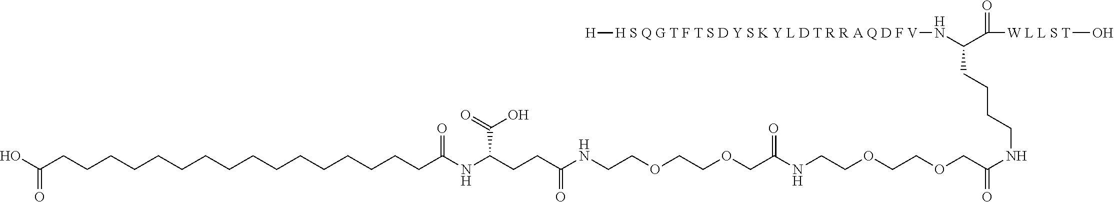 | 0.016 |
| Exam- ple 14 | 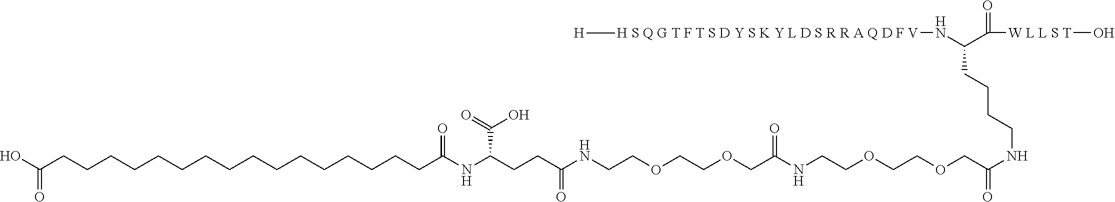 | 0.020 |
| Exam- ple 15 | 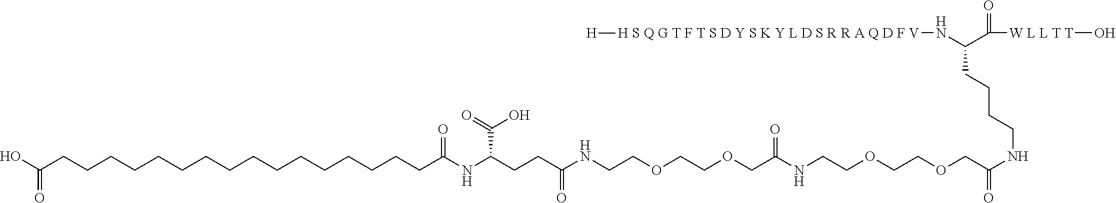 | 0.024 |
| Exam- ple 16 | 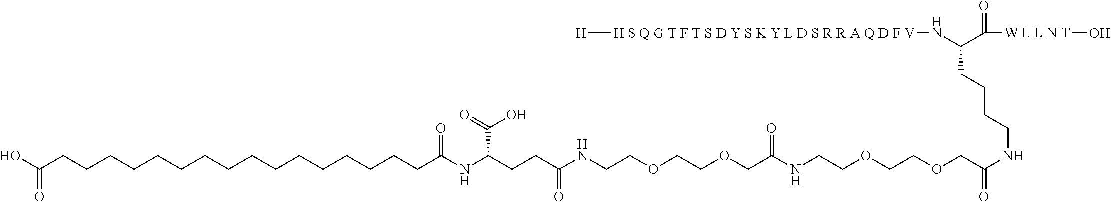 | 0.017 |
| Exam- ple 17 | 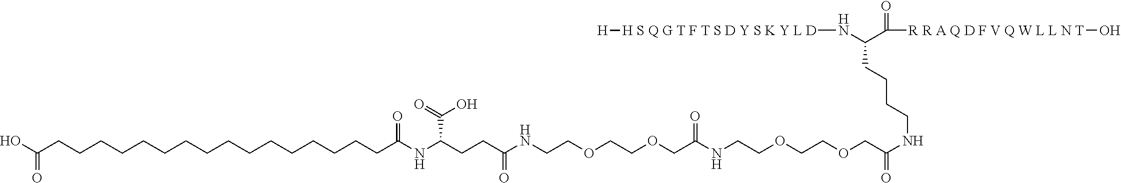 | 0.003 |
| Exam- ple 18 | 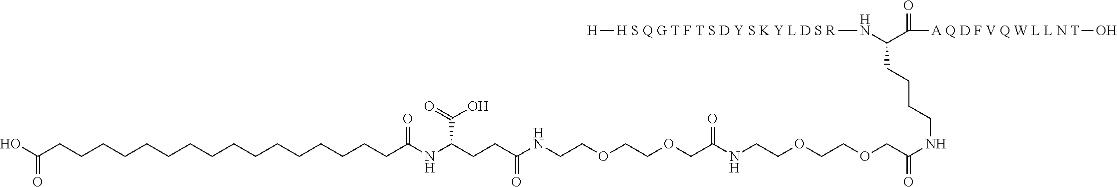 | 0.206 |
| Exam- ple 19 | 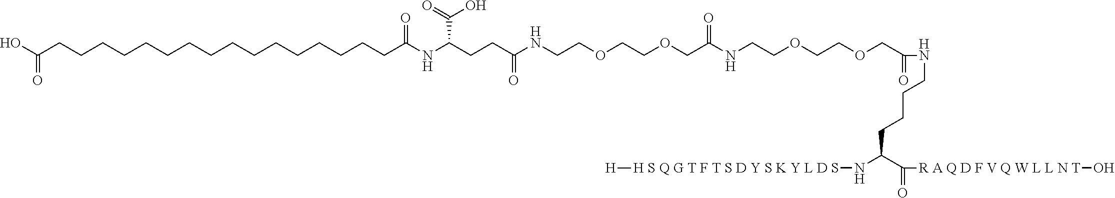 | 0.094 |
| Exam- ple 20 | 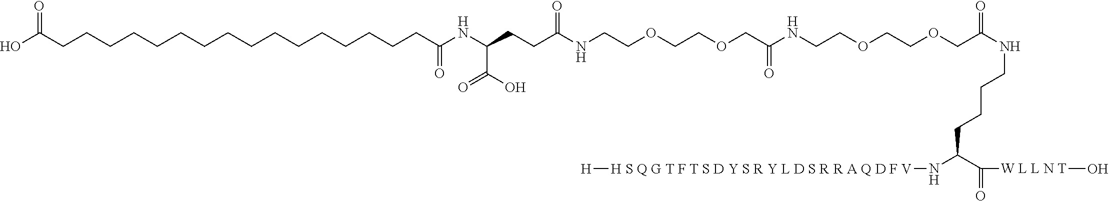 | 0.109 |
| Exam- ple 21 | 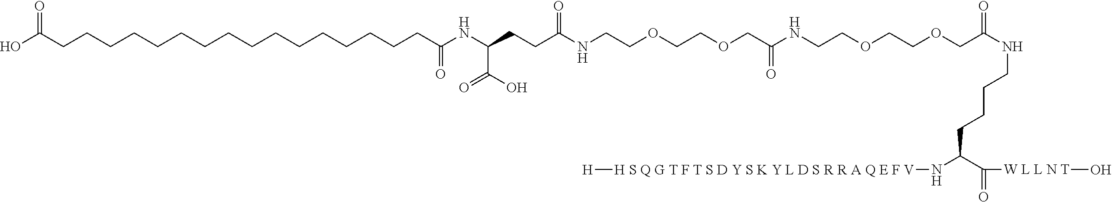 | 0.021 |
| Exam- ple 22 | 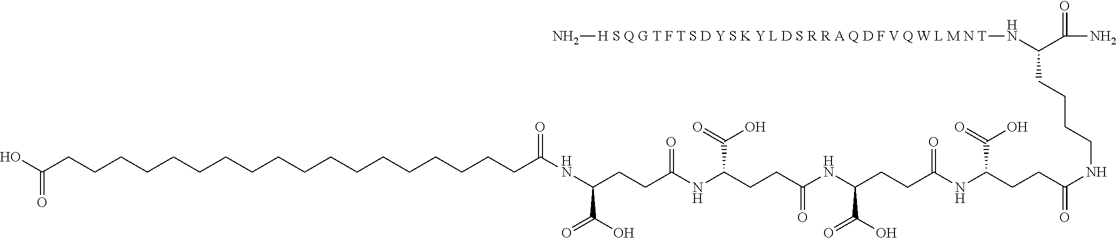 | 0.960 |
| Exam- ple 23 | 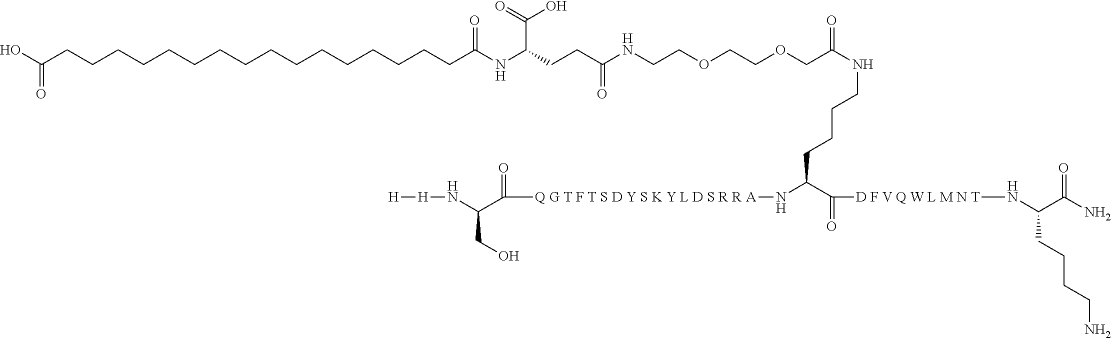 | 0.540 |
| Exam- ple 24 | 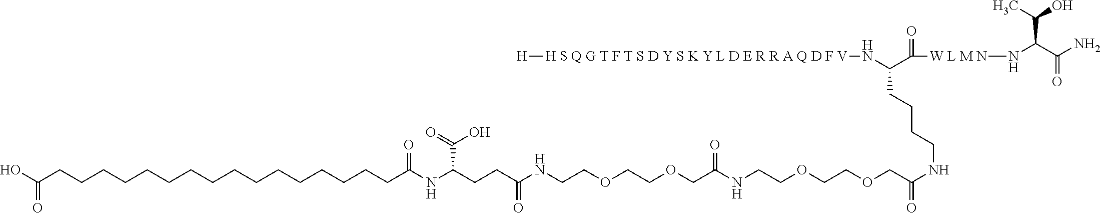 | 0.027 |
| Exam- ple 25 | 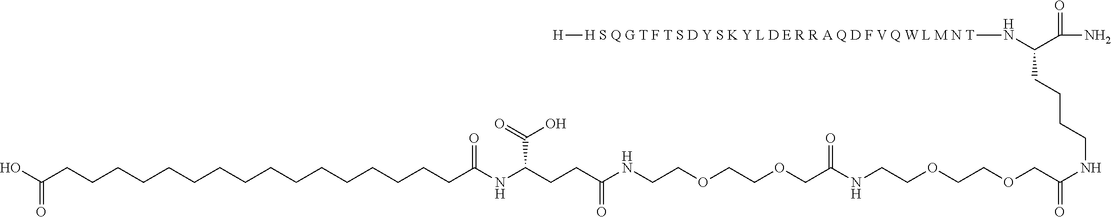 | 0.397 |
| Exam- ple 26 | 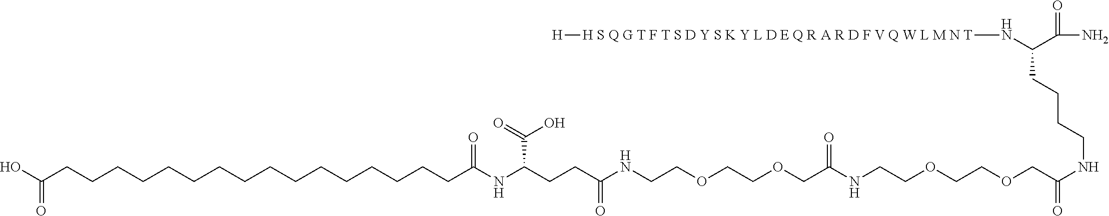 | 0.192 |
| Exam- ple 27 | 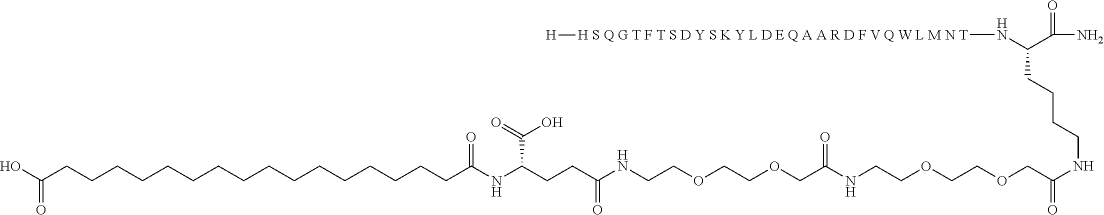 | 0.406 |
| Exam- ple 28 | 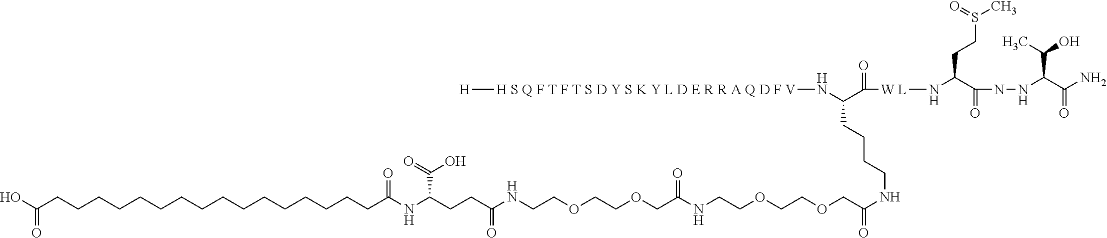 | 0.027 |
| Exam- ple 29 | 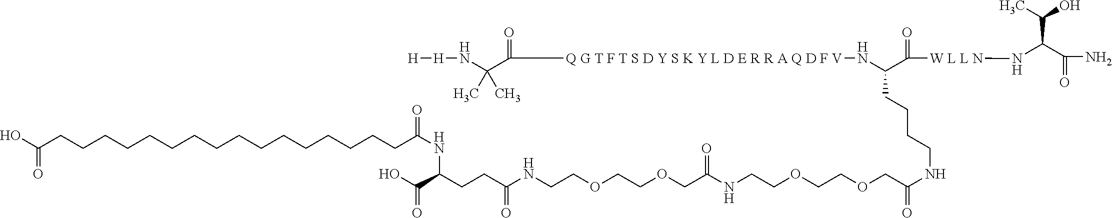 | 0.135 |
| Exam- ple 30 | 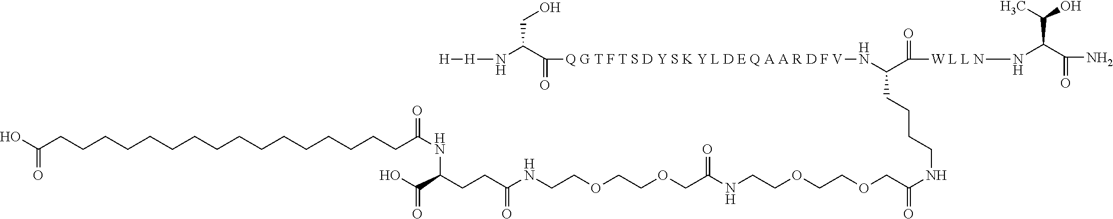 | 0.137 |
| Exam- ple 31 | 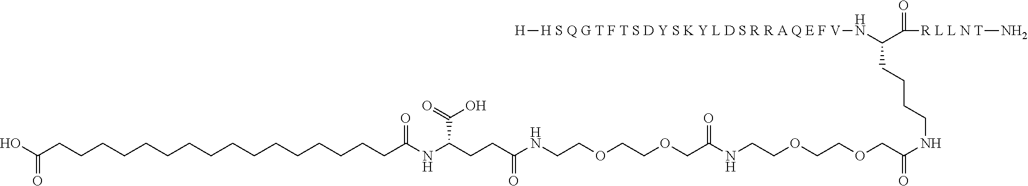 | 0.043 |
| Exam- ple 32 | 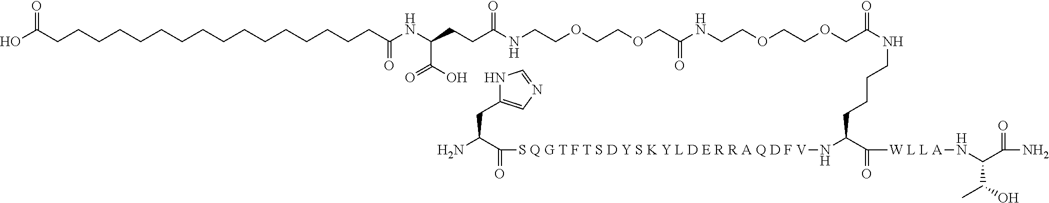 | 0.0235 |
| Exam- ple 33 | 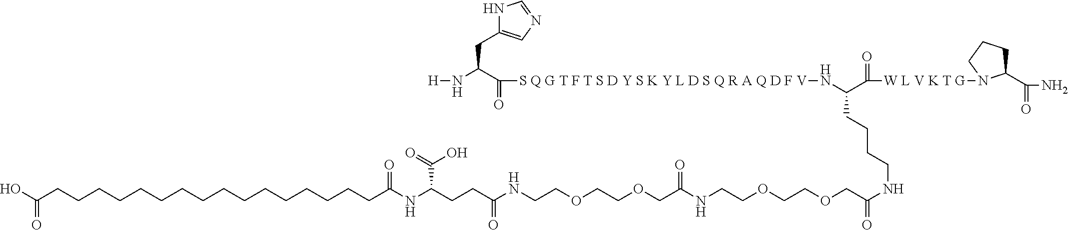 | 0.942 |
| Exam- ple 34 | 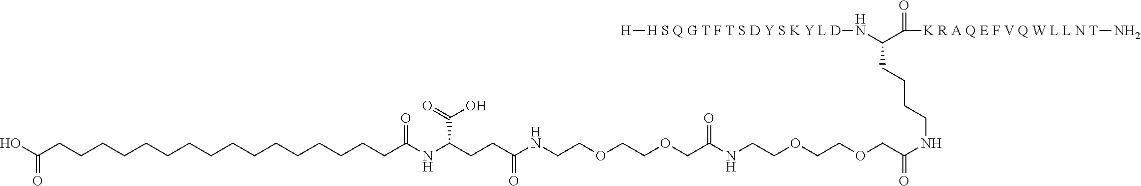 | 0.018 |
| Exam- ple 35 | 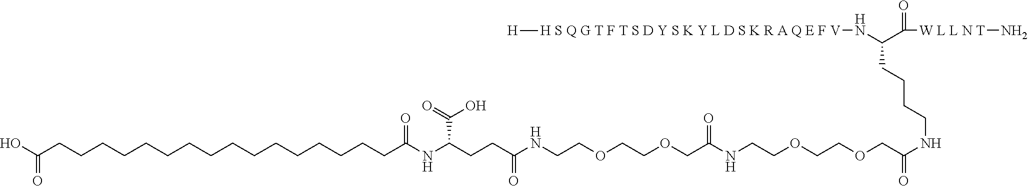 | 0.016 |
| Exam- ple 36 | 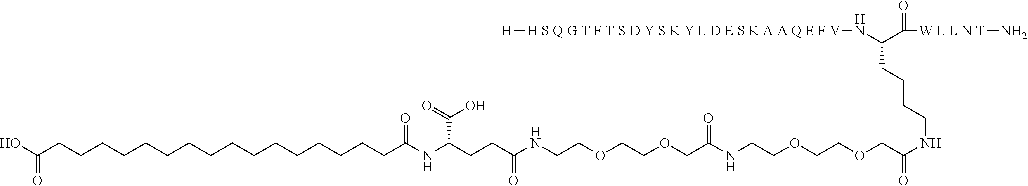 | 0.048 |
| Exam- ple 37 | 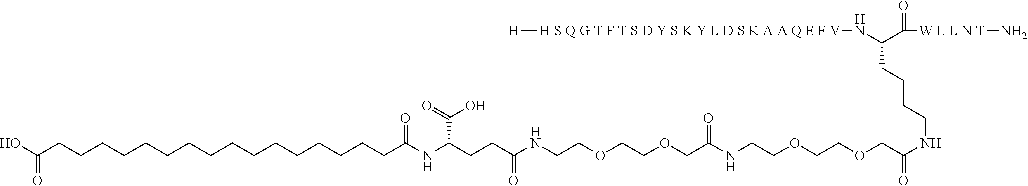 | 0.033 |
| Exam- ple 38 | 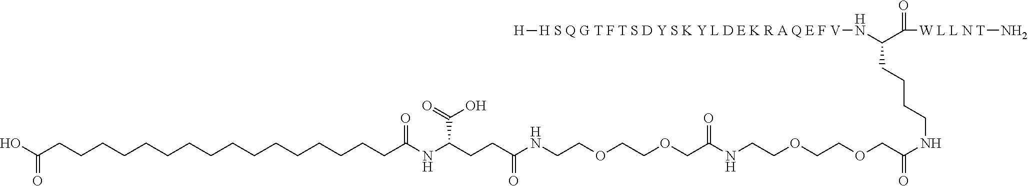 | 0.015 |
| Exam- ple 39 | 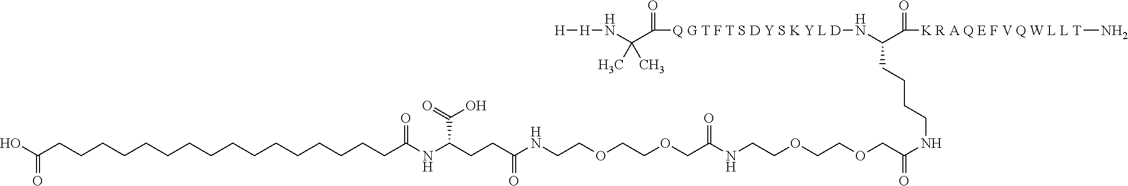 | 0.007 |
| Exam- ple 40 | 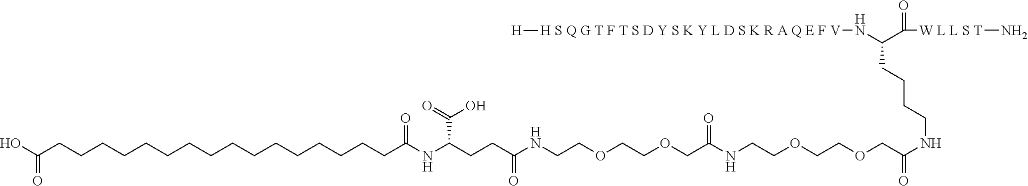 | 0.007 |
| Exam- ple 41 | 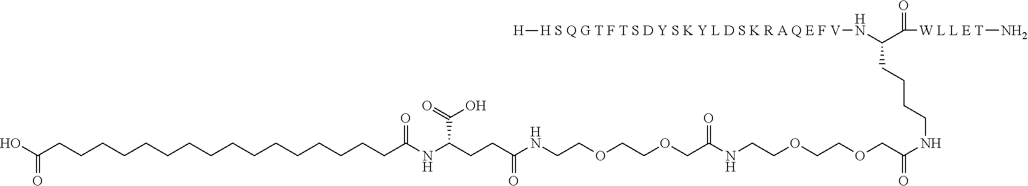 | 0.003 |
| Exam- ple 42 | 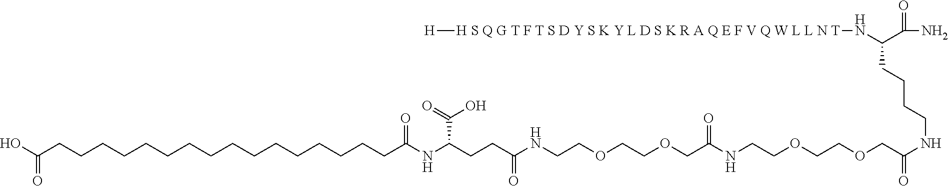 | 0.017 |
| Exam- ple 43 | 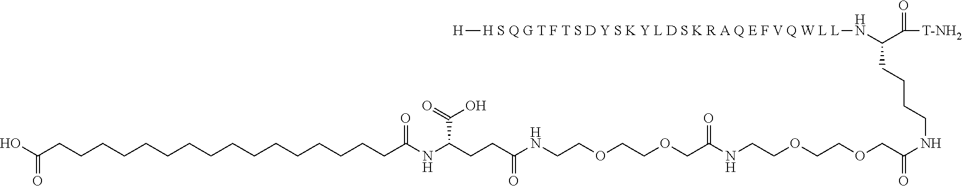 | 0.003 |
| Exam- ple 44 | 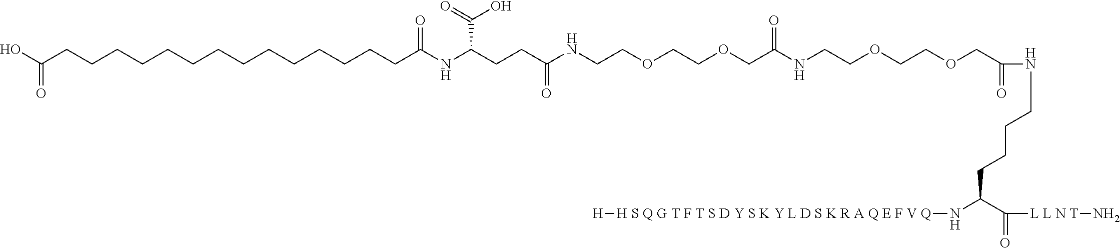 | 0.012 |
| Exam- ple 45 | 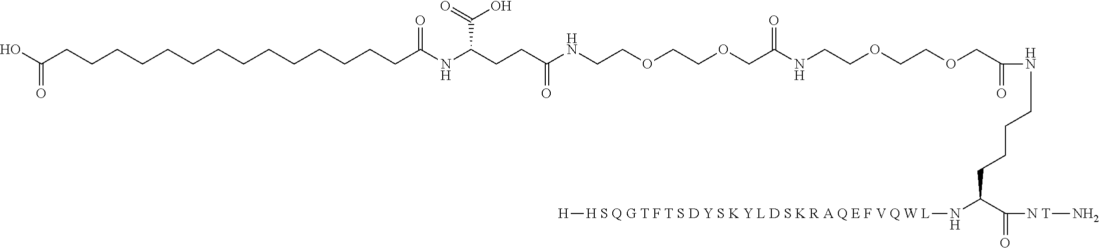 | 0.007 |
| Exam- ple 46 | 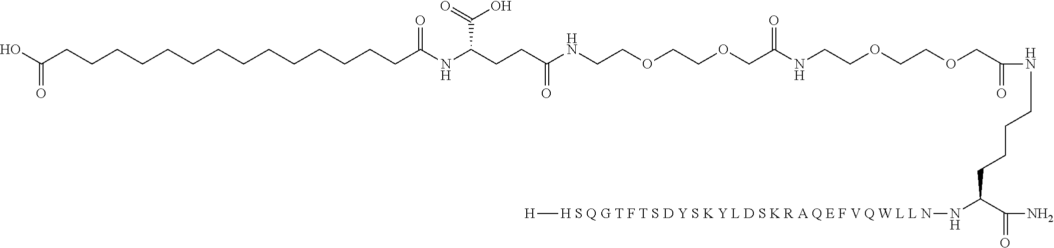 | 0.003 |
| Exam- ple 47 | 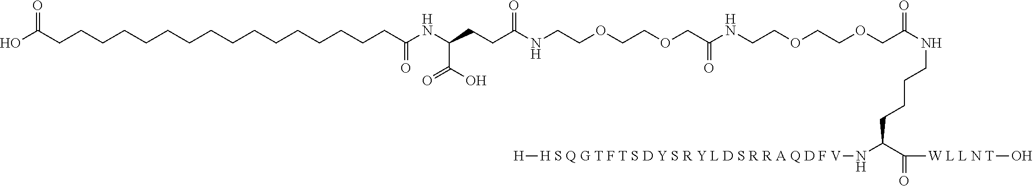 | 0.109 |
| Exam- ple 48 | 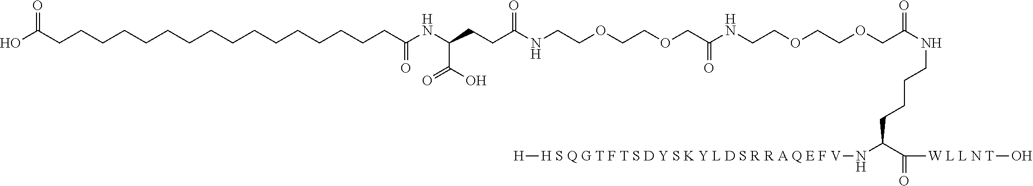 | 0.021 |
| Exam- ple 49 | 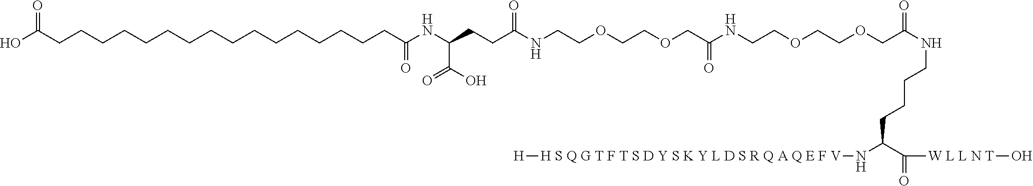 | 0.150 |
| Exam- ple 50 | 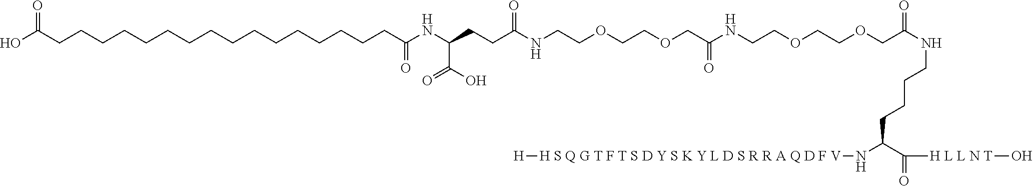 | 0.194 |
| Exam- ple 51 | 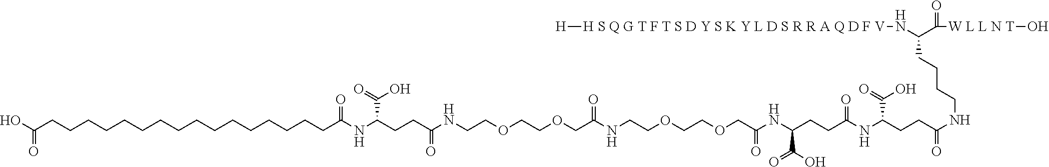 | 0.051 |
| Exam- ple 52 | 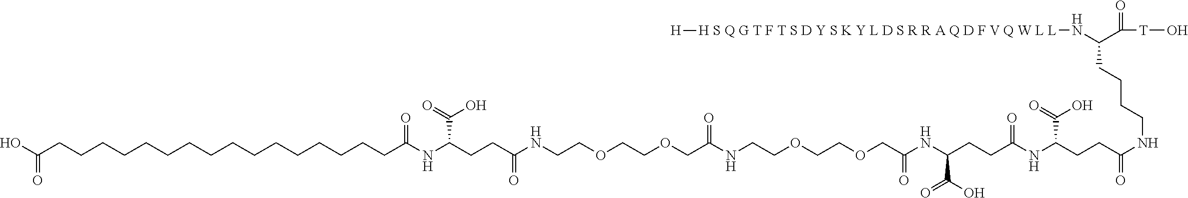 | 0.055 |
| Exam- ple 53 | 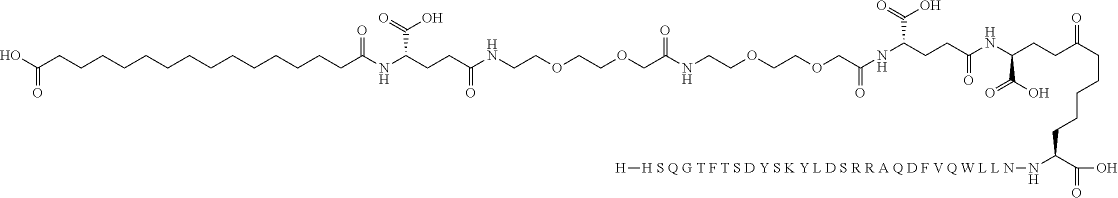 | 0.095 |
| Exam- ple 54 | 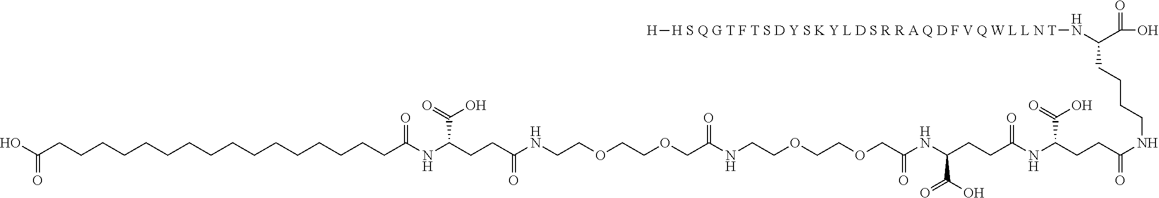 | 0.056 |
| Exam- ple 55 | 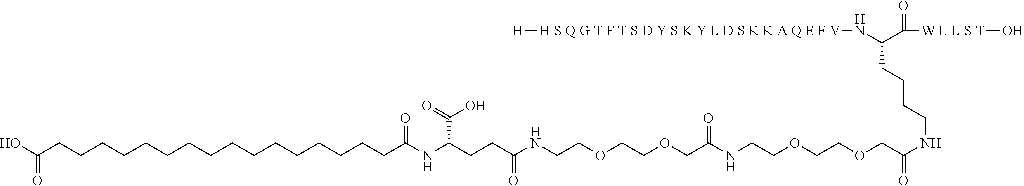 | 0.009 |
| Exam- ple 56 | 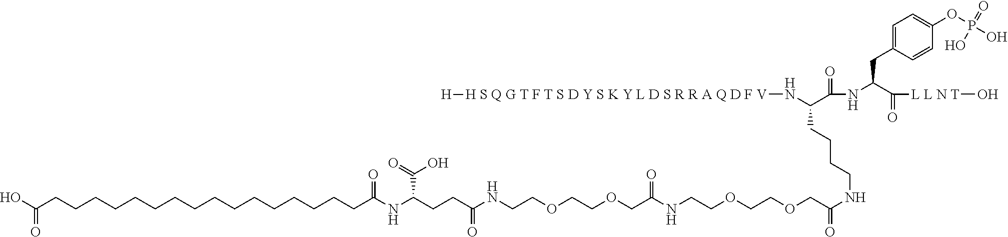 | 0.171 |
| Exam- ple 58 | 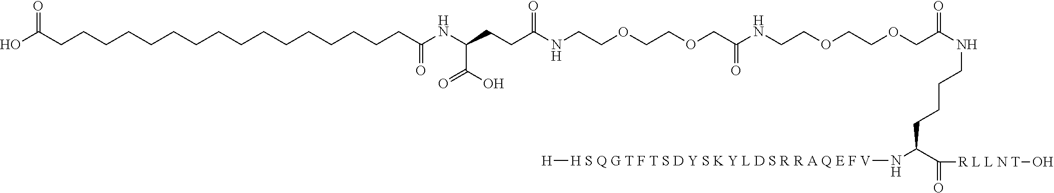 | 0.074 |

TABLE 2
In vitro data on receptor binding, ThT assay lag time and recovery

| | | Assay | | |
|---|---|---|---|---|
| | | (I) | ThT | ThT |
| | | Gluc- | assay | assay |
| | | agon | [Lag | [Reco- |
| Exam- | | [EC50] | time] | very] |
| ple | | (nM) | (h) | (%) |
| hGluc- | H—HSQGTFTSDYSKYLDSRRAQDFVQWLMNT—OH | 0.011 | 1.5 | 2.5 |
| agon | | | | |
| K10 (γGlu- γGlu- C16) Glu- cagon- NH2 | 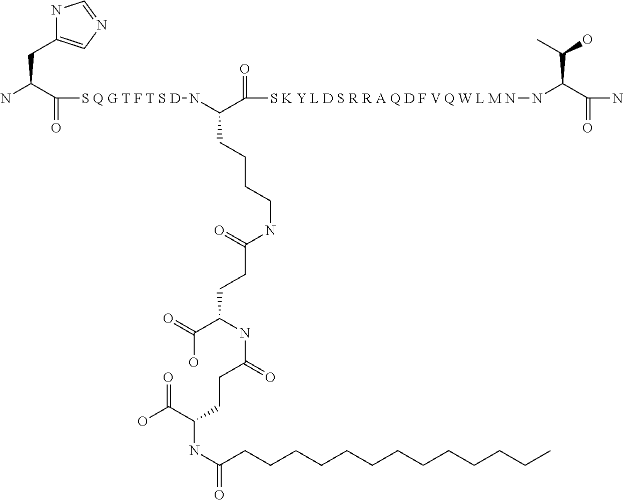 | 0.006 | 14 | 0 |
| 16 | 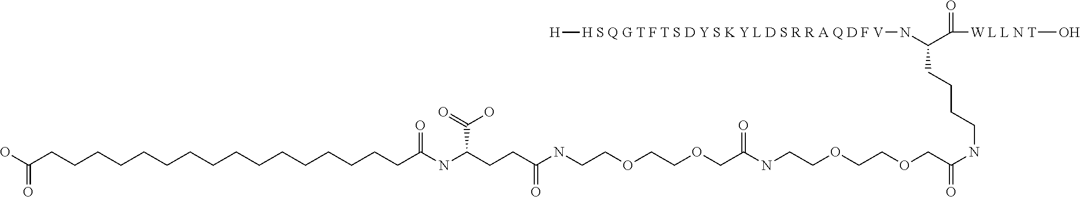 | 0.017 | 1.3 | 0 |
| 51 | 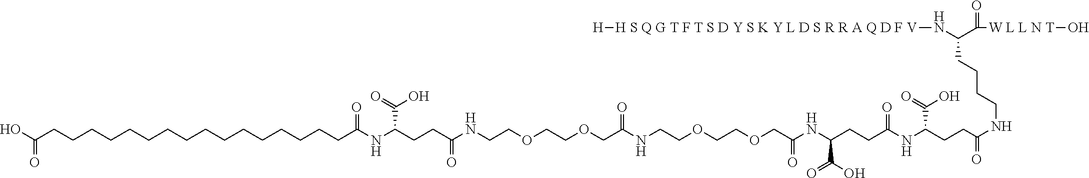 | 0.051 | 45 | 100 |
| 52 | 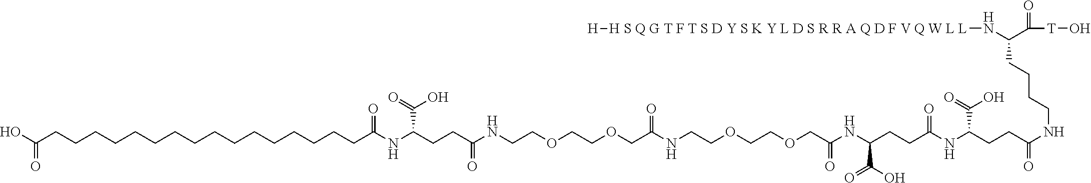 | 0.055 | 30 | 95 |
| 53 | 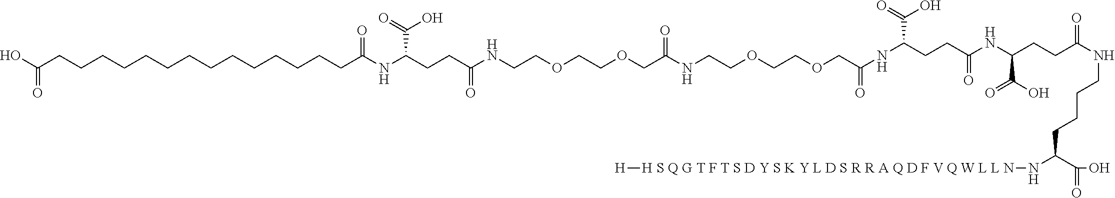 | 0.095 | 12 | 91 |
| 54 | 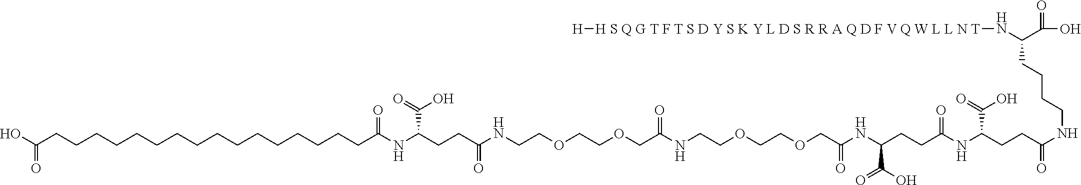 | 0.056 | 5 | 87 |
| 66 | 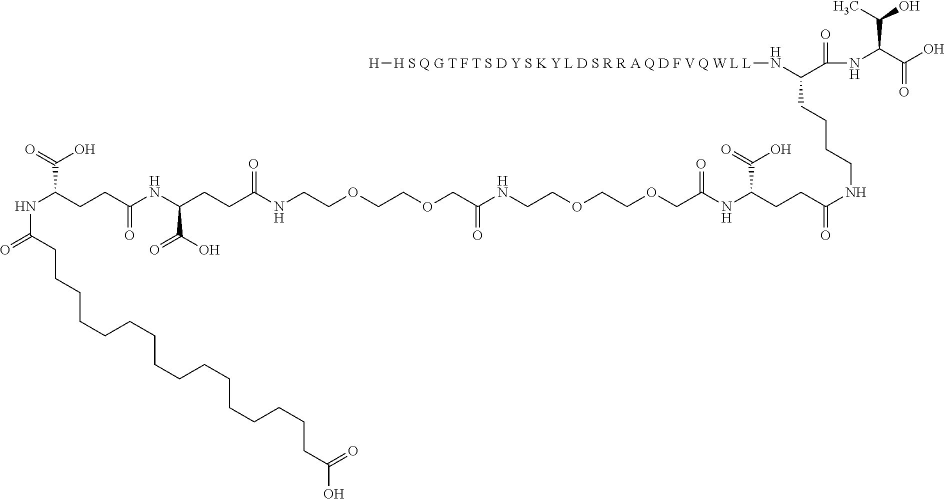 | 0.093 | 45 | 100 |
| 67 | 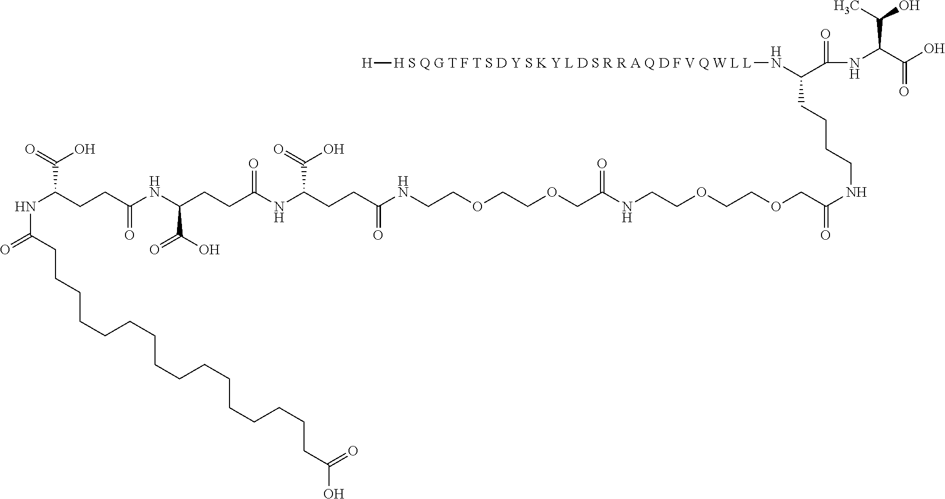 | 0.116 | 15 | 94 |
| 68 | 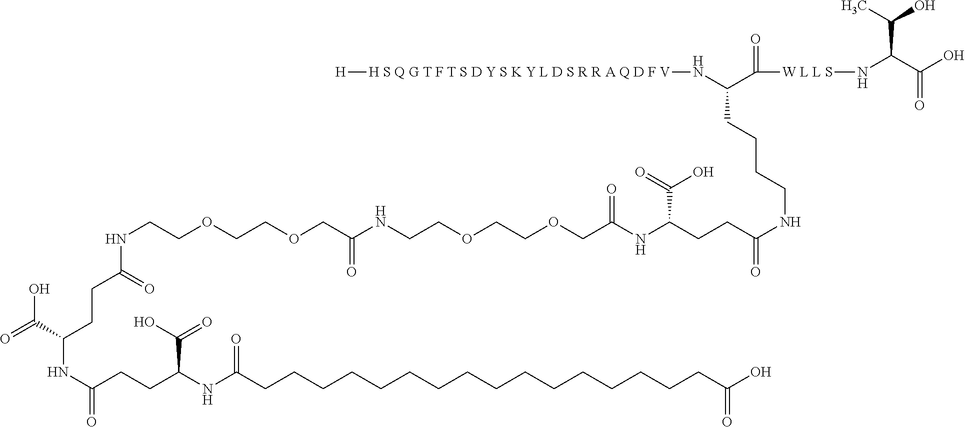 | 0.106 | 45 | 100 |
| 69 | 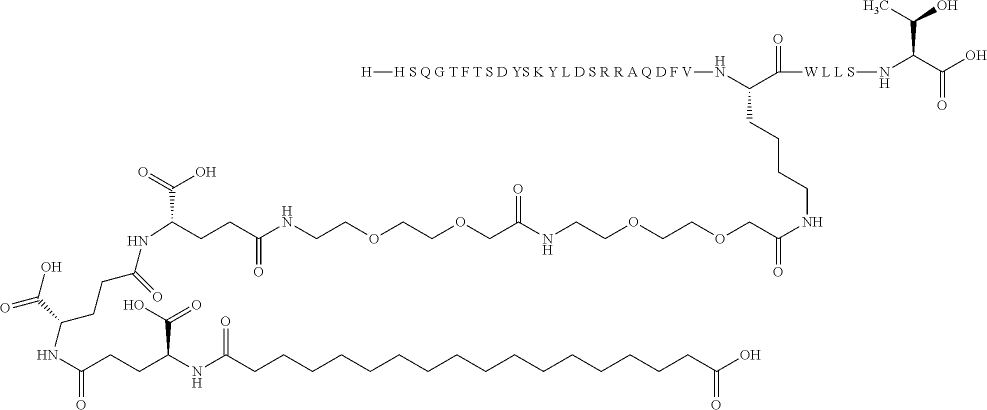 | 0.115 | 45 | 100 |
| 70 | 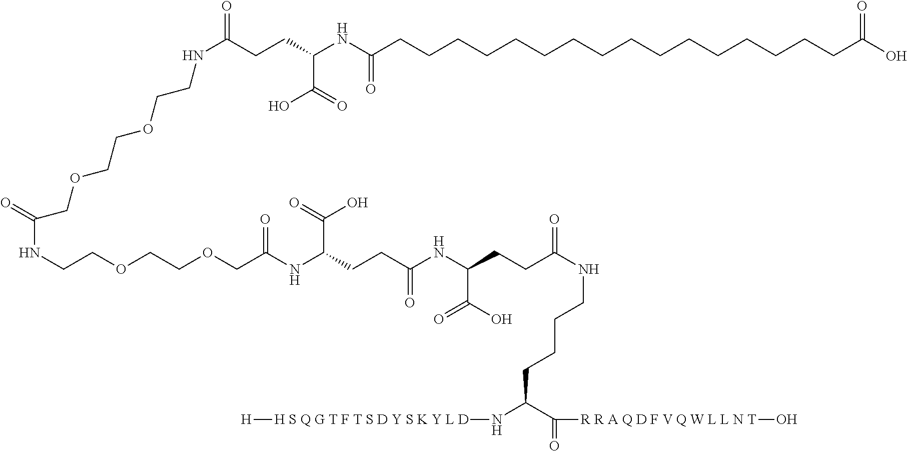 | 0.105 | 15 | 44 |
| 71 | 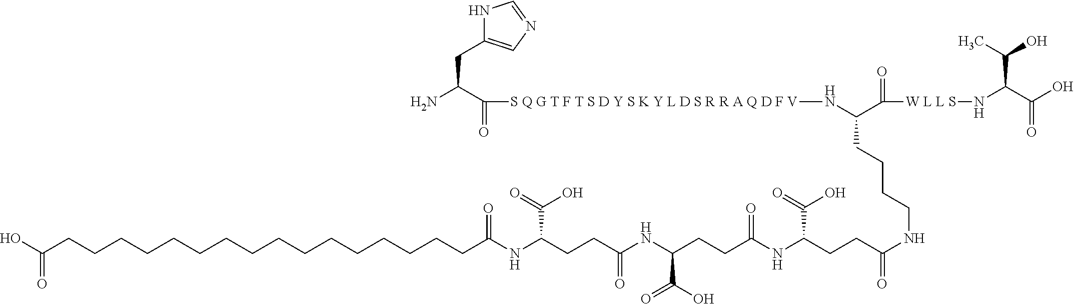 | 0.094 | 45 | 100 |
| 72 | 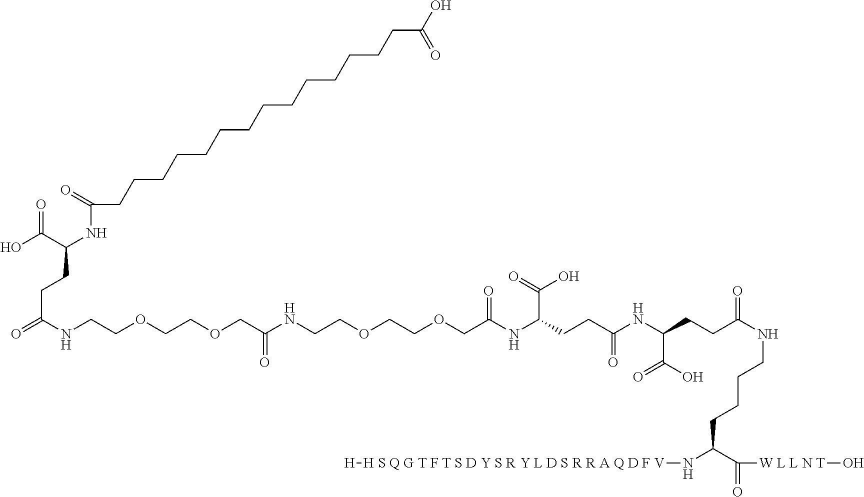 | 0.127 | 39 | 100 |
| 73 | 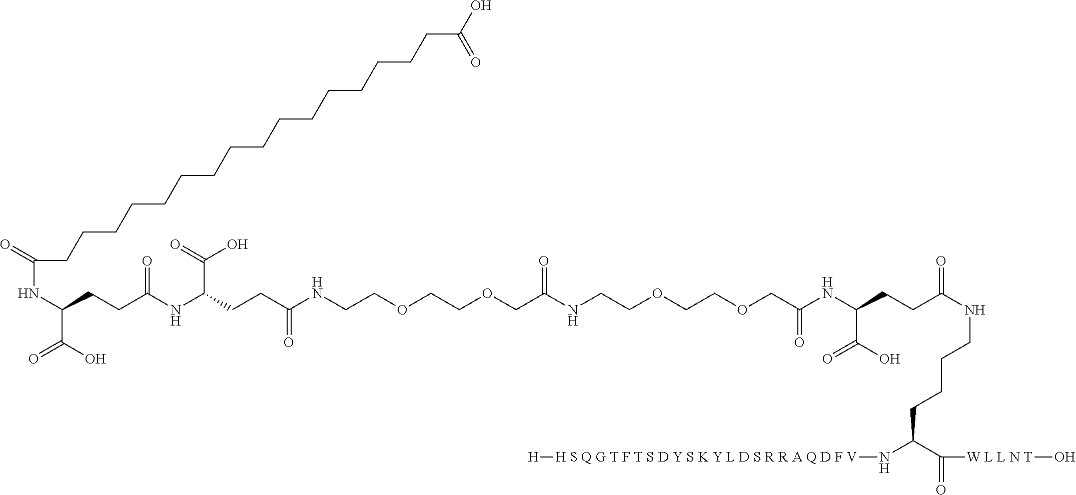 | 0.079 | 45 | 100 |
| 74 | 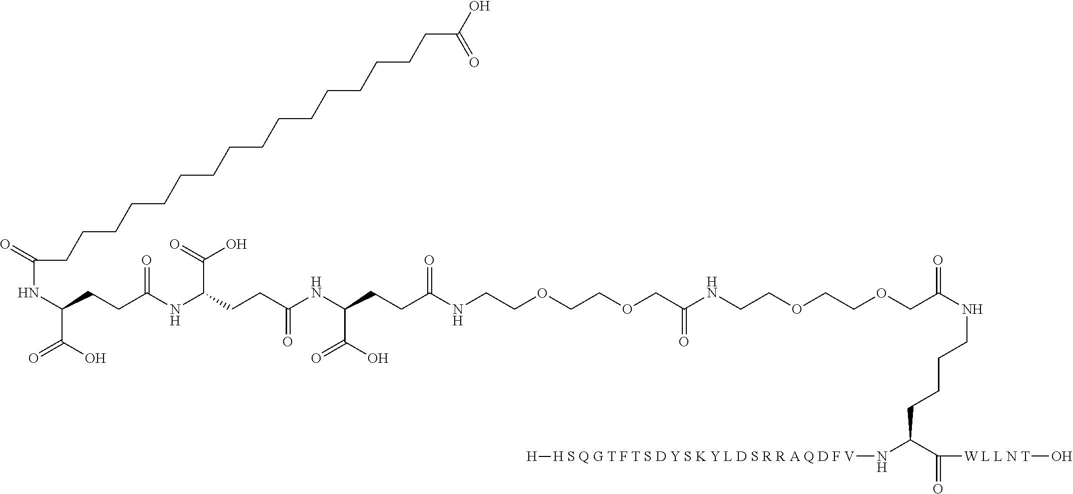 | 0.093 | 45 | 100 |
| 75 | 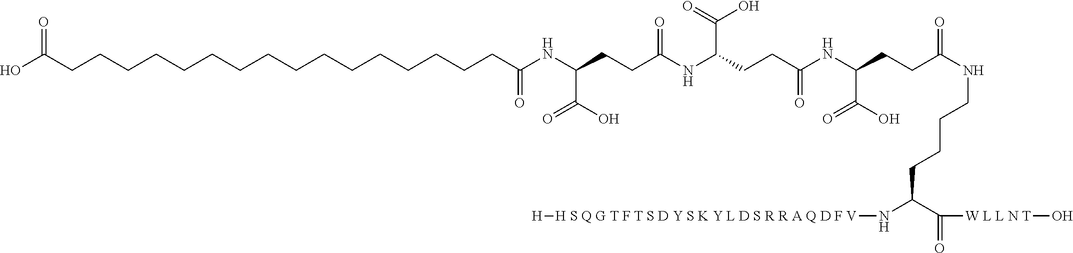 | 0.170 | 45 | 100 |
| 76 | 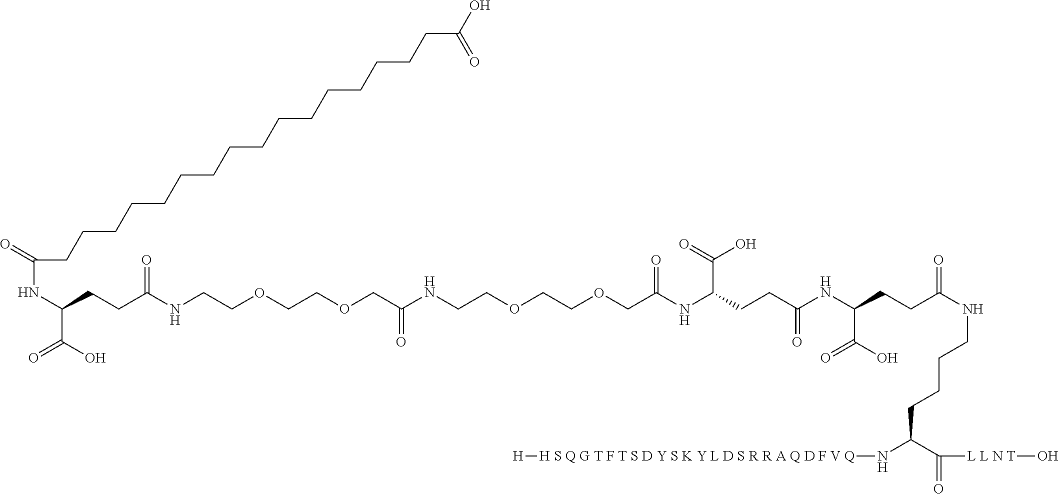 | 0.988 | 30 | 97 |
| 77 | 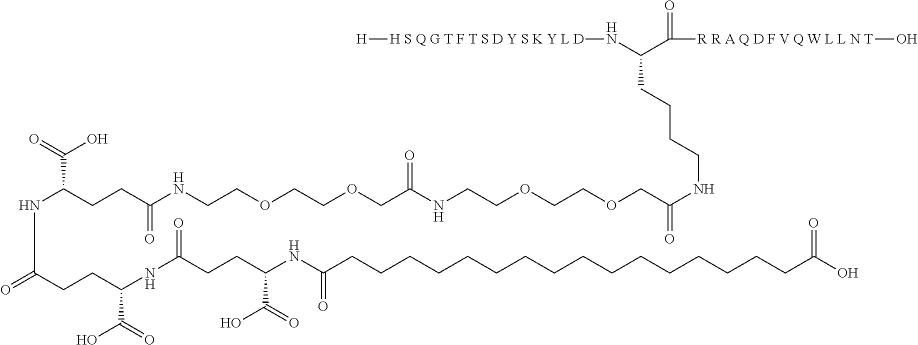 | 0.081 | 2 | 59 |
| 78 | 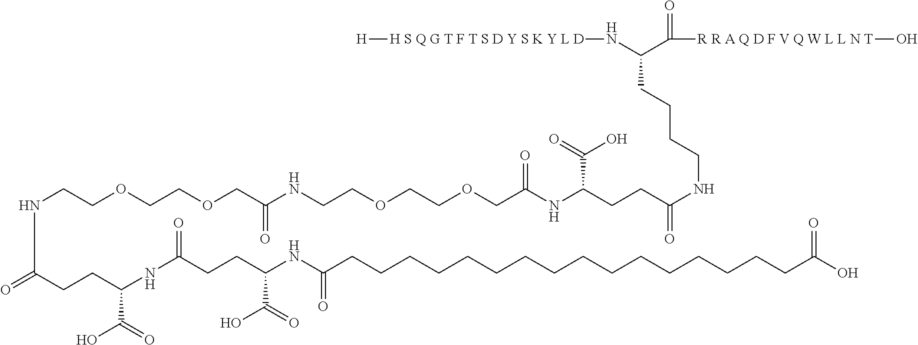 | 0.122 | 4 | 59 |
| 79 | 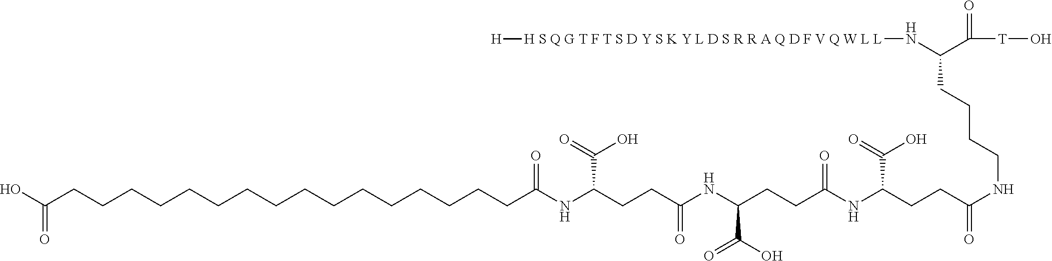 | 0.141 | 8 | 68 |
| 80 | 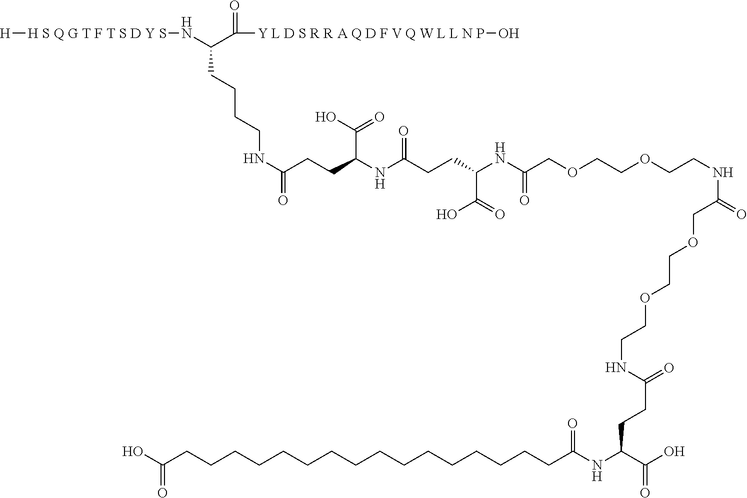 | 1.577 | n.d | n.d |
| 81 | 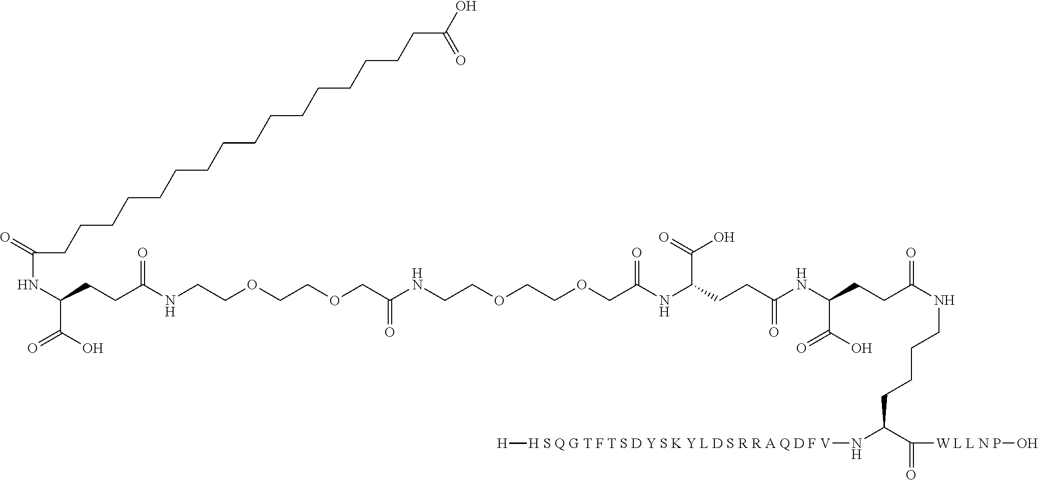 | 0.156 | 40 | 100 |
| 82 | 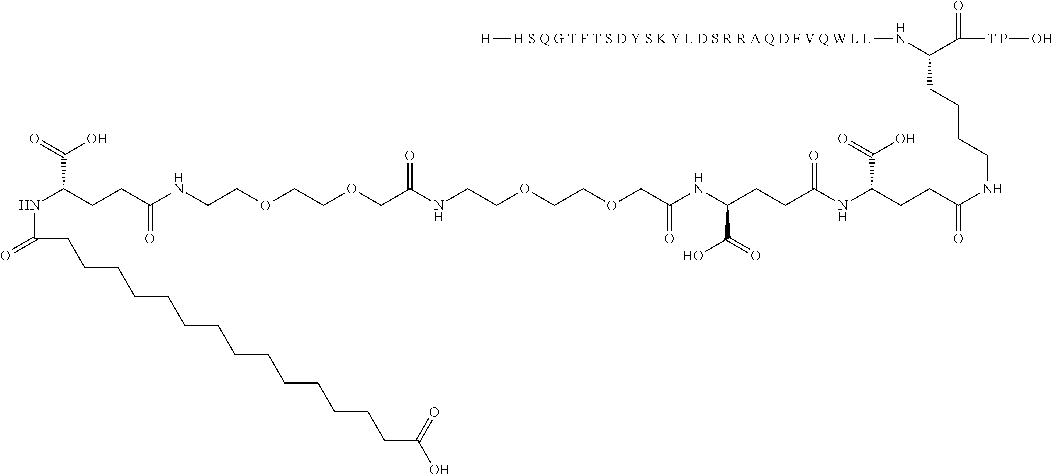 | 0.128 | 45 | 100 |
| 83 | 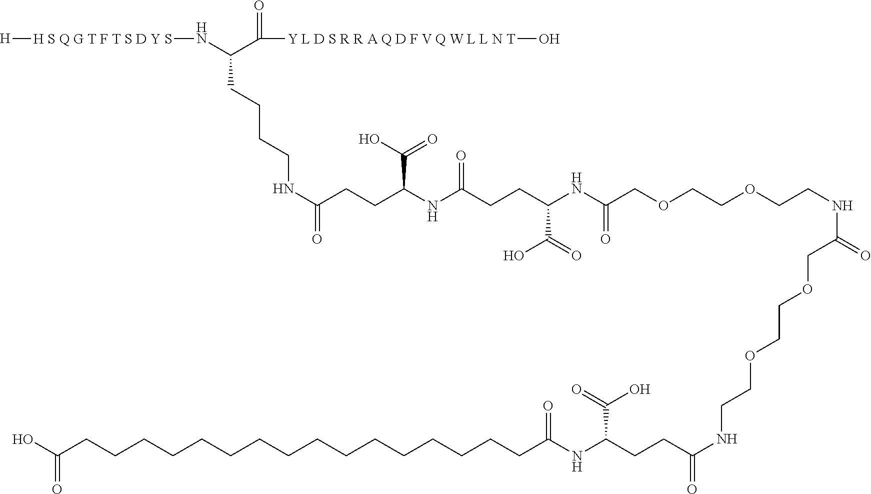 | 1.878 | 45 | 100 |
| 84 | 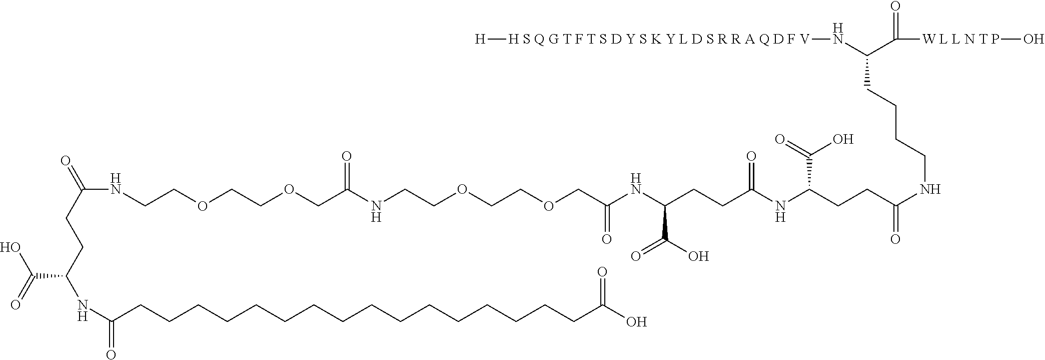 | 0.142 | 45 | 100 |
| 85 | 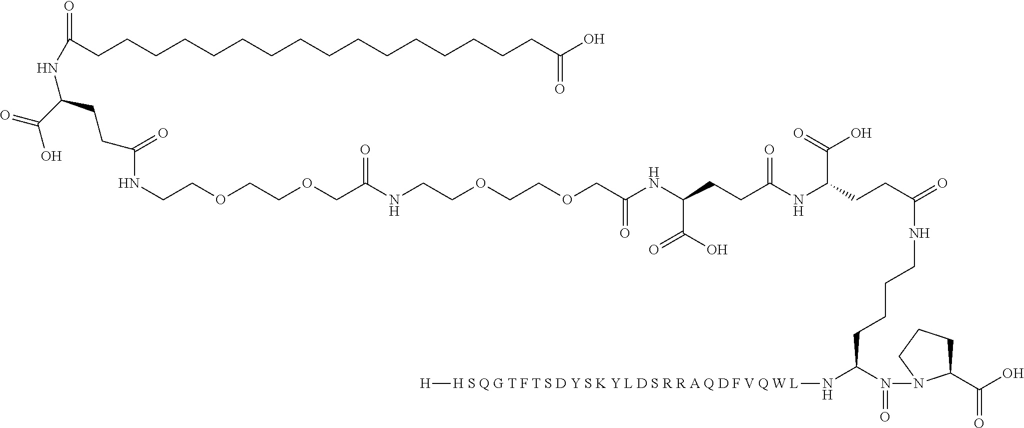 | 1.173 | 7 | 100 |
| 86 | 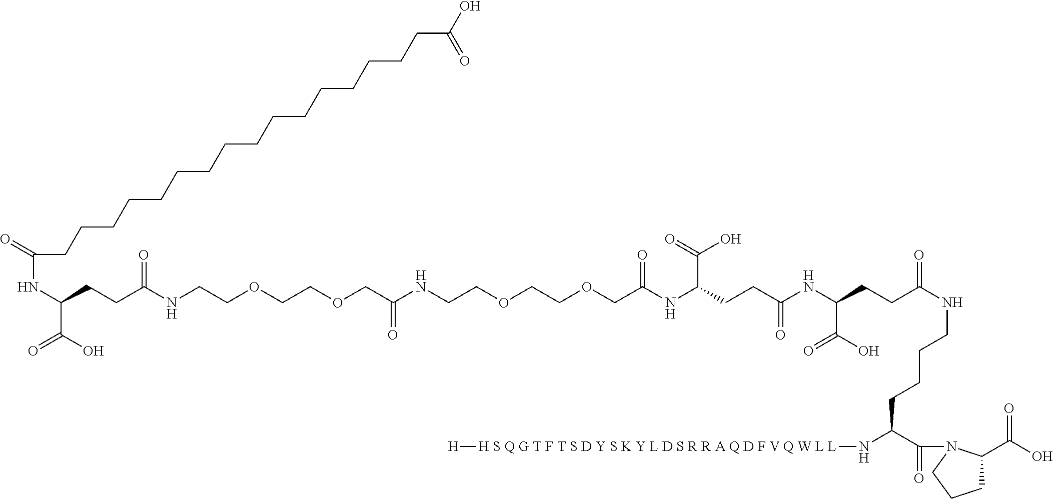 | 0.189 | 10 | 82 |
| 87 | 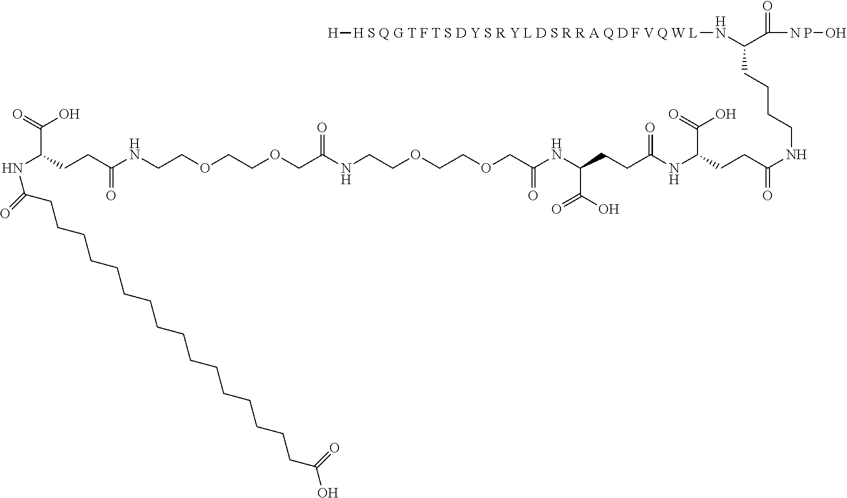 | 2.114 | 0 | 95 |
| 88 | 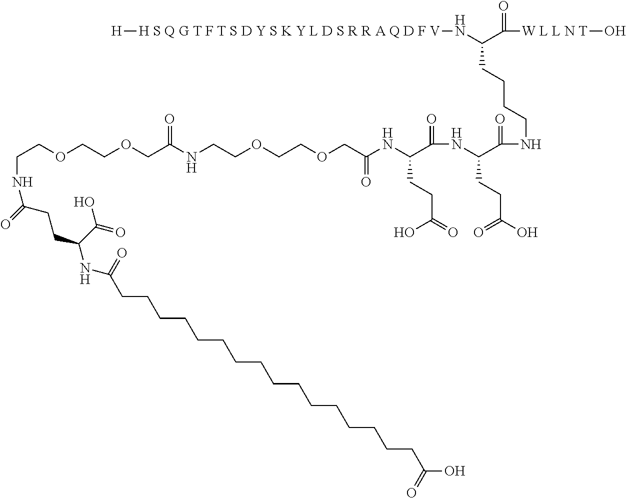 | 0.037 | 45 | 100 |
| 89 | 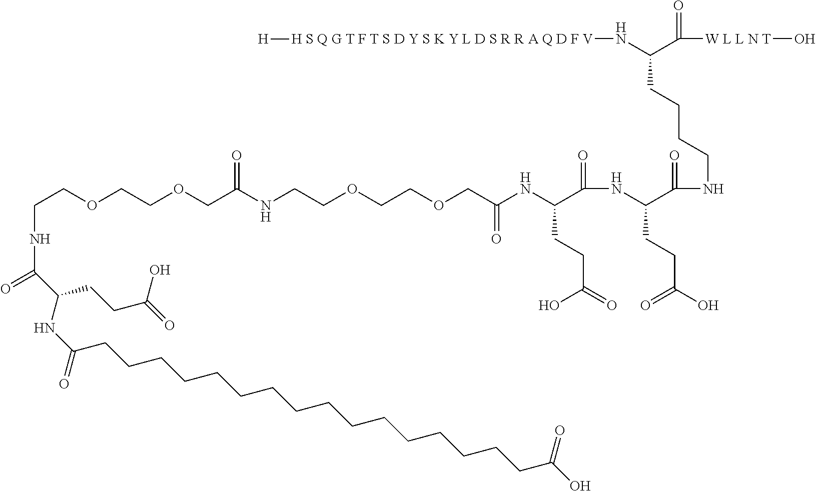 | 0.087 | 45 | 100 |
| 90 | 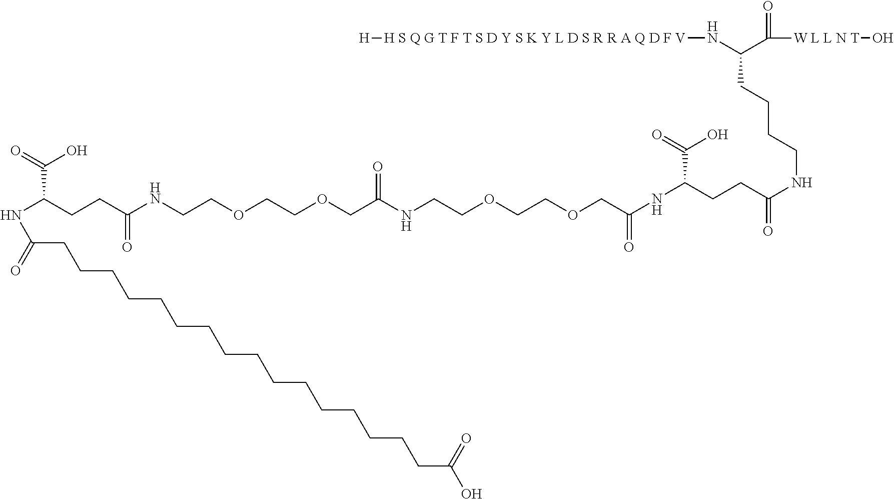 | 0.018 | 29 | 84 |
| 91 | 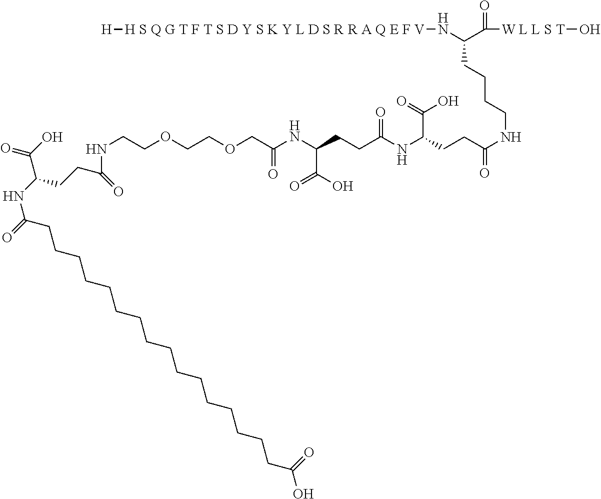 | 0.053 | 45 | 100 |
| 92 | 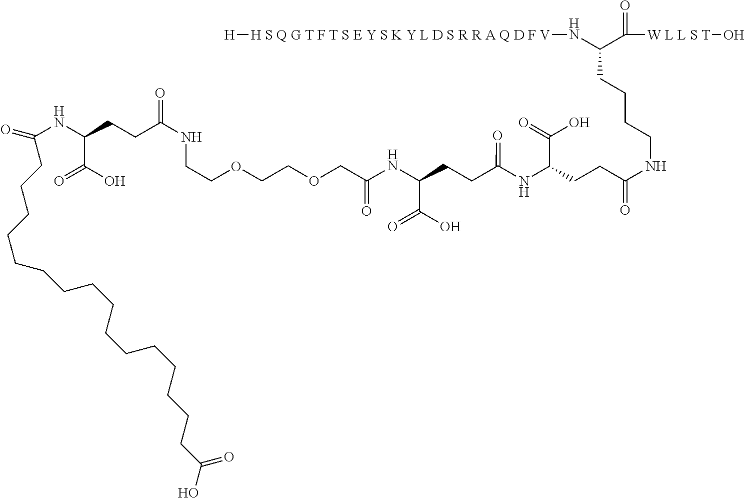 | 2.2 | 45 | 100 |
| 93 | 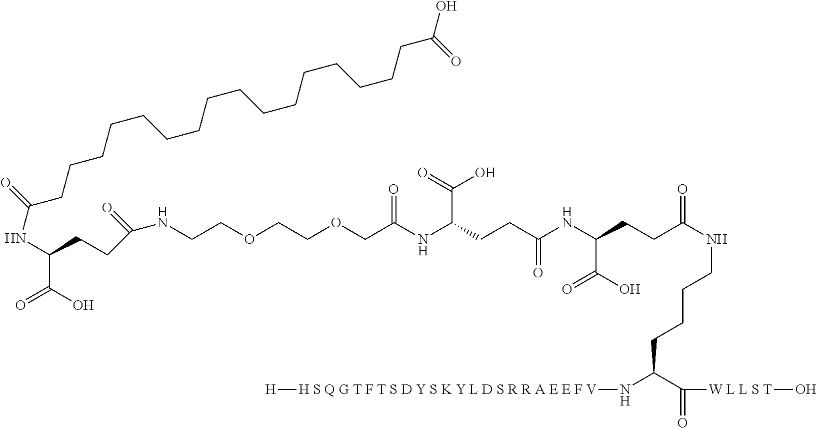 | 0.12 | 45 | 100 |
| 94 | 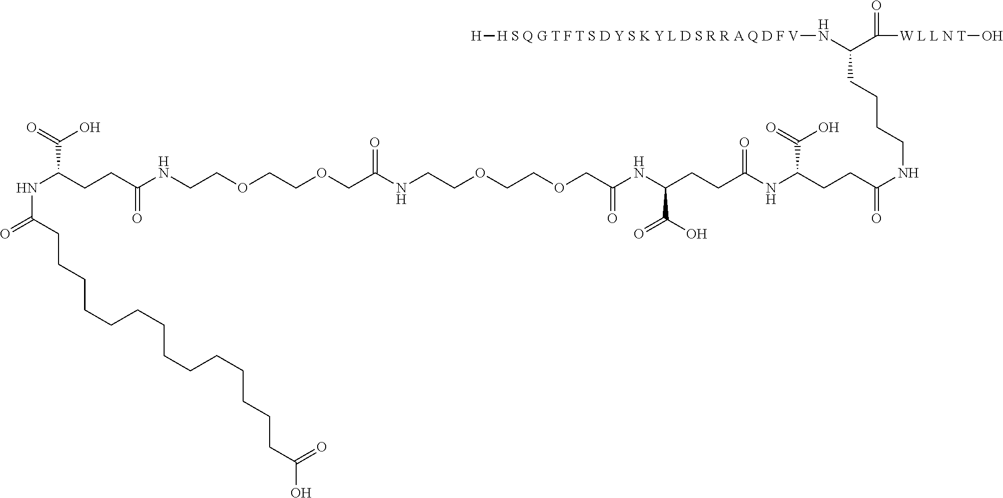 | 0.009 | 45 | 100 |
| 95 | 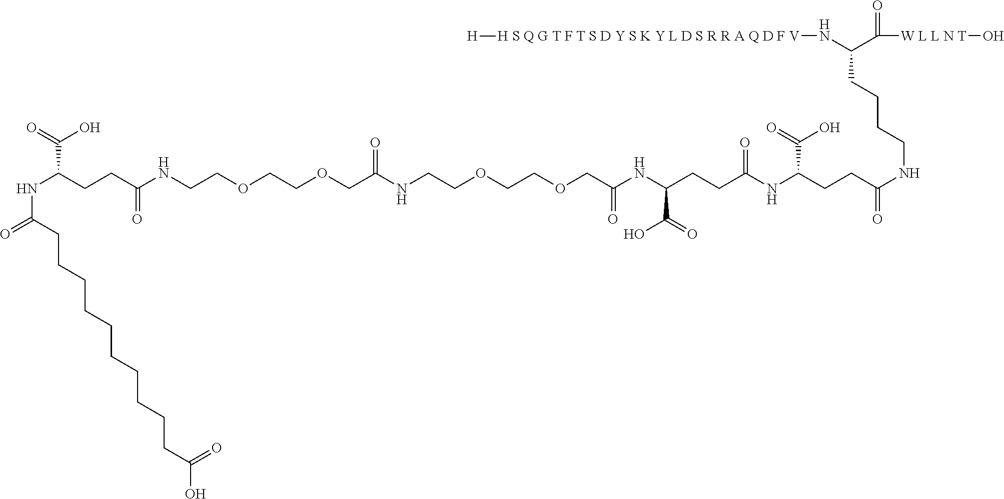 | 0.009 | 45 | 100 |
| 96 | 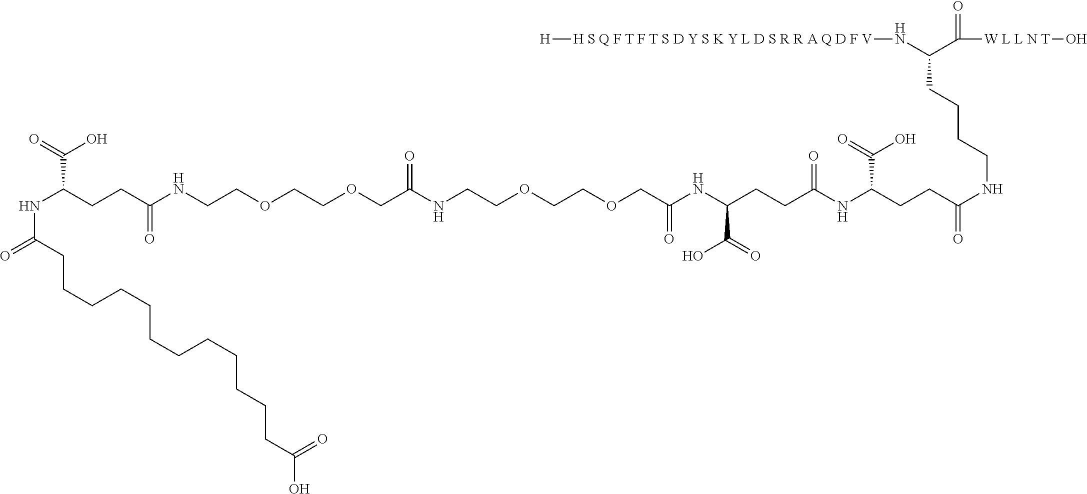 | 0.010 | 45 | 100 |
| 97 | 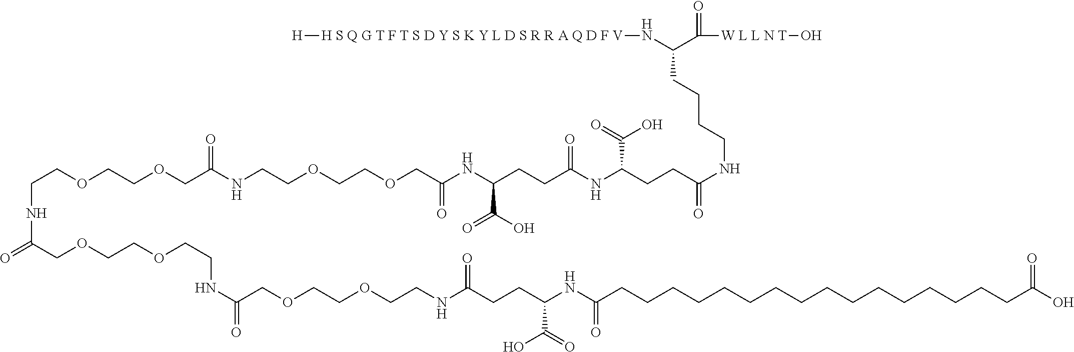 | 0.033 | 45 | 100 |
| 98 | 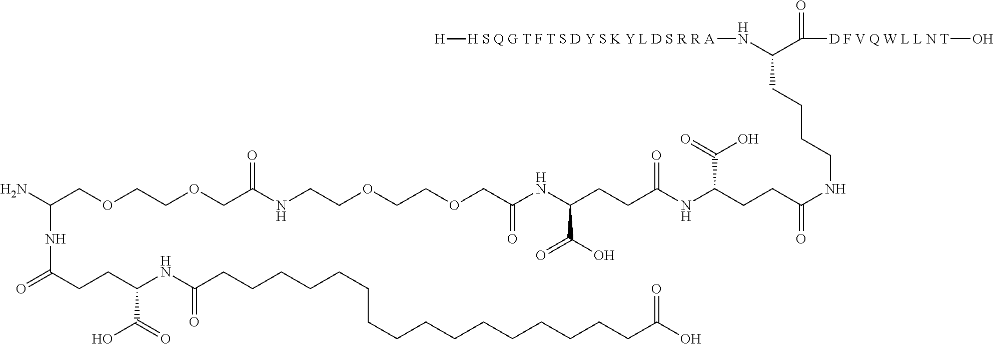 | 0.058 | 1 | 26 |
| 99 | 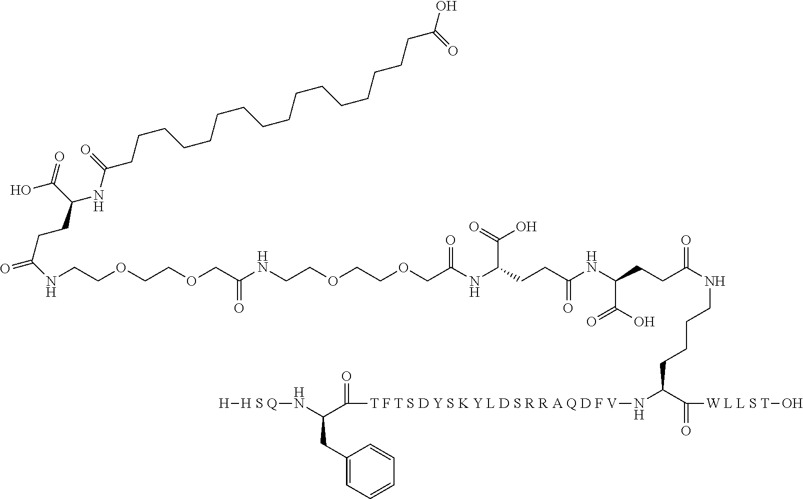 | 0.044 | 45 | 100 |
| 100 | 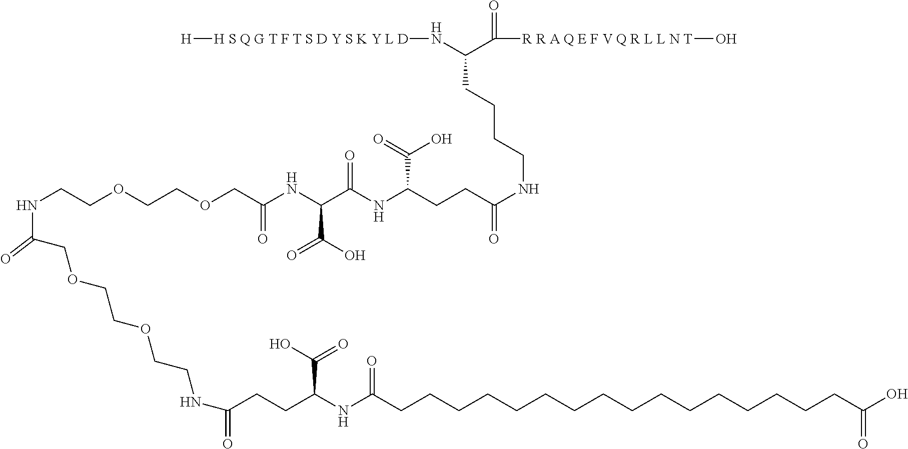 | 0.450 | 45 | 100 |
| 101 | 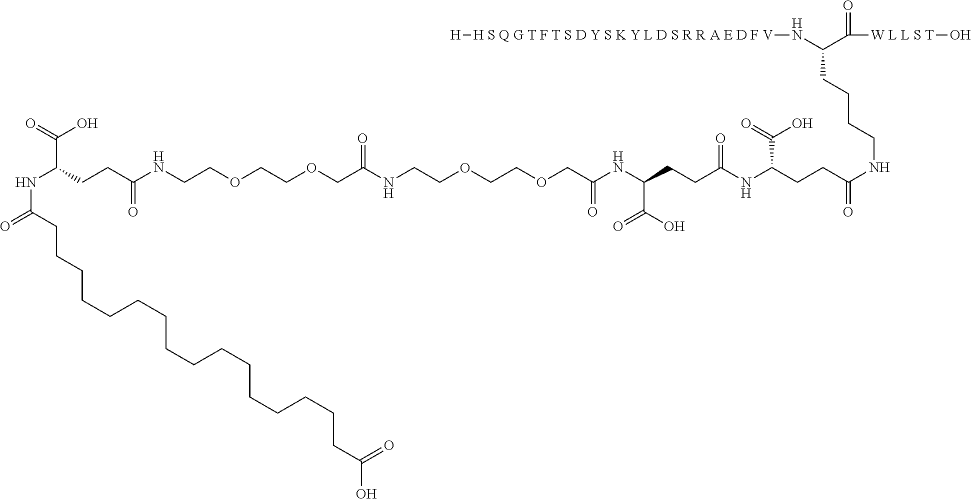 | 0.307 | 45 | 100 |
| 102 | 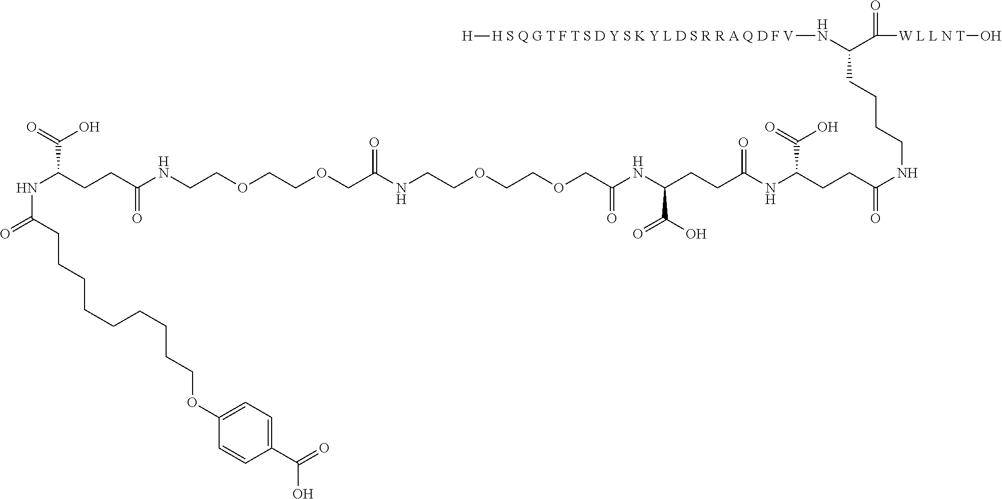 | 0.007 | 45 | 100 |
| 103 | 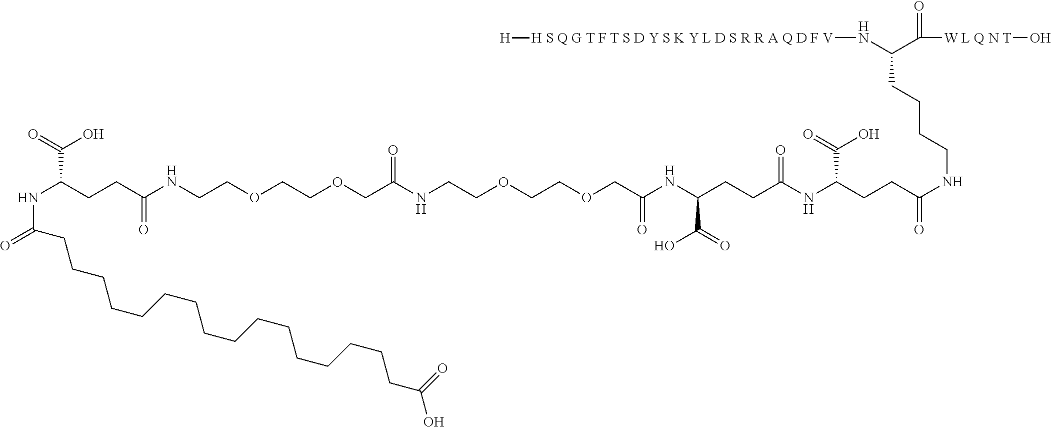 | 0.118 | 45 | 100 |
| 104 | 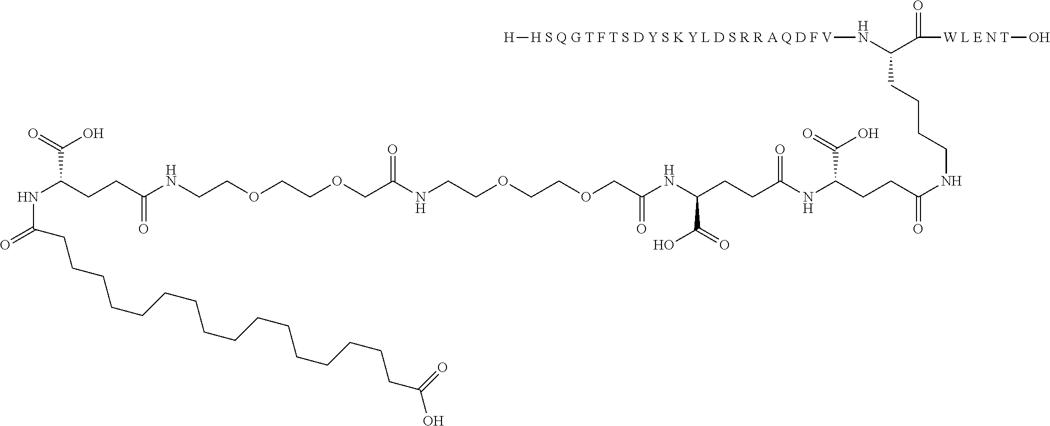 | 0.101 | 32 | 100 |
| 105 | 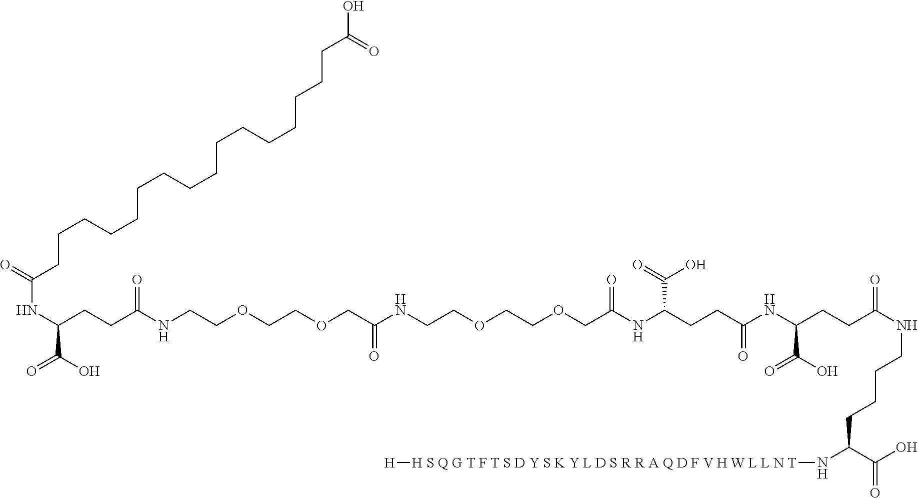 | 0.100 | 1.3 | 16 |
| 106 | 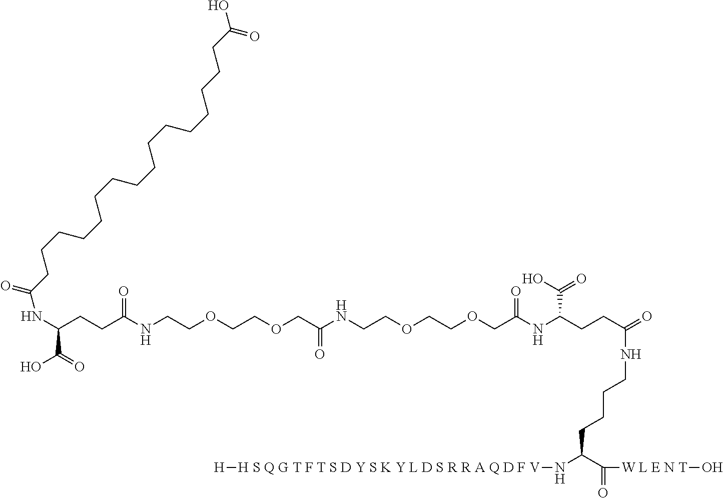 | 0.067 | 2.3 | 63 |
| 107 | 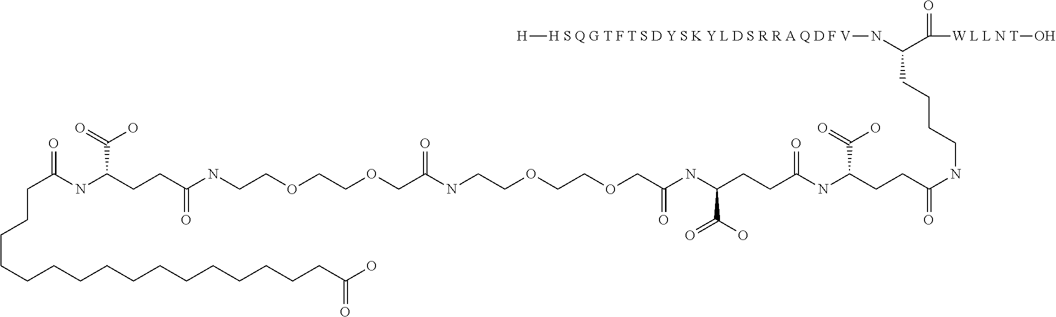 | 0.116 | 9 | 100 |
| 108 | 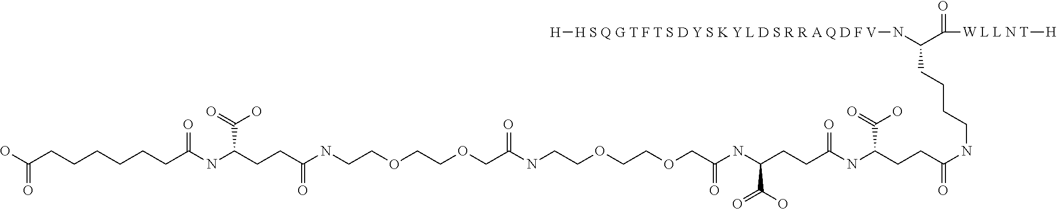 | 1.011 | 45 | 100 |

### Assay (II)

### GLP-1 Activity

The GLP-1 receptor was cloned into HEK-293 cells having a membrane bound cAMP biosensor (ACTOne™). The cells (14000 per well) were incubated (37° C., 5% CO2) overnight in 384-well plates. Next day the cells were loaded with a calcium responsive dye that only distributed into the cytoplasm. Probenecid, an inhibitor of the organic anion transporter, was added to prevent the dye from leaving the cell. A PDE inhibitor was added to prevent formatted cAMP from being degraded. The plates were placed into a FLIPRTETRA and the glucagon analogues were added. End point data were collected after 6 minutes. An increase in intracellular cAMP was proportional to an increased in calcium concentrations in the cytoplasm. When calcium was bound the dry a fluorescence signal was generated. EC50-values were calculated in Prism5.

### Assay (III)

### LOCI Assay

Samples were analyzed for peptide using Luminescence Oxygen Channeling Immunoassay (LOCI). The donor beads were coated with streptavidin, while acceptor beads were conjugated with a monoclonal antibody (1F120) specific for glucagon. The other glucagon-binding monoclonal antibody (2F7) was biotinylated. Three reactants were combined with the analyte and formed a two-sited immuno-complex. Illumination of the complex released singlet oxygen atoms from the donor beads. They were channeled into the acceptor beads and triggered chemiluminescence which was measured in the EnVision plate reader. The amount of emitted light was proportional to the concentration of peptide.

One μL sample/calibrator/control was applied to the wells of 384-well LOCI plates followed by a 15 μL mixture of the antibody-coated acceptor beads (0.5 μg/well) and the biotinylated antibody. The plates were incubated for 1 h at 21-22° C. Then 30 μL the streptavidin-coated donor-beads (2 μg/well) were added to each well and incubated for 30 minutes at 21-22° C. The plates were red in an Envision plate reader at 21-22° C. with a filter having a bandwidth of 520-645 nm after excitation by a 680 nm laser. The total measurement time per well was 210 ms including a 70 ms excitation time.

### Assay (IV)

### Body Weight Loss in Diet Induced Obese Rats

Sixtyfour high fat (Research Diet D12492) fed and eight low fat (Research Diet D12450B) fed Sprague Dawley rats from Taconic Europe were used for this study. The rats weighed app. 970 g and 730 g, respectively before dosing. Rats had ad lib access to water and were housed individually to allow daily monitoring of food intake. Lights were turned off from 10 AM to 10 PM.

Rats were divided into groups of eight and dosed subcutaneously (sc) once daily with two test substances for 15 days, dose volume was 0.5 ml/kg. Before dosing was initiated rats were handled daily and trained for sc. dosing for 5 days. The rats were dosed with glucagon analogue N-epsilon24-([2-[2-[2-[[(4S)-5-hydroxy-4-[(18-hydroxy-18-oxooctadecanoyl)amino]5-oxopentanoyl]amino]ethoxy]ethoxy]acetyl]amino]-ethoxy]ethoxy]acetyl]])[Lys¹⁷,Lys¹⁸,Glu21,Lys²⁴,Leu²⁷]-Glucagon (Example 3) or G3.

The high fat fed test groups were as follows: group1: vehicle (received two vehicle injections), group 2: glucagon analogue (Example 3) 30 nmol/kg and one vehicle injection; group 3: glucagon analogue (Example 3) 300 nmol/kg and one vehicle injection; group 4: G3 1 nmol/kg and one vehicle injection; group 5: glucagon analogue (Example 3) 30 nmol/kg and G3 1 nmol/kg; group 6: glucagon analogue (Example 3) 300 nmol/kg and G3 1 nmol/kg; group 7: two vehicle injections and pair fed to group 6. Group 8 was fed a low fat diet and received two vehicle injections. At the 5^{th} dosing day the doses of glucagon analogue (Example 3) were adjusted from 30 nmol/kg to 3 nmol/kg and from 300 nmol/kg to 30 nmol/kg due to the dramatic weight loss curve experienced in the rats.

At day 11 the rats were subjected to a blood glucose profiling. Rats were terminated either at day 15 or day 16, and blood was sampled for measurement of insulin and cholesterol.

### Assay (V)

Experimental Protocol for Efficacy Testing on Appetite with a Glucagon Derivative, Using an Ad Libitum Fed Rat Model

Sprague Dawley (SD) rats from Taconic Europe, Denmark are used for the experiments. The rats had a body weight 200-250 g at the start of experiment. The rats arrived 14 days before start of experiment to allow acclimatization to experimental settings. During this period the animals were handled two times. After arrival rats were housed individually for one week in a reversed light/dark phase (meaning that lights are off during day time and on during night time) for two weeks. Since rats are normally active and eat their major part of their daily food intake during the dark period, rats are dosed in the morning right before lights are turned off. This set-up results in the lowest data variation and highst test sensitivity. The experiment was conducted in the rats' home cages and rats had free access to food and water throughout the acclimatization period and the experiment period. Each dose of derivative was tested in a group of 5 rats. A vehicle group of 6-7 rats was included in each set of testing. Rats were dosed once according to body weight with a 0.01-3 mg/kg solution administered subcutaneously (sc.).

After dosing, the rats were returned to their home cages, where they had access to food and water. The food consumption was recorded individually continuously by on-line registration or manually every hour for 7 hours, and then after 24 h and again after 48 h. At the end of the experimental session, the animals were euthanised.

The individual data were recorded in Microsoft excel sheets. Outliers were excluded after applying the Grubbs statistical evaluation test for outliers. Data was reported as acumulated food intake as functions of time. Comparisons were made between vehicle group and test groups using Student's t-test or one-way ANOVA.

### Assay (VI)

### DPP-IV Stability Assay

10 μM of peptide was incubated with DPP-IV (2 μg/ml) in duplicate at 37° C. in a HEPES buffer to which 0.005% Tween20 were added. In the experiment human GLP-1 was used as a postive control. Aliqouts of sample were taken at 3, 15, 30, 60, 120 and 240 min and three volumes of ethanol were added to stop the reaction. The samples were analysed by LC-MS for parent peptide. Data were plotted according to 1^{st} kinetics and the stability was reported as half-lives.

### Assay (VII)

### PK Profile

Fifteen male rats (Sprague Dawley, 400 g, Taconic Europe) were divided into three groups of five rats. The rats were dosed at t=0 with either 15 nmol/kg IV, 30 nmol/kg SC, or 100 nmol/kg, respectively. The IV dosing was performed via the tail vein while the rats were shortly under isoflurane anaesthesia. Blood samples were obtained via the sublingual vein at times t=−15 min, 5 min (only IV dosed rats), 15 min, 30 min, 1 h, 1½ h, 2 h, 4 h, 6 h, 12 h, 24 h, 48 h and 72 h. Plasma samples were stored on freeze until analysed by LCMS.

### Assay (VIII)

### pH Dependent Solubility

The solubility of peptides and proteins depends on the pH of the solution. Often a protein or peptide precipitates at or close to its isoelectric point (pI), at which its net charge is zero. At low pH (i.e. lower than the pI) proteins and peptides are typically positively charged, at pH higher than the pI they are negatively charged.

It is advantageous for a therapeutic peptide if it is soluble in a sufficient concentration at a given pH, which is suitable for both formulating a stable drug product and for administrating the drug product to the patient e.g. by subcutaneous injection.

Solubility versus pH curves were measured as described: A formulation or a peptide solution in water was prepared and aliquots were adjusted to pH values in the desired range by adding HCl and NaOH. These samples were left equilibrating at room temperature for 2-4 days. Then the samples were centrifuged. A small aliquot of each sample was withdrawn for reverse HPLC analysis for determination of the concentration of the proteins in solution. The pH of each sample was measured after the centrifugation, and the concentration of each protein was depicted versus the measured pH.

1. A glucagon peptide comprising:
a polypeptide consisting of an amino acid sequence set forth as His-Ser-Gln-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr (SEQ ID NO 1) modified by one to seven amino acid substitutions, wherein the substitution or substitutions are at an amino acid position or positions selected from the group consisting of X₂, X₄, X₉, X₁₀, X₁₂, X₁₆, X₁₇, X₁₈, X₂₀, X₂₁, X₂₅, X₂₇, X₂₈, and X₂₉; and
a substituent comprising three or more negatively charged moieties, wherein said substituent is attached to the polypeptide at the epsilon position of a Lys residue, at the delta position of an Orn residue, or at the sulphur of a Cys residue, and wherein said substituent is attached at an amino acid position selected from the group consisting of X₁₀, X₁₂, X₁₆, X₁₇, X₁₈, X₂₀, X₂₁, X₂₅, X₂₇, X₂₈, and X₂₉, and wherein said substituent comprises formula II:
Z₁—Z₂—Z₃—Z₄ [II];
wherein Z₁ represents a structure according to one of the formulas IIa, IIb or IIc: 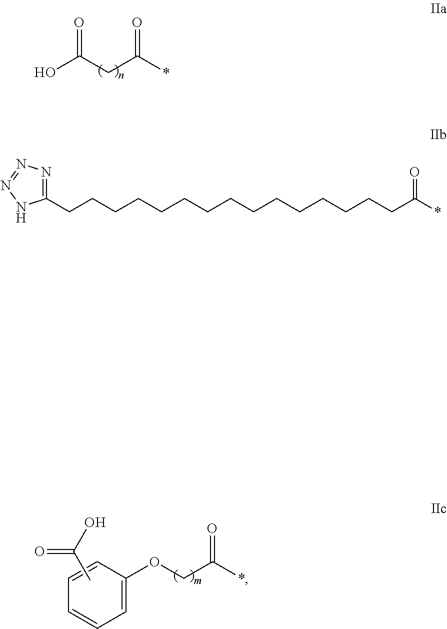
wherein n in formula IIa is 6-20,
m in formula IIc is 5-11, and
the COOH group in formula IIc can be attached to position 2, 3 or 4 on the phenyl ring;
wherein Z₂ is absent or represents a structure according to one of the formulas IId, IIe, IIf, IIg, IIh, IIi, IIj or IIk: 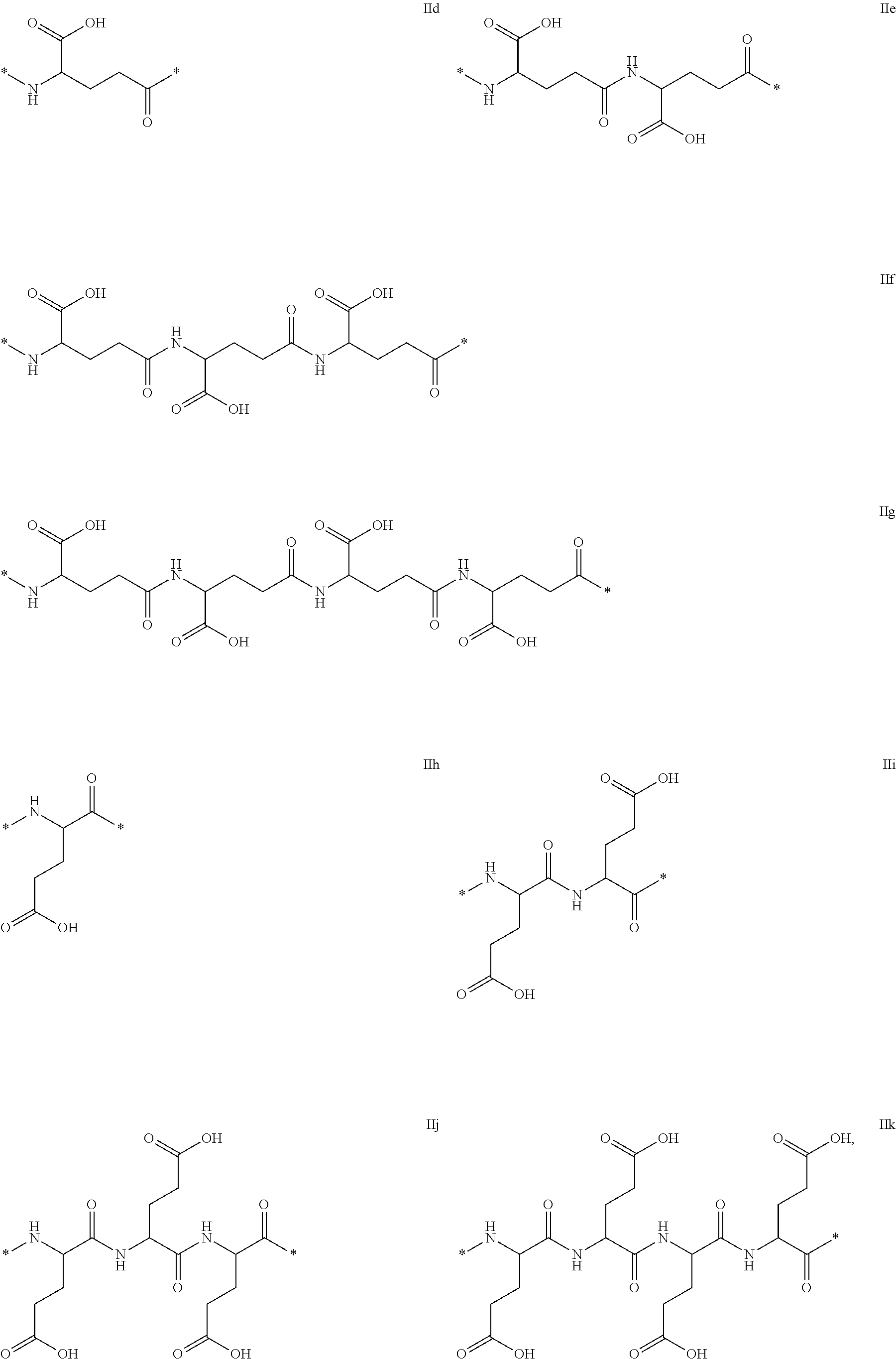
wherein each amino acid moiety independently has the stereochemistry L or D;
wherein Z₃ is absent or represents a structure according to one of the formulas IIm, IIn, IIo or IIp: 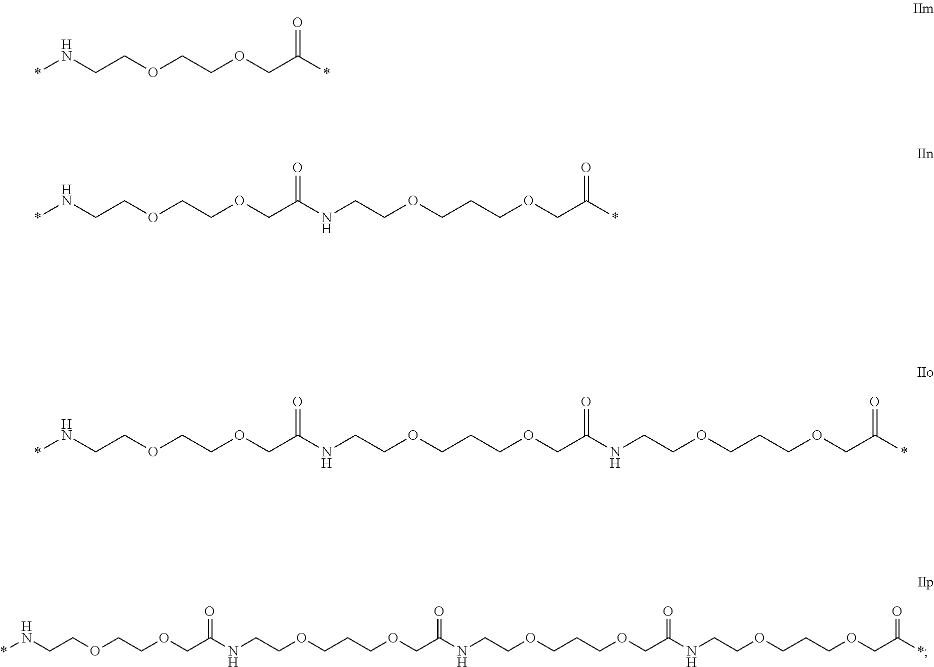
wherein Z₄ is absent or represents a structure according to one of the formulas IId, IIe, IIf, IIg, IIh, IIi, IIj or IIk, wherein each amino acid moiety is independently either L or D;
wherein the symbol * in formula IIa, IIb and IIc of Z₁ represents the attachment point to the nitrogen in Z₂ at symbol *, the nitrogen on Z₃ at symbol *, or the nitrogen on Z₄ at symbol *;
wherein the carbon atom denoted * on Z₂ represents the attachment point to the nitrogen on Z₃ at symbol *, the nitrogen on Z₄ at symbol *, the epsilon nitrogen of a Lys residue on the polypeptide, the delta nitrogen of an Orn residue on the polypeptide, or the sulphur of a Cys residue on the polypeptide;
wherein the carbon atom denoted * on Z₃ represents the attachment point to the nitrogen of Z₄ at symbol *, the epsilon nitrogen of a Lys residue on the polypeptide, the delta nitrogen of an Orn residue on the polypeptide, or the sulphur of a Cys residue on the polypeptide;
wherein the carbon atom denoted * on Z₄ represents the attachment point to the epsilon nitrogen of a Lys residue on the polypeptide, the delta nitrogen of an Orn residue on the polypeptide, or the sulphur of a Cys residue on the polypeptide; and
wherein the glucagon peptide is a glucagon agonist;
or a pharmaceutically acceptable salt, amide, or acid thereof.

2. The glucagon peptide according to claim 1, wherein said substituent comprises a structure having a formula selected from the group consisting of IIIa, IIIb, IIIc, IIId, IIIe, IIIf, IIIg, IIIh, IIIi, IIIj, IIIk, IIIl, IIIm, IIIn, and IIIo:

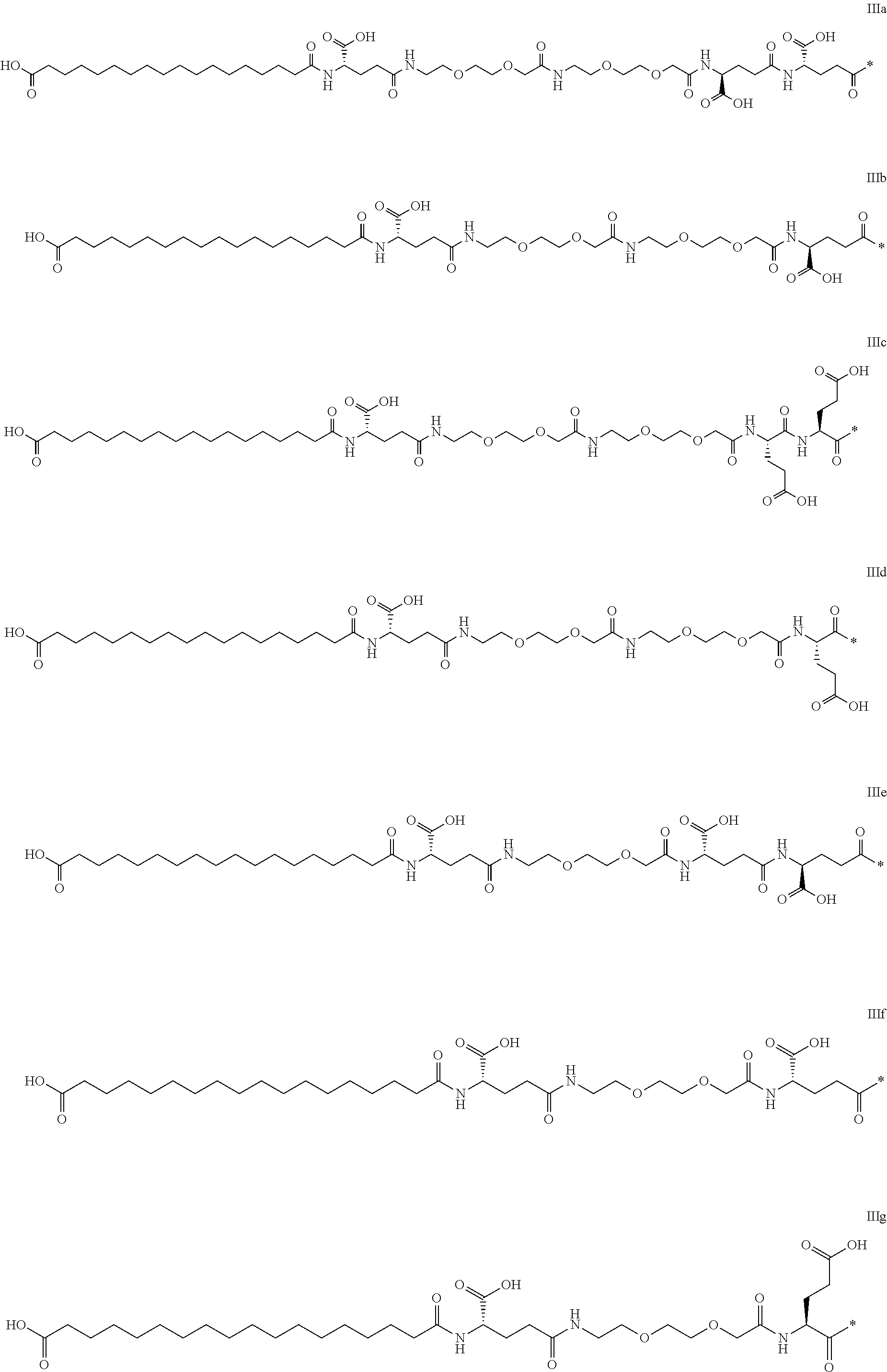

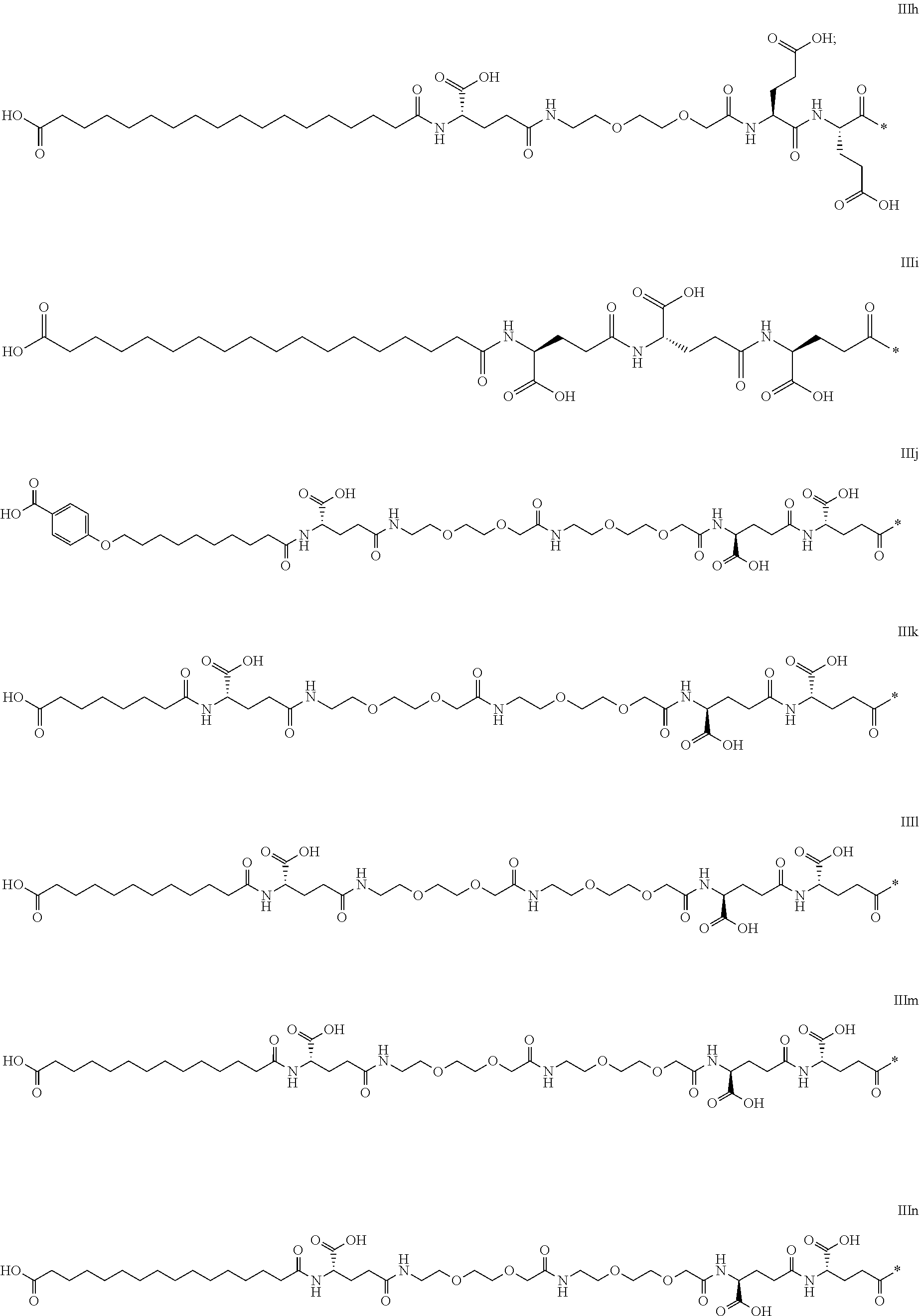

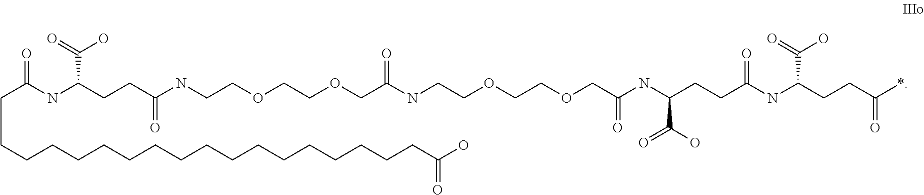

3. The glucagon peptide according to claim 1, wherein said substituent is attached to the polypeptide at an amino acid position selected from the group consisting of X₁₂, X₁₆, X₂₀, X₂₅, X₂₈, and X₂₉.

4. The glucagon peptide according to claim 1, wherein said substituent is attached to the polypeptide at an amino acid position selected from the group consisting of X₁₆ and X₂₈.

5. The glucagon peptide according to claim 1, wherein said substituent is attached to the polypeptide at amino acid position X₂₈ of said glucagon peptide.

6. The glucagon peptide according to claim 1, wherein the glucagon peptide is selected from the group consisting of:
N^{ε28}-([(4S)-5-hydroxy-4-[[(4S)-5-hydroxy-4-[[2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl])-[Leu²⁷,Lys²⁸]-Glucagon 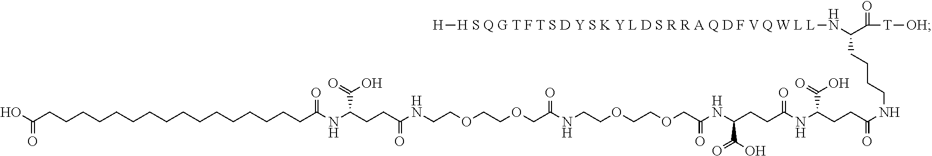
N^{ε29}-([(4S)-5-hydroxy-4-[[(4S)-5-hydroxy-4-[[2-[2-[2-[[2-[2-[2-[[(4S)-5-hydroxy-4-[(18- hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanyl])-[Leu²⁷, Lys²⁹]-Glucagon 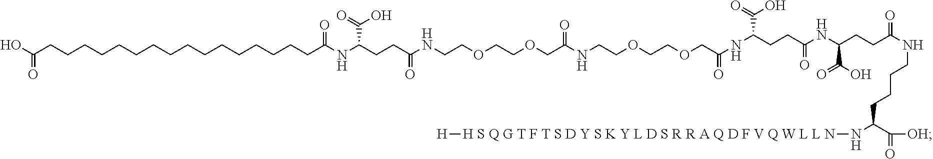
N^{α}-([Leu²⁷]Glucagonyl) N^{ε}-([(4S)-5-hydroxy-4-[[(4S)-5-hydroxy-4-[[2-[2-[2-[[2-[2-[2-[[(4S)-5-hydroxy-4-[(18-hydroxy-18-oxooctadecanoyl)amino]-5-oxopentanoyl]amino]ethoxy]ethoxy]acetyl]amino]ethoxy]ethoxy]acetyl]amino]-5-oxopentanoyl]amino]-5-oxopentanoyl]) Lysine 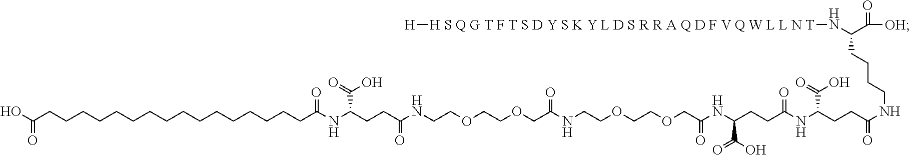
N^{ε28}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Leu²⁷,Lys²⁸]-Glucagon 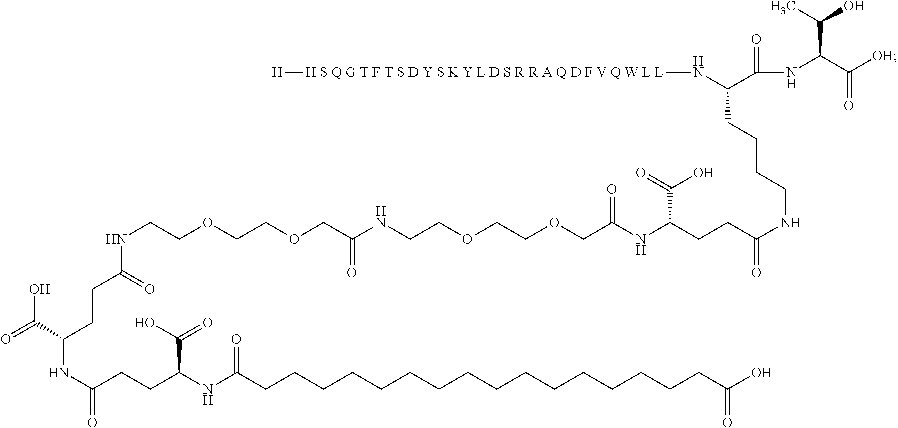
N^{ε28}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Leu²⁷,Lys²⁸]-Glucagon 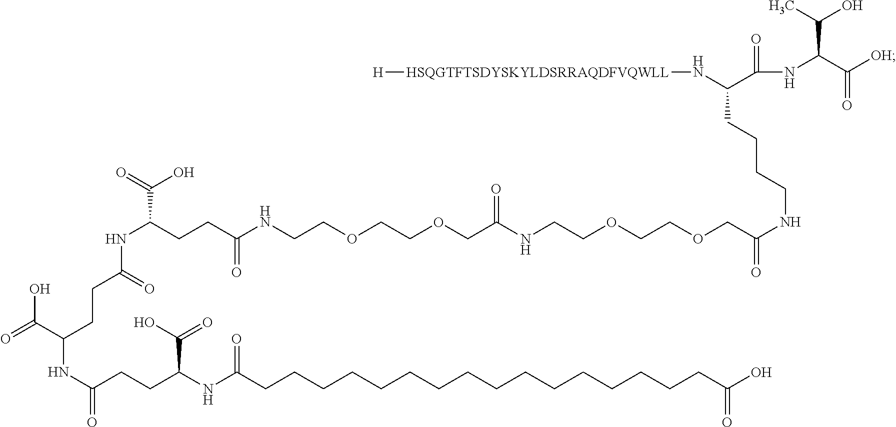
N^{ε16}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Lys¹⁶,Leu²⁷]-Glucagon 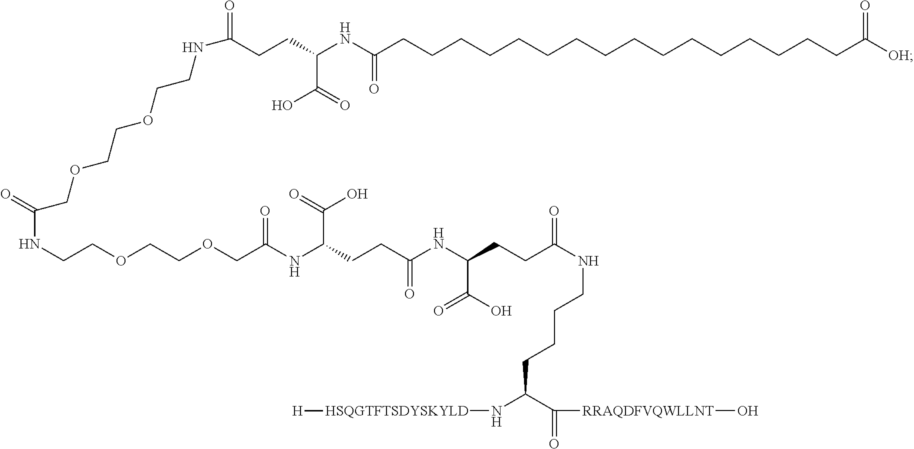
N^{ε25}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Lys²⁵,Leu²⁷]-Glucagon 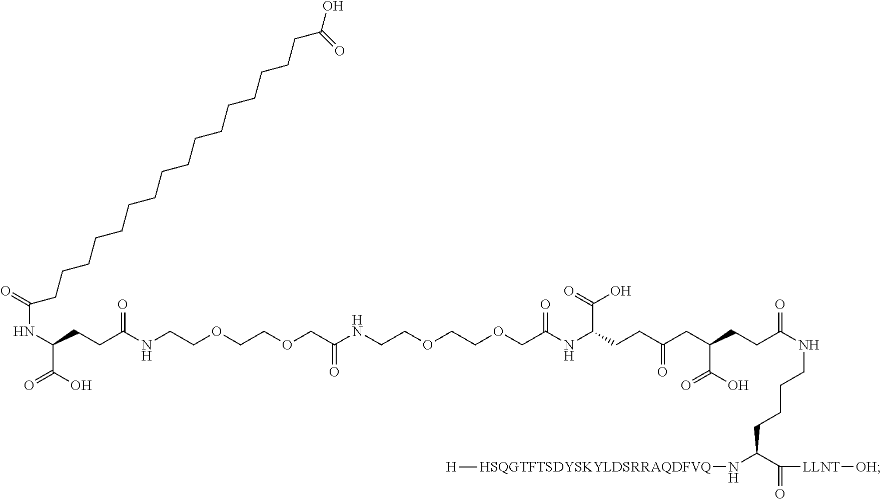
N^{ε16}-[2-[2-[2-[[2-[2-[2-[[(4S)-4-carboxy-4-[[(4S)-4-carboxy-4-[[(4S)-4-carboxy-4-(17-carboxyheptadecanoylamino)butanoyl]amino]butanoyl]amino]butanoyl]amino]ethoxy]ethoxy]acetyl]amino]ethoxy]ethoxy]acetyl]-[Lys¹⁶,Leu²⁷]-Glucagon 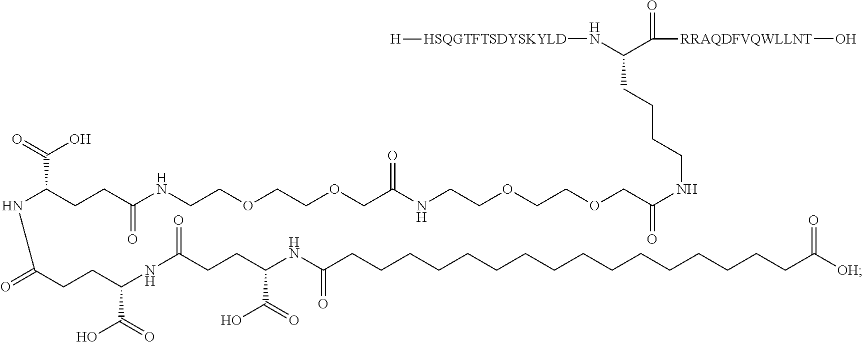
N^{ε16}-[(4S)-4-carboxy-4-[[2-[2-[2-[[2-[2-[2-[[(4S)-4-carboxy-4-[[(4S)-4-carboxy-4-(17-carboxyheptadecanoylamino)butanoyl]amino]butanoyl]amino]ethoxy]ethoxy]acetyl]amino]ethoxy]ethoxy]acetyl]amino]butanoyl]-[Lys¹⁶,Leu²⁷]-Glucagon 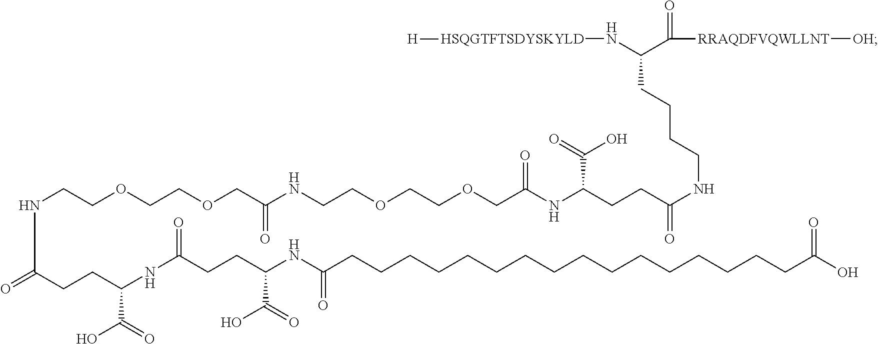
N^{ε28}-[(4S)-4-carboxy-4-[[(4S)-4-carboxy-4-[[(4S)-4-carboxy-4-(17-carboxyheptadecanoylamino)butanoyl]amino]butanoyl]amino]butanoyl]-[Leu²⁷,Lys²⁸]-Glucagon 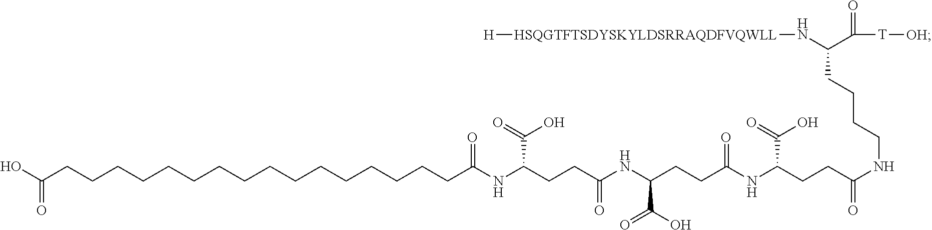
N^{ε12}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Leu²⁷,Pro²⁹]-Glucagon 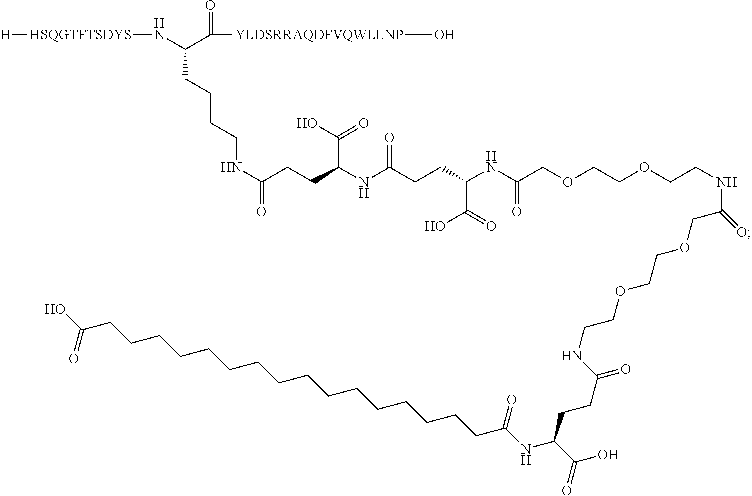
N^{ε28}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Leu²⁷,Lys²⁸]-Glucagonyl-Pro 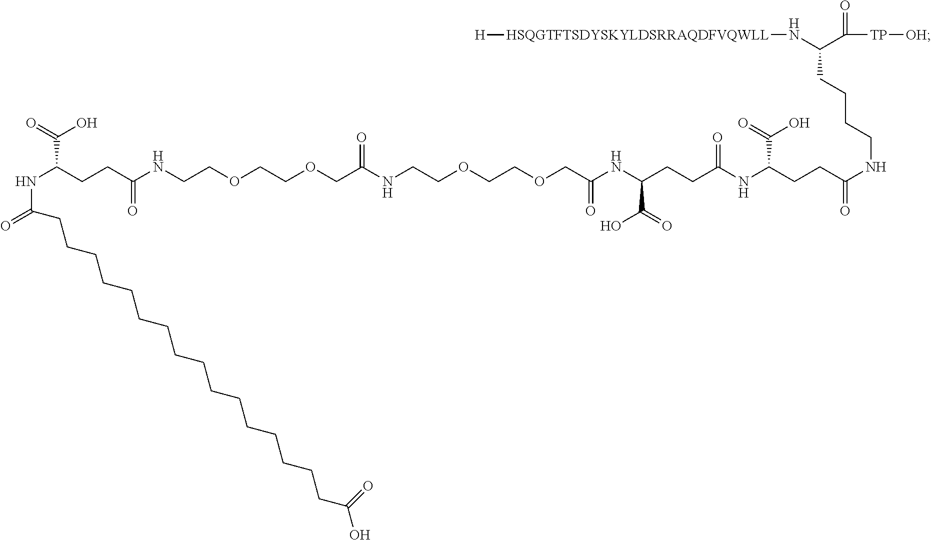
N^{ε12}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Leu²⁷]-Glucagon 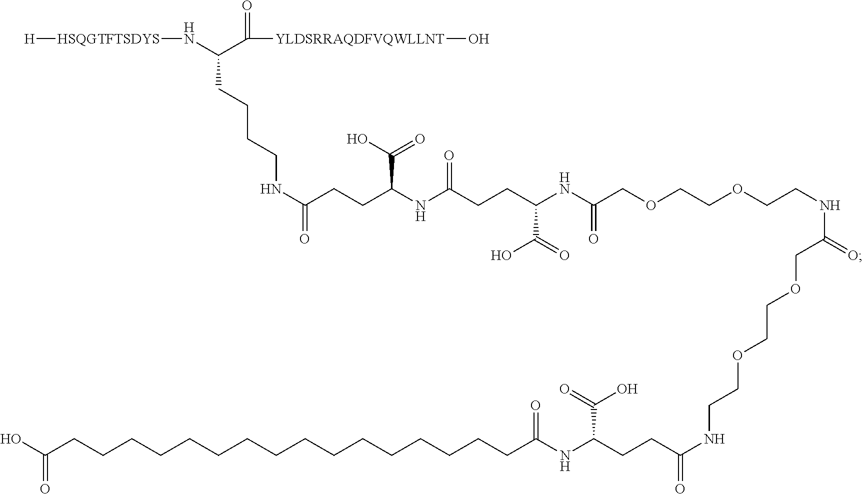
N^{ε27}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Lys²⁷,Pro²⁹]-Glucagon 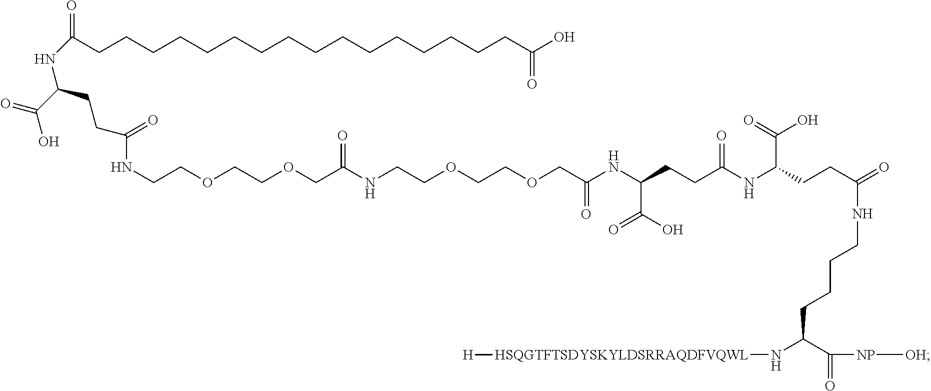
N^{ε28}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Leu²⁷,Lys²⁸,Pro²⁹]-Glucagon 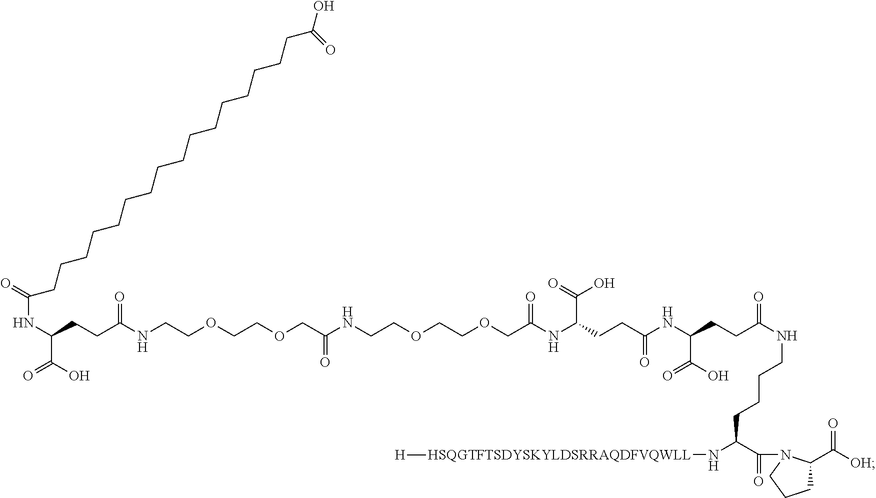
N^{ε27}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Arg¹²,Lys²⁷,Pro²⁹]-Glucagon 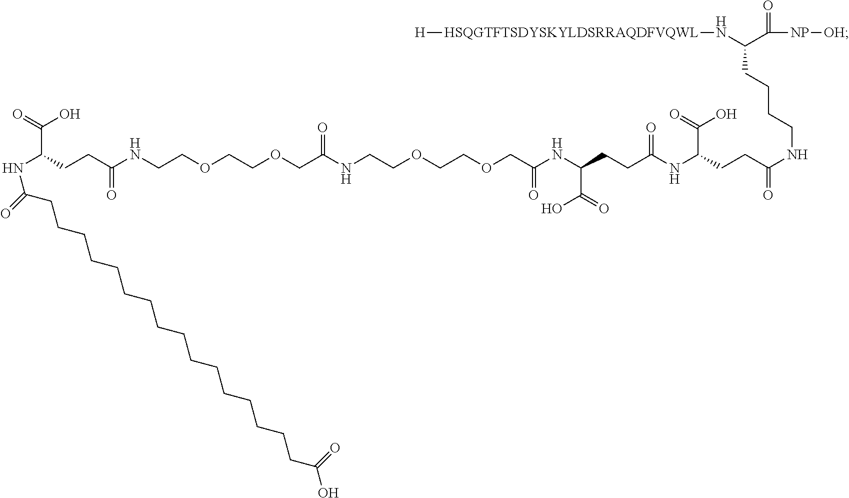
N^{ε20}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Lys²⁰,Leu²⁷]-Glucagon 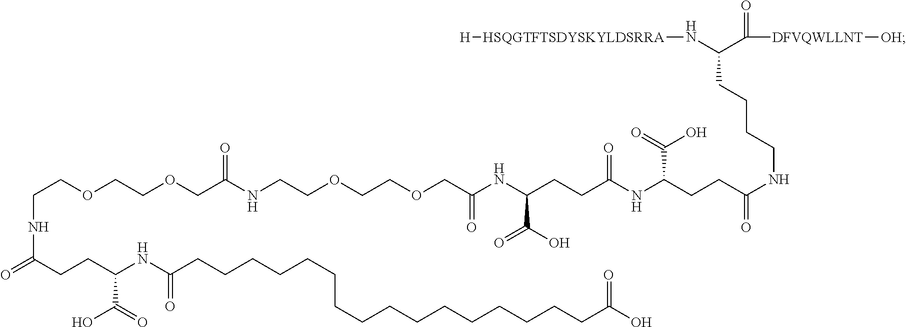
N^{ε16}-[(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]-[Lys¹⁶,Glu²¹,Arg²⁵,Leu²⁷]-Glucagon 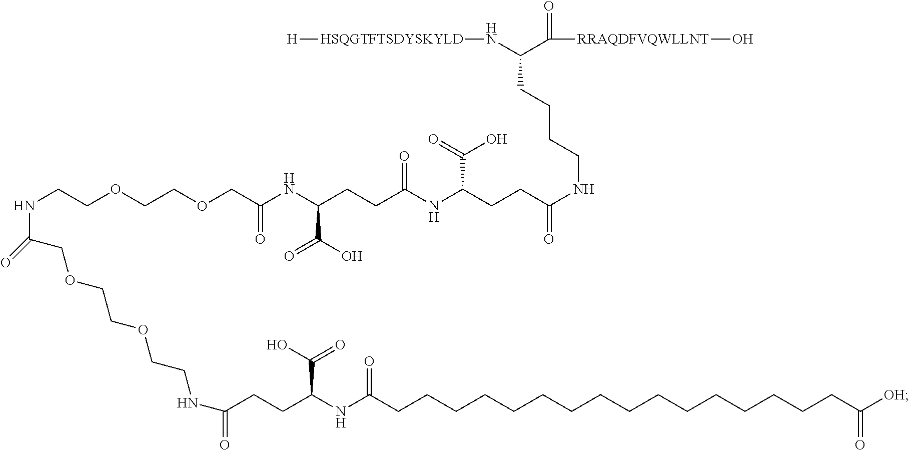
and
N^{α}[His²⁴,Leu²⁷]-Glucagonyl)-N^{ε}(4S)-4-carboxy-4-[[(4S)-4-carboxy-4-[[2-[2-[2-[[2-[2-[2-[[(4S)-4-carboxy-4-(17-carboxyheptadecanoylamino)butanoyl]amino]ethoxy]ethoxy]acetyl]amino]ethoxy]ethoxy]acetyl]amino] butanoyl]amino]butanoyl]Lys

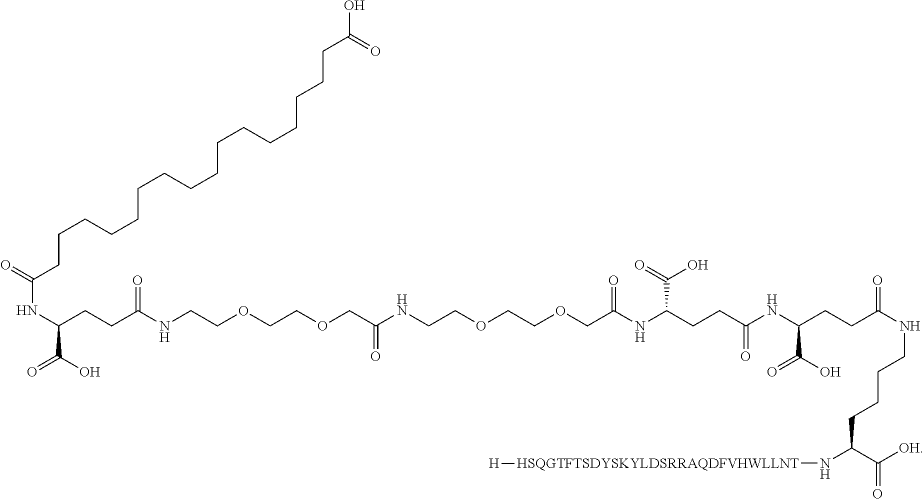

7. A pharmaceutical composition comprising a glucagon peptide according to claim 1.

8. The pharmaceutical composition according to claim 7, further comprising one or more additional therapeutically active compounds or substances.

9. The pharmaceutical composition according to claim 7, further comprising a GLP-1 compound.

10. The pharmaceutical composition according to claim 7, further comprising an insulin compound.

11. The pharmaceutical composition according to claim 7, wherein the composition is suitable for parenteral administration.

12. The pharmaceutical composition according to claim 9, further comprising an insulin compound.

13. A method for treating a disease selected from the group consisting of hyperglycemia, type 2 diabetes, impaired glucose tolerance, type 1 diabetes and obesity, comprising administering to a patient in need thereof, an effective amount of a glucagon peptide according to claim 1.

14. A method for treating obesity, comprising administering to a patient in need thereof, an effective amount of a glucagon peptide according to claim 1.

15. A method for decreasing food intake, comprising administering to a patient in need thereof, an effective amount of a glucagon peptide according to claim 1.

16. A method for reducing body weight, comprising administering to a patient in need thereof, an effective amount of a glucagon peptide according to claim 1.


SEQUENCE LISTING (1 sequence)

SEQ ID NO: 1 - protein, 29 aa, Homo sapiens
HSQGTFTSDYSKYLDSRRAQDFVQWLMNT